(19) United States
(12) Patent Application Publication
(10) Pub. No.: US 2021/0238139 A1
(43) Pub. Date: Aug. 5, 2021

(54) MODULATORS OF TRYPTOPHAN CATABOLISM

(71) Applicant: E-Therapeutics PLC, Long Hanborough (GB)

(72) Inventors: Tanja Poljak, Zagreb (HR); Marina Modric, Zagreb (HR); Srinivasamurthy Vadlamudi, Long Hanborough Oxfordshire (GB); Colin Stubberfield, Long Hanborough Oxfordshire (GB)

(73) Assignee: E-Therapeutics PLC, Long Hanborough (GB)

(21) Appl. No.: 17/054,885

(22) PCT Filed: May 31, 2019
(86) PCT No.: PCT/GB2019/051505

§ 371 Date: Nov. 12, 2020

(30) Foreign Application Priority Data
Jun. 1, 2018 (GB) 1809050.6

(51) Int. Cl. C07D 213/56; C07D 333/24; C07D 409/12; C07D 405/12; C07D 307/54; C07D 277/56; C07C 275/30; C07D 231/16; C07C 275/32; C07D 277/34; A61K 45/06

(52) U.S. Cl. CPC C07D 213/56; C07D 333/24; C07D 409/12; C07D 405/12; C07D 307/54; A61K 45/06; C07C 275/30; C07D 231/16; C07C 275/32; C07D 277/34; C07D 277/56

(57) ABSTRACT

There are described compounds of formula (I): (I) and their use as a medicament in the treatment of diseases associated with the abnormal or elevated catabolism of tryptophan, such as, cancer, immunosuppression, viral infection, depression, a neurodegenerative disorder, trauma, age-related cataracts, organ transplant rejection, or an autoimmune disorder in a patient.

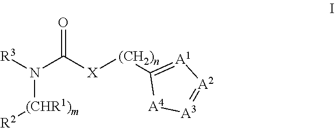

### FIELD OF THE INVENTION

The present invention relates to compounds that are modulators of tryptophan (Trp) catabolism, and their use in the treatment of diseases and/or conditions associated with the abnormal or elevated catabolism of tryptophan.

In particular, compounds of the invention are modulators of tryptophan catabolism. The present invention also relates to methods for the preparation of the compounds of the invention, to intermediates for their preparation, to pharmaceutical compositions comprising a compound of the invention, to the use of a compound of the invention as therapeutic agents, and to methods for the treatment of diseases and/or conditions associated with the elevated catabolism of tryptophan by administering a compound of the invention.

### BACKGROUND OF THE INVENTION

The immune system can recognise cancerous cells and to stop or control their development by a long-term process known as immunosurveillance. However, during the progression to malignancy, cancer cells acquire key capabilities to aid their survival. These capabilities are referred to as the hallmarks of cancer; one of these is the ability of malignant cells to avoid destruction by the immune system (Hanahan & Weinberg, 2011). This means that in many cancers, malignant progression is accompanied by profound immunosuppression that interferes with an effective anti-tumour response and tumour elimination. The principle of immuno-oncology and immuno-therapeutics is to stimulate the patient's own immune system to generate or augment an anti-tumour immune response in order to counteract this immunosuppression and ultimately control or eradicate the cancerous cells.

Tryptophan (Trp) is an essential amino acid that must be obtained through the diet as it cannot be synthesised within the body. It is required for the biosynthesis of proteins, niacin and the neurotransmitter 5-hydroxytryptamine (serotonin). This essential nature of Trp means that any disruption to its metabolism will have a profound effect on the physiological role that it fulfils. The kynurenine pathway is responsible for the metabolism of approximately 95% of all mammalian dietary tryptophan (Adams et al., 2012).

The first and rate-limiting step in this pathway is the conversion of Trp to N-formyl kynurenine. This reaction is performed by the haem-containing enzymes indoleamine 2, 3-dioxygenase (IDO) and tryptophan 2, 3-dioxygenase (TDO) (Adams et al., 2012).

IDO has a ubiquitous pattern of expression and is also able to metabolise various Trp derivatives (Ball et al., 2014). Conversely, TDO is located primarily in the liver and is highly specific for the substrate tryptophan (Ball et al., 2014). There are two paralogs of IDO (IDO1 and ID02) which share significant identity at the amino acid level (43% for human and mouse proteins), but are structurally unrelated to the TDO protein (Ball et al., 2009).

In healthy humans, the activity of IDO and TDO remains low, exerting few physiological effects. However, under pathological conditions including allergic inflammation and infection, IDO and TDO become overexpressed. Overexpression of IDO occurs in response to inflammatory cytokines, the most potent inducer being interferon-γ (IFN-γ) which switches on gene expression and activity (Werner-Felmayer et al., 1990), whilst TDO becomes overexpressed in response to tryptophan and metabolic steroids (Sainio, 1997). It is speculated the overexpression of these key Trp metabolising enzymes serves to deplete the local supply of tryptophan to pathogens, arresting the growth of Trp-dependent intracellular pathogens such as Toxoplasma gondii and Chlamydia trachomatis.

IDO is believed to play a role in the immunosuppressive processes that prevent foetal rejection in utero. During pregnancy, the genetically disparate mammalian conceptus survives despite what would be predicted by tissue transplantation immunology. IDO expression at the maternal-foetal interface increases tryptophan catabolism, the mammalian conceptus appears to suppresses T-cell activity and defends itself against rejection.

Upregulated Trp metabolism has also been identified as a key mechanism used by cancer cells to avoid immune recognition. Many cancer cells are found to overexpress IDO and TDO. Ultimately, this overexpression leads to increased Trp metabolism and depletion in the tumour microenvironment which acts to maintain the immunosuppressive capabilities of the tumour environment by two distinct methods; Firstly, the decrease in available Trp directly inhibits activation and proliferation of effector T cells (Munn et al., 2005). T cells are extremely sensitive to tryptophan shortage and stop proliferating under such conditions. T cell cycle arrest is initiated when uncharged tRNAs detect low Trp concentrations (below 0.5-1 μM) and activate the stress kinase General Control Non-Derepressible 2 (GCN2). This initiates an amino acid starvation response which blocks the cell cycle in the G1 phase resulting in cell cycle arrest and cell death (Munn et al., 1999). Secondly, the metabolism of Trp also indirectly impacts on T cells by causing the accumulation of Trp metabolites such as 3-hydroxyanthranilic acid and quinolinic acid which act to promote the differentiation of regulatory T cells. Regulatory T cells function to supress effector T cell induction and proliferation, thereby further impacting the ability of the immune system to mount a response against the tumour (Munn et al., 1999; Fallarino et al., 2006; Mezrich et al., 2010). There is also evidence that these metabolites can directly induce T cell apoptosis (Terness et al., 2002; Fallarino et al., 2002). Combined, these mechanisms mean that T Cells are unable to launch an effective immune response in the tumour microenvironment, thus favouring tumour progression.

Moreover, Trp depletion is involved in induction of immune tolerance more generally. Accelerated Trp catabolism has been observed in diseases and disorders associated with cellular immune activation, such as infection, autoimmune diseases and AIDS, as well as during malignancy. For example, increased levels of IFNs and elevated levels of urinary Trp metabolites have been observed in autoimmune diseases; it has been postulated that systemic or local depletion of Trp occurring in autoimmune diseases may relate to the degeneration and wasting symptoms of these diseases. In support of this hypothesis, high levels of IDO were observed in cells isolated from the synovia of arthritic joints. IFNs are also elevated in human immunodeficiency virus (HIV) patients and increasing IFN levels are associated with a worsening prognosis. Thus, it was proposed that IDO is induced chronically by HIV infection, and is further increased by opportunistic infections, and that the chronic loss of Trp initiates mechanisms responsible for cachexia, dementia and diarrhoea and possibly immunosuppression of AIDS patients (Brown, et al., 1991, Adv. Exp. Med. Biol., 294: 425-35).

Further evidence for a tumoural immune resistance mechanism based on tryptophan depletion comes from the observation that most human tumours constitutively express IDO, and that expression of IDO by immunogenic mouse tumour cells prevents their rejection by preimmunized mice. This effect is accompanied by a lack of accumulation of specific T cells at the tumour site and can be partly reverted by systemic treatment of mice with an inhibitor of IDO, in the absence of noticeable toxicity. It has also been shown that the IDO inhibitor, 1-methyl-tryptophan (1-MT), can synergize with chemotherapeutic agents to reduce tumour growth in mice, (Muller et al., 2005, Nature Med., 11: 312-9), suggesting that a reduction in Trp catabolism may also enhance the anti-tumour activity of other cancer therapies.

IDO degrades the indole moiety of tryptophan, serotonin and melatonin, and initiates the production of neuroactive and immunoregulatory metabolites, collectively known as kynurenines. Tryptophan metabolism and kynurenine production might represent a crucial interface between the immune and nervous systems (Grohmann, et al., 2003, Trends Immunol., 24: 242-8). In states of persistent immune activation, availability of free serum Trp is diminished and, as a consequence of reduced serotonin production, serotonergic functions may also be affected (Wirleitner, et al., 2003, Curr. Med. Chem., 10: 1581-91). Tryptophan depletion has been associated with mood and psychiatric disorders such as schizophrenia, depression, panic disorder, seasonal affective disorder.

Tryptophan metabolites such as kynurenine, produced by IDO1, inhibit immunosurveillance in cancer by arresting T cells in the G1 phase of the cell cycle, promoting T-cell and dendritic cell apoptosis, and supporting regulatory T-cell generation. In addition, tryptophan metabolites have been found to negatively affect natural killer cell function.

Activation of the kynurenine pathways and production of neuroactive metabolites of tryptophan has been shown to be involved in Huntingdon's disease, Parkinson's disease, Alzheimer's disease, amyotrophic lateral sclerosis, multiple sclerosis, AIDS dementia complex, stroke and epilepsy.

Tryptophan is the precursor of serotonin (5-HT), thus increased tryptophan catabolism may play a role in the neuropsychiatric side effects caused by reducing central 5-HT synthesis, such as depressive symptoms and changes in cognitive function. Furthermore, kynurenine metabolites such as 3-hydroxy-kynurenine (3-OH-KYN) and quinolinic acid (QUIN) have toxic effects on brain function. 3-OH-KYN is able to produce oxidative stress by increasing the production of reactive oxygen species (ROS), and QUIN may produce overstimulation of hippocampal N-methyl-D-aspartate (NMDA) receptors, which leads to apoptosis and hippocampal atrophy. Both ROS overproduction and hippocampal atrophy caused by NMDA overstimulation have been associated with depression (Wichers and Maes, 2004, J. Psychiatry Neurosci., 29: 11-17). Thus, increased tryptophan catabolism activity may play a role in depression.

To date, the majority of research has focussed on direct inhibition of IDO as a means to reducing tryptophan catabolism, increasing Trp and decreasing kynurenine. For example, oxadiazole and other heterocyclic IDO inhibitors are reported in US 2006/0258719 and US 2007/0185165. Methods of measuring tryptophan levels and tryptophan degradation are routine in the art (Huang et al, 2013).

Further research has focussed on direct inhibition of TDO as a means of reducing Trp catabolism, such as reported in PCT/EP2014/076311.

Considering the experimental data indicating a role for Trp catabolism in immunosuppression, tumour resistance and/or rejection, chronic infections, HIV-infection, AIDS, autoimmune diseases or disorders, and immunologic tolerance and prevention of foetal rejection in utero, therapeutic agents aimed at suppression of tryptophan degradation are desirable. Suppression of tryptophan catabolism can be used to activate T cells and therefore enhance T cell activation when the T cells are suppressed by pregnancy, malignancy or a virus such as HIV. Suppression of tryptophan catabolism may also be an important treatment strategy for patients with neurological or neuropsychiatric diseases or disorders such as depression.

It has now been identified that some IDO inhibitors increase expression of IDO protein and such increased expression can continue for periods of time after the removal of the compound. Such activity would be considered unfavourable by a person skilled in the art as elevated expression would classically result in a greater level of Trp catabolism. A more favourable solution would be to identify compounds that have the ability to reduce the expression of IDO. More preferably a favourable solution would be to identify compounds that are able to reduce expression of IDO for durations after removal of the compound.

It has now been identified that the compounds of the invention are capable of reducing tryptophan catabolism, increasing Trp and decreasing kynurenine. More particularly, it has been identified that a compound of the invention is useful in the treatment of conditions associated with the abnormal or elevated catabolism of tryptophan.

Checkpoint Inhibitor—Combinations Background Under normal physiological conditions, immune checkpoints are crucial for the maintenance of self-tolerance (i.e. prevention of autoimmunity) and also to protect tissues from damage when the immune system is responding to pathogenic infection.

The expression of immune-checkpoint proteins can be dysregulated by tumours as another important immune resistance mechanism.

Direct T-cell recognition of tumour cells requires the presentation of antigenic peptides by MHC (major histocompatibility complex) molecules. These peptides are generated by proteasomal digestion and transported to the endoplasmic reticulum, where they are first loaded onto nascent MIIC molecules, which ultimately transport them to the cell membrane.

CD28 is the master costimulatory receptor expressed on T cells and enhances T-cell activation upon antigen recognition when the antigen presenting cell (APC) expresses its ligands, B7-1 and B7-2. Tumour antigen must be processed and presented by the MIIC complex to activate T cells. CTLA-4 is rapidly expressed on T cells once antigen is recognized, and it binds the same ligands (B7.1/2) as CD28 but at higher affinity, thereby counterbalancing the costimulatory effects of CD28 on T-cell activation. Tumour-specific T-cell activation leads to proliferations and effector function, but also the upregulation of PD-1. After trafficking to the tumour microenvironment, PD-1+ T cells might encounter PD-1 ligands, which can inhibit them from mediating their killing function. Thus, the CTLA-4 and PD-1 pathways provide complementary mechanisms to regulate antitumor effector T cells, and blocking each one may prove to be synergistic.

PD iand CTLA4 checkpoints seem to modulate very distinct components of T-cell immunity. CTLA-4 counterbalances the costimulatory signals delivered by CD28 during T-cell activation-both bind the B7 family ligands, B7.1 and B7.2. PD-1 is also induced upon T-cell activation but seems to predominantly down modulate T-cell responses in tissues. The PD-1 ligands, PD-L1 and PD-L2, are induced by distinct inflammatory cytokines—while PD-L1 expression can be induced on diverse epithelial and hematopoietic cell types, PD-L2 is predominantly expressed on dendritic cells and macrophages.

CTLA4

CTLA4, the first immune-checkpoint receptor to be clinically targeted, is expressed exclusively on T cells where it primarily regulates the amplitude of the early stages of T cell activation. Primarily, CTLA4 counteracts the activity of the T cell co-stimulatory receptor, CD28. CD28 does not affect T cell activation unless the TCR is first engaged by cognate antigen. Once antigen recognition occurs, CD28 signalling strongly amplifies TCR signalling to activate T cells. CD28 and CTLA4 share identical ligands: CD80 (also known as B7.1) and CD86 (also known as B7.2). The specific signalling pathways by which CTLA4 blocks T cell activation are still under investigation, although a number of studies suggest that activation of the protein phosphatases, SHP2 (also known as PTPN11) and PP2A, are important in counteracting kinase signals that are induced by TCR and CD28. However, CTLA4 also confers ‘signalling-independent’ T cell inhibition through the sequestration of CD80 and CD86 from CD28 engagement, as well as active removal of CD80 and CD86 from the antigen-presenting cell (APC) surface.

Even though CTLA4 is expressed by activated CD8+ effector T cells, the major physiological role of CTLA4 seems to be through distinct effects on the two major subsets of CD4+ T cells: downmodulation of helper T cell activity and enhancement of regulatory T (TReg) cell immunosuppressive activity.

PD1

Another immune-checkpoint receptor, PD1, is emerging as a promising target, thus emphasizing the diversity of potential molecularly defined immune manipulations that are capable of inducing anti-tumour immune responses by the patient's own immune system.

In contrast to CTLA4, the major role of PD1 is to limit the activity of T cells in peripheral tissues at the time of an inflammatory response to infection. This translates into a major immune resistance mechanism within the tumour microenvironment. PD1 expression is induced when T cells become activated. When engaged by one of its ligands, PD1 inhibits kinases that are involved in T cell activation through the phosphatase SHP2, although additional signalling pathways are also probably induced. Also, because PD1 engagement inhibits the TCR‘stop signal’, this pathway could modify the duration of T cell-APC or T cell-target cell contact.

Similarly to CTLA4, PD1 is highly expressed on TReg cells, where it may enhance their proliferation in the presence of ligand. Because many tumours are highly infiltrated with TReg cells that probably further suppress effector immune responses, blockade of the PD1 pathway may also enhance anti-tumour immune responses by diminishing the number and/or suppressive activity of intratumoural TReg cells.

The two ligands for PD1 are PD1 ligand 1 (PDL1; also known as B7-H1 and CD274) and PDL2 (also known as B7-DC and CD273). These ligands are induced by distinct inflammatory cytokines—while PD-L1 expression can be induced on diverse epithelial and hematopoietic cell types, PD-L2 is predominantly expressed on dendritic cells and macrophages.

PD1 is more broadly expressed than CTLA4: it is induced on other activated non-T lymphocyte subsets, including B cells and natural killer (NK) cells, which limits their lytic activity. Therefore, although PD1 blockade is typically viewed as enhancing the activity of effector T cells in tissues and in the tumour microenvironment, it also probably enhances NK cell activity in tumours and tissues and may also enhance antibody production either indirectly or through direct effects on PD1+ B cells.

Although the major role of the PD1 pathway is in limiting immune effector responses in tissues (and tumours), it can also shift the balance from T cell activation to tolerance at the early stages of T cell responses to antigens within secondary lymphoid tissues (that is, at a similar stage as CTLA4). Taken together, these findings imply a complex set of mechanisms of action for PD1-pathway blockade.

Other

Various ligand-receptor interactions exist between T cells and antigen-presenting cells (APCs) that regulate the T cell response to antigen (which is mediated by peptide-major histocompatibility complex (MHC) molecule complexes that are recognized by the T cell receptor (TCR)). These responses can occur at the initiation of T cell responses in lymph nodes (where the major APCs are dendritic cells) or in peripheral tissues or tumours (where effector responses are regulated). In general, T cells do not respond to these ligand-receptor interactions unless they first recognize their cognate antigen through the TCR. Many of the ligands bind to multiple receptors, some of which deliver co-stimulatory signals and others deliver inhibitory signals. In general, pairs of co-stimulatory-inhibitory receptors that bind the same ligand or ligands such as CD28 and cytotoxic T-lymphocyte-associated antigen 4 (CTLA4) display distinct kinetics of expression with the co-stimulatory receptor expressed on naive and resting T cells, but the inhibitory receptor is commonly upregulated after T cell activation. One important family of membrane-bound ligands that bind both co-stimulatory and inhibitory receptors is the B7 family. All of the B7 family members and their known ligands belong to the immunoglobulin superfamily. Many of the receptors for more recently identified B7 family members have not yet been identified. Tumour necrosis factor (TNF) family members that bind to cognate TNF receptor family molecules represent a second family of regulatory ligand-receptor pairs. These receptors predominantly deliver co-stimulatory signals when engaged by their cognate ligands. Another major category of signals that regulate the activation of T cells comes from soluble cytokines in the microenvironment. Communication between T cells and APCs is bidirectional. In some cases, this occurs when ligands themselves signal to the APC. In other cases, activated T cells upregulate ligands, such as CD40L, that engage cognate receptors on APCs.

The principle of combining an inhibitor of IDO with an inhibitor of CTLA4, PD1/PDL1, IL7 or CD25 has been documented in PCT/US13/66936.

### SUMMARY OF THE INVENTION

The present invention is based on the identification that a compound of the invention may be useful as a medicament in the treatment of diseases and/or conditions associated with the abnormal or elevated catabolism of tryptophan. In a particular aspect, a compound of the invention is a modulator tryptophan catabolism. More particularly, a compound of the invention is useful in the treatment of conditions associated with the abnormal or elevated catabolism of tryptophan.

The present invention also relates to methods for the preparation of the compounds of the invention, to intermediates for their preparation, to pharmaceutical compositions comprising a compound of the invention, to the use of a compound of the invention as therapeutic agents, and to methods for the treatment of diseases and/or conditions associated with the abnormal or elevated catabolism of tryptophan by administering a compound of the invention.

In a one aspect the invention relates to a compound of Formula (I):

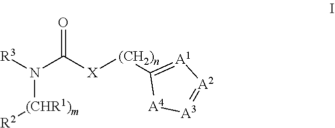

or a pharmaceutically acceptable salt, or a solvate, or a solvate of the salt thereof for use as a medicament, wherein:

m is 0 or 1;

n is 0, 1 or 2;

X is —NR⁸;

R¹ is H, C₁₋₆alkyl or a 6-10 membered aryl;

R² is a 5-6-membered heteroaryl, a fused 9-10 membered bicyclic heteroaryl, a 6-10 membered aryl, a 5-6 membered monocyclic heterocycloalkyl, a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy or phenyl;

R³ is H or C₁₋₆alkyl; or a 3-10 membered cycloalkyl, a 5-11 membered spiroalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆ alkyl, halogen, —CN or —C(═O)OC₁₋₆alkyl;

A¹ is —N— or —CR⁶—;

A² is —N— or —CR⁵—;

A³ is —N— or —CR⁷—;

A⁴ is —N—, —O—, —S—, —CH═N— or —CH═CR⁴—;

R⁴, R⁵, R⁶ and R⁷, which may be the same or different, are each selected from —H, —OH, —C₁₋₆alkyl, halogen, haloC₁₋₆alkyl, —CN, —C₂₋₆alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆ cycloalkyl, —C₂₋₆alkynyl-C₁₋₆alkyl-NR¹¹R¹², —C₂₋₆alkynyl-C₁₋₆alkyl-OR¹³, —C(═O)C₁₋₆ alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;

or R⁵ and R⁷ together form a ring —CH═CH—CH═CH—, —OCH₂O— or —CH₂CH₂CH₂—; or the moiety

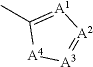

may be fused with oxopyrrolidine;

and

R⁸, R⁹, R¹⁰, R¹¹, R¹², and R¹³, which may be the same or different, are each selected from H or C₁₋₆alkyl.

In one aspect the invention relates to a compound of the invention according to Formula (I):

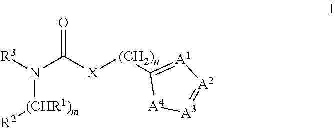

or a pharmaceutically acceptable salt, or a solvate, or a solvate of the salt thereof,

wherein:

m is 0 or 1;

n is 0, 1 or 2;

X is —NR⁸

R¹ is H, C₁₋₆alkyl or a 6-10 membered aryl;

R² is a 5-6-membered heteroaryl, a fused 9-10 membered bicyclic heteroaryl, a 6-10 membered aryl, a 5-6 membered monocyclic heterocycloalkyl, a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy and phenyl;

R³ is H or C₁₋₆alkyl; or a 3-10 membered cycloalkyl, a 5-11 membered spiroalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆ alkyl, halogen, —CN and —C(═O)OC₁₋₆alkyl;

A¹ is —N— or —CR⁶—;

A² is —N— or —CR⁵—;

A³ is —N— or —CR⁷—;

A⁴ is —N—, —O—, —S—, —CH═N— or —CH═CR⁴—;

R⁴, R⁵, R⁶ and R⁷, which may be the same or different, are each selected from —H, —OH, —C₁₋₆alkyl, halogen, haloC₁₋₆alkyl, haloC₁₋₆alkylO—, —CN, —C₁₋₆alkyl-CN, —OC₁₋₆ alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, —C(═O)C₁₋₆alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;

or R⁵ and R⁷ together form a ring —CH═CH—CH═CH—;

or the moiety

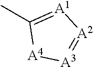

may be fused with oxopyrrolidine; and

R⁸, R⁹, R¹⁰, R¹¹, R¹², and R¹³, which may be the same or different, are each selected from H or C₁₋₆alkyl;

provided that the compound of formula I is not 1-(4-chlorobenzyl)-1-cyclopentyl-3-phenylurea;
N-(3,5-dimethylphenyl)-3-ethyl-2-methyl-7-phenyl-5,7-dihydro-4H-thieno[2,3-c]pyridine-6-carboxamide; [194]
1-cyclopentyl-3-phenyl-1-(2-thienylmethyl)urea; [195]
1-(4-chlorophenyl)-3-phenyl-1-(2-thienylmethyl)urea; [196]
1-[1-(4-fluorophenyl)ethyl]-3-phenyl-urea; [197]
1-(4-chlorophenyl)-3-[1-(5-chloro-2-thienyl)ethyl]urea; [199]
3-(3,4-dichlorophenyl)-1-methyl-1-(2-thienylmethyl)urea; [200]
1-[(5-methyl-2-phenyl-oxazol-4-yl)methyl]-3-phenyl-urea; [203] and
1-(3-chlorophenyl)-3-[(3-chloro-2-thienyl)methyl]urea; [204].

In another aspect of the invention a compound of formula I is not:
1-(4-chlorophenyl)-3-[phenyl(2-thienyl)methyl]urea; [198] and
1-[(4-ethylphenyl)methyl]-3-(2-methoxyphenyl)-1-(3-pyridylmethyl)urea; [205].

In one aspect of the invention m is 1.

In one aspect of the invention n is 0. In another aspect of the invention n is 2.

In one aspect of the invention R¹ is H.

In one aspect of the invention X is —NH—.

In one aspect of the invention R⁸ is H.

In one aspect of the invention R² is a 5-6-membered heteroaryl or a fused 9-10 membered bicyclic heteroaryl. According to this aspect of the invention the 5-6-membered heteroaryl may be selected from the group consisting of furan, isoxazole, oxazole, pyrazine, pyrazole, pyridine, pyrimidine, thiazole and thiophene. The fused 9-10 membered bicyclic heteroaryl may be selected from the group consisting of benzofuran, benzothiadiazole, benzothiazole, benzoxazole, furo-pyridine, imidazo-pyrazine, imidazo-pyridine, imidazo-pyrimidine, imidazo-thiazole, indazole and pyrazolo-pyridine; each of which may independently be optionally substituted by one or more groups independently selected from C₁₋₆alkyl, halogen, haloC₁₋₆alkyl, —OC₁₋₆ alkyl, —CN, —C(═O)OC₁₋₆alkyl, —SO₂—C₁₋₆alkyl, —C(═O)NH₂, haloC₁₋₆alkyloxy and phenyl.

In one aspect of the invention R³ is C₁₋₆alkyl, a 3-10 membered cycloalkyl or a 5-11 membered spiroalkyl. When R³ is C₁₋₆alkyl, a 3-10 membered cycloalkyl or a 5-11 membered spiroalkyl, R³ may be selected from the group consisting of methyl, ethyl, propyl, cyclopropyl, cyclobutyl, cyclopentyl and cyclohexyl. In another one aspect of the invention R³ is a 5-6-membered heteroaryl which may be selected from the group consisting of imidazole, isoxazole, isothiazole, oxadiazole, oxazole, pyrazole, pyridazine, pyridine, pyrimidine, thiazole and triazole.

When R³ is a 3-10 membered cycloalkyl or a 5-11 membered spiroalkyl, the cycloalkyl or spiroalkyl may optionally be substituted by C₁₋₆alkyl, e.g. methyl.

In one aspect of the invention when R⁷ is a 3-10 membered cycloalkyl, then R³ does not represent a 3-5 membered cycloalkyl.

In one aspect of the invention, when R³ is C₁₋₆alkyl or a 3-10 membered cycloalkyl, then R⁷ may be selected from —H, —OH, —C₁₋₆alkyl, halogen, haloC₁₋₆alkyl, —CN, —C₁₋₆ alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, —C(═O)C₁₋₆alkyl, —C(═O)NH₂, a 6-10 membered aryl, a 5-6 membered heteroaryl, each of which may independently be optionally substituted by one or more groups independently selected from —C₁₋₆alkyl, C₁₋₆alkyl-NR⁹R¹⁰, —C₁₋₆ alkyl-OH, —C(═O)OC₁₋₆alkyl or oxopyrrolidine;

In another one aspect of the invention R³ is a 4-6 membered monocyclic heterocycloalkyl which may be selected from the group consisting of oxetane, piperidine and tetrahydropyran; each of which may independently be optionally substituted by one or more groups independently selected from —C₁₋₆alkyl, —OC₁₋₆alkyl, halogen, —CN and —C(═O)OC₁₋₆alkyl.

In one aspect of the invention the moiety

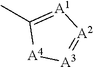

is selected from the group consisting of benzothiazole, indane, oxadiazole, phenyl, pyridine, pyrimidine, thiazole and thiophene; each of which may independently be optionally substituted by one or more groups independently selected from OH, —C₁₋₆alkyl, C₃₋₆cycloalkyl halogen, haloC₁₋₆alkyl, —CN, —C₁₋₆alkyl-CN, —C₁₋₆alkyl-OH, —OC₁₋₆alkyl, —C₂₋₆alkynyl, —C₁₋₆ alkyl-OC₁₋₆alkyl, haloC₁₋₆alkyl-O—, —C₁₋₆alkyl-O—NH₂, C₂₋₆alkynyl-OC₁₋₆alkyl; or a 3-10 membered cycloalkyl, a 6-10 membered aryl, a 5-6 membered heteroaryl, a 4-6 membered monocyclic heterocycloalkyl, a fused 8-10 membered partially unsaturated bicyclic heterocyclyl or a fused 9-10 membered bicyclic heteroaryl, each of which may independently be optionally substituted by one or more groups independently selected from —C₁₋₆alkyl, C₁₋₆alkyl-NR⁹R¹⁰, —C(═O)C₁₋₆alkyl, —C(═O)OC₁₋₆alkyl, —C₁₋₆ alkyl-OH, C₂₋₆alkynyl-C₁₋₆alkyl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, C₂₋₆alkynyl-aryl, C₂₋₆alkynyl-C₁₋₆alkyl-aryl, —C(═O)NH₂ or —C(═O)OC₁₋₆alkyl.

In another aspect of the invention the moiety

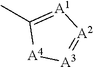

is phenyl, which may independently be optionally substituted by one or more groups independently selected from OH, —C₁₋₆alkyl, C₃₋₆cycloalkyl halogen, haloC₁₋₆alkyl, —CN, —C₁₋₆alkyl-CN, —C₁₋₆alkyl-OH, —OC₁₋₆alkyl, —C₂₋₆alkynyl, —C₁₋₆alkyl-OC₁₋₆alkyl, haloC₁₋₆alkyl-O—, —C₁₋₆ alkyl-O—NH₂, C₂₋₆alkynyl-OC₁₋₆alkyl; a 3-10 membered cycloalkyl, a 6-10 membered aryl, a 5-6 membered heteroaryl, a 4-6 membered monocyclic heterocycloalkyl, a fused 8-10 membered partially unsaturated bicyclic heterocyclyl or a fused 9-10 membered bicyclic heteroaryl, each of which may independently be optionally substituted by one or more groups independently selected from —C₁₋₆alkyl, C₁₋₆alkyl-NR⁹R¹⁰, —C(═O)C₁₋₆alkyl, —C(═O)OC₁₋₆alkyl, —C₁₋₆alkyl-OH, C₂₋₆alkynyl-C₁₋₆alkyl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, C₂₋₆alkynyl-aryl, C₂₋₆alkynyl-C₁₋₆alkyl-aryl, —C(═O)NH₂ and —C(═O)OC₁₋₆alkyl.

In one aspect of the invention A³ is —CR⁷—, wherein R⁷ is selected from the group consisting of the following ring structures:

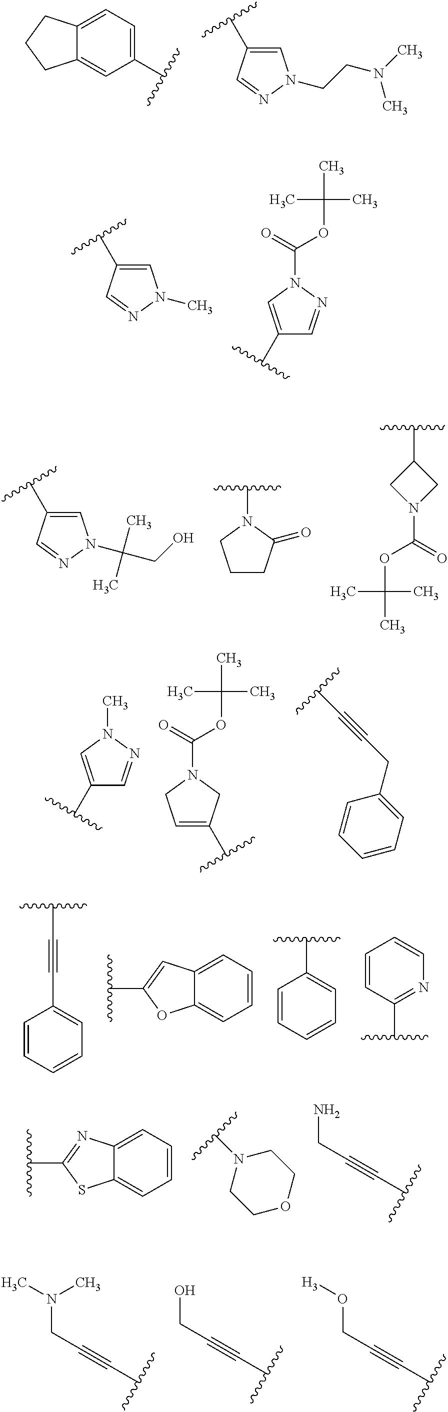

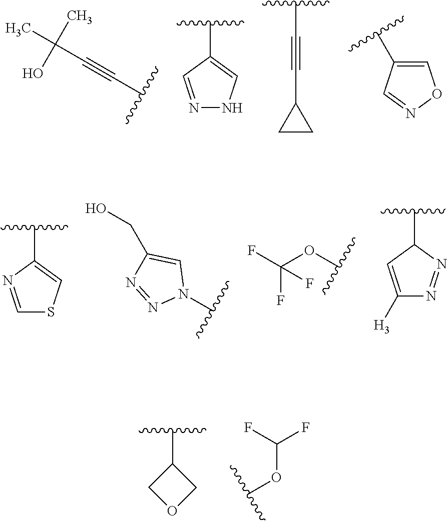

In one aspect of the invention R⁵ is H or halogen.

In one aspect of the invention R⁶ is H or —C₁₋₆alkyl.

The present invention also relates to pharmaceutical compositions comprising a compound of the invention.

In a further aspect, the present invention provides a pharmaceutical composition comprising a compound of the invention and a pharmaceutical carrier, excipient or diluent.

According to the invention there is further provided a pharmaceutical composition comprising a compound of the invention for use in the treatment of conditions involving abnormal or elevated catabolism of tryptophan, whereby the condition involving abnormal or elevated catabolism of tryptophan is one or more of cancer, immune-suppression, viral infection, depression, a neurodegenerative disorder, trauma, age-related cataracts, organ transplant rejection, or an autoimmune disorder in a patient.

According to this aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is cancer, as herein defined.

According to a further aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is viral infection, as herein defined.

According to a further aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is depression, as herein defined.

According to a further aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is a neurodegenerative disorder, as herein defined.

According to a further aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is autoimmune disorder, as herein defined.

According to a further aspect of the invention there is further provided a pharmaceutical composition comprising a compound of the invention for use in the treatment of conditions involving abnormal or elevated catabolism of tryptophan whereby the condition involving abnormal or elevated catabolism of tryptophan is immunosuppression, as herein defined.

According to another aspect of the invention there is further provided a pharmaceutical composition comprising a compound of the invention for use in the treatment of conditions involving reduced levels of tryptophan, whereby the condition involving elevated levels of is one or more of cancer, immune-suppression, viral infection, depression, or a neurodegenerative disorder in a patient.

According to this aspect of the invention there is further provided a pharmaceutical composition comprising a compound of the invention for use in the treatment of conditions involving reduced levels of tryptophan whereby the condition involving reduced levels of tryptophan is cancer, as herein defined.

According to a further aspect of the invention there is further provided a pharmaceutical composition comprising a compound of the invention for use in the treatment of conditions involving reduced levels of tryptophan whereby the condition involving reduced levels of tryptophan is viral infection, as herein defined.

According to a further aspect of the invention there is further provided a pharmaceutical composition comprising a compound of the invention for use in the treatment of conditions involving reduced levels of tryptophan whereby the condition involving reduced levels of tryptophan is depression, as herein defined.

According to a further aspect of the invention there is further provided a pharmaceutical composition comprising a compound of the invention for use in the treatment of conditions involving reduced levels of tryptophan whereby the condition involving reduced levels of tryptophan is a neurodegenerative disorder, as herein defined.

According to another aspect of the invention there is further provided a pharmaceutical composition comprising a compound of the invention for use in the treatment of conditions involving elevated levels of kynurenine, whereby the condition involving elevated levels of is one or more of cancer, immune-suppression, viral infection, depression, or a neurodegenerative disorder in a patient.

According to this aspect of the invention there is further provided a pharmaceutical composition comprising a compound of the invention for use in the treatment of conditions involving elevated levels of kynurenine whereby the condition involving elevated levels of kynurenine is cancer, as herein defined.

According to a further aspect of the invention there is further provided a pharmaceutical composition comprising a compound of the invention for use in the treatment of conditions elevated levels of kynurenine whereby the condition involving elevated levels of kynurenine is viral infection, as herein defined.

According to a further aspect of the invention there is further provided a pharmaceutical composition comprising a compound of the invention for use in the treatment of conditions involving elevated levels of kynurenine whereby the condition involving elevated levels of kynurenine is depression, as herein defined.

According to a further aspect of the invention there is further provided a pharmaceutical composition comprising a compound of the invention for use in the treatment of conditions involving elevated levels of kynurenine whereby the condition involving elevated levels of kynurenine is a neurodegenerative disorder, as herein defined.

In a further aspect, the present invention provides a pharmaceutical composition comprising a compound of the invention for use in inhibiting immunosuppression.

In a further aspect, the present invention provides a pharmaceutical composition comprising a compound of the invention for use in inhibiting immunosuppression such as IDO-mediated immunosuppression.

In a further aspect, the present invention provides a pharmaceutical composition comprising a compound of the invention for use in inhibiting immunosuppression such as TDO-mediated immunosuppression.

In a further aspect, the present invention provides a pharmaceutical composition comprising a compound of the invention for use in inhibiting immunosuppression such as tryptophan catabolism mediated immunosuppression.

In a further aspect, the present invention provides a pharmaceutical composition comprising a compound of the invention for use in inhibiting immunosuppression such as immunosuppression caused by abnormal or elevated tryptophan catabolism.

In a further aspect, the present invention provides a pharmaceutical composition comprising a compound of the invention for use in inhibiting immunosuppression such as immunosuppression caused by reduced levels of tryptophan.

In a further aspect, the present invention provides a pharmaceutical composition comprising a compound of the invention for use in inhibiting immunosuppression such as immunosuppression caused by elevated levels of kynurenine.

In a further aspect, the present invention provides a pharmaceutical composition comprising a compound of the invention for use in increasing the levels of tryptophan and decreasing the levels of kynurenines individually, simultaneously or sequentially.

In a further aspect, the present invention provides a pharmaceutical composition comprising a compound of the invention for use as previously described without inhibition of IDO.

In a further aspect, the present invention provides a pharmaceutical composition comprising a compound of the invention for use as previously described without inhibition of TDO.

In a further aspect, the present invention provides a pharmaceutical composition comprising a compound of the invention for use as previously described without inhibition of IDO or TDO individually, simultaneously or sequentially.

In a further aspect, the present invention provides a pharmaceutical composition comprising a compound of the invention for use as an IDO inhibitor.

In a further aspect, the present invention provides a pharmaceutical composition comprising a compound of the invention for use as a TDO inhibitor.

In a further aspect, the present invention provides a pharmaceutical composition comprising a compound of the invention for use as an IDO inhibitor and TDO inhibitor individually, simultaneously or sequentially.

In a further aspect, the present invention provides a pharmaceutical composition comprising a compound of the invention for inhibiting the degradation of tryptophan.

In a further aspect, the present invention provides a pharmaceutical composition comprising a compound of the invention for altering (e.g., increasing) extracellular tryptophan levels.

In a further aspect, the present invention provides a pharmaceutical composition comprising a compound of the invention for altering (e.g., increasing) intracellular tryptophan levels.

In a further aspect, the present invention provides a pharmaceutical composition comprising a compound of the invention for altering (e.g., decreasing) intracellular kynurenine levels.

In a further aspect, the present invention provides a pharmaceutical composition comprising a compound of the invention for altering (e.g. decreasing) intracellular levels of IDO1 protein in cancer cells.

According to this aspect of the invention, decreases of intracellular levels of IDO1 protein in cancer cells may occur without direct IDO1 inhibition.

According to this aspect of the invention, decreases of intracellular levels of IDO1 protein in cancer cells may occur simultaneously or sequentially with direct IDO1 inhibition.

In a further aspect, the present invention provides a pharmaceutical composition comprising a compound of the invention for altering (e.g. decreasing) intracellular levels of IDO1 protein in cancer cells and decreasing kynurenine production individually simultaneously or sequentially.

In a further aspect, the present invention provides a pharmaceutical composition comprising a compound of the invention for increasing tumour cell killing.

According to this aspect of the invention increased tumour cell killing may be a consequence of activation of immune cells.

In a particular aspect, the pharmaceutical composition may additionally comprise a second therapeutically active ingredient suitable for use in combination with compounds of the invention.

Moreover, the compounds of the invention, useful in the pharmaceutical compositions and treatment methods disclosed herein, are pharmaceutically acceptable as prepared and used.

In another aspect, the invention relates to a compound of the invention for use in therapy.

In a further aspect, the invention relates to a compound of Formula I for use in the manufacture of a medicament for the treatment of diseases and/or conditions associated with the abnormal or elevated catabolism of tryptophan.

In another aspect, the invention relates to the use of a compound of the invention in the manufacture of a medicament for the treatment of diseases and/or conditions associated with the abnormal or elevated catabolism of tryptophan.

According to this aspect of the invention the disease or condition associated with the abnormal or elevated catabolism of tryptophan is one or more of cancer, immunosuppression, viral infection, depression, a neurodegenerative disorder, trauma, age-related cataracts, organ transplant rejection, or an autoimmune disorder in a patient.

According to a further aspect of the invention there is provided a compound of Formula I for use in the treatment of conditions involving abnormal or elevated catabolism of tryptophan whereby the condition involving abnormal or elevated catabolism of tryptophan is viral infection, as herein defined.

According to a further aspect of the invention there is provided a compound of Formula I for use in the treatment of conditions involving abnormal or elevated catabolism of tryptophan whereby the condition involving abnormal or elevated catabolism of tryptophan is depression, as herein defined.

According to a further aspect of the invention there is provided a compound of Formula I for use in the treatment of conditions involving abnormal or elevated catabolism of tryptophan whereby the condition involving abnormal or elevated catabolism of tryptophan is a neurodegenerative disorder, as herein defined.

According to a further aspect of the invention there is provided a compound of Formula I for use in the treatment of conditions involving abnormal or elevated catabolism of tryptophan whereby the condition involving abnormal or elevated catabolism of tryptophan is autoimmune disorder, as herein defined.

According to a further aspect of the invention there is provided a compound of Formula I for use in the treatment of conditions involving reduced levels of tryptophan, whereby the condition involving reduced levels of tryptophan of is one or more of cancer, immune-suppression, viral infection, depression, or a neurodegenerative disorder in a patient.

According to a further aspect of the invention there is provided a compound of Formula I for use in the treatment of conditions involving reduced levels of tryptophan whereby the condition involving reduced levels of tryptophan is cancer, as herein defined.

According to a further aspect of the invention there is provided a compound of Formula I for use in the treatment of conditions involving reduced levels of tryptophan whereby the condition involving reduced levels of tryptophan is viral infection, as herein defined.

According to a further aspect of the invention there is provided a compound of Formula I for use in the treatment of conditions involving reduced levels of tryptophan whereby the condition involving reduced levels of tryptophan is depression, as herein defined.

According to a further aspect of the invention there is provided a compound of Formula I for use in the treatment of conditions involving reduced levels of tryptophan whereby the condition involving reduced levels of tryptophan is a neurodegenerative disorder, as herein defined.

According to a further aspect of the invention there is provided a compound of Formula I for use in the treatment of conditions involving elevated levels of kynurenine, whereby the condition involving elevated levels of is one or more of cancer, immune-suppression, viral infection, depression, or a neurodegenerative disorder in a patient.

According to a further aspect of the invention there is provided a compound of Formula I for use in the treatment of conditions involving elevated levels of kynurenine whereby the condition involving elevated levels of kynurenine is cancer, as herein defined.

According to a further aspect of the invention there is provided a compound of Formula I for use in the treatment of conditions involving elevated levels of kynurenine whereby the condition involving elevated levels of kynurenine is viral infection, as herein defined.

According to a further aspect of the invention there is provided a compound of Formula I for use in the treatment of conditions involving elevated levels of kynurenine whereby the condition involving elevated levels of kynurenine is depression, as herein defined.

According to a further aspect of the invention there is provided a compound of Formula I for use in the treatment of conditions involving elevated levels of kynurenine whereby the condition involving elevated levels of kynurenine is a neurodegenerative disorder, as herein defined.

According to a further aspect of the invention there is provided a compound of Formula I for use in the treatment of immunosuppression.

According to a further aspect of the invention there is provided a compound of Formula I for use in the treatment of immunosuppression whereby the immunosuppression is IDO-mediated immunosuppression.

According to a further aspect of the invention there is provided a compound of Formula I for use in the treatment of immunosuppression whereby the immunosuppression is TDO-mediated immunosuppression.

According to a further aspect of the invention there is provided a compound of Formula I for use in the treatment of immunosuppression whereby the immunosuppression is tryptophan catabolism mediated immunosuppression.

According to a further aspect of the invention there is provided a compound of Formula I for use in the treatment of immunosuppression such as immunosuppression caused by abnormal or elevated tryptophan catabolism.

According to a further aspect of the invention there is provided a compound of Formula I for use in the treatment of immunosuppression such as immunosuppression caused by reduced levels of tryptophan.

According to a further aspect of the invention there is provided a compound of Formula I for use in the treatment of immunosuppression such as immunosuppression caused by elevated levels of kynurenine.

According to a further aspect of the invention there is provided a compound of Formula I for use in increasing the levels of tryptophan and decreasing the levels of kynurenines individually, simultaneously or sequentially.

According to a further aspect of the invention there is provided a compound of Formula I for use in inhibiting the degradation of tryptophan and reducing kynurenine production individually, simultaneously or sequentially.

According to a further aspect of the invention there is provided a compound of Formula I for use as an IDO inhibitor.

According to a further aspect of the invention there is provided a compound of Formula I for use as a TDO inhibitor.

According to a further aspect of the invention there is provided a compound of Formula I for use as an IDO inhibitor and TDO inhibitor individually, simultaneously or sequentially.

According to a further aspect of the invention there is provided a compound of Formula I for use as a regulator of (e.g. decreasing) intracellular levels of IDO1 protein in cancer cells.

According to a further aspect of the invention there is provided a compound of Formula I for use as a regulator of (e.g. decreasing) intracellular levels of IDO1 protein in cancer cells without direct IDO1 inhibition.

According to a further aspect of the invention there is provided a compound of Formula I for use as a regulator of (e.g. decreasing) intracellular levels of IDO1 protein in cancer cells and direct IDO1 inhibition.

According to a further aspect of the invention there is provided a compound of Formula I for use as a regulator of (e.g. decreasing) intracellular levels of IDO1 protein in cancer cells and decreasing kynurenine production individually simultaneously or sequentially.

According to a further aspect of the invention there is provided a compound of Formula I for use in tumour cell killing.

According to this aspect of the invention increased tumour cell killing may be a consequence of activation of immune cells.

In a further aspect, the invention relates to methods of the treatment of diseases and/or conditions associated with the abnormal or elevated catabolism of tryptophan by administering of an effective amount of a compound of the invention or one or more pharmaceutical compositions of the invention.

In another aspect of the invention, this invention provides methods of treatment of a subject, in particular humans, susceptible to or afflicted with diseases and/or conditions associated with the abnormal or elevated catabolism of tryptophan selected from among those listed herein, and particularly proliferative diseases, which methods comprise the administration of an effective amount of a compound of the invention or one or more pharmaceutical compositions of the invention.

In additional aspects, this invention provides methods for synthesizing the compounds of the invention, with representative synthetic protocols and pathways disclosed later on herein.

Other objects and advantages will become apparent to those skilled in the art from a consideration of the ensuing detailed description.

### DETAILED DESCRIPTION OF THE INVENTION

### Definitions

It will be understood that the present invention covers all combinations of aspects, suitable, convenient and preferred groups described herein.

When describing the invention, which may include processes, compounds, pharmaceutical compositions containing such compounds and methods of using such compounds and compositions, the following terms, if present, have the following meanings unless otherwise indicated. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

When ranges are referred to herein, for example but without limitation, C₀₋₆alkyl, the citation of a range should be considered a representation of each member of said range. By way of example C₀alkyl means that alkyl group is absent. Thus, for example, selected member C₀alkyl-aryl of a range C₀₋₆alkyl-aryl means that aryl group is directly attached without an alkyl spacer.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

Examples of aliphatic carbonyls include, but are not limited to, acetyl, propionyl, 2-fluoroacetyl, butyryl, 2-hydroxylacetyl, and the like.

The term “alkyl” as used herein as a group or a part of a group refers to a straight or branched aliphatic hydrocarbon having the specified number of carbon atoms. Particular alkyl groups have 1 to 18 carbon atoms; more particular alkyl groups have 1 to 6 carbon atoms, and even more particular alkyl groups have 1 to 4 carbon atoms. Suitably alkyl groups have 1 or 2 carbon atoms. Branched means that one or more alkyl groups such as methyl, ethyl or propyl is attached to a linear alkyl chain. Exemplary branched chain groups include isopropyl, iso-butyl, t-butyl and isoamyl. Examples of alkyl groups as used herein include methyl, ethyl, n-propyl, isopropyl, n-butyl, tert-butyl, sec-butyl, n-pentyl, n-hexyl, 1,2-dimethylbutyl, octyl, decyl, undecyl, dodecyl tridecyl, tetradecyl, pentadecyl, hexadecyl, heptadecyl and octadecyl.

The term “alkyloxy” or “alkoxy”, as used herein, refers to a straight or branched chain alkyl group, as previously defined, attached to the parent molecular moiety through an oxygen atom containing the specified number of carbon atoms. Particular alkoxy groups have between 1 and 6 carbon atoms. More particular alkoxy groups have between 1 and 4 carbon atoms. For example, C₄alkoxy means a straight or branched alkoxy containing at least 1, and at most 4, carbon atoms. Examples of “alkoxy” as used herein include, but are not limited to, methoxy, ethoxy, n-propoxy, isopropoxy, n-butoxy, tert-butoxy, sec-butoxy, n-pentoxy, n-hexoxy, and 1,2-dimethylbutoxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Particular “alkenyl” groups have 2 to 4 carbon atoms and containing at least one double bond. Examples of “alkenyl” as used herein include ethenyl, 2-propenyl, 3-butenyl, 2-butenyl, 2-pentenyl, 3-pentenyl, 3-methyl-2-butenyl, 3-methyl but-2-enyl, 3-hexenyl and 1,1-dimethylbut-2-enyl.

The term “alkynyl” as used herein as a group or a part of a group refers to a straight or branched hydrocarbon chain containing the specified number of carbon atoms and containing at least one triple bond. For example, the term “C₂₋₆alkynyl” means a straight or branched alkynyl containing at least 2, and at most 6, carbon atoms and containing at least one triple bond. Examples of “alkynyl” as used herein include, but are not limited to, propynyl, 1-butynyl, 2-butynyl, 1-pentynyl and 3-methyl-1-butynyl.

The term “alkylene” as used herein as a group or a part of a group refers to a branched or straight chained alkyl group containing from 1 to 6 carbon atoms, having single bonds for attachment to other groups at two different carbon atoms. Examples of such alkylene groups include methylene, ethylene, n-propylene, isopropylene, n-butylene, isobutylene, pentylene, and hexylene. Particular alkylene groups have between 1 and 4 carbon atoms. More particular it is methylene (—CH₂—) or ethylene (—CH₂—CH₂-).

The term “amino” refers to the radical —NH₂.

The term “carbamoyl” refers to the radical —C(O)NH₂.

The term “comprise”, and variations such as “comprises” and “comprising”, throughout the specification and the claims which follow, unless the context requires otherwise, will be understood to imply the inclusion of a stated integer or step or group of integers but not to the exclusion of any other integer or step or group of integers or steps.

The term “compound(s) of the invention” or “compound(s) according to the invention”, and equivalent expressions refers to compounds of Formula (I) (whether in solvated or unsolvated form), as herein described, including any subset or embodiment of compounds of Formula (I), or their pharmaceutically acceptable salts (whether in solvated or unsolvated form). Suitably, said expression includes the pharmaceutically acceptable salts, and solvates (e.g. hydrates) thereof. The compound(s) of the invention may possess one or more asymmetric centres; such compounds can therefore be produced as individual (R)- or (S)-stereoisomers or as mixtures thereof. Where stereochemistry is not defined in the relevant Formula(e), then the term compound(s) of the invention includes enantiomers and diastereoisomers of these compounds.

The term “cyano” to the radical —CN.

The term “cycloalkyl” as used herein, refers to a monocyclic or polycyclic saturated hydrocarbon ring (including spiro compounds) containing the stated number of carbon atoms, for example, 3 to 10 carbon atoms. Particular “cycloalkyl” groups are monocyclic or four connected cyclohexane ring like in case of adamantane. Examples of “cycloalkyl” groups include cyclopropyl, cyclobutyl, cyclopentyl, cyclohexyl, cycloheptyl, bicyclo[2.2.1]heptyl, cyclooctyl, cyclononyl, cyclodecycl, and adamantly. Particular cycloalkyl groups include cyclopropyl, cyclobutyl, cyclopentyl, cyclohexyl and adamantyl.

The term “halogen” or “halo” or “Hal” refers to fluoro (F), chloro (Cl), bromo (Br) and iodo (I). Particular halo groups are either fluoro or chloro. More particular halo group is chloro.

The term “hetero” when used to describe a compound or a group present on a compound means that one or more carbon atoms in the compound or group have been replaced by a nitrogen, oxygen, or sulfur heteroatom. For example, having from 1 to 4 heteroatoms, particularly from 1 to 3 heteroatoms, and more typically 1 or 2 heteroatoms, for example a single heteroatom.

The term “heteroaryl” or “heteroaromatic” as used herein refers to a 5-6 membered monocyclic aromatic ring or a fused 9-10 membered bicyclic aromatic ring containing up to four heteroatoms independently selected from nitrogen, sulphur and oxygen and the number of ring members specified. Monocyclic heteroaryl ring may contain up to three heteroatoms. Typically, monocyclic heteroaryl will contain up to 3 heteroatoms, usually up to 2, for example a single heteroatom. The bicyclic heteroaryl may contain up to four heteroatoms. Typically, bicyclic heteroaryl will contain up to 4 heteroatoms, more typically up to 3 heteroatoms, more usually up to 2, for example a single heteroatom. In one embodiment, the heteroaryl ring contains at least one or two nitrogen atoms. The nitrogen atoms in the heteroaryl rings can be basic, as in the case of an imidazole or pyridine, or essentially non-basic as in the case of a pyrrole nitrogen. In general, the number of basic nitrogen atoms present in the heteroaryl group, including any amino group substituents of the ring, will be less than five.

Examples of five membered monocyclic heteroaryl groups include but are not limited to pyrrolyl, furanyl, thiophenyl, imidazolyl, furazanyl, oxazolyl, oxadiazolyl, oxatriazolyl, isoxazolyl, thiazolyl, isothiazolyl, pyrazolyl, triazolyl, thiadiazolyl and tetrazolyl groups. Examples of six membered monocyclic heteroaryl groups include but are not limited to pyridinyl, pyrazinyl, pyridazinyl, pyrimidinyl, triazinyl and tetrazinyl. Particular monocyclic heteroaryl groups are those derived from imidazole, pyrazole and pyridine.

Examples of fused heteroaryl rings include pyrrolopyridine, pyrrolopyrimidine, pyrazolopyridine, thienopyridine, furopyridine, azaindole, diazaindole, imidazopyridine, benzothiazole, quinoline, isoquinoline, quinazoline, quinoxaline, pteridine, cinnoline, phthalazine, naphthyridine, indole, isoindole, indazole, purine, benzofurane, isobenzofurane, benzoimidazole, benzoxazole, benzoisoxazole, benzoisothiazole, benzoxadiazole, benzothiadiazole, and the like. Particular fused heteroaryl groups are derived from pyrrolopyridine, pyrrolopyrimidine, pyrazolopyridine, thienopyridine, furopyridine, indole, azaindole, diazaindole, imidazopyridine, benzothiazole, quinoline, in particular pyrrolopyridine.

“Heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, (including spiro compounds), saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen) and preferably 1 to 4 heteroatoms.

The term “heterocyclic” as used herein, refers to a stable non-aromatic 3-, 4-, 5-, 6- or 7-membered monocyclic ring or a 7-, 8-, 9-, 10-, 11- or 12-membered bicyclic ring or a 10-, 11-, 12-, 13-, 14- or 15-membered tricyclic ring; each of which may be saturated or partially unsaturated containing at least one, e.g. 1 to 3, heteroatoms selected from oxygen, nitrogen or sulphur, where in a 8-12 membered bicyclic heterocycle one ring may be aromatic but the other one has to be fully saturated and one ring may be carbocyclic and need to include one heterocyclic ring. Monocyclic heterocycle ring may contain up to three heteroatoms. Typically, monocyclic heterocycle will contain up to 3 heteroatoms, usually up to 2, for example a single heteroatom. The bicyclic heterocycle may contain up to four heteroatoms. Typically, bicyclic heterocycle will contain up to 4 heteroatoms, more typically up to 3 heteroatoms, more usually up to 2, for example a single heteroatom. In one embodiment, the heterocycle ring contains at least one or two heteroatoms. The nitrogen and sulfur heteroatoms may optionally be oxidized, and the nitrogen heteroatom may optionally be quaternized. Examples of monocyclic rings include azetidine, pyrrolidine, pyrazolidine, oxazolidine, piperidine, piperazine, pyrane, morpholine, thiomorpholine, thiazolidine, oxirane, oxetane, dioxolane, dioxane, oxathiolane, oxathiane, dithiane, dihydrofurane, tetrahydrofurane, dihydropyrane, tetrahydropyrane, tetrahydropyridine, tetrahydropyrimidine, tetrahydrothiophene, tetrahydrothiopyrane and the like. Particular monocyclic heterocyclic groups include pyrrolidinyl, piperidinyl, piperazinyl, tetrahydropyranyl, morpholinyl and azetidinyl. Examples of bicyclic rings include 6,8-dihydro-5H-imidazo[1,2-a]pyrazine, 6,7-dihydro-5H-pyrrolo[1,2-a]imidazole, 5,6,7,8-tetrahydro-imidazo[1,2-a]pyridine, 2,3-dihydro-furo[3,2-b]pyridine, indoline, isoindoline, benzodioxole, tetrahydroisoquinoline and the like.

As used herein, the term “heterocycloalkyl” refers to a stable non-aromatic ring structure, mono-cyclic or polycyclic, containing one or more heteroatoms, particularly one or two heteroatoms independently selected from N, O and S and the number of ring atoms specified. The heterocycloalkyl ring structure may have from 3 to 7 ring members. A fused heterocyclic ring system may include carbocyclic rings and need only include one heterocyclic ring. Examples of heterocyclic rings include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone (2-pyrrolidone or 3-pyrrolidone), tetrahydrofuran, tetrahydrothiophene, dioxane, tetrahydropyran (e.g. 4-tetrahydro pyranyl), imidazolidinone, pyrazolidine, piperazine, and N-alkyl piperazines such as N-methyl piperazine and the like. Further examples include thiomorpholine and its S-oxide and S,S-dioxide (particularly thiomorpholine). Still further examples include azetidine, piperidone, piperazone, and N-alkyl piperidines such as N-methyl piperidine. Particular “heterocycloalkyl” groups are monocyclic. Particular heterocycloalkyl groups include pyrrolidinyl, piperidinyl, piperazinyl, tetrahydropyranyl, morpholinyl and azetidinyl.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

The term “hydroxy” or “hydroxyl” refers to the radical —OH.

The term “hydroxy protecting group” refers to a substituent on an functional hydroxyl group which prevent undesired reactions and degradations during synthetic procedures, and which may be selectively removed after certain synthetic step. Examples of ‘hydroxy protecting group’ include: ester and ether hydroxyl protecting group. Examples of ester hydroxyl protecting group include: formyl, —OC(O)C₁₋₄alkyl such as acetyl (Ac or —C(O)CH₃), methoxyacetyl, chloroacetyl, dichloroacetyl, trichloroacety, trifluoroacetyl, triphenylmethoxyacetyl, phenoxyacetyl, benzoylformyl, benzoyl (Bz or —C(O)C₆H₅), benzyloxycarbonyl (Cbz or —C(O)—O—CH₂C₆H₅), methoxycarbonyl, tert-butoxycarbonyl, isopropoxycarbonyl, diphenylmethoxycarbonyl or 2-(trimethylsilyl)ethoxycarbonyl and the like. Examples of ether hydroxyl protecting group include: alkyl silyl groups such as trimethylsilyl (TMS), tert-butyldimethylsilyl, triethylsilyl, triisopropylsilyl and the like. Examples of suitable “hydroxy protecting group” include; —OC(O)C₁₋₄alkyl such as acetyl (Ac or —C(O)CH₃), benzoyl (Bz), benzyloxycarbonyl (Cbz) and trimethylsilyl (TMS). Suitably, “hydroxy protecting group” is: triethylsilyl or acetyl (Ac or —C(O)CH₃). Conveniently, “hydroxy protecting group” is: Ac or Cbz.

The term “sulfonamide” refers to the —NR—SO₂—R wherein each R is independently H, alkyl, carbocycle, heterocycle, carbocycloalkyl or heterocycloalkyl), a carbocycle or a heterocycle. Particular sulfonamide groups are alkylsulfonamide (e.g. —NH—SO₂-alkyl), for example methylsulfonamide; arylsulfonamdie (i.e. —NH—SO₂-aryl) for example phenylsulfonamide; aralkylsulfonamide, for example benzylsulfonamide.

The term “sulfonyl” means a —SO₂—R group wherein R is alkyl, carbocycle, heterocycle, carbocycloalkyl or heterocycloalkyl. Particular sulfonyl groups are alkylsulfonyl (i.e. —SO₂-alkyl), for example methylsulfonyl; arylsulfonyl, for example phenylsulfonyl; aralkylsulfonyl, for example benzylsulfonyl.

The term “nitro” refers to the radical —NO₂.

The term “cyano” refers to the radical —CN.

The term “partially unsaturated” refers to a ring moiety that includes at least one double or triple bond between ring atoms but is not aromatic. The term “partially unsaturated” is intended to encompass rings having multiple sites of unsaturation, but is not intended to include aryl or heteroaryl moieties. Only in case of fused 8-9 membered heterocycle one of the ring moieties may be aromatic but in that case the other ring of such fused 8-9-memberd heterocycle has to be saturated.

The term “intermediates(s) of the invention” or “intermediate(s) according to the invention”, and equivalent expressions refers to compounds of formulae (II), (III), (IV) and (V) (whether in solvated or unsolvated form), as herein described, including any subset or embodiment of compounds of formulae (II), (III), (IV) and (V), or their salts (whether in solvated or unsolvated form). Suitably, said expression includes the salts, and solvates (e.g. hydrates) thereof. The intermediate(s) of the invention may possess one or more asymmetric centres; such intermediate(s) can therefore be produced as individual (R)- or (S)-stereoisomers or as mixtures thereof. Where stereochemistry is not defined in the relevant Formula(e), then the term intermediate(s) of the invention includes enantiomers and diastereoisomers of these compounds.

The term “inert solvent” or “solvent inert to the reaction”, as used herein, refers to a solvent that cannot react with the dissolved compounds including non-polar solvent such as hexane, toluene, diethyl ether, diisopropylether, chloroform, ethyl acetate, THF, dichloromethane; polar aprotic solvents such as acetonitrile, acetone, N,N-dimethylformamide, N,N-dimethylacetamide, dimethyl sulfoxide, pyridine, and polar protic solvents such as lower alcohol, acetic acid, formic acid and water.

The term “lower alcohol”, as used herein, refers to a C₁₋₄alcohol, such as for example, methanol, ethanol, propanol, isopropanol, butanol, t-butanol, and the like.

The term “substituted” refers to a group in which one or more hydrogen atoms are each independently replaced with the same or different substituent(s).

As used herein, the term “substituted with one or more” refers to one to four substituents. In one embodiment it refers to one to three substituents. In further embodiment it refers to one or two substituents. In a yet further embodiment it refers to one substituent.

The term “pharmaceutically acceptable”, as used herein, refers to salts, molecular entities and other ingredients of compositions that are generally physiologically tolerable and do not typically produce untoward reactions when administered to a mammal (e.g., human). Suitably, as used herein, the term “pharmaceutically acceptable” means approved by a regulatory agency of the Federal or a state government or the corresponding agency in countries other than the United States or listed in the U.S. Pharmacopoeia or other generally recognized pharmacopoeia for use in mammals, and more particularly in humans.

“Pharmaceutically acceptable salt” refers to a salt of a compound that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminium ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

The term “pharmaceutically acceptable ester” refers to esters which hydrolyse in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

“Pharmaceutically acceptable vehicle” refers to a diluent, adjuvant, excipient or carrier with which a compound of the invention is administered.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

The term “prodrug” or “pharmaceutically acceptable prodrug” as used herein refers to compounds, including derivatives of the compounds of the invention, which have metabolically cleavable groups and are converted within the body e.g. by solvolysis or under physiological conditions into the compounds of the invention which are pharmaceutically active in vivo. Pharmaceutically acceptable prodrugs are described in: Bundgard, H. Design of Prodrugs, pp. 7-9, 21-24, Elsevier, Amsterdam 1985, T. Higuchi and V. Stella, “Prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130. Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁-C₈ alkyl, C₂-C₈ alkenyl, aryl and arylalkyl esters of the compounds of the invention.

The term “solvate” refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

The term “isotopic variant” refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

The term “isomer(s)” refers to compounds that have the same molecular formula but differ in the nature or sequence of bonding of their atoms or the arrangement of their atoms in space. Isomers that differ in the arrangement of their atoms in space are termed “stereoisomers”.

“Diastereomers” are stereoisomers that are not mirror images of one another and those that are non-superimposable mirror images of each other are termed ‘enantiomers’. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of 7 electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

The term “subject” refers to an animal, in particular a mammal and more particular to a human or a domestic animal serving as a model for a disease (for example guinea pigs, mice, rats, gerbils, fish, birds, cats, rabbits, dogs, horses, cows, monkeys, chimpanzees or like). Specifically, the subject is a human. The terms “patient” and “subject” are used interchangeably herein.

‘Co-administration’ includes any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

“Effective amount” means the amount of a compound that, when administered to a subject for the prophylaxis or treatment of a disease and/or condition, is sufficient to affect such prophylaxis or such treatment for the disease and/or condition. The “effective amount” can vary depending on the compound, the disease and/or condition and its severity, and the age, weight, etc., of the subject.

“Preventing” or “prevention” refers to a reduction in risk of acquiring or developing a disease and/or condition (i.e., causing at least one of the clinical symptoms of the disease and/or condition not to develop in a subject that may be exposed to a disease and/or condition-causing agent, or predisposed to the disease and/or condition in advance of disease and/or condition onset).

The term “prophylaxis” is related to “prevention”, and refers to a measure or procedure the purpose of which is to prevent, rather than to treat or cure a disease.

“Treating” or “treatment” of any disease and/or condition refers, in one embodiment, to ameliorating the disease and/or condition (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease and/or condition, either physically, (e.g., stabilization of a discernible symptom), physiologically, (e.g., stabilization of a physical parameter), or both. In a further embodiment, “treating” or “treatment” relates to slowing the progression of the disease and/or condition.

“Maintenance therapy” refers to a preventive therapy that follows successful initial treatment of the acute phase of the illness where regular (usually smaller) doses of the drug are delivered to the patient to prevent recurrence and worsening of the disease.

Modulators of tryptophan catabolism include modulators of a pathway involved in tryptophan catabolism; and includes inhibitors of tryptophan catabolism, in which the inhibition may be complete or partial.

The term ‘modulators of tryptophan catabolism’ as used herein includes those compounds which result in increased levels of tryptophan and/or reduced levels of tryptophan catabolites.

The term ‘modulators of tryptophan catabolism’ as used herein may be modulators of any proteins within tryptophan catabolism pathway which result in increased levels of tryptophan and/or reduced levels of tryptophan catabolites.

The term ‘modulators of tryptophan catabolism’ as used herein may inhibit the degradation of tryptophan and reduce kynurenine production individually, simultaneously or sequentially.

The term ‘modulators of tryptophan catabolism’ as used herein may inhibit the degradation of tryptophan and reduce kynurenine production individually, simultaneously or sequentially without regulating IDO or TDO activity.

The term ‘modulators of tryptophan catabolism’ as used herein may inhibit the degradation of tryptophan and reduce kynurenine production individually, simultaneously or sequentially without regulating TDO activity.

The term ‘modulators of tryptophan catabolism’ as used herein may inhibit the degradation of tryptophan and reduce kynurenine production individually, simultaneously or sequentially without regulating IDO activity.

The term ‘modulators of tryptophan catabolism’ as used herein may include inhibitors of IDO, inhibitors of TDO, or modulators of any other proteins within tryptophan catabolism pathway which result in increased levels of tryptophan and/or reduced levels of tryptophan catabolites.

Modulators of tryptophan catabolism may differentially regulate the activity of IDO or TDO or any other proteins within tryptophan catabolism pathway which result in increased levels of tryptophan and/or reduced levels of tryptophan catabolites.

Modulators of tryptophan catabolism may differentially regulate the level of IDO protein or TDO protein or any other proteins within tryptophan catabolism pathway which result in increased levels of tryptophan and/or reduced levels of tryptophan catabolites.

The term ‘tryptophan catabolites’ as used herein may include kynurenines such as N-formyl kynurenine.

The term IDO as used herein refers to the haem-containing enzyme indoleamine 2, 3-dioxygenase. Unless otherwise stated the term IDO encompasses both paralogs of IDO (IDO1 and IDO2).

The term TDO as used herein refers to the haem-containing enzyme tryptophan 2, 3-dioxygenase.

As used herein the term “diseases and/or conditions associated with the abnormal or elevated catabolism of tryptophan” refers to group of conditions including cancer, immunosuppression, viral infection, depression, a neurodegenerative disorder, trauma, age-related cataracts, organ transplant rejection, or an autoimmune disorder in a patient, as herein defined.

The term ‘abnormal activation’ used herein refers to aberrant activation, reduced inhibition, increased expression, increased signalling or inappropriate activation.

The term “amidation” used herein refers to a chemical process of formal union of carboxylic acids and amines and formation of amide functionality. It is necessary to first activate the carboxylic acid, in a process that usually takes place by converting the—OH of the acid into a good leaving group prior to treatment with the amine in the presence of a base. Suitable methods for activation of carboxylic groups are, but not limited to, formation of acyl halides, acyl azides, mixed anhydrides, activated esters and the like. Acyl halides may be prepared in non-protic solvents treating the carboxylic acid with halide sources like, but not limited to, thionyl chloride, oxalyl chloride, phosphorus pentachloride, triphosgene, cyanuric fluoride, cyanuric chloride, BoP-Cl, PyBroP and the like. Mixed anhydrides may be prepared in non-protic solvents with reagents like, but not limited to, pivaloyl chloride, EEDQ and the like. Suitable coupling reagents used in the process of amidation via active esters are, but not limited to, carbodiimides like DCC, DIC, EDAC, uronium salts like HATU, TATU, HBTU, TBTU, TDBTU, phosphonium salts like PyBoP, BoP, DEPBT. These coupling reagents can be used as stand-alone activators or in the presence of additives like, but not limited to, HOAt, HOBt and the like. Other suitable amidation coupling reagents that operate on different mechanism of carboxylic group activation are, but not limited to, DPPA, T3P®, CDI, Mukaiyama reagent and the like. Activation can also be performed by using solid supported versions of the abovementioned coupling reagents like, but not limited to, PS-CDI, PS-EDC, PS-BoP and the like. Suitable bases used in amidation process are, but not limited to, sodium hydrocarbonate, potassium hydrocarbonate, sodium carbonate, potassium carbonate, TEA, DIPEA, DBU, DBN, DABCO and the like. A more thorough discussion of amidation can be found in Valeur, E., et al. Chem. Soc. Rev. (2009), 38, 606.

The term “esterification” used herein refers to a chemical process of formal union of carboxylic acids and alcohols and formation of ester functionality. Suitable methods for synthesis of esters are Fisher, Mitsunobu, Steglich conditions, transesterification, acylation with appropriate acyl halides, decarboxylative esterification, oxidative esterification and redox esterification. Acyl halides may be prepared in non-protic solvents treating the carboxylic acid with halide sources like, but not limited to, thionyl chloride, oxalyl chloride, phosphorus pentachloride, triphosgene, fluoride, cyanuric chloride and the like. Suitable coupling reagents used in the process of esterification are, but not limited to, p-nitrophenylchloroformate, thiopyridyl chloroformate, 2,2′-(4-t-Bu-N-alkylimidazolyl)disulfide, Mukaiyama salts, 2,4,6-trichlorobenzoyl chloride, DEAD/PPh3, TFFH, DCC, TBTU, TATU, COMU and the like. Suitable bases used in esterification process are, but not limited to, sodium hydrocarbonate, potassium hydrocarbonate, sodium carbonate, potassium carbonate, TEA, DIPEA, DBU, DBN, DABCO and the like.

The term “reductive amination” used herein refers to chemical process of conversion of a carbonyl group and an amine to higher substituted amine via an intermediate imine. The carbonyl group is most commonly a ketone or an aldehyde. The imine intermediate is reduced to the amine by various reducing agents including, but not limited to, sodium borohydride, sodium triacetoxyborohydride, sodium cyanoborohydride, zinc and hydrochloric acid, hydrogen and transition metal catalyst, formic acid and its organic or inorganic salts, iron pentacarbonyl. Generally alcoholic solvents are used. Preferred conditions are sodium cyanoborohydride in methanolic media in the presence of acetic acid.

### DETAILED DESCRIPTION

The present invention is based on the identification that a compound of the invention may be useful as a medicament in the treatment of diseases and/or conditions associated with the abnormal or elevated catabolism of tryptophan. In a particular aspect, a compound of the invention is a modulator of tryptophan catabolism. More particularly, a compound of the invention is useful in the treatment of proliferative diseases. The present invention also relates to methods for the preparation of the compounds of the invention, to intermediates for their preparation, to pharmaceutical compositions comprising a compound of the invention, to the use of a compound of the invention as therapeutic agents, and to methods for the treatment of diseases and/or conditions associated with the abnormal or elevated catabolism of tryptophan by administering a compound of the invention.

Accordingly, in another aspect the present invention relates to a compound of Formula (I):

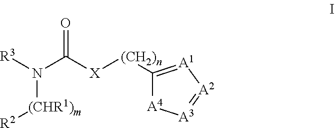

or a pharmaceutically acceptable salt, or a solvate, or a solvate of the salt thereof,

wherein:

m is 0 or 1;

n is 0, 1 or 2;

X is —NR⁸;

R¹ is H, C₁₋₆alkyl or a 6-10 membered aryl;

R² is a 5-6-membered heteroaryl, a fused 9-10 membered bicyclic heteroaryl, a 6-10 membered aryl, a 5-6 membered monocyclic heterocycloalkyl a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy and phenyl;

R³ is H or C₁₋₆alkyl; or a 3-10 membered cycloalkyl, a 5-11 membered spiroalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆ alkyl, halogen, —CN and —C(═O)OC₁₋₆alkyl;

A¹ is —N— or —CR⁶—;

A² is —N— or —CR⁵—;

A³ is —N— or —CR⁷—;

A⁴ is —N—, —O—, —S—, —CH═N— or —CH═CR⁴—;

R⁴, R⁵, R⁶ and R⁷, which may be the same or different, are each selected from —H, —OH, —C₁₋₆alkyl, halogen, haloC₁₋₆alkyl, —CN, —C₁₋₆alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆ cycloalkyl, —C₂₋₆alkynyl-C₁₋₆alkyl-NR¹¹R¹², —C₂₋₆alkynyl-C₁₋₆alkyl-OR¹³, —C(═O)C₁₋₆ alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;

or R⁵ and R⁷ together form a ring —CH═CH—CH═CH—;

or the moiety

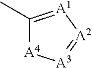

may be fused with oxopyrrolidine; and

R⁸, R⁹, R¹⁰, R¹¹, R¹², and R¹³, which may be the same or different, are each selected from H or C₁₋₆alkyl;

provided that the compound of formula I is not 1-(4-chlorobenzyl)-1-cyclopentyl-3-phenylurea;
N-(3,5-dimethylphenyl)-3-ethyl-2-methyl-7-phenyl-5,7-dihydro-4H-thieno[2,3-c]pyridine-6-carboxamide; [194]
1-cyclopentyl-3-phenyl-1-(2-thienylmethyl)urea; [195]
1-(4-chlorophenyl)-3-phenyl-1-(2-thienylmethyl)urea; [196]
1-[1-(4-fluorophenyl)ethyl]-3-phenyl-urea; [197]
1-(4-chlorophenyl)-3-[1-(5-chloro-2-thienyl)ethyl]urea; [199]
3-(3,4-dichlorophenyl)-1-methyl-1-(2-thienylmethyl)urea; [200]
1-[(5-methyl-2-phenyl-oxazol-4-yl)methyl]-3-phenyl-urea; [203] and
1-(3-chlorophenyl)-3-[(3-chloro-2-thienyl)methyl]urea; [204].

In another aspect of the invention a compound of formula I is not:
1-(4-chlorophenyl)-3-[phenyl(2-thienyl)methyl]urea; [198] and
1-[(4-ethylphenyl)methyl]-3-(2-methoxyphenyl)-1-(3-pyridylmethyl)urea; [205].

In one embodiment, the compound of the invention is selected amongst the compounds 1 to 328.

Specific compounds of formula I according to this aspect of the invention which may be mentioned include those selected from the group consisting of
1-cyclopentyl-3-(2-phenylethyl)-1-(2-thienylmethyl)urea; [1]
3-(2-chlorophenyl)-1-cyclopentyl-1-(2-thienylmethyl)urea; [2]
1-cyclopentyl-3-(4-ethylphenyl)-1-(2-thienylmethyl)urea; [3]
1-cyclopentyl-3-(3,4-difluorophenyl)-1-(2-thienylmethyl)urea; [4]
1-cyclopentyl-3-(2,4-dimethylphenyl)-1-(2-thienylmethyl)urea; [5]
3-[4-(cyanomethyl)phenyl]-1-cyclopentyl-1-(2-thienylmethyl)urea; [6]
3-(1,3-benzodioxol-5-yl)-1-cyclopentyl-1-(2-thienylmethyl)urea; [7]
1-cyclopentyl-3-[(4-fluorophenyl)methyl]-1-(2-thienylmethyl)urea; [8]
1-cyclopentyl-3-indan-5-yl-1-(2-thienylmethyl)urea; [9]
“1-cyclopentyl-3-(2,6-dichloro-4-pyridyl)-1-(2-thienylmethyl)urea; [10]”
1-cyclopentyl-3-(4-pyridyl)-1-(2-thienylmethyl)urea; [11]
1-cyclopentyl-1-(2-thienylmethyl)-3-[4-(trifluoromethyl)phenyl]urea; [13]
1-cyclopentyl-3-(4-methoxyphenyl)-1-(2-thienylmethyl)urea; [14]
3-allyl-1-cyclopentyl-1-(2-thienylmethyl)urea; [15]
“1-cyclopentyl-3-(5-ethynyl-2-pyridyl)-1-[(5-methyl-2-furyl)methyl]urea; [16]”
“1-cyclopentyl-3-(5-ethynylpyrimidin-2-yl)-1-[(5-methyl-2-furyl)methyl]urea; [17]”
1-cyclopentyl-3-(2,4-dimethoxyphenyl)-1-(2-thienylmethyl)urea; [18]
1-cyclopentyl-3-phenyl-1-(2-thienylmethyl)urea; [19]
1-cyclohexyl-3-(2-phenylethyl)-1-(2-pyridylmethyl)urea; [20]
1-cyclohexyl-3-(4-ethylphenyl)-1-(2-pyridylmethyl)urea; [21]
3-(4-acetylphenyl)-1-cyclopentyl-1-(2-thienylmethyl)urea; [22]
1-cyclopentyl-3-methyl-3-phenyl-1-(2-thienylmethyl)urea; [23]
1-cyclopentyl-1-[(5-methyl-2-thienyl)methyl]-3-phenyl-urea; [24]
1-cyclopentyl-3-(4-ethylphenyl)-1-[(5-methyl-2-thienyl)methyl]urea; [25]
1-cyclopentyl-1-[(5-methyl-2-thienyl)methyl]-3-(2-phenylethyl)urea; [26]
“1-cyclopentyl-3-(3,4-difluorophenyl)-1-[(5-methyl-2-thienyl)methyl]urea; [27]”
1-cyclopentyl-1-(2-furylmethyl)-3-phenyl-urea; [28]
1-cyclopentyl-1-(2-furylmethyl)-3-(2-phenylethyl)urea; [29]
1-cyclopentyl-3-(3,4-difluorophenyl)-1-(2-furylmethyl)urea; [30]
1-cyclopentyl-3-(3,4-difluorophenyl)-1-[(5-methyl-2-furyl)methyl]urea; [31]
1-cyclohexyl-3-phenyl-1-(2-pyridylmethyl)urea; [32]
1-cyclohexyl-3-(3,4-difluorophenyl)-1-(2-pyridylmethyl)urea; [33]
1-cyclopentyl-3-(4-fluorophenyl)-1-(2-thienylmethyl)urea; [34]
1-cyclopentyl-3-phenyl-1-(thiazol-2-ylmethyl)urea; [35]
1-cyclopentyl-3-(4-ethylphenyl)-1-(thiazol-2-ylmethyl)urea; [36]
1-cyclopentyl-3-(3,4-difluorophenyl)-1-(thiazol-2-ylmethyl)urea; [37]
1-[(2-chlorophenyl)methyl]-1-cyclopentyl-3-phenyl-urea; [38]
1-[(2-chlorophenyl)methyl]-1-cyclopentyl-3-(4-ethylphenyl)urea; [39]
1-[(2-chlorophenyl)methyl]-1-cyclopentyl-3-(3,4-difluorophenyl)urea; [40]
1-[(5-chloro-1-methyl-pyrazol-4-yl)methyl]-1-cyclopentyl-3-phenyl-urea; [41]
1-[(5-chloro-1-methyl-pyrazol-4-yl)methyl]-1-cyclopentyl-3-(4-ethylphenyl)urea; [42]
1-[(5-chloro-1-methyl-pyrazol-4-yl)methyl]-1-cyclopentyl-3-(3,4-difluorophenyl) urea; [43]
1-[(5-chloro-1-methyl-pyrazol-4-yl)methyl]-1-cyclopentyl-3-(4-fluorophenyl) urea; [44]
1-cyclopentyl-1-[(4-methoxy-3-methyl-phenyl)methyl]-3-phenyl-urea; [45]
1-cyclopentyl-1-[(4-methoxy-3-methyl-phenyl)methyl]-3-(2-phenylethyl)urea; [46]
1-cyclopentyl-3-(3,4-difluorophenyl)-1-[(4-methoxy-3-methyl-phenyl)methyl]urea; [47]
1-cyclopentyl-3-(4-fluorophenyl)-1-[(4-methoxy-3-methyl-phenyl)methyl]urea; [48]
1-cyclopentyl-1-[(2-methoxythiazol-5-yl)methyl]-3-phenyl-urea; [49]
1-[(3-cyano-4-fluoro-phenyl)methyl]-1-cyclopentyl-3-phenyl-urea; [50]
1-[(3-cyano-4-fluoro-phenyl)methyl]-1-cyclopentyl-3-(2-phenylethyl)urea; [51]
1-[(3-cyano-4-fluoro-phenyl)methyl]-1-cyclopentyl-3-(3,4-difluorophenyl)urea; [52]
1-[(3-cyano-4-fluoro-phenyl)methyl]-1-cyclopentyl-3-(4-fluorophenyl)urea; [53]
1-cyclopentyl-3-(2-fluorophenyl)-1-(2-thienylmethyl)urea; [54]
1-cyclopentyl-3-(3-fluorophenyl)-1-(2-thienylmethyl)urea; [55]
3-(4-chlorophenyl)-1-cyclopentyl-1-(2-thienylmethyl)urea; [56]
1-cyclopentyl-3-(3-pyridyl)-1-(2-thienylmethyl)urea; [57]
1-cyclopentyl-1-phenyl-3-(2-thienyl)urea; [58]
1-cyclopentyl-3-(2,4-dichlorophenyl)-1-(2-thienylmethyl)urea; [59]
1-[(5-cyano-2-furyl)methyl]-1-cyclopentyl-3-phenyl-urea; [60]
1-[(5-cyano-2-furyl)methyl]-1-cyclopentyl-3-(4-fluorophenyl)urea; [61]
1-cyclopentyl-3-(4-fluorophenyl)-1-(isoxazol-4-ylmethyl)urea; [62]
3-(4-chlorophenyl)-1-cyclopentyl-1-(isoxazol-4-ylmethyl)urea; [63]
1-cyclopentyl-1-(3-furylmethyl)-3-phenyl-urea; [64]
1-cyclopentyl-3-phenyl-1-(3-pyridylmethyl)urea; [65]
3-(4-chlorophenyl)-1-cyclopentyl-1-(3-pyridylmethyl)urea; [66]
1-cyclopentyl-3-phenyl-1-(2-pyridylmethyl)urea; [67]
1-cyclopentyl-3-(4-fluorophenyl)-1-(2-pyridylmethyl)urea; [68]
3-(4-chlorophenyl)-1-cyclopentyl-1-(2-pyridylmethyl)urea; [69]
“1-cyclopentyl-3-(4-fluorophenyl)-1-(pyrazin-2-ylmethyl)urea; [70]”
3-(4-chlorophenyl)-1-cyclopentyl-1-(pyrazin-2-ylmethyl)urea; [71]
1-cyclopentyl-3-(4-fluorophenyl)-1-(pyrimidin-2-ylmethyl)urea; [72]
3-(4-chlorophenyl)-1-cyclopentyl-1-(pyrimidin-2-ylmethyl)urea; [73]
1-cyclopentyl-3-phenyl-1-(4-pyridylmethyl)urea; [74]
“1-cyclopentyl-3-(4-fluorophenyl)-1-(4-pyridylmethyl)urea; [75]”
3-(4-chlorophenyl)-1-cyclopentyl-1-(4-pyridylmethyl)urea; [76]
tert-butyl 4-[[cyclopentyl(phenylcarbamoyl)amino]methyl]-4-methyl-piperidine-1-carboxylate; [77]
“tert-butyl 4-[[cyclopentyl-[(4-fluorophenyl)carbamoyl]amino]methyl]-4-methyl-piperidine-1-carboxylate; [78]”
tert-butyl 4-[[(4-chlorophenyl)carbamoyl-cyclopentyl-amino]methyl]-4-methyl-piperidine-1-carboxylate; [79]
3-(4-cyanophenyl)-1-cyclopentyl-1-[(5-methyl-2-furyl)methyl]urea; [80]
1-cyclopentyl-1-[(5-methyl-2-furyl)methyl]-3-(4-pyridyl)urea; [81]
1-cyclobutyl-3-(4-fluorophenyl)-1-[(5-methyl-2-furyl)methyl]urea; [82]
3-(4-chlorophenyl)-1-cyclobutyl-1-[(5-methyl-2-furyl)methyl]urea; [83]
3-(4-cyanophenyl)-1-cyclobutyl-1-[(5-methyl-2-furyl)methyl]urea; [84]
1-cyclopentyl-1-[(4-methyl-4-piperidyl)methyl]-3-phenyl-urea; [85]
1-cyclopentyl-3-(4-fluorophenyl)-1-[(4-methyl-4-piperidyl)methyl]urea; [86]
3-(4-chlorophenyl)-1-cyclopentyl-1-[(4-methyl-4-piperidyl)methyl]urea; [87]
“1-cyclopentyl-3-(4-pyridyl)-1-[[3-(trifluoromethyl)phenyl]methyl]urea; [88]”
“3-(4-cyanophenyl)-1-cyclopentyl-1-[[3-(trifluoromethyl)phenyl]methyl]urea; [89]”
3-(4-chlorophenyl)-1-cyclobutyl-1-[[3-(trifluoromethyl)phenyl]methyl]urea; [90]
3-(4-cyanophenyl)-1-cyclobutyl-1-[[3-(trifluoromethyl)phenyl]methyl]urea; [91]
3-(6-chloro-3-pyridyl)-1-cyclopentyl-1-[(5-methyl-2-furyl)methyl]urea; [92]
3-(3-cyanophenyl)-1-cyclopentyl-1-[(5-methyl-2-furyl)methyl]urea; [93]
3-(4-acetylphenyl)-1-cyclopentyl-1-[(5-methyl-2-furyl)methyl]urea; [94]
3-(2-cyanophenyl)-1-cyclopentyl-1-[(5-methyl-2-furyl)methyl]urea; [95]
“3-[(4-cyanophenyl)methyl]-1-cyclopentyl-1-[(5-methyl-2-furyl)methyl]urea; [96]”
1-[(1-acetyl-4-methyl-4-piperidyl)methyl]-1-cyclopentyl-3-phenyl-urea; [97]
1-cyclopentyl-1-[(5-methyl-2-furyl)methyl]-3-[4-(1-methylpyrazol-4-yl)phenyl]urea; [98]
“tert-butyl 4-[4-[[cyclopentyl-[(5-methyl-2-furyl)methyl]carbamoyl]amino]phenyl]pyrazole-1-carboxylate; [99]”
1-cyclopentyl-3-[4-[1-[2-(dimethylamino)ethyl]pyrazol-4-yl]phenyl]-1-[(5-methyl-2-furyl)methyl]urea; [100]
1-cyclopentyl-3-[4-[1-(2-hydroxy-1,1-dimethyl-ethyl)pyrazol-4-yl]phenyl]-1-[(5-methyl-2-furyl)methyl]urea; [101]
“1-cyclopentyl-3-(4-ethynylphenyl)-1-[(5-methyl-2-furyl)methyl]urea; [102]”
“1-cyclopentyl-1-[(5-methyl-2-furyl)methyl]-3-[4-(2-oxopyrrolidin-1-yl)phenyl]urea; [103]”
“1-cyclopentyl-3-(4-fluoro-3-hydroxy-phenyl)-1-[(5-methyl-2-furyl)methyl]urea; [104]”
“1-cyclopentyl-3-(4-isoxazol-4-ylphenyl)-1-[(5-methyl-2-furyl)methyl]urea; [105]”
1-cyclopentyl-1-[(5-methyl-2-furyl)methyl]-3-(4-thiazol-4-ylphenyl)urea; [106]
1-cyclopentyl-3-[4-(2-cyclopropylethynyl)phenyl]-1-[(5-methyl-2-furyl)methyl]urea; [107]
1-cyclopentyl-1-[(5-methyl-2-furyl)methyl]-3-[4-(1H-pyrazol-4-yl)phenyl]urea; [108]
1-cyclopentyl-3-[4-(3-hydroxy-3-methyl-but-1-ynyl)phenyl]-1-[(5-methyl-2-furyl) methyl]urea; [109]
3-[4-(3-aminoprop-1-ynyl)phenyl]-1-cyclopentyl-1-[(5-methyl-2-furyl)methyl]urea; [110]
1-cyclopentyl-3-[4-(3-hydroxyprop-1-ynyl)phenyl]-1-[(5-methyl-2-furyl)methyl]urea; [111]
1-cyclopentyl-3-[4-[3-(dimethylamino)prop-1-ynyl]phenyl]-1-[(5-methyl-2-furyl) methyl]urea; [112]
“1-cyclopentyl-3-(4-fluorophenyl)-1-[(4-methyl-1-methylsulfonyl-4-piperidyl)methyl]urea; [113]”
3-(4-chlorophenyl)-1-[(2-cyano-4-pyridyl)methyl]-1-cyclopentyl-urea; [114]
3-(4-chlorophenyl)-1-[(5-cyano-3-pyridyl)methyl]-1-cyclopentyl-urea; [115]
3-(4-chlorophenyl)-1-[(4-cyano-2-pyridyl)methyl]-1-cyclopentyl-urea; [116]
4-[[cyclopentyl-[(5-methyl-2-furyl)methyl]carbamoyl]amino]benzamide; [117]
tert-butyl 4-[[cyclopentyl(phenylcarbamoyl)amino]methyl]piperidine-1-carboxylate; [118]
tert-butyl 4-[[cyclopentyl-[(4-fluorophenyl)carbamoyl]amino]methyl]piperidine-1-carboxylate; [119]
tert-butyl 4-[[(4-chlorophenyl)carbamoyl-cyclopentyl-amino]methyl]piperidine-1-carboxylate; [120]
3-(5-cyano-2-pyridyl)-1-cyclopentyl-1-[(5-methyl-2-furyl)methyl]urea; [121]
5-[[cyclopentyl-[(4-fluorophenyl)carbamoyl]amino]methyl]-2-fluoro-benzamide; [122]
1-cyclopentyl-1-[(5-methyl-2-furyl)methyl]-3-(4-morpholinophenyl)urea; [123]
3-(6-cyano-3-pyridyl)-1-cyclopentyl-1-[(5-methyl-2-furyl)methyl]urea; [124]
1-[(6-cyano-2-pyridyl)methyl]-1-cyclopentyl-3-phenyl-urea; [125]
1-[(6-cyano-2-pyridyl)methyl]-1-cyclopentyl-3-(4-fluorophenyl)urea; [126]
1-cyclopentyl-3-(4-fluorophenyl)-1-(isoxazol-5-ylmethyl)urea; [127]
3-(4-chlorophenyl)-1-cyclopentyl-1-(isoxazol-5-ylmethyl)urea; [128]
1-cyclopentyl-1-[(5-methyl-2-furyl)methyl]-3-(4-phenylthiazol-2-yl)urea; [129]
1-(benzofuran-2-ylmethyl)-3-(4-chlorophenyl)-1-cyclopentyl-urea; [130]
1-(benzofuran-2-ylmethyl)-1-cyclopentyl-3-(4-ethynylphenyl)urea; [131]
“1-(benzofuran-2-ylmethyl)-3-(4-cyanophenyl)-1-cyclopentyl-urea; [132]”
“1-cyclopentyl-1-[(5-methyl-2-furyl)methyl]-3-[4-(2-pyridyl)thiazol-2-yl]urea; [133]”
3-(1,3-benzothiazol-2-yl)-1-cyclopentyl-1-[(5-methyl-2-furyl)methyl]urea; [134]
“3-(4-cyanothiazol-2-yl)-1-cyclopentyl-1-[(5-methyl-2-furyl)methyl]urea; [135]”
“tert-butyl 4-[(5-methyl-2-furyl)methyl-(phenylcarbamoyl)amino]piperidine-1-carboxylate; [136]”
3-(4-chlorophenyl)-1-cyclopentyl-1-[(5-methyl-2-furyl)methyl]urea; [137]
1-isopropyl-1-[(5-methyl-2-furyl)methyl]-3-phenyl-urea; [138]
1-cyclopentyl-3-(3,4-difluorophenyl)-1-[[4-fluoro-3-(trifluoromethyl)phenyl]methyl]urea; [139]
1-cyclopentyl-3-(3,4-difluorophenyl)-1-[[3(trifluoromethyl)phenyl]methyl]urea; [140]
1-cyclopentyl-3-(3,4-difluorophenyl)-1-[[4-fluoro-3 (trifluoromethoxy)phenyl]methyl]urea; [141]
1-cyclopentyl-3-(3,4-difluorophenyl)-1-[[3 (trifluoromethoxy)phenyl]methyl]urea; [142]
1-cyclopentyl-1-[(2-methyloxazol-5-yl)methyl]-3-phenyl-urea; [143]
3-(4-chlorophenyl)-1-cyclopentyl-1-[(2-methyloxazol-5-yl)methyl]urea; [144]
1-cyclopentyl-3-(4-fluorophenyl)-1-[(2-methyloxazol-5-yl)methyl]urea; [145]
3-(4-chlorophenyl)-1-[(3-cyano-4-fluoro-phenyl)methyl]-1-cyclopentyl-urea; [146]
3-(4-chlorophenyl)-1-[(3-cyanophenyl)methyl]-1-cyclopentyl-urea; [147]
3-(4-chlorophenyl)-1-cyclopentyl-1-[[2-(trifluoromethyl)-4-pyridyl]methyl]urea; [148]
3-(4-chlorophenyl)-1-cyclopentyl-1-(isoxazol-3-ylmethyl)urea; [149]
1-cyclopentyl-1-[(5-methyl-2-furyl)methyl]-3-(4-phenylphenyl)urea; [150]
3-[5-(benzofuran-2-yl)-1,3,4-oxadiazol-2-yl]-1-cyclopentyl-1-[(5-methyl-2-furyl) methyl]urea; [151]
3-(4-cyanophenyl)-1-cyclobutyl-1-[[2-(trifluoromethyl)-4-pyridyl]methyl]urea; [152]
1-cyclobutyl-3-(4-ethynylphenyl)-1-[[2-(trifluoromethyl)-4-pyridyl]methyl]urea; [153]
1-cyclobutyl-3-(4-prop-1-ynylphenyl)-1-[[2-(trifluoromethyl)-4-pyridyl]methyl]urea; [154]
1-cyclopentyl-1-[(5-methyl-2-furyl)methyl]-3-[4-(2-phenylethynyl)phenyl]urea; [155]
1-cyclopentyl-1-[(5-methyl-2-furyl)methyl]-3-[4-(3-phenylprop-1-ynyl)phenyl]urea; [156]
3-(4-chlorophenyl)-1-[(6-cyano-2-pyridyl)methyl]-1-cyclopentyl-urea; [157]
tert-butyl 3-[4-[[cyclopentyl-[(5-methyl-2-furyl)methyl]carbamoyl]amino]phenyl]-2,5-dihydropyrrole-1-carboxylate; [158]
tert-butyl 3-[4-[[cyclopentyl-[(5-methyl-2-furyl)methyl]carbamoyl]amino]phenyl]azetidine-1-carboxylate; [159]
1-cyclopentyl-1-[(5-methyl-2-furyl)methyl]-3-[6-(1-methylpyrazol-4-yl)-3-pyridyl]urea; [160]
3-(5-bromothiazol-2-yl)-1-cyclopentyl-1-[(5-methyl-2-furyl)methyl]urea; [161]
1-cyclopentyl-3-[4-(3-methoxyprop-1-ynyl)phenyl]-1-[(5-methyl-2-furyl)methyl]urea; [162]
1-cyclopentyl-3-(4-ethynylphenyl)-1-(isoxazol-5-ylmethyl)urea; [163]
1-cyclopentyl-3-[4-[4-(hydroxymethyl)triazol-1-yl]phenyl]-1-[(5-methyl-2-furyl) methyl]urea; [164]
“1-ethyl-1-[(5-methyl-2-furyl)methyl]-3-phenyl-urea; [165]”
“1-[(5-methyl-2-furyl)methyl]-3-phenyl-1-propyl-urea; [166]”
“1-cyclopropyl-1-[(5-methyl-2-furyl)methyl]-3-phenyl-urea; [167]”
1-cyclopentyl-1-[(5-methyl-2-furyl)methyl]-3-[5-(2-thienyl)-1,3,4-oxadiazol-2-yl]urea; [168]
3-(5-cyclohexyl-1,3,4-oxadiazol-2-yl)-1-cyclopentyl-1-[(5-methyl-2-furyl)methyl]urea; [169]
“1-cyclopentyl-1-[(2,4-dimethylthiazol-5-yl)methyl]-3-phenyl-urea; [170]”
“1-cyclopentyl-1-[(2,4-dimethylthiazol-5-yl)methyl]-3-(3,4-difluorophenyl)-urea; [171]”
3-(4-chloro-2-fluoro-phenyl)-1-cyclopentyl-1-[(5-methyl-2-furyl)methyl]urea; [172]
3-(4-chlorophenyl)-1-[1-(3-cyanophenyl)ethyl]-1-cyclopentyl-urea; [173]
3-(4-chlorophenyl)-1-cyclopentyl-1-[1-(2-pyridyl)ethyl]urea; [174]
1-cyclopentyl-1-[(5-methyl-2-furyl)methyl]-3-(5-phenyl-1,3,4-oxadiazol-2-yl) urea; [175]
“1-cyclopentyl-1-[(5-methyl-2-furyl)methyl]-3-phenyl-urea; [176]”
“1-cyclopentyl-3-(4-ethylphenyl)-1-[(5-methyl-2-furyl)methyl]urea; [177]”
1-cyclobutyl-1-[(5-methyl-2-furyl)methyl]-3-phenyl-urea; [178]
1-[(5-methyl-2-furyl)methyl]-1-oxazol-2-yl-3-phenyl-urea; [179]
3-(4-fluorophenyl)-1-[(5-methyl-2-furyl)methyl]-1-oxazol-2-yl-urea; [180]
“1-cyclopentyl-1-[(5-methyl-2-furyl)methyl]-3-(2-phenylethyl)urea; [181]”
3-(4-chlorophenyl)-1-[(5-methyl-2-furyl)methyl]-1-(1-methylimidazol-2-yl) urea; [182]
“3-(4-chlorophenyl)-1-(1H-imidazol-5-yl)-1-[(5-methyl-2-furyl)methyl]urea; [183]”
3-(4-cyanophenyl)-1-[1-(3-cyanophenyl)ethyl]-1-cyclobutyl-urea; [184]
3-(4-chlorophenyl)-1-cyclopentyl-1-[1-(5-methyl-2-furyl)ethyl]urea; [185]
1-[1-(3-cyanophenyl)ethyl]-1-cyclopentyl-3-phenyl-urea; [186]
1-[1-(3-cyanophenyl)ethyl]-1-cyclopentyl-3-(4-fluorophenyl)urea; [187]
3-(4-cyanophenyl)-1-[1-(3-cyanophenyl)ethyl]-1-cyclopentyl-urea; [188]
1-[1-(3-cyanophenyl)ethyl]-1-cyclobutyl-3-(4-fluorophenyl)urea; [189]
3-(4-cyanophenyl)-1-cyclopentyl-1-[1-(5-methyl-2-furyl)ethyl]urea; [190]
1-cyclopentyl-3-(4-fluorophenyl)-1-[1-(5-methyl-2-furyl)ethyl]urea; [191]
3-(4-chlorophenyl)-1-[1-(3-cyanophenyl)ethyl]-1-cyclobutyl-urea; [192]
3-(4-chlorophenyl)-1-[(5-methyl-2-furyl)methyl]-1-(3-methylimidazol-4-yl)urea;
3-(4-chlorophenyl)-1-[(2-cyano-4-pyridyl)methyl]-1-(3-methylisoxazol-4-yl)urea[198]
3-(4-cyano-3-methoxy-phenyl)-1-cyclopentyl-1-[(5-methyl-2-furyl)methyl]urea[205];
3-(4-chlorophenyl)-1-(2,2-difluorocyclopentyl)-1-[(5-methyl-2-furyl)methyl]urea; [206]
3-(4-cyanophenyl)-1-cyclobutyl-1-(pyrazolo[1,5-a]pyridin-2-ylmethyl)urea; [207]
3-(4-cyanophenyl)-1-(3-fluorocyclobutyl)-1-[(5-methyl-2-furyl)methyl]urea; [208]
1-(1,3-benzoxazol-6-ylmethyl)-3-(4-cyanophenyl)-1-cyclobutyl-urea; [209]
3-(4-cyanophenyl)-1-cyclobutyl-1-(imidazo[1,2-a]pyridin-2-ylmethyl)urea; [210]
3-(4-cyanophenyl)-1-cyclobutyl-1-(imidazo[1,2-a]pyrazin-2-ylmethyl)urea; [211]
3-(4-chlorophenyl)-1-cyclobutyl-1-(imidazo[1,2-a]pyrazin-2-ylmethyl)urea; [212]
1-(1,3-benzothiazol-6-ylmethyl)-3-(4-cyanophenyl)-1-cyclobutyl-urea; [213]
3-(4-cyanophenyl)-1-(2,2-difluorocyclopentyl)-1-[(5-methyl-2-furyl)methyl]urea; [214]
3-(4-cyanophenyl)-1-[(5-methyl-2-furyl)methyl]-1-(2-methylpyrazol-3-yl)urea; [215]
3-(4-cyanophenyl)-1-(3-methoxycyclobutyl)-1-[(5-methyl-2-furyl)methyl]urea; [216]
3-(4-cyanophenyl)-1-cyclobutyl-1-[(1-methylindazol-6-yl)methyl]urea; [217]
3-(4-cyanophenyl)-1-[(5-methyl-2-furyl)methyl]-1-(1-methylpyrazol-4-yl)urea; [218]
1-(2-cyanocyclopentyl)-3-(4-cyanophenyl)-1-[(5-methyl-2-furyl)methyl]urea; [219]
3-(4-chlorophenyl)-1-[(5-methyl-2-furyl)methyl]-1-(2-methylpyrazol-3-yl)urea; [220]
3-(4-cyanophenyl)-1-[(5-methyl-2-furyl)methyl]-1-(1-methylpyrazol-3-yl)urea; [221]
3-(4-chlorophenyl)-1-(3-fluorocyclobutyl)-1-[(5-methyl-2-furyl)methyl]urea; [222]
1-(1,3-benzoxazol-5-ylmethyl)-3-(4-cyanophenyl)-1-cyclobutyl-urea; [223]
1-(3-cyanocyclopentyl)-3-(4-cyanophenyl)-1-[(5-methyl-2-furyl)methyl]urea; [224]
3-(4-chlorophenyl)-1-[(5-methyl-2-furyl)methyl]-1-(1-methylpyrazol-4-yl)urea; [225]
3-(4-chlorophenyl)-1-[(5-methyl-2-furyl)methyl]-1-(1-methylpyrazol-3-yl)urea; [226]
3-(4-chlorophenyl)-1-(2-cyanocyclopentyl)-1-[(5-methyl-2-furyl)methyl]urea; [227]
1-cyclopentyl-1-[(5-methyl-2-furyl)methyl]-3-[6-(trifluoromethyl)-3-pyridyl]urea; [228]
1-(1,3-benzothiazol-2-ylmethyl)-3-(4-cyanophenyl)-1-cyclobutyl-urea; [229]
3-(4-cyanophenyl)-1-(3,5-dimethylisoxazol-4-yl)-1-[(5-methyl-2-furyl)methyl]urea; [230]
3-(4-cyanophenyl)-1-[(5-methyl-2-furyl)methyl]-1-(3-methylimidazol-4-yl)urea; [231]
1-(1,3-benzoxazol-5-ylmethyl)-3-(4-chlorophenyl)-1-cyclobutyl-urea; [232]
3-(4-cyanophenyl)-1-[(5-methyl-2-furyl)methyl]-1-(1-methylimidazol-2-yl)urea; [233]
1-(benzofuran-2-ylmethyl)-3-(4-cyanophenyl)-1-cyclobutyl-urea; [234]
3-(4-chlorophenyl)-1-cyclobutyl-1-[(1-methylindazol-6-yl)methyl]urea; [235]
3-(4-ethynylphenyl)-1-[(5-methyl-2-furyl)methyl]-1-(oxetan-3-yl)urea; [236]
1-(1,3-benzoxazol-6-ylmethyl)-3-(4-chlorophenyl)-1-cyclobutyl-urea; [237]
3-(4-chlorophenyl)-1-(3,5-dimethylisoxazol-4-yl)-1-[(5-methyl-2-furyl)methyl]urea; [238]
3-(4-chlorophenyl)-1-cyclobutyl-1-(pyrazolo[1,5-a]pyridin-2-ylmethyl)urea; [239]
1-(2,1,3-benzothiadiazol-5-ylmethyl)-3-(4-chlorophenyl)-1-cyclobutyl-urea; [240]
3-(4-cyanophenyl)-1-(3,3-difluorocyclobutyl)-1-[(5-methyl-2-furyl)methyl]urea; [241]
3-(4-chlorophenyl)-1-[(5-methyl-2-furyl)methyl]-1-(3-methylimidazol-4-yl) urea; [242]
1-(1,3-benzothiazol-6-ylmethyl)-3-(4-chlorophenyl)-1-cyclobutyl-urea; [243]
3-(4-cyanophenyl)-1-(3,3-difluorocyclopentyl)-1-[(5-methyl-2-furyl)methyl]urea; [244]
3-(4-chlorophenyl)-1-(3,3-difluorocyclopentyl)-1-[(5-methyl-2-furyl)methyl]urea; [245]
1-cyclopentyl-1-[(5-methyl-2-furyl)methyl]-3-(6-methyl-3-pyridyl)urea; [246]
3-(4-chlorophenyl)-1-(3,3-difluorocyclobutyl)-1-[(5-methyl-2-furyl)methyl]urea; [247]
1-(1,3-benzothiazol-2-ylmethyl)-3-(4-chlorophenyl)-1-cyclobutyl-urea; [248]
3-(4-cyanophenyl)-1-cyclobutyl-1-(furo[3,2-b]pyridin-2-ylmethyl)urea; [249]
3-(4-chlorophenyl)-1-cyclobutyl-1-(imidazo[1,2-a]pyridin-2-ylmethyl)urea; [250]
3-(4-chlorophenyl)-1-(3-methoxycyclobutyl)-1-[(5-methyl-2-furyl)methyl]urea; [251]
3-(4-chlorophenyl)-1-cyclobutyl-1-(furo[3,2-b]pyridin-2-ylmethyl)urea; [252]
1-cyclopentyl-3-(6-methoxy-3-pyridyl)-1-[(5-methyl-2-furyl)methyl]urea; [253]
3-(4-chlorophenyl)-1-(3-cyanocyclopentyl)-1-[(5-methyl-2-furyl)methyl]urea; [254]
1-(2,1,3-benzothiadiazol-5-ylmethyl)-3-(4-cyanophenyl)-1-cyclobutyl-urea; [255]
3-(4-chlorophenyl)-1-[(5-methyl-2-furyl)methyl]-1-(5-methylisoxazol-4-yl)urea; [256]
3-(4-cyanophenyl)-1-[(5-methyl-2-furyl)methyl]-1-(5-methylisoxazol-4-yl)urea; [257]
3-(4-ethynylphenyl)-1-[(5-methyl-2-furyl)methyl]-1-(1-methyl-4-piperidyl)urea; [258]
3-(4-ethynylphenyl)-1-[(5-methyl-2-furyl)methyl]-1-tetrahydropyran-4-yl-urea; [259]
1-(1,3-benzothiazol-5-ylmethyl)-3-(4-chlorophenyl)-1-cyclobutyl-urea; [260]
1-(1,3-benzothiazol-5-ylmethyl)-3-(4-cyanophenyl)-1-cyclobutyl-urea; [261]
1-(1,3-benzothiazol-7-ylmethyl)-3-(4-chlorophenyl)-1-cyclobutyl-urea; [262]
1-(1,3-benzothiazol-7-ylmethyl)-3-(4-cyanophenyl)-1-cyclobutyl-urea; [263]
1-(1,3-benzothiazol-6-ylmethyl)-3-(4-cyanophenyl)-1-(1-methyl-4-piperidyl) urea; [264]
1-(1,3-benzothiazol-6-ylmethyl)-3-(4-cyanophenyl)-1-tetrahydropyran-4-yl-urea; [265]
3-(4-cyanophenyl)-1-[(5-methyl-2-furyl)methyl]-1-(2-methyl-1,2,4-triazol-3-yl) urea[266];
3-(4-chlorophenyl)-1-[(5-methyl-2-furyl)methyl]-1-(3-methyltriazol-4-yl)urea[267];
3-(4-cyanophenyl)-1-[(5-methyl-2-furyl)methyl]-1-(3-methyltriazol-4-yl)urea[268];
3-(4-chlorophenyl)-1-cyclobutyl-1-(imidazo[2,1-b]thiazol-6-ylmethyl)urea[269];
3-(4-cyanophenyl)-1-cyclobutyl-1-(imidazo[2,1-b]thiazol-6-ylmethyl)urea[270];
3-(4-chlorophenyl)-1-(3-fluorocyclobutyl)-1-[(5-methyl-2-furyl)methyl]urea[271];
3-(4-cyanophenyl)-1-(3-fluorocyclobutyl)-1-[(5-methyl-2-furyl)methyl]urea[272];
3-(4-chlorophenyl)-1-(3-fluorocyclobutyl)-1-[(5-methyl-2-furyl)methyl]urea[273];
3-(4-cyanophenyl)-1-cyclobutyl-1-(imidazo[1,2-a]pyrimidin-7-ylmethyl)urea[274];
3-(4-chlorophenyl)-1-cyclobutyl-1-(imidazo[1,2-a]pyrimidin-7-ylmethyl)urea[275];
1-(1,3-benzothiazol-6-ylmethyl)-3-(4-cyanophenyl)-1-(2-methyl-iH-pyrazol-3-yl) urea[276];
3-(4-chlorophenyl)-1-[(5-methyl-2-furyl)methyl]-1-(3-methylisoxazol-4-yl)urea[277];
3-(4-cyanophenyl)-1-[(5-methyl-2-furyl)methyl]-1-(3-methylisoxazol-4-yl)urea[278];
3-[4-(3-methoxyprop-1-ynyl)phenyl]-1-[(5-methyl-2-furyl)methyl]-1-(5-methyl isoxazol-4-yl)urea[279];
1-(1,3-benzothiazol-6-ylmethyl)-3-[4-(3-hydroxyprop-1-ynyl)phenyl]-1-(2-methylpyrazol-3-yl)urea[280];
1-(1,3-benzothiazol-5-ylmethyl)-3-(4-cyanophenyl)-1-(5-methylisoxazol-4-yl)urea[281];
3-(4-chlorophenyl)-1-[(5-methyl-2-furyl)methyl]-1-(2-pyridyl)urea[282];
3-(4-cyanophenyl)-1-[(5-methyl-2-furyl)methyl]-1-(2-pyridyl)urea[283];
1-(1-bicyclo[1.1.1]pentanyl)-3-(4-chlorophenyl)-1-[(5-methyl-2-furyl)methyl]urea[284];
1-(1-bicyclo[1.1.1]pentanyl)-3-(4-cyanophenyl)-1-[(5-methyl-2-furyl)methyl]urea[285];
3-(4-chlorophenyl)-1-[(5-methyl-2-furyl)methyl]-1-(2-oxaspiro[3.3]heptan-6-yl)urea[286];
3-(4-cyanophenyl)-1-[(5-methyl-2-furyl)methyl]-1-(2-oxaspiro[3.3]heptan-6-yl)urea[287];
3-(4-cyanophenyl)-1-(5-methylisoxazol-4-yl)-1-[[2-(trifluoromethyl)-4-pyridyl]methyl]urea[288];
1-cyclopentyl-1-[(5-methyl-2-furyl)methyl]-3-[4-(trifluoromethoxy)phenyl]urea[289];
1-cyclopentyl-3-(4-methoxyphenyl)-1-[(5-methyl-2-furyl)methyl]urea[290];
3-(4-cyanophenyl)-1-cyclopentyl-1-[(2-methyloxazol-5-yl)methyl]urea[291];
1-[(5-methyl-2-furyl)methyl]-1-(5-methylisoxazol-4-yl)-3-[4-(trifluoromethoxy)phenyl]urea[292];
3-(4-cyanophenyl)-1-(5-methylisoxazol-4-yl)-1-[[3-(trifluoromethyl)phenyl]methyl]urea[293];
1-cyclopentyl-3-[4-(3-hydroxyprop-1-ynyl)phenyl]-1-[(2-methyloxazol-5-yl)methyl]urea[294];
3-(4-cyanophenyl)-1-cyclopentyl-1-[(5-ethyl-2-furyl)methyl]urea[295];
1-cyclobutyl-3-[4-(3-hydroxyprop-1-ynyl)phenyl]-1-[[2-(trifluoromethyl)-4-pyridyl]methyl]urea[296];
1-cyclobutyl-3-[4-(3-hydroxyprop-1-ynyl)phenyl]-1-[(5-methyl-2-furyl)methyl]urea[297];
3-(4-chlorophenyl)-1-[(5-methyl-2-furyl)methyl]-1-(4-methyl-1,2,5-oxadiazol-3-yl)urea[298];
3-(4-cyanophenyl)-1-[(5-ethyl-2-furyl)methyl]-1-(5-methylisoxazol-4-yl)urea[299];
1-(1,3-benzothiazol-2-yl)-3-(4-chlorophenyl)-1-[(5-methyl-2-furyl)methyl]urea[300];
3-[4-(difluoromethoxy)phenyl]-1-[(5-methyl-2-furyl)methyl]-1-(5-methylisoxazol-4-yl)urea[301];
3-(4-chlorophenyl)-1-(2-methylpyrazol-3-yl)-1-[[2-(trifluoromethyl)-4-pyridyl]methyl]urea[302];
1-cyclopentyl-3-(4-hydroxyphenyl)-1-[(5-methyl-2-furyl)methyl]urea[303];
3-(4-cyanophenyl)-1-isoxazol-4-yl-1-[(5-methyl-2-furyl)methyl]urea[304];
3-(4-chlorophenyl)-1-isoxazol-4-yl-1-[(5-methyl-2-furyl)methyl]urea[305];
1-cyclobutyl-3-[4-(3-hydroxyprop-1-ynyl)phenyl]-3-methyl-1-[(5-methyl-2-furyl)methyl]urea[306];
1-cyclopentyl-1-[(5-methyl-2-furyl)methyl]-3-(1-oxoisoindolin-5-yl)urea[307];
3-[4-[4-(hydroxymethyl)triazol-1-yl]phenyl]-1-(3-methylisoxazol-4-yl)-1-[[3-(trifluoromethyl)phenyl]methyl]urea[308];
3-[4-(3-hydroxyprop-1-ynyl)phenyl]-1-[(5-methyl-2-furyl)methyl]-1-(1-methylimidazol-2-yl)urea[309];
3-(4-cyanophenyl)-1-cyclopentyl-1-(6,7-dihydro-4H-pyrano[4,3-d]thiazol-2-ylmethyl)urea[310];
3-(4-chlorophenyl)-1-cyclopentyl-1-(6,7-dihydro-4H-pyrano[4,3-d]thiazol-2-ylmethyl)urea[311];
1-cyclopentyl-1-[(5-methyl-2-furyl)methyl]-3-(2-methyl-1-oxo-isoindolin-5-yl)urea[312];
3-(4-chlorophenyl)-1-[(6-cyano-2-pyridyl)methyl]-1-(3-methylisoxazol-4-yl)urea[313];
3-(4-cyanophenyl)-1-[(5-methyl-2-furyl)methyl]-1-(3-methyl-2-pyridyl)urea[314];
3-[4-(3-hydroxyprop-1-ynyl)phenyl]-1-(3-methylisoxazol-4-yl)-1-[[3-(trifluoromethyl)phenyl]methyl]urea[315];
3-(4-cyanophenyl)-1-(3-methylisoxazol-4-yl)-1-[(2-methyloxazol-5-yl)methyl]urea[316];
1-cyclopentyl-1-[(5-methyl-2-furyl)methyl]-3-[4-(oxetan-3-yl)phenyl]urea[317];
3-(4-chlorophenyl)-1-[(5-methyl-2-furyl)methyl]-1-(3-methyl-2-pyridyl)urea[318];
1-[(6-cyano-2-pyridyl)methyl]-1-cyclobutyl-3-[4-(3-hydroxyprop-1-ynyl)phenyl]urea[319];
1-cyclopentyl-1-[(5-methyl-2-furyl)methyl]-3-[4-(4-methyltriazol-1-yl)phenyl]urea[320];
3-[4-(3-hydroxyprop-1-ynyl)phenyl]-1-(3-methylisoxazol-4-yl)-1-[(2-methyloxazol-5-yl)methyl]urea[321];
3-(4-cyanophenyl)-1-[(5-methyl-2-furyl)methyl]-1-(4-methyl-1,2,5-oxadiazol-3-yl)urea[322];
1-cyclopentyl-3-[4-[4-(hydroxymethyl)triazol-1-yl]-3-methoxy-phenyl]-1-[(5-methyl-2-furyl)methyl]urea[323];
3-(4-cyanophenyl)-1-[(5-methyl-2-furyl)methyl]-1-pyrimidin-2-yl-urea[324];
3-(4-cyanophenyl)-1-(3-methylisoxazol-4-yl)-1-(2-oxaspiro[3.5]nonan-7-ylmethyl)urea[325];
3-(4-chlorophenyl)-1-(3-methylisoxazol-4-yl)-1-(2-oxaspiro[3.5]nonan-7-ylmethyl)urea[326];
3-(4-chlorophenyl)-1-[(5-methyl-2-furyl)methyl]-1-pyrimidin-2-yl-urea[327]; and
3-(4-chlorophenyl)-1-[(5-methyl-2-furyl)methyl]-1-(3-methylisothiazol-4-yl)urea[328];

or a pharmaceutically acceptable salt, or a solvate, or a solvate of the salt thereof.

Specific compounds of formula I for use as a medicament according to the invention which may be mentioned include those aforementioned compounds and selected from the group consisting of
N-(3,5-dimethylphenyl)-3-ethyl-2-methyl-7-phenyl-5,7-dihydro-4H-thieno[2,3-c]pyridine-6-carboxamide; [194]
1-cyclopentyl-3-phenyl-1-(2-thienylmethyl)urea; [195]
1-(4-chlorophenyl)-3-phenyl-1-(2-thienylmethyl)urea; [196]
1-[1-(4-fluorophenyl)ethyl]-3-phenyl-urea; [197]
1-(4-chlorophenyl)-3-[1-(5-chloro-2-thienyl)ethyl]urea; [199]
3-(3,4-dichlorophenyl)-1-methyl-1-(2-thienylmethyl)urea; [200]
1-[(5-methyl-2-phenyl-oxazol-4-yl)methyl]-3-phenyl-urea; [203] and
1-(3-chlorophenyl)-3-[(3-chloro-2-thienyl)methyl]urea; [204];

or a pharmaceutically acceptable salt, or a solvate, or a solvate of the salt thereof.

In one aspect of the invention there is provided compounds of formula I:

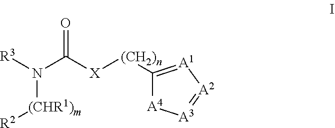

or a pharmaceutically acceptable salt, or a solvate, or a solvate of the salt thereof,

wherein:

X, n and R¹ are each as herein defined;

m is 1;

R² is a 5-6-membered heteroaryl, a fused 9-10 membered bicyclic heteroaryl, phenyl; each of which may independently be optionally substituted by one or more groups independently selected from C₁₋₆alkyl, halogen, haloC₁₋₆alkyl, —CN and haloC₁₋₆ alkyloxy;

R³ is a 4 or 5 membered cycloalkyl, a 5-6-membered heteroaryl or an isoxazole; each of which may independently be optionally substituted by one or more groups independently selected from —C₁₋₆alkyl, —OC₁₋₆alkyl, halogen and —CN;

the moiety

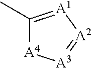

is phenyl or thiazole, each of which may independently be optionally substituted by one or more groups independently selected from —C₁₋₆alkyl, halogen, haloC₁₋₆alkyl, haloC₁₋₆alkyl-O—, —CN, —C₁₋₆alkyl-OH, —C₂₋₆alkynyl, C₂₋₆alkynyl-C₁₋₆alkyl, —C₂₋₆alkynyl-C₃₋₆cycloalkyl, —C₂₋₆alkynyl-C₁₋₆alkyl-OR¹³ and a 5-6 membered heteroaryl; and

R¹³ is H or C₁₋₆alkyl.

According to this aspect of the invention, specific compounds of formula I which may be mentioned include those selected from the group consisting of Namely compounds 26, 52, 56, 80, 83, 84, 89, 90, 91, 92, 102, 107, 111, 130, 131, 137, 139, 140, 141, 146, 147, 148, 153, 154, 161, 162, 163, 164, 173, 177, 185, 192, 206, 213, 214, 216, 227, 232, 234, 235, 240, 256, 257, 260, 261, 262, 277, 278, 279, 289, 294, 295, 297, 298, 301, 305, 315, 318, 319, 322 and 328.

In one aspect of the invention there is provided compounds of formula I:

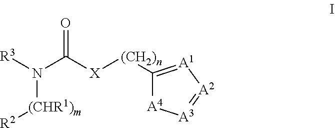

or a pharmaceutically acceptable salt, or a solvate, or a solvate of the salt thereof,

wherein:

X and R¹ is as herein defined;

m is 1;

n is 0;

R² is a 5-6-membered heteroaryl, a fused 9-10 membered bicyclic heteroaryl, a 6-membered aryl, a 5-6 membered monocyclic heterocycloalkyl or a fused 8-10 membered partially unsaturated bicyclic heterocyclyl; each of which may independently be optionally substituted by one or more groups independently selected from C₁₋₆alkyl, haloC₁₋₆alkyl, halogen, —OC₁₋₆alkyl, —CN and —C(═O)OC₁₋₆alkyl;

R³ is a 4 or 5 membered cycloalkyl, a 5-6 membered heteroaryl or a 4-6 membered monocyclic heterocycloalkyl each of which may independently be optionally substituted by one or more groups independently selected from C₁₋₆alkyl, —OC₁₋₆alkyl, halogen, and —CN;

the moiety

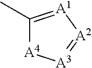

is phenyl, pyridine, pyrimidine or thiazole, each of which may independently be optionally substituted by one or more groups independently selected from —C₁₋₆alkyl, halogen, haloC₁₋₆alkyl, haloC₁₋₆alkyl-O—, —CN, —OC₁₋₆alkyl, —C₂₋₆alkynyl, —C₂₋₆alkynyl-C₁₋₆alkyl-NR¹¹R¹², —C₂₋₆alkynyl-C₁₋₆alkyl-OR¹³, a 6-10 membered aryl, a 5-6 membered heteroaryl, each of which may independently be optionally substituted by one or more groups independently selected from —C₁₋₆alkyl or —C₁₋₆alkyl-OH; and

R¹¹, R¹² and R¹³, which may be the same or different, are each selected from H and C₁₋₆alkyl.

According to this aspect of the invention, specific compounds of formula I which may be mentioned include those selected from the group consisting of:

Namely compounds 3, 16, 17, 25, 27, 30, 31, 34, 40, 42, 45, 47, 48, 50, 53, 66, 69, 77, 78, 79, 82, 98, 105, 108, 110, 114, 115, 116, 120, 124, 132, 133, 144, 150, 152, 157, 172, 174, 176, 182, 184, 186, 187, 188, 189, 190, 207, 217, 219, 222, 223, 224, 225, 226, 228, 229, 230, 237, 238, 239, 242, 243, 244, 245, 247, 248, 253, 255, 259, 263, 267, 269, 271, 273, 194, 195, 205, 284, 285, 290, 292, 293, 296, 299, 304, 309, 311, 313, 314, 320 and 323.

In one aspect of the invention there is provided compounds of formula I:

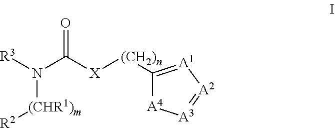

or a pharmaceutically acceptable salt, or a solvate, or a solvate of the salt thereof,

wherein:

X is as herein defined;

m is 1;

n is 0;

R¹ is H or C₁₋₆alkyl;

R² is a thiophene, furan, pyrazine, pyridine, isoxazole, benzoxazole, imidazothiazole or phenyl; each of which may independently be optionally substituted by one or more groups independently selected from C₁₋₆alkyl, halogen, haloC₁₋₆alkyl, —CN;

R³ is H, 4 or 5 membered cycloalkyl, imidazole, or oxetane; each of which may independently be optionally substituted by one or more groups independently selected from —C₁₋₆alkyl, —OC₁₋₆alkyl, halogen and —CN;

the moiety

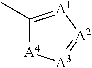

is phenyl, pyridine, benzothiazole, benzofuran, each of which may independently be optionally substituted by one or more groups independently selected from —C₁₋₆alkyl, halogen, —CN, —C₂₋₆alkynyl, —C₂₋₆alkynyl-aryl, —C₂₋₆alkynyl-C₁₋₆alkyl-aryl, —C₂₋₆alkynyl-C₁₋₆alkyl-NR¹¹R¹²; or a 5-6 membered heteroaryl, which may be optionally substituted by one or more groups independently selected from —C(═O)OC₁₋₆alkyl, thiophene, phenyl and —C₁₋₆alkyl-OH; and

R¹¹ and R¹², which may be the same or different, are each selected from H and C₁₋₆ alkyl.

In one aspect of the invention, specific compounds of formula I which may be mentioned include those selected from the group consisting of:

Namely compounds 4, 19, 28, 38, 44, 55, 59, 60, 71, 76, 81, 88, 99, 106, 112, 119, 121, 126, 128, 134, 149, 151, 155, 156, 158, 166, 168, 175, 191, 193, 208, 209, 218, 221, 236, 241, 246, 251, 254, 268, 270, 272, 199, 282, 291 and 308.

In one aspect of the invention there is provided compounds of formula I:

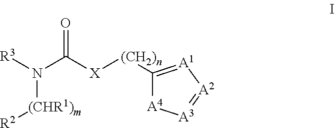

or a pharmaceutically acceptable salt, or a solvate, or a solvate of the salt thereof,

wherein:

X is as herein defined;

m is 1;

n is 0 or 2;

R¹ is H or C₁₋₆alkyl;

R² is a 5 or 6 membered heteroaryl, a fused 9 or 10 membered bicyclic heteroaryl, a 6 membered aryl, a 5 or 6 membered monocyclic heterocycloalkyl or a fused 8-10 membered partially unsaturated bicyclic heterocyclyl; each of which may independently be optionally substituted by one or more groups independently selected from C₁₋₆alkyl, halogen, —OC₁₋₆alkyl, —CN, —C(═O)C₁₋₆alkyl —C(═O)OC₁₋₆alkyl, —SO₂—C₁₋₆alkyl, —C(═O)NH₂, haloC₁₋₆alkyloxy and phenyl;

R³ is H or C₁₋₆alkyl; or a 3-6 membered cycloalkyl, a 6 membered aryl, a 5-6 membered heteroaryl, a fused 9-10 membered bicyclic heteroaryl, a 4-6 membered monocyclic heterocycloalkyl or a 5-11 membered spiroheteroalkyl; each of which may independently be optionally substituted by one or more —C₁₋₆alkyl;

the moiety

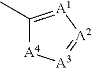

is phenyl, benzodioxole, indane, pyridine, thiophene or thiazole, each of which may independently be optionally substituted by one or more groups independently selected from —C₁₋₆alkyl, halogen, haloC₁₋₆alkyl, —CN, —OC₁₋₆ alkyl, —C₁₋₆alkyl-CN, —C₂₋₆alkynyl-C₁₋₆alkyl-OR¹³, —C(═O)C₁₋₆alkyl, —C(═O)NH₂, —C(═O)OC₁₋₆alkyl and oxopyrrolidine a 5 or 6 membered cycloalkyl, a 4-6 membered monocyclic heterocycloalkyl, a 6 membered aryl, a 5 or 6 membered heteroaryl, a 5 or 6 membered heteroC₃₋₆cycloalkyl, each of which may independently be optionally substituted by one or more groups independently selected from —C₁₋₆alkyl, —C(═O)OC₁₋₆alkyl; and

R¹³ is each selected from H and C₁₋₆alkyl.

According to this aspect of the invention, specific compounds of formula I which may be mentioned include those selected from the group consisting of:

Namely compounds 1, 2, 5, 6, 7, 9, 10, 11, 13, 15, 18, 21, 35, 36, 37, 41, 43, 46, 49, 54, 58, 63, 64, 65, 67, 68, 73, 85, 86, 93, 94, 95, 97, 103, 109, 113, 117, 118, 122, 125, 129, 135, 138, 142, 143, 145, 159, 160, 165, 167, 169, 170, 171, 178, 181, 183, 210, 212, 231, 233, 250, 265, 266, 274, 275, 276, 196, 197, 198, 200, 203, 280, 281, 283, 286, 288, 300, 302, 306, 310, 312, 317, 321 and 327.

In one aspect of the invention there is provided compounds of formula I:

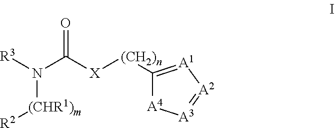

or a pharmaceutically acceptable salt, or a solvate, or a solvate of the salt thereof for use as a medicament, wherein:

X and n are each as herein defined;

m is 1;

R¹ is H or C₁₋₆alkyl;

R² is a 5-6-membered heteroaryl, a fused 9-10 membered bicyclic heteroaryl, a 6 membered aryl, a 5-6 membered monocyclic heterocycloalkyl or a 5-11 membered spiroheteroalkyl; each of which may independently be optionally substituted by one or more groups independently selected from C₁₋₆alkyl, halogen, —CN;

R³ is H or a 5 or 6 membered cycloalkyl, a 5 membered heteroaryl, a 6 membered monocyclic heterocycloalkyl, a 5-11 membered spiroheteroalkyl or a —C₁₋₆alkyl-heteroaryl; each of which may independently be optionally substituted by one or more groups independently selected from —C₁₋₆alkyl, halogen and —C(═O)OC₁₋₆alkyl;

the moiety

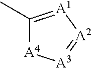

is phenyl, pyridine or phenyl fused with oxopyrrolidine, each of which may independently be optionally substituted by one or more groups independently selected from —OH, —C₁₋₆alkyl, halogen, —CN, —OC₁₋₆alkyl, —C₂₋₆alkynyl, —C(═O)C₁₋₆alkyl, a 5-6 membered heteroaryl, a 5-6 membered heteroC₃₋₆cycloalkyl, each of which may independently be optionally substituted by one or more groups independently selected from —C₁₋₆alkyl-NR⁹R¹⁰ and —C₁₋₆alkyl-OH;

R⁹ and R¹⁰, which may be the same or different, are each selected from H and C₁₋₆ alkyl.

According to this aspect of the invention, specific compounds of formula I which may be mentioned include those selected from the group consisting of:

Namely compounds 8, 14, 20, 22, 23, 24, 29, 32, 33, 39, 51, 57, 61, 62, 70, 72, 74, 75, 87, 96, 100, 101, 104, 123, 127, 136, 179, 180, 211, 215, 220, 249, 252, 258, 264, 204, 287, 303, 307, 316, 324, 325 and 326.

In one embodiment the compound and intermediate of the invention is not an isotopic variant.

In one aspect a compound and intermediate of the invention according to any one of the embodiments herein described is a free base.

In one aspect a compound and intermediate of the invention according to any one of the embodiments herein described is a salt.

In one aspect a compound of the invention according to any one of the embodiments herein described is a pharmaceutically acceptable salt.

In one aspect a compound and intermediate of the invention according to any one of the embodiments herein described is a solvate of the compound.

In one aspect a compound of the invention according to any one of the embodiments herein described is a solvate of a salt of a compound, in particular a solvate of a pharmaceutically acceptable salt.

Similarly, reference to intermediates of the invention, whether or not they themselves are claimed, is meant to embrace their salts, and solvates, where the context so permits.

With regard to stereoisomers, the compounds and intermediates of the invention have more than one asymmetric carbon atom. In the general formula(e) as drawn, the solid wedge shaped bond indicates that the bond is above the plane of the paper. The broken bond indicates that the bond is below the plane of the paper.

It will be appreciated that the substituents on the compounds and intermediates of the invention may also have one or more asymmetric carbon atoms. Thus, the compounds and intermediates of the invention may occur as individual enantiomers or diastereomers. All such isomeric forms are included within the present invention, including mixtures thereof.

Where a compound and intermediate of the invention contains an alkenyl group, cis (Z) and trans (E) isomerism may also occur. The present invention includes the individual stereoisomers of the compound and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A stereoisomeric mixture of the agent may also be prepared from a corresponding optically pure intermediate or by resolution, such as by HPLC, of the corresponding mixture using a suitable chiral support or by fractional crystallisation of the diastereoisomeric salts formed by reaction of the corresponding mixture with a suitable optically active acid or base, as appropriate.

Unless otherwise stated, in formulae disclosed herein a bond drawn without any attached group means a methyl group.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

While specified groups for each embodiment have generally been listed above separately, a compound and intermediate of the invention may be one for which one or more variables (R groups and/or integers) is selected from one or more embodiments according to any of the Formula(e) listed above. Therefore, the present invention is intended to include all combinations of variables from any of the disclosed embodiments within its scope.

Alternatively, the exclusion of one or more of the specified variables from a group or an embodiment, or combinations thereof is also contemplated by the present invention.

In certain aspects, the present invention provides prodrugs and derivatives of the compounds of the invention according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H. Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C1 to C8 alkyl, C2-C8 alkenyl, aryl, substituted aryl, and arylalkyl esters of the compounds of the invention.

A person of skill in the art will appreciate that when administered in vivo compounds of the invention may be metabolised and that some of these biological metabolites may be active. In one aspect, the present invention therefore provides for biologically active metabolites of compounds of the invention.

Pharmaceutical Compositions

While it is possible that, for use in the methods of the invention, a compound of the invention may be administered as the bulk substance, it is preferable to present the active ingredient in a pharmaceutical formulation as a pharmaceutical composition. Thus, when employed as a pharmaceutical, a compound of the invention is typically administered in the form of a pharmaceutical composition. Such compositions can be prepared in a manner well known in the pharmaceutical art and comprise at least one active compound. Generally, a compound of this invention is administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, subcutaneous, intra-articular, intravenous, intramuscular, and intranasal. Depending on the intended route of delivery, a compound of this invention is preferably formulated as either injectable or oral compositions or as salves, as lotions or as patches all for transdermal administration. The compounds of the invention can be administered for immediate-, delayed-, modified-, sustained-, pulsed- or controlled-release applications.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

In one aspect, oral compositions are slow, delayed or positioned release (e.g., enteric especially colonic release) tablets or capsules. This release profile can be achieved for example, by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of methacrylic acid and its esters, and combinations thereof. Plasticizers such as, but not limited to polyethylene glycol, dibutylphthalate, triacetin and castor oil may be used.

A pigment may also be used to colour the film. Suppositories are be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C8-C18 triglycerides), esterification of glycerol and specific fatty acids, or polyglycosylated glycerides, and witepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated castor oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and methacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilisers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminium silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminium silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable flavourings for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the compound of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and flavourings.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (IFA 134AT) or 1,1,1,2,3,3,3-heptafluoropropane (IFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10% w/w, more preferably 0.01-1% w/w of the active compound.

A therapeutically effective amount of the compound of the present invention can be determined by methods known in the art. The therapeutically effective quantities may vary and will depend on the severity of the disease, the age and the general physiological condition of the subject, the potency of the compound, the route of administration and the pharmaceutical formulation used. The therapeutic doses will generally be from about 10 to 2000 mg/day and suitably from about 30 to 1500 mg/day. Other ranges may be used, including, for example, 50-500 mg/day, 50-300 mg/day, 100-200 mg/day. Thus, the therapeutic dose may be about 10 mg/day, about 10 mg/day, about 50 mg/day, about 100 mg/day, about 150 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 1,000 mg/day, about 1,050 mg/day, about 1,100 mg/day, about 1,150 mg/day, about 1,200 mg/day, about 1,250 mg/day, about 1,300 mg/day, about 1,350 mg/day, about 1,400 mg/day, about 1,450 mg/day, about 1,500 mg/day, about 1,550 mg/day, about 1,600 mg/day, about 1,650 mg/day, about 1,700 mg/day, about 1,750 mg/day, about 1,800 mg/day, about 1,850 mg/day, about 1,900 mg/day, about 1,950 mg/day or about 2,000. The daily dose as employed for acute human treatment will range from 0.01 to 40 mg/kg body weight, suitably 2 to 20 mg/kg body weight, or suitably 5 to 10 mg/kg body weight, which may be administered in one to four daily doses, for example, depending on the route of administration and the condition of the subject. When the composition comprises dosage units, each unit may contain 10 mg to 2 g of active ingredient, suitably 200 mg to 1 g of active ingredient.

Administration may be once a day, twice a day, or more often, and may be decreased during a maintenance phase of treatment of the disease, e.g. once every second or third day instead of every day or twice a day. The dose and the administration frequency will depend on the clinical signs with the reduction or absence of at least one or more, preferably more than one, clinical signs of the acute phase known to the person skilled in the art. In one aspect of the present invention, administration is once daily oral dosing.

The present invention is related to a pharmaceutical composition comprising from about 10 to 2000 mg of a compound of Formula (I), or a pharmaceutically acceptable salt thereof, and at least one pharmaceutically acceptable excipient, e.g. from about 0.1 to 2 g of one or more pharmaceutically acceptable excipients.

Methods of Treatment

It has been identified that compounds of the invention modulate tryptophan catabolism and are useful for the treatment of diseases and/or conditions associated with the abnormal or elevated catabolism of tryptophan. Such diseases and conditions include cancer, immunosuppression, viral infection, depression, a neurodegenerative disorder, trauma, age-related cataracts, organ transplant rejection, or an autoimmune disorder in a patient.

In one embodiment, the present invention provides novel compounds of the invention, or pharmaceutical compositions comprising a compound of the invention, for use as a medicament. In a particular embodiment, the present invention provides novel compounds of the invention or pharmaceutical compositions comprising a compound of the invention, for use in the treatment of conditions involving abnormal or elevated catabolism of tryptophan.

In one embodiment, the present invention provides novel compounds of the invention, or pharmaceutical compositions comprising a compound of the invention, for use as a medicament. In a particular embodiment, the present invention provides novel compounds of the invention or pharmaceutical compositions comprising a compound of the invention, for use in the treatment of conditions involving reduced levels of tryptophan.

In one embodiment, the present invention provides novel compounds of the invention, or pharmaceutical compositions comprising a compound of the invention, for use as a medicament. In a particular embodiment, the present invention provides novel compounds of the invention or pharmaceutical compositions comprising a compound of the invention, for use in the treatment of conditions involving elevated levels of kynurenine,

In one embodiment of the invention we provide a method of treatment of a disease or condition associated with, abnormal or elevated catabolism of tryptophan, reduced levels of tryptophan, or elevated levels of kynurenine, which comprises the administration of a therapeutically effective amount of a compound of Formula I to a patient suffering from such a disease or condition:

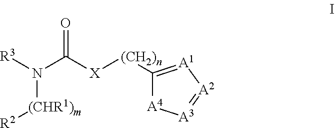

or a pharmaceutically acceptable salt, or a solvate, or a solvate of the salt thereof,

wherein:

m is 0 or 1;

n is 0, 1 or 2;

X is —NR⁸;

R¹ is H, C₁₋₆alkyl or a 6-10 membered aryl;

R² is a 5-6-membered heteroaryl, a fused 9-10 membered bicyclic heteroaryl, a 6-10 membered aryl, a 5-6 membered monocyclic heterocycloalkyl, a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy or phenyl;

R³ is H or C₁₋₆alkyl; or a 3-10 membered cycloalkyl, a 5-11 membered spiroalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆ alkyl, halogen, —CN or —C(═O)OC₁₋₆alkyl;

A¹ is —N— or —CR⁶—;

A² is —N— or —CR⁵—;

A³ is —N— or —CR⁷—;

A⁴ is —N—, —O—, —S—, —CH═N— or —CH═CR⁴—;

R⁴, R⁵, R⁶ and R⁷, which may be the same or different, are each selected from —H, —OH, —C₁₋₆alkyl, halogen, haloC₁₋₆alkyl, —CN, —C₁₋₆alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆ cycloalkyl, —C₂₋₆alkynyl-C₁₋₆alkyl-NR¹¹R¹², —C₂₋₆alkynyl-C₁₋₆alkyl-OR¹³, —C(═O)C₁₋₆ alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;

or R⁵ and R⁷ together form a ring —CH═CH—CH═CH—, —OCH₂O— or —CH₂CH₂CH₂—;

or the moiety

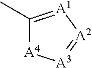

may be fused with oxopyrrolidine; and and

R⁸, R⁹, R¹⁰, R¹¹, R¹², and R¹³, which may be the same or different, are each selected from H or C₁₋₆alkyl.

In one embodiment of the invention the disease or condition associated with the abnormal or elevated catabolism of tryptophan is one or more of cancer, immunosuppression, viral infection, depression, a neurodegenerative disorder, trauma, age-related cataracts, organ transplant rejection, or an autoimmune disorder in a patient.

According to a further aspect of the invention we provide a method treatment of conditions involving the abnormal or elevated catabolism of tryptophan whereby the condition involving abnormal or elevated catabolism of tryptophan is cancer.

According to a further aspect of the invention we provide a method treatment of conditions involving reduced levels of tryptophan whereby the condition involving abnormal or elevated catabolism of tryptophan is cancer.

According to a further aspect of the invention we provide a method treatment of conditions involving the elevated levels of kynurenine whereby the condition involving abnormal or elevated catabolism of tryptophan is cancer.

According to a further aspect of the invention there is provided a method of treatment of cancer, by modulating the catabolism of tryptophan, which comprises the administration of a therapeutically effective amount of a novel compound of the invention.

According to a yet further aspect of the invention we provide a method of treating cancer as hereinbefore described wherein the cancer is selected from one or more of basal cell carcinoma, neuroectodermal tumours such as medullablastoma, meningioma, hemangioma, glioblastoma, pancreatic adenocarcinoma, squamous lung carcinoma, small-cell lung carcinoma, non-small cell lung carcinoma, chondrosarcoma, breast carcinoma, rhabdomyosarcoma, oesophageal cancer, stomach cancer, biliary tract cancer, renal carcinoma, thyroid carcinoma, primary cancer, breast cancer, colon cancer, prostate cancer, non-small cell lung cancer, glioblastoma, lymphoma, melanoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma, endometrial carcinoma, kidney cancer, renal cell cancer, and metastatic cancers.

In one preferred embodiment of the invention there is provided a method of treating cancer as hereinbefore described wherein the cancer is selected from one or more of colon cancer, endometrial carcinoma, kidney (renal) cancer, pancreatic cancer, prostate cancer, small-cell lung cancer, non-small cell lung cancer, brain cancer, ovarian cancer, cervical cancer, testicular cancer, renal cancer, head and neck cancer, lymphoma, leukaemia, and melanoma.

In a further preferred embodiment of the invention there is provided a method of treating cancer as hereinbefore described wherein the cancer is selected from one or more of endometrial carcinoma, kidney (renal) cancer, cervical cancer, non-small cell lung cancer, ovarian cancer, head and neck, pancreatic, colorectal, melanoma and bladder cancer.

According to a further aspect of the invention we provide a method treatment of conditions involving the abnormal or elevated catabolism of tryptophan whereby the condition involving abnormal or elevated catabolism of tryptophan is immunosuppression.

According to a yet further aspect of the invention we provide a method of treating immunosuppression as hereinbefore described wherein the immunosuppression is caused by one or more of IDO-mediated immunosuppression, TDO-mediated immunosuppression tryptophan catabolism mediated immunosuppression, abnormal or elevated tryptophan catabolism, reduced levels of tryptophan or elevated levels of kynurenine.

According to a yet further aspect of the invention we provide a method of treating immunosuppression as hereinbefore described wherein the immunosuppression is caused by one or more of cancer, cancer treatment, chemotherapy, radiation, viral infection, malnutrition, Ataxia-telangiectasia, Complement deficiencies, DiGeorge syndrome, Hypogammaglobulinemia, Job syndrome, Leukocyte adhesion defects, Bruton disease, Wiskott-Aldrich syndrome, Down's Syndrome, X-linked agammaglobulinemia, common variable immunodeficiency, severe combined immunodeficiency (SCID) or diabetes.

According to a further aspect of the invention we provide a method treatment of conditions involving the abnormal or elevated catabolism of tryptophan whereby the condition involving abnormal or elevated catabolism of tryptophan is viral infection. According to a yet further aspect of the invention we provide a method of treating a viral infection as hereinbefore described wherein the viral infection is selected from one or more of HIV infection, HCV infection, EBV, herpesvirus, Kaposi sarcoma-associated virus, HBV and hepatitis.

According to a further aspect of the invention we provide a method treatment of conditions involving the abnormal or elevated catabolism of tryptophan whereby the condition involving abnormal or elevated catabolism of tryptophan is a neurodegenerative disorder.

According to a yet further aspect of the invention we provide a method of treating a neurodegenerative disorder as hereinbefore described wherein the neurodegenerative disorder is selected from one or more of Huntingdon's disease, Parkinson's disease, Alzheimer's disease, Lewy body disease, amyotrophic lateral sclerosis, multiple sclerosis, AIDS dementia complex, dementia, motor neurone disease, spinal muscular atrophy, spinocerebellar ataxia stroke and epilepsy.

According to a further aspect of the invention we provide a method treatment of conditions involving the abnormal or elevated catabolism of tryptophan whereby the condition involving abnormal or elevated catabolism of tryptophan is a mood disorder such as depression.

According to a further aspect of the invention we provide a method treatment of conditions involving the abnormal or elevated catabolism of tryptophan whereby the condition involving abnormal or elevated catabolism of tryptophan is an autoimmune disorder.

According to a yet further aspect of the invention we provide a method of treating an autoimmune disorder as hereinbefore described wherein the autoimmune disorder is selected from one or more of asthma, rheumatoid arthritis, multiple sclerosis, allergic inflammation, inflammatory bowel disease, psoriasis and systemic lupus erythematosus.

A compound of the invention can be administered as the sole active agent or it can be administered in combination with a second therapeutic agent, including other compounds that demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

Thus, according to this aspect of the invention there is provided a compound of Formula I, or a pharmaceutically acceptable salt thereof, as herein described, in combination with a second therapeutically active ingredient.

In one embodiment, a compound of the invention or a pharmaceutical composition comprising the compound of the invention is administered as a medicament. In a specific embodiment, said pharmaceutical composition additionally comprises a further active ingredient. Thus, according to this aspect of the invention there is further provided a pharmaceutical composition comprising a compound of Formula I, or a pharmaceutically acceptable salt thereof, in combination with a second therapeutically active ingredient, optionally in association with a pharmaceutically acceptable adjuvant, diluent or carrier.

In one embodiment, a compound of the invention is co-administered with another therapeutic agent for the treatment of conditions and/or diseases involving abnormal or elevated catabolism of tryptophan.

According to a further aspect of the invention the second therapeutic agent may be a IDO or TDO inhibitor.

Examples of IDO inhibitors include oxadiazole and other heterocyclic IDO inhibitors are reported in US 2006/0258719 and US 2007/0185165 and U.S. Pat. Nos. 8,088,803 and 8,796,319. PCT Publication WO 99/29310 reports methods for altering T cell-mediated immunity comprising altering local extracellular concentrations of tryptophan and tryptophan metabolites, using an inhibitor of IDO such as 1-methyl-DL-tryptophan, p-(3-benzofuranyl)-DL-alanine, p-[3-benzo(b)thienyl]-DL-alanine, and 6-nitro-L-tryptophan) (Munn, 1999). Reported in WO 03/087347 are methods of making antigen-presenting cells for enhancing or reducing T cell tolerance (Munn, 2003). Compounds having indoleamine-2,3-dioxygenase (IDO) inhibitory activity are further reported in WO 2004/094409; and U.S. Patent Application Publication No. 2004/0234623 is directed to methods of treating a subject with a cancer or an infection by the administration of an inhibitor of indoleamine-2,3-dioxygenase in combination with other therapeutic modalities.

Preferred examples of IDO inhibitors are Epadcadostat, BMS-986205, GDC0918, GDC0119, 1-methyltryptophan, rosmarinic acid, COX2 inhibitors.

Examples of TDO inhibitors include those presented in WO/2017107979, in WO/2015082499, in WO/2017075341, in WO/2017034420, in WO/2014141110 or in WO/2016165613.

When the condition involving abnormal or elevated catabolism of tryptophan is cancer particular agents include, but are not limited to: other anticancer treatments such a chemotherapeutic agent, an immunotherapeutic agent, a gene therapy agent, and a radiotherapeutic agent.

According to a this aspect of the invention the second therapy is selected from the group consisting of one or more of a chemotherapeutic agent; an alkylating agent, such as carmustine or temozolamide; a mitotic inhibitor, such as taxanes, (e.g. paclitaxol or docetaxol) or vinca alkaloids (e.g. vinblastine, vincristine, vindestine or vinorelbine); platinum derived compounds (e.g. carboplatin, cisplatin, nedaplatin, oxaliplatin, triplatin tetranitrate or satraplatin); dihydrofolate reductase inhibitors (e.g. aminopterin, methotrexate, pemetrexed or pralatrexate); a DNA polymerase inhibitor (e.g. cytarabine); a ribonucleotide reductase inhibitor (e.g. gemcitabine); a thymidylate synthase inhibitors (e.g. fluorouracil, capecitabine, tegafur, carmofur or floxuridine); aspirin; a non-steroidal anti-inflammatory agent (e.g. ibuprofen); a steroidal anti-inflammatory agent (e.g. a corticosteroid, such as, prednisolone or cortisol); a non-drug oncology therapeutic agent; radiotherapy; tumour embolisation; surgery; and ultrasound.

More preferably the second therapeutic agent may comprise: alemtuzumab, ipilimumab, nivolumab, ofatumumab, rituximab, actinomycin, azacitidine, azathioprin, carboplatin, capecitabin, cisplatin, cyclophosphamide, cytarabine, daunorubicin, docetaxel, doxifluridine, doxorubicin, epirubicin, etoposide, fluorouracil, gemcitabine, hydroxyurea, idarubicin, imatinib, imiquimod, irinotecan, mechlorethamine, mercaptopurin, methotrexate, mitoxantrone, oxaliplatin, paclitaxel, pembrolizumab, pemetrexed, sorafenib, temozolomide, teniposide, tioguanine, topotecan, valrubicin, vinblastine, vincristine, vindesine, vinorelbine or vismodegib.

In one aspect of the invention the additional therapeutic agent may be an immunotherapeutic agent.

The immunotherapeutic agent may consist of one or more of CAR-T cells, vectors, vaccines, armed antibodies; an agent capable of enhancing use of the immune system to treat cancer; an agent of the monoclonal antibody class capable of enhancing use of the immune system to treat cancer; an agent of the interferon class capable of enhancing use of the immune system to treat cancer.

In one aspect of the invention the immunotherapeutic agent consists of one or more of CAR-T cells, vectors, vaccines, and armed antibodies.

In another aspect of the invention the immunotherapeutic agent consists of any agent capable of enhancing use of the immune system to treat cancer.

In another aspect of the invention the immunotherapeutic agent consists of any agent of the monoclonal antibody class capable of enhancing use of the immune system to treat cancer.

In another aspect of the invention the immunotherapeutic agent consists of any agent of the interferon class capable of enhancing use of the immune system to treat cancer.

In another aspect of the invention the immunotherapeutic agent consists of any agent of the interleukin class capable of enhancing use of the immune system to treat cancer.

Such an immunotherapeutic agent may be checkpoint inhibitor as herein described, e.g. an agent which targets immune checkpoints, wherein immune checkpoints are those pathways within the system for maintaining self-tolerance and modulating the duration and amplitude of physiological immune responses.

According to this aspect of the invention the checkpoint inhibitor may be an agent which targets, i.e. inhibits, one or more of CTLA4, PD1, PDL1, PDL2, CD80, CD86, CD28, B7RP1, ICOS, B7-H3, B7-H4, HVEM, BTLA, MHC-Class 1, MHC-Class 2, KIR, TCR, LAG3, CD137L, CD137, OX40L, OX40, CD70, CD27, CD40, CD40L, GAL9, TIM3, A2aR, CD52, CD20, CD274 and CD279.

In a preferred aspect of the invention checkpoint inhibitor is one or more of a CTLA4, PD1 or PDL1 inhibitor.

Such an inhibitor of CTLA-4 may be any effective inhibitor of CTLA-4. The inhibitor of CTLA-4 may be an anti-CTLA-4 antibody. The anti-CTLA-4 antibody may be a monoclonal antibody. The anti-CTLA-4 antibody monoclonal antibody may be a human antibody or a humanized antibody.

Inhibitors of the CTLA4 pathway include, but are not limited to antibodies, peptides, nucleic acid molecules (including, for example, an antisense molecule, a PNA, or an RNAi), peptidomimetics, small molecules, a soluble CTLA4 ligand polypeptide, or a chimeric polypeptide (for example, a chimeric CTLA4 ligand/immunoglobulin molecule). An antibody may be an intact antibody, an antibody binding fragment, or a chimeric antibody. A chimeric antibody may include both human and non-human portions. An antibody may be a polyclonal or a monoclonal antibody. An antibody may be derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes. In some embodiments, a mixture or cocktail of various inhibitors of the CTLA4 pathway may be administered.

Examples of CTLA4 inhibitor, include, but shall not be limited to, one or more of ipilimumab, rituximab, pembrolizumab, ofatumumab, tremelimumab, BMS-936559, MedI-4736, MPDL-3280A, MSB0010718C, pidilizumab and MK-3475.

Other anti-CTLA4 antibodies include, but are not limited to, those taught in U.S. Pat. Nos. 7,311,910; 7,307,064; 7,132,281; 7,109,003; 7,034,121; 6,984,720; and 6,682,736.

Such an inhibitor of the PD-L1/PD-1 pathway may be any effective inhibitor of the PD-L1/PD-1 pathway. In some embodiments, the inhibitor of the PD-L1/PD-1 pathway is an anti-PD-L1 antibody or an anti-PD-1 antibody. In some embodiments, the anti-PD-L1 or anti-PD-1 antibody is a monoclonal antibody. In some embodiments, the monoclonal antibody is a human antibody. In some embodiments, 10 the anti-PD-L1 or anti-PD-1 antibody is a humanized antibody.

Inhibitors of the PD-L1/PD-1 pathway include, but are not limited to, antibodies, peptides, nucleic acid molecules (including, for example, an antisense molecule, a PNA, or an RNAi), peptidomimetics, small molecules, a soluble PD-1 ligand polypeptide, or a chimeric polypeptide (for example, a chimeric PD-1 ligand/Immunoglobulin molecule). An antibody may be an intact antibody, an antibody binding fragment, or a chimeric antibody. A chimeric antibody may include both human and non-human portions. An antibody may be a polyclonal or a monoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes.

Examples of the PD Iinhibitor, include, but shall not be limited to, one or more of nivolumab, pidilizumab and MK-3475., BMS-936559 or BMS-936558 from Bristol-Myers Squibb, MPDL3280A from Genentech, MK-3475 from Merck, CT-011 from Curetech, and MEDI4736 from MedImmune.

Examples of PDL1 inhibitor, include, but shall not be limited to, one or more of BMS-936559, MedI-4736, MPDL-3280A BMS-936558, MK-3475, CT-011, MED14736 and MSB0010718C.

The compounds of the invention may be administered prior to, during or post-surgery, whereby surgery may be palliative or curative.

When the condition involving abnormal or elevated catabolism of tryptophan is viral infection particular agents include, but are not limited to:
an antiviral drug, such as, idoxuridine, acyclovir, vidarabine, gancyclovir; and
an anti-HIV agent, such as, zidovudine, didanosine, zalcitabine, indinavir sulfate ethanolate, ritonavir.

Co-administration includes any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

### Synthetic Procedures

General

Compounds of Formula (I), and salts and solvates thereof, and intermediates of formulae (II), (III), (IV) and (V) may be prepared by the general methods outlined herein or any method known in the art, said methods constituting a further aspect of the invention. In the following description, the groups R¹, R², R³, W¹, W², W^{3A}, W^{3B}, W^{4A}, W^{4B}, n and m have the meaning defined herein for the compounds of Formula (I) as herein described, unless otherwise stated.

A compound of the invention, as well as intermediate compounds of the invention, can be prepared from readily available starting materials using the following general methods and procedures. It will be appreciated that where typical or preferred process conditions (i.e. reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Third Edition, Wiley, New York, 1999, and references cited therein.

The following methods are presented with details as to the preparation of a compound of the invention as well as intermediate of the invention as defined hereinabove and the comparative examples.

Compounds of the invention, as well as intermediates of the invention, may be prepared from known or commercially available starting materials and reagents by one skilled in the art of organic synthesis, using methods known to the person skilled in the art or by methods described herein.

All reagents were of commercial grade and were used as received without further purification, unless otherwise stated. Commercially available anhydrous solvents were used for reactions conducted under inert atmosphere. Reagent grade solvents were used in all other cases, unless otherwise specified.

A compound of the invention as well as intermediate of the invention can be separated from a reaction mixture and further purified by a method such as column chromatography, high pressure liquid chromatography, or recrystallization. Column chromatography is performed on silica gel 60 (70-200 μm). Flash chromatography is carried out using prepacked columns with 15 or 50 μm particle size silica gel. Preparative thin-layer chromatography is carried out using pre-coated silica gel 2000 micron UV254 nm plates (thickness 2.0 mm).

Thin-layer chromatography is performed using pre-coated silica gel 60F-254 plates (thickness 0.25 mm).

NMR spectra are recorded on Bruker DPX 300 MHz equipped with a 5 mm BBI probe, Bruker AV400 MHz equipped with a 5 mm PABBO probe, Bruker DRX 500 MHz equipped with a 5 mm PABBI probe and Bruker Avance III 600 spectrometer equipped with a 5 mm RT BBI probe. The samples are recorded at 25° C. using DMSO-d₆ or CDCl₃ as a solvent, unless otherwise stated. Chemical shifts (6) for ¹H NMR spectra are reported in parts per million (ppm) relative to tetramethylsilane (S 0.00) as internal reference.

Electrospray MS spectra are obtained on Waters Acquity UPLC with Waters Acquity PDA detector and SQD mass spectrometer. Columns used: UPLC BEH C18 1.7 μm, 2.1×5 mm VanGuard Pre-column with Acquity UPLC BEH C18 1.7 μm, 2.1×50 mm Column or Acquity UPLC CSH C18 1.7 μm, 2.1×50 mm Column. All the methods are using MeCN/H₂O gradients. MeCN and H₂O contains either 0.1% Formic Acid or 10 mM NH₄HCO₃.

For preparative purification HPLC Waters Mass Directed Autopurification System is used. The system is composed of Waters Sample Manager 2767, Waters System Fluid Organizer, Waters Binary Gradient Module 2545, Waters 515 HPLC Pump, Waters Photodiode Array Detector 2998 and Waters Micromass ZQ MS detector. Software used: FractionLynx and MassLynx v4.1. General HPLC method parameters: gradient mobile phase of 0.1% formic acid in H₂O and MeCN or 10 mM NH₄HCO₃ pH=10 and MeCN. Column XBridge 30×150 mm, 5 μm. PDA detector settings: wavelength: 210-400 nm, resolution: 1.2 nm, sampling rate: 1.0 points/sec, filter response: 1.

Microwave heating is performed with a Biotage Initiator.

Pharmaceutically acceptable acid addition salts, which also represent an object of the present invention, may be obtained by reaction of a compound of Formula (I) with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzenesulfonic acid, methane sulfonic acid, laurylsulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar co-solvent.

The present invention will now be described by way of example only with reference to the accompanying figures in which:

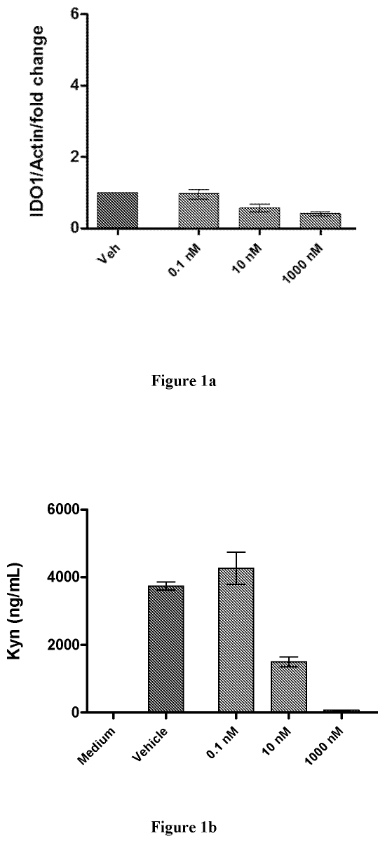

FIG. 1a is a graph of dose-dependent reduction in IDO1 protein in SKOV-3 cells after 24 hours exposure to compound 102;

FIG. 1b is a graph of dose-dependent inhibition of kynurenine production by SKOV-3 after 24 hours exposure to compound 102; and

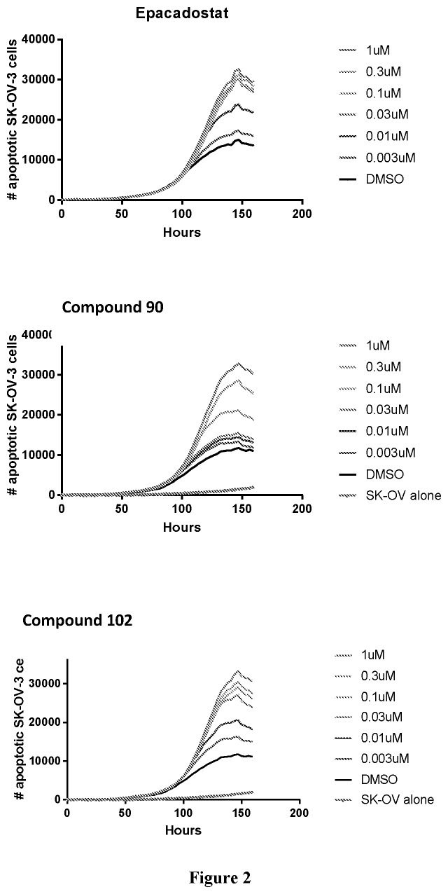

FIG. 2 illustrates the activity of Epacadostat and compounds 90 and 102. NucLight™ Red transfected SK-OV-3 ovarian cancer cells seeded in 96-well flat-bottomed plates 24 hours prior to addition of PBMC, rhIL-2, anti-CD3 and anti-CD28 plus Epacadostat, example compound of the invention or DMSO. Cells were incubated in the Incucyte Zoom® and images were taken at 3-hourly intervals. Data are shown as mean number of apoptotic SK-OV-3 cells (n=4 biological replicates), error bars have been removed for clarity.

The following abbreviations listed in Table 1 are used in the Examples and other parts of the description.

TABLE 1
List of abbreviations used in experimental section:

| Abbreviation | Definition |
|---|---|
| μL | Microliter |
| AcCl | Acetyl chloride |
| anhyd. | anhydrous |
| aq. | aqueous |
| BINAP | 2,2′-Bis(diphenylphosphino)-1,1′-binaphtyl |
| Boc | tert-Butyloxycarbonyl |
| Bn | Benzyl |
| BPin | Boronic acid pinacol ester |
| br. s. | broad singlet |
| CDI | 1,1′-Carbonyldiimidazole |
| Cpd | Compound |
| Cpd# | Compound number |
| CPhos | 2-(2-dicyclohexylphosphanylphenyl)-N1,N1,N3,N3- |
| | tetramethyl-benzene-1,3-diamine |
| CuSO₄x5H₂O | Copper (II)sulfate pentahydrate |
| d | doublet |
| DBU | 1,8-diazabicyclo(5.4.0)undec-7-ene |
| DCM | Dichloromethane |
| Deoxo-Fluor | Bis(2-methoxyethyl)aminosulfur trifluoride |
| DIPEA | N,N-diisopropylethylamine |
| DMAP | N,N-Dimethylpyridine-4-amine |
| DMF | N,N-Dimethylformamide |
| DMSO | Dimethylsulfoxide |
| equiv. | Equivalents |
| Et₂O | Diethyl ether |
| EtOAc | Ethyl acetate |
| EtOH | Ethanol |
| ES⁺ | positive electrospray ionisation mass spectrometry |
| g | gram |
| h | hour(s) |
| Hal | Halogen |
| HATU | 1-[Bis(dimethylamino)methylene]-1H-1,2,3-triazolo[4,5- |
| | b]pyridinium 3-oxid hexafluorophosphate |
| HPLC | High-performance liquid chromatography |
| Int | Intermediate |
| iPrOH | Isopropyl alcohol |
| LCMS | Liquid Chromatography-Mass Spectrometry |
| m | multiplet |
| M | moldm⁻³ |
| MeCN | Acetonitrile |
| MeI | Methyl iodide |
| MeOH | Methanol |
| mg | milligram |
| min | minute |
| mL | millilitre |
| mmol | millimoles |
| MsCl | Mesyl chloride; methanesulfonyl chloride |
| Mtd | Method |
| MW | Molecular weight |
| m/z | mass divided by charge number |
| Na₂SO₄ | sodium sulphate |
| NMR | Nuclear Magnetic Resonance |
| Pd(OH)₂/C | Palladium hydroxide on Carbon, Pearlman's catalyst |
| pH | potential of hydrogen (scale of acidity) |
| PhMe | Toluene |
| Pd/C | Palladium on Carbon 10 wt % |
| Pd(OAc)₂ | Palladium(II) acetate |
| Pd(PPh₃)₄ | Tetrakis(triphenylphosphine)palladium(0) |
| Pd(PPh₃)₂Cl₂ | Bis(triphenylphosphine)palladium(II) dichloride |
| pTsOH xH₂O | p-Toluenesulfonic acid monohydrate |
| quint | quintet |
| RT | Room temperature |
| tBu | tert-Butyl |
| s | singlet |
| sat. aq. sol. | saturated aqueous solution |
| SCX | Strong Cation Exchange |
| t | triplet |
| TBAFxH₂O | tetrabutylammonium fluoride hydrate |
| TBDMSCl | tert-Butyldimethylsilyl chloride |
| TEA | Triethyl amine |
| TFA | Trifluoroacetic acid |
| THF | Tetrahydrofuran |
| TLC | Thin-layer chromatography |
| UPLC | Ultra-performance liquid chromatography |
| XPhos | 2-Dicyclohexylphosphino-2′,4′,6′-triisopropylbiphenyl |
| XPhos-Pd-G1 | (2-Dicyclohexylphosphino-2′,4′,6′-triisopropyl-1,1′- |
| | biphenyl)[2-(2-aminoethyl)phenyl)]palladium(II) chloride |
| wt. % | weight percent |

The compounds and processes of the present invention will be better understood in connection with the following examples, which are intended as anillustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to.

A compound of the invention as well as intermediate of the invention can be produced according to the following procedures.

### Synthetic Preparation of the Compound of the Invention

### INTERMEDIATES

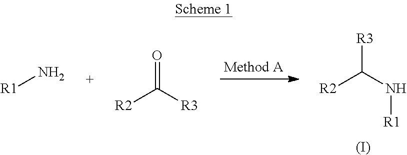

Method A: General Procedure for the Preparation of Intermediates of Formula (I) by Reductive Amination

The reaction is typically performed by combining an appropriate alkyl, cycloalkyl, substituted cycloalkyl or heteroaryl amine of formula R1-NH₂ (1-1.2 equiv.) and a carbonyl compound (aldehyde or methyl-ketone) (0.67-3 equiv.) in the presence of an appropriate base (typically, trimethylamine or sodium acetate), or without a presence of base, and acetic acid (1-3 equiv.) or pTsOH×H₂O or without addition of acetic acid or pTsOH×H₂O in a suitable solvent (typically, methanol or toluene or dichloromethane) in the presence or without molecular sieves or magnesium perchlorate (0.3 equiv.) or sodium sulphate or magnesium sulphate. The resulting mixture is stirred at 0° C. to room temperature to 110° C. for 2 h to 5 days. To the reaction temperature lowered to 0° C. or at room temperature, a reducing agent (1.2-16 equiv.) is added (typically, sodium borohydride or sodium cyanoborohydride or lithium borohydride). The resulting mixture is stirred at room temperature for 1-72 hours. The expected Intermediate of formula (I) may be isolated and, if desired, further purified by methods known to one skilled in the art.

Alternatively, a mixture of alkyl, cycloalkyl, substituted cycloalkyl or heterocyclic amine (R1-NH₂) (1-1.2 equiv.), a carbonyl compound (0.8-2 equiv.), an appropriate base (typically, trimethylamine), or without a presence of base, and Lewis acid (2.1-2.4 equiv.) (typically, Ti(iPrO)₄) in an appropriate solvent, such as dichloromethane, is stirred at room temperature for 12-20 hours or under microwave irradiation at 70° C. for 15 minutes. The reaction mixture is evaporated till dryness, dissolved in an appropriate solvent, such as methanol, and then a reducing agent, such as sodium borohydride, (2-5 equiv.) is added portionwise to give the corresponding intermediate of formula (I), which is isolated and, if desired, may be further purified by methods known to one skilled in the art.

### Example A.1

### Illustrative Synthesis of N-[(5-methyl-2-furyl)methyl]cyclopentanamine

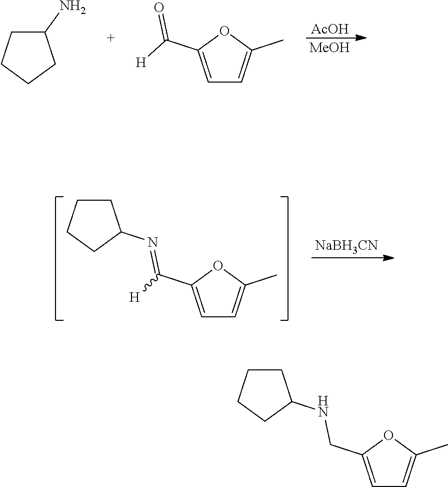

To a stirred solution of 5-methylfuran-2-carbaldehyde (2.5 g, 1.1 equiv.) and cyclopentamine (2.01 mL, 1 equiv.) in dry methanol (200 mL) was added glacial acetic acid (3.47 mL, 3 equiv.) and the mixture was stirred at 55° C. for 2 hours. The reaction mixture was cooled to 0° C. before NaBH₃CN (3.31 g, 2.5 equiv.) was added and the mixture was stirred at room temperature for 16 hours. Volatiles were evaporated. The concentrated mixture was transferred to a separatory funnel containing 1N NaOH (150 mL), and extracted with DCM (2×150 mL). The combined organic extracts were dried over Na₂SO₄, were filtered, and the solvent was removed in vacuo to yield the crude product, which was dissolved in MeOH (20 mL) and applied to a SCX column and eluted with MeOH followed by 2M ammonia in MeOH. Ammonia/MeOH fractions were combined and evaporated to afford the expected product (3.4 g). LCMS: MW (calcd): 179.26; MS (ES⁺, m/z): 180.53 [M+H]⁺.

### Example A.2

### Illustrative Synthesis of N-[(5-methyl-2-furyl)methyl]-1H-imidazol-5-amine

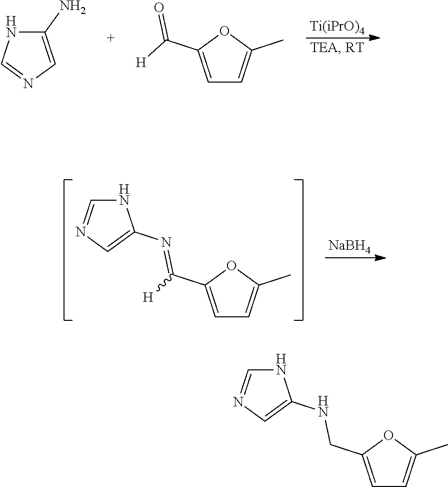

To a stirred solution of 5-methylfuran-2-carbaldehyde (200 mg, 2 equiv.), 2-aminoimidazole hemisulfate (240.1 mg, 1 equiv.) and TEA (281 μL, 2.2 equiv.) in dichloromethane (6 mL) was added Ti(iPrO)₄ (619.5 mg, 2.4 equiv.) and the mixture was stirred at room temperature for 16 hours. Volatiles were evaporated. The concentrated mixture was dissolved in MeOH (6 mL) and NaBH₄ (172 mg, 5 equiv.) was added portionwise every 30 minutes until almost complete conversion to the wanted amine. The reaction mixture was transferred to a separatory funnel containing NaHCO₃ (15 mL), and extracted with DCM (2×15 mL). The combined organic extracts were dried over Na₂SO₄, were filtered, and the solvent was removed in vacuo to yield the crude product (222 mg). LCMS: MW (calcd): 177.20; MS (ES⁺, m/z): 178.08 [M+H]⁺.

### Example A.3

### Illustrative Synthesis of 5-methyl-N-[(5-methyl-2-furyl)methyl]isoxazol-4-amine

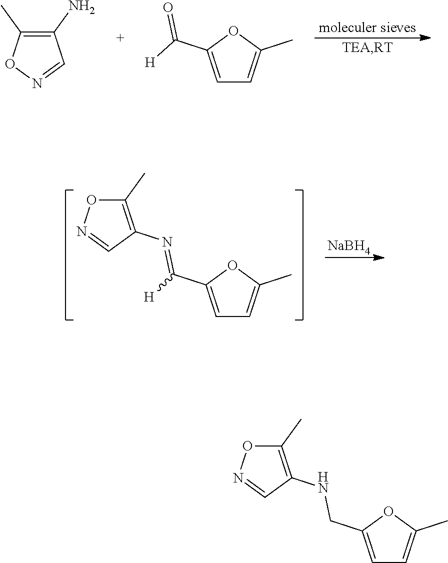

To a stirred solution of 5-methylfuran-2-carbaldehyde (199.1 mg, 1.0 equiv.) in methanol (5 mL), molecular sieves 3 Å, 5-methylisoxazol-4-amine hydrochloride (250.0 mg, 1.03 equiv.) and TEA (0.27 mL, 1.07 equiv.) were added. Under the Argon atmosphere, the mixture was stirred at room temperature for 2 hours. The reaction mixture was cooled to 0° C. and diluted with methanol (20 mL) before NaBH₄ (1.03 g, 15.06 equiv.) was added portionwise under Argon atmosphere. The mixture was stirred at 0° C. for 2 hours. To the reaction mixture at 0° C., aqueous solution of iN HCl (40 mL) was added. The resulting solution was extracted with DCM (3×50 mL). The combined organic extracts were dried over Na₂SO₄, were filtered, and the solvent was removed in vacuo to yield the crude product (296 mg). LCMS: MW (calcd): 192.09; MS (ES⁺, m/z): 192.48 [M+H]⁺.

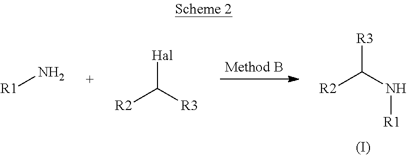

### Method B: General Procedure for the Preparation of Intermediates of Formula (I) by Nucleophilic Substitution

The reaction is typically performed by combining an appropriate alkyl or heteroaryl amine of formula R1-NH₂ (1-1.2 equiv.) and an alkyl halide or aryl halide compound (bromide, chloride, iodide, fluoride) (1 equiv.) in the presence of an appropriate base (typically, N,N-diisopropylethylamine) in a suitable solvent (typically, tetrahydrofuran). The resulting mixture is stirred at room temperature for 17 hours or at 80° C. from 2 to 18 hours. The expected Intermediate of formula (I) may be isolated and, if desired, further purified by methods known to one skilled in the art.

### Example B.1

### Illustrative Synthesis of 5-methyl-N-[[2-(trifluoromethyl)-4-pyridyl]methyl]isoxazol-4-amine

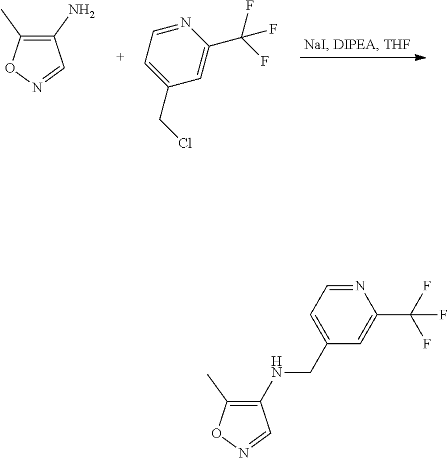

To a solution of 4-(chloromethyl)-2-(trifluoromethyl)pyridine (97.8 mg, 1 equiv.), 5-methylisoxazol-4-amine (49.1 mg, 1 equiv.), sodium iodide (225 mg, 3 equiv.) and DIPEA (0.261 mL, 3 equiv.) were added. The reaction mixture was stirred at room temperature for 16 hours. The reaction mixture was filtered and concentrated in vacuo to yield the crude product. The obtained residue was purified by flash chromatography on silica gel (eluting with DCM/2.5% of MeOH in DCM gradient; 0-40% of 2.5% of MeOH in DCM) to afford the expected product (68 mg). LCMS: MW (calcd): 257.21; MS (ES⁺, m/z): 258.06 [M+H]⁺.

### Example B.2

### Illustrative Synthesis of N-[(5-methyl-2-furyl methy]-1,3-benzothiazol-2-amine

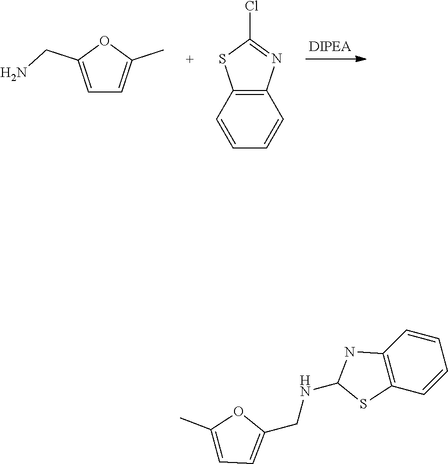

To a solution of (5-methyl-2-furyl) methanamine (50 mg, 1 equiv.) in DIPEA (0.12 mL, 1.5 equiv.), 2-chloro-1,3-benzothiazole (75 mg, 1 equiv.) was added and stirred at 80° C. overnight. The reaction mixture was diluted with EtOAc (30 mL) and washed with aqueous saturated solution of NaHCO₃ (15 mL) and brine (15 mL). Layers were separated, organic phase was dried over Na₂SO₄, filtered and concentrated in vacuo to yield the crude. The obtained residue was purified by flash chromatography on silica gel (eluting with cyclohexane/EtOAc gradient; 0-60% of EtOAc) to afford the expected product (53 mg). LCMS: MW (calcd): 244.31; MS (ES⁺, m/z): 245.05 [M+H]⁺.

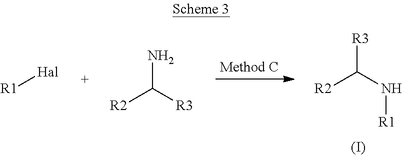

### Method C: General Procedure for the Preparation of Intermediates of Formula (I) by Buchwald-Hartwig Amination

The reaction is typically performed by combining an appropriate aryl or heteroaryl halide of formula R1-Hal (bromide, chloride) (1 equiv.) and an alkyl amine (1-1.2 equiv.) in the presence of a palladium catalyst (0.1 equiv.), such as Pd(OAc)₂ or any other suitable catalyst, with or without a suitable ligand (0.1 equiv.), such as BINAP and a base (3-4 equiv.), such as potassium carbonate, in suitable solvent (typically toluene). The resulting mixture is stirred at temperature typically 120-140° C. for 3 hours. The expected Intermediate of formula (I) may be isolated and, if desired, further purified by methods known to one skilled in the art.

### Example C.1

### Illustrative Synthesis of 3-methyl-N-[(5-methyl-2-furyl)methyl]pyridin-2-amine

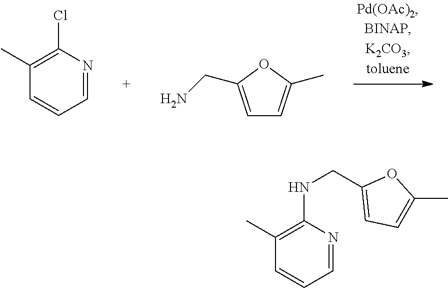

A suspension of 2-chloro-3-methyl-pyridine (120 mg, 1 equiv.), (5-methyl-2-furyl) methanamine (125 mg, 1.2 equiv.), potassium carbonate (545 mg, 3.5 equiv.), BINAP (70 mg, 0.1 equiv.) and Pd (OAc) 2 (30 mg, 0.1 equiv.) in toluene (3 mL) was heated at 130° C. for 3 hours. The reaction mixture was diluted with EtOAc (40 mL) and extracted with aqueous saturated solution of NaHCO₃ (15 mL) and brine (15 mL). Organic phase was separated, dried over Na₂SO₄, filtered and evaporated in vacuo to yield the crude product. The obtained residue was purified by flash chromatography on silica gel (eluting with cyclohexane/EtOAc gradient; 0-30% of EtOAc in cyclohexane) to afford the expected product (125 mg). LCMS: MW (calcd): 202.25; MS (ES⁺, m/z): 203.13 [M+H]⁺.

### Compounds

### Method D: General Procedures for Preparation of Urea Compounds of Formula (II)

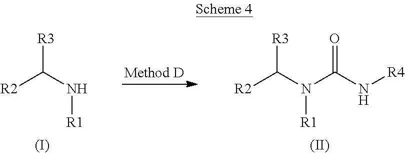

### Method D1: Isocyanate

The reaction is typically performed by adding 1-3 equiv. of isocyanate to a solution of an appropriate compound of formula (I) (0.9-1 equiv.) in a suitable solvent, such as DCM or toluene. The reaction mixture is stirred at room temperature for 30 min to 72 h or at 100-150° C. for 40 min to 3 h using microwave irradiation. The expected product of formula (II) may be isolated and, if desired, further purified by methods known to one skilled in the art.

### Example D1.1

### Illustrative Synthesis of 3-(4-chlorophenyl)-1-cyclobutyl-1-[[3-(trifluoromethyl)-phenyl]methyl]urea (Compound 90)

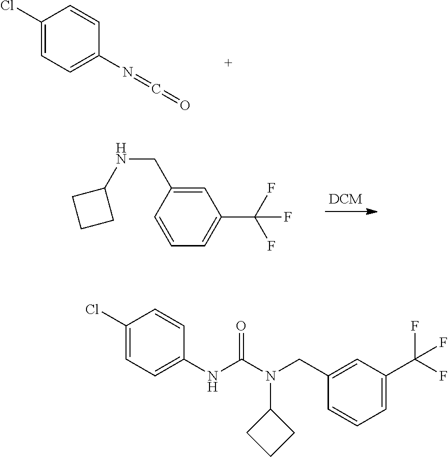 [SMILES: O=C=Nc1ccc(Cl)cc1 ; FC(F)(F)c1cccc(CNC2CCC2)c1 ; O=C(Nc1ccc(Cl)cc1)N(Cc1cccc(C(F)(F)F)c1)C1CCC1]

4-Chlorophenyl isocyanate (120.3 mg, 1 equiv.) and N-[[3-(trifluoromethyl)-phenyl]methyl]cyclobutanamine (crude product, 1 equiv.) were dissolved in DCM (2.5 mL) and stirred at room temperature for 16 h. Solvent was removed in vacuo and the obtained crude product was further purified by preparative LC-MS to afford the expected product (89 mg). LCMS: MW (calcd): 382.81; MS (ES⁺, m/z): 383.11 [M+H]⁺.

### Method D2: Triphosgene

The reaction is typically performed by adding an appropriate amine (1.3 equiv.) and base (4.3-5.6 equiv.) (such as TEA or DIPEA) to a solution of triphosgene (0.5 equiv.) in a suitable solvent, such as THF, at 0° C. to room temperature. The resulting mixture is stirred for 15 min to 1 h at 0° C. to room temperature, then mixed with THF solution (or suspension) of an appropriate compound of formula (I) (1 equiv.) to which, if required, additional amount of base, such as TEA or DIPEA, may be added. The reaction mixture is stirred at room temperature for 1-24 h. The expected product of formula (II) may be isolated and, if desired, further purified by methods known to one skilled in the art.

Alternatively, to a solution of triphosgene (0.5 equiv.) in a suitable solvent, such as EtOAc with molecular sieves at 0° C., a solution of an appropriate amine (1.0 equiv.) in a suitable solvent, such as EtOAc was added dropwise. The expected isocyanate may be isolated and, if desired, further purified by methods known to one skilled in the art. The reaction of urea formation was typically performed by adding 1-3 equiv. of prepared isocyanate to a solution of an appropriate compound of formula (I) (1 equiv.) in a suitable solvent, such as DCM. The reaction mixture is stirred at room temperature for 30 min to 24 h. The expected product of formula (II) may be isolated and, if desired, further purified by methods known to one skilled in the art.

### Example D2.1

### Illustrative Synthesis of 1-cyclopentyl-3-(4-ethynylphenyl)-1-[(5-methyl-2-furyl)methyl]urea (Compound 102)

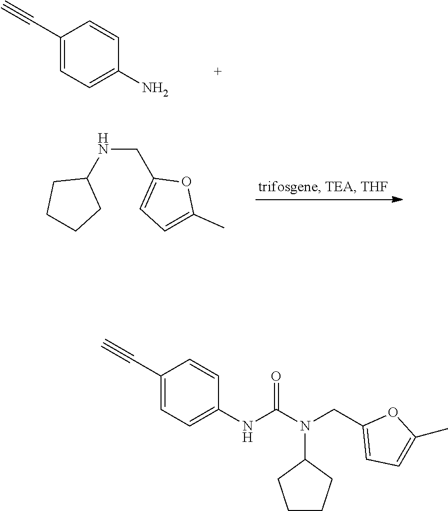

A solution of 4-ethynylaniline (170 mg, 1.3 equiv.) and TEA (0.68 mL, 4.3 equiv.) in dry THE (4 mL) was added to a solution of triphosgene (167.6 mg, 0.5 equiv.) in dry THE (4 mL) at room temperature. After 30 minutes of stirring, a solution of N-[(5-methyl-2-furyl)methyl] cyclopentanamine (200 mg, 1 equiv.) and TEA (0.2 mL, 1.3 equiv.) in THE (4 mL) was added dropwise. The reaction mixture was stirred at room temperature for 16 hours and then was diluted with DCM, and washed with saturated aqueous solution of NaHCO₃. The organic layer was dried and concentrated under reduced pressure. The obtained residue was purified by flash chromatography on silica gel (eluting with EtOAc/cyclohexane gradient; 0-15% of EtOAc) to afford the expected product (290 mg). LCMS: MW (calcd): 322.40; MS (ES⁺, m/z): 323.20 [M+H]⁺.

### Example D2.2

### Illustrative Synthesis of 3-[4-(difluoromethoxy)phenyl]-1-[(5-methyl-2-furyl)methyl]-1-(5-methylisoxazol-4-yl)urea (Compound 289)

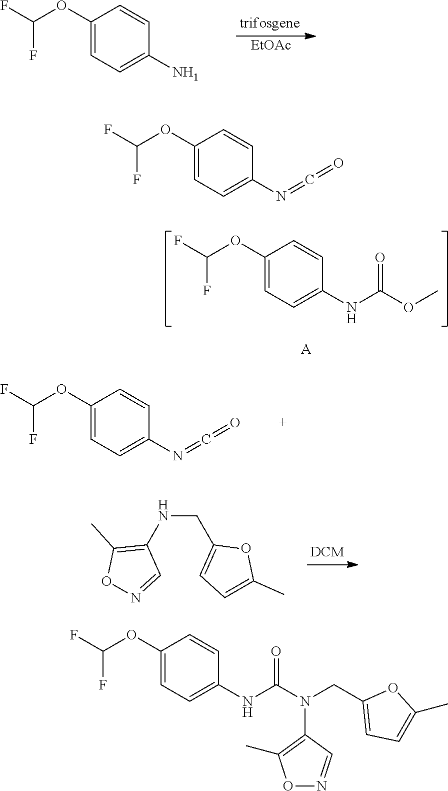

To a solution of triphosgene (65 mg, 0.5 equiv.) in EtOAc (11 mL), molecular sieves were added. To the resulting solution previously cooled to 0° C., a solution of 4-(difluoromethoxy) aniline (70 mg, 1.0 equiv.) in EtOAc (11 mL) was added dropwise. The reaction mixture was stirred at 0° C. for 2 hours and washed with saturated aqueous solution of NaHCO₃ and brine. The organic layer was dried over Na₂SO₄, filtered and evaporated in vacuo to afford the expected product (49 mg). In order to confirm the structure, 3 mg of product was dissolved in MeOH and evaporated to dryness to obtain Intermediate A. 1H NMR (300 MHz, DMSO-d6) δ=9.70 (s, 1H), 7.46 (d, J=8.7 Hz, 2H), 7.10 (d, J=8.7 Hz, 2H), 7.09 (t, J=74.1 Hz, 1H), 3.65 (s, 3H) ppm.

Previously prepared 4-(difluoromethoxy) phenyl isocyanate (43 mg, 0.9 equiv.) and 5-methyl-N-[(5-methyl-2-furyl)methyl]isoxazol-4-amine (crude product, 1 equiv.) were dissolved in DCM (2.0 mL) and stirred at room temperature overnight. Solvent was removed in vacuo and the obtained crude product was further purified by flash chromatography on silica gel (eluting with EtOAc/cyclohexane gradient; 0-30% of EtOAc) to afford the expected product (49 mg). LCMS: MW (calcd): 377.34; MS (ES⁺, m/z): 378.19 [M+H]⁺.

### Method D3: CDI

The reaction is typically performed by adding CDI (1-1.1 equiv.) to a solution of the corresponding amine (1 equiv.) in a suitable solvent, such as DMF or DCM, in the presence of a suitable base (typically TEA, 2 equiv.), or without presence of base. The reaction mixture is stirred at room temperature for 1-2 h, then an appropriate compound of formula (I) (1 equiv.) is added and resulting mixture stirred at RT for 16 h. The expected product of formula (II) may be isolated and, if desired, further purified by methods known to one skilled in the art.

### Example D3.1

### Illustrative Synthesis of 3-(4-chloro-2-fluoro-phenyl)-1-cyclopentyl-1-[(5-methyl-2-furyl)methyl]urea (Compound 172)

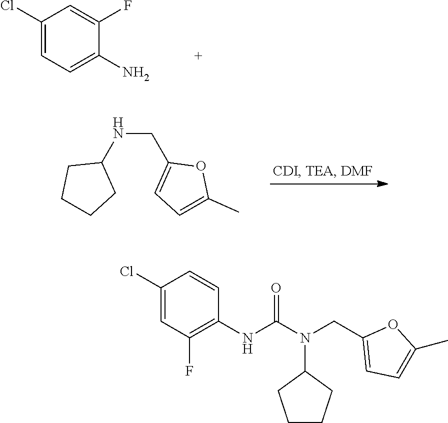 [SMILES: Nc1ccc(Cl)cc1F ; Cc1ccc(CNC2CCCC2)o1 ; Cc1ccc(CN(C(=O)Nc2ccc(Cl)cc2F)C2CCCC2)o1]

To a solution of 4-chloro-2-fluoro aniline (0.76 mL, 1 equiv.) in DMF (2.5 mL), TEA (0.19 mL, 2 equiv.) and CDI (110.2 mg, 1 equiv.) were added and the reaction mixture was stirred at room temperature for 1 h. Then N-[(5-methyl-2-furyl)methyl]-cyclopentanamine (162 mg, 1 equiv.) was added and stirring continued at room temperature for 16 hours. The reaction mixture was diluted with EtOAc (20 mL), was transferred to a separatory funnel and washed with sat. aq. sol. NaHCO₃ (3×5 mL). Organic layer was dried over Na₂SO₄ (anhyd.), was filtered and evaporated in vacuo to yield the crude product, which was purified by flash chromatography on silica gel (eluting with DCM:cyclohexane=1:1/cyclohexane gradient; 0-100% of DCM:cyclohexane=1:1) to afford the expected product (84.2 mg). LCMS: MW (calcd): 350.82; MS (ES⁺, m/z): 351.80 [M+H]⁺.

### Method D4: Phosgene

Typically, to a solution of an appropriate amine (1 equiv.) and product of formula (I) (1 equiv.) in THF at RT is added base, typically TEA (4 equiv.), followed by phosgene (15 wt % solution in PhMe, 1 equiv.). Reaction mixture is stirred at room temperature for 16 hours. The expected product of formula (II) may be isolated and, if desired, further purified by methods familiar to one skilled in the art.

### Example D4.1

### Illustrative Synthesis of 1-(benzofuran-2-ylmethyl)-1-cyclopentyl-3-(4-ethynylphenyl)urea (Compound 131)

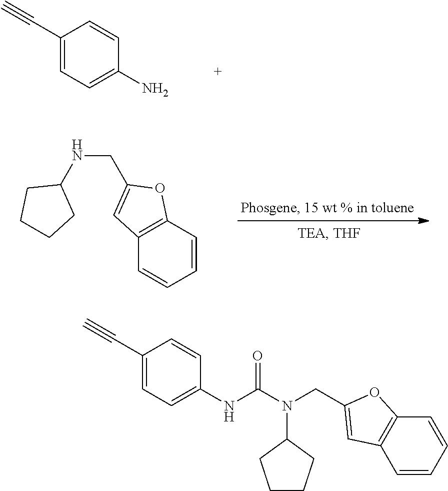

To a stirred solution of N-(benzofuran-2-ylmethyl)cyclopentanamine (50 mg, 1 equiv.), 4-ethynylaniline (32.5 mg, 1 equiv.) and TEA (156 μL, 4 equiv.) in THE (7 mL), a phosgene solution 15 wt % in toluene (199 μL, 1 equiv.) was added. The reaction mixture was stirred at room temperature for 16 hours. Volatiles were evaporated. The concentrated reaction mixture was transferred to a separatory funnel containing distilled water (50 mL), and was extracted with EtOAc (3×50 mL). Combined organic extracts were evaporated in vacuo to yield the crude product, which was purified by flash chromatography on silica gel (eluting with EtOAc/cyclohexane gradient; 0-50% of EtOAc) to afford the expected product (5.0 mg). LCMS: MW (calcd): 358.43; MS (ES⁺, m/z): 359.71 [M+H]⁺.

### Method D5: Phenyl Carbamate

Typically, an appropriate amine (1 equiv.) and phenyl carbamate (1-2.8 equiv.) are dissolved in a suitable solvent, such as DMSO, with or without a presence of base (1.3 equiv.), such as TEA. The reaction mixture is stirred at room temperature for 16 hours. The expected compound of formula (II) may be isolated and, if desired, further purified by methods known to one skilled in the art.

### Example D5.1

### Illustrative Synthesis of 1-cyclopentyl-3-(5-ethynyl-2-pyridyl)-1-[(5-methyl-2-furyl)methyl]urea (Compound 16)

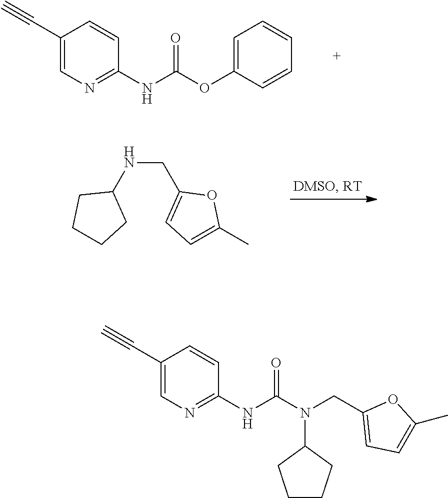 [SMILES: C#Cc1ccc(NC(=O)Oc2ccccc2)nc1 ; Cc1ccc(CNC2CCCC2)o1 ; C#Cc1ccc(NC(=O)N(Cc2ccc(C)o2)C2CCCC2)nc1]

To a stirred solution of phenyl N-(5-ethynyl-2-pyridyl)carbamate (74 mg, 1.1 equiv.) in DMSO (1 mL) was added N-[(5-methyl-2-furyl)methyl]cyclopentanamine (50 mg, 1 equiv.). The reaction mixture was stirred at room temperature for 16 hours. The reaction mixture was further purified by preparative LC-MS (ES⁺ mode, high pH conditions) to afford the expected product (28 mg). LCMS: MW (calcd): 323.39; MS (ES⁺, m/z): 324.73 [M+H]⁺.

### Method D6: Isopropenyl Carbamate

Typically, an appropriate amine (1-2 equiv.) and isopropenyl carbamate (1.1 equiv.) are dissolved in a suitable solvent, such as 1,4-dioxane, with or without a presence of base (0.2-0.3 equiv.), such as DBU. The reaction mixture is stirred at 80° C. for 2 hours. The expected compound of formula (II) may be isolated and, if desired, further purified by methods known to one skilled in the art.

### Example D6.1

### Illustrative Synthesis of 3-[4-[3-[tert-butyl(dimethyl)silyl]oxyprop-1-ynyl]phenyl]-1-cyclobutyl-1-[(5-methyl-2-furyl)methyl]urea

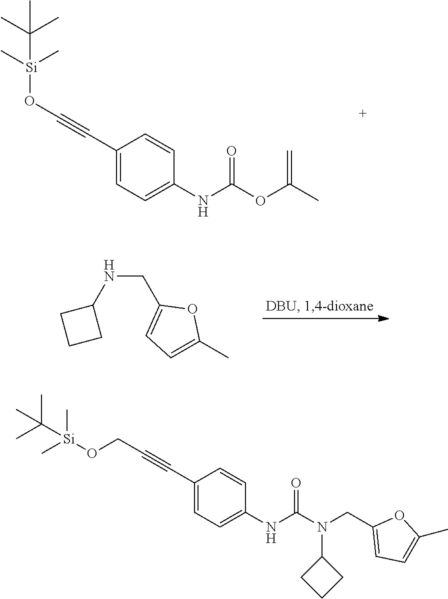

To a stirred solution of isopropenyl N-[4-[2-[tert-butyl(dimethyl)silyl]oxyethynyl]phenyl]carbamate (150 mg, 1.0 equiv.), N-[(5-methyl-2-furyl)methyl]cyclobutanamine (85 mg, 1.09 equiv.) and DBU (15.3 mg, 0.23 equiv) were dissolved in 1,4-dioxane (1.5 mL) and stirred at 80° C. for 2 hours. To the reaction mixture water and brine were added and resulting solution was extracted with DCM (3×15 mL). Combined organic extracts were filtered through phase separator and evaporated in vacuo to yield the crude product, which was purified by flash chromatography on silica gel (eluting with EtOAc/cyclohexane gradient; 0-20% of EtOAc) to afford the expected product (173 mg). LCMS: MW (calcd): 452.66; MS (ES⁺, m/z): 453.32 [M+H]⁺.

### Method E: Suzuki Coupling

The reaction is typically performed by combining an appropriate aryl or heteroaryl halide (1 equiv.) and aryl boronic acid or aryl boronic acid pinacol ester (1-1.5 equiv.) in the presence of a palladium catalyst (0.05-0.2 equiv.), such as Pd(PPh₃)₄, XPhos-Pd-G1 or any other suitable catalyst, with or without a suitable ligand (0.1 equiv.), such as XPhos, and a base (2-3 equiv.), such as potassium carbonate, in suitable solvent or mixture of solvents (typically mixture of dioxane and water) under inert atmosphere. The resulting mixture is stirred at temperature of typically 80-100° C. for 30 minutes to 18 h by using conventional heating. The expected compound may be isolated and, if desired, further purified by methods known to one skilled in the art.

### Example E.1

### Illustrative Synthesis of 1-cyclopentyl-1-[(5-methyl-2-furyl)methyl]-3-[4-(1H-pyrazol-4-yl)phenyl]urea (Compound 108)

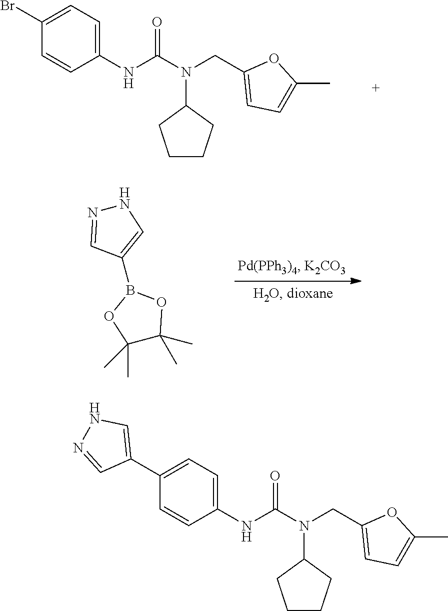 [SMILES: Cc1ccc(CN(C(=O)Nc2ccc(Br)cc2)C2CCCC2)o1 ; CC1(C)OB(c2cn[nH]c2)OC1(C)C ; Cc1ccc(CN(C(=O)Nc2ccc(-c3cn[nH]c3)cc2)C2CCCC2)o1]

To the solution of 4-(4,4,5,5-tetramethyl-1,3,2-dioxaborolan-2-yl)-1H-pyrazole (1.84 g, 1.5 equiv.), 3-(4-bromophenyl)-1-cyclopentyl-1-[(5-methyl-2-furyl)methyl]urea (2.39 g, 1 equiv.), and potassium carbonate (1.75 g, 2 equiv.) in degassed dioxane/water 5:1 (120 mL), Pd(PPh₃)₄ (1.46 g, 0.2 equiv.) was added and the solution was further degassed by bubbling argon for 10 minutes. The flask was capped with the septum and heated at 80° C. in a sand bath for 9 hours. Solution was cooled to RT, transferred to a separatory funnel containing distilled water, and extracted with EtOAc (3×300 mL) The combined organic extracts were dried over Na₂SO₄, filtered, and solvent was removed in vacuo to yield the crude product, which was purified by flash chromatography on silica gel (eluting with: 5% MeOH in DCM/DCM gradient; 0-70% of 5% MeOH in DCM) to afford the expected product, which was further recrystallized from DCM. Mother liquor was evaporated till dryness and then triturated with Et₂O to afford the wanted product (171 mg). LCMS: MW (calcd): 364.4; MS (ES⁺, m/z): 365.8 [M+H]⁺.

### Example E.2

### Illustrative Synthesis of 1-cyclopentyl-1-[(5-methyl-2-furyl)methyl]-3-[6-(1-methylpyrazol-4-yl)-3-pyridyl]urea (Compound 160)

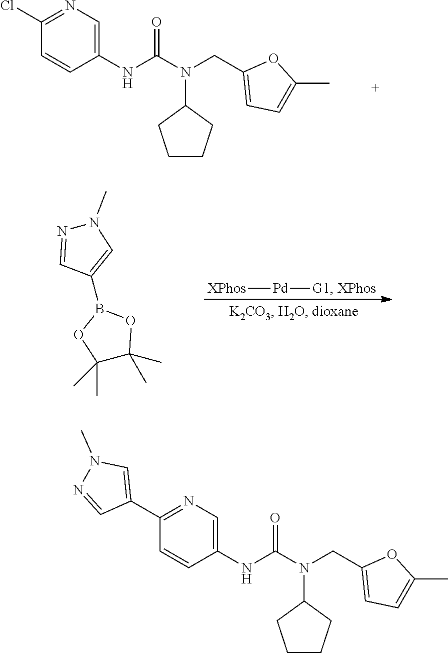

To the solution of 1-methyl-4-(4,4,5,5-tetramethyl-1,3,2-dioxaborolan-2-yl)pyrazole (59 mg, 1.5 equiv.), 3-(6-chloro-3-pyridyl)-1-cyclopentyl-1-[(5-methyl-2-furyl)methyl]urea (66 mg, 1 equiv.), and potassium carbonate (82.8 mg, 3 equiv.) in degassed dioxane/water 5:1 (4 mL), XPhos-Pd-G1 (7.4 mg, 0.05 equiv.) and XPhos (9.5 mg, 0.1 equiv.) were added and the reaction mixture was heated at 80° C. for 45 minutes. Solution was cooled to RT, transferred to a separatory funnel containing distilled water (10 mL), and extracted with DCM (3×10 mL). The combined organic extracts were dried, and the solvent was removed in vacuo to yield the crude product, which was purified by flash chromatography on silica gel (eluting with: 10% MeOH in DCM/DCM gradient; 0-10% of 10% MeOH in DCM) to afford the expected product (64 mg). LCMS: MW (calcd): 379.5; MS (ES⁺, m/z): 380.8 [M+H]⁺.

### Method F: Sonogashira Coupling

The reaction is typically performed by combining an appropriate aryl iodide (1 equiv.) and a terminal alkyne (1.3-2 equiv.) in the presence of a palladium catalyst (0.05-0.075 equiv.), such as Pd(PPh₃)₂Cl₂, or any other suitable catalyst, and copper catalyst (0.05 equiv.), such as CuI, or any other suitable catalyst, and a base, such as TEA, or any other suitable base, which also acts as a solvent, in a suitable solvent, typically acetonitrile under inert atmosphere. The resulting mixture is stirred at room temperature for 2.5-5 hours. The expected compound may be isolated and, if desired, further purified by methods known to one skilled in the art.

### Example F.1

### Illustrative Synthesis of 1-cyclopentyl-1-[(5-methyl-2-furyl)methyl]-3-[6-(1-methylpyrazol-4-yl)-3-pyridyl]urea (Compound 110)

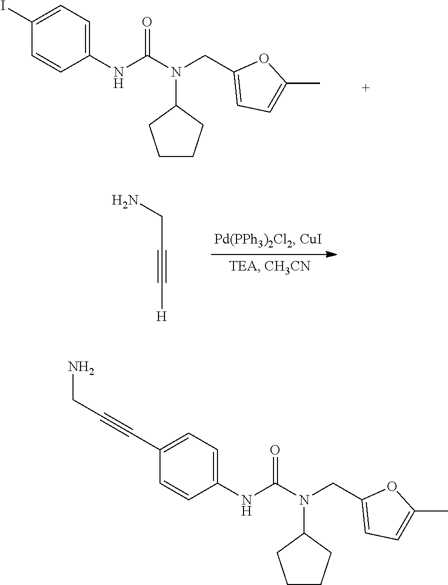

To the solution of 1-cyclopentyl-3-(4-iodophenyl)-1-[(5-methyl-2-furyl)methyl]urea (100 mg, 1 equiv.), Pd(PPh₃)₂Cl₂ (8.4 mg, 0.05 equiv.), and CuI (2.4 mg, 0.05 equiv.) in acetonitrile/TEA 1:1 (1.2 mL), prop-2-yn-1-amine (19.2 μL, 1.3 equiv.) was added and the reaction mixture was stirred at room temperature for 3 hours. Second portion of catalysts (2.5% Pd(PPh₃)₂Cl₂ and 2.5% CuI) and prop-yn-1-amine (10 μL, 0.7 equiv.) were added and stirring continued at room temperature for 2 h. The reaction mixture was transferred to a separatory funnel containing sat. aq. sol. NaHCO₃ (10 mL) and extracted with DCM (3×10 mL). Organic layers were dried, and solvent was removed in vacuo to yield the crude product, which was purified by preparative LC-MS to afford the expected product (14 mg). LCMS: MW (calcd): 351.4; MS (ES⁺, m/z): 352.8 [M+H]⁺.

### Method G: General Procedure for N-Boc Deprotection

Typically, to a solution of an appropriate compound, in suitable solvent or mixture of solvents, such as DCM, TFA (5 equiv.) is added and the reaction mixture is stirred at 0° C. or room temperature for 30 minutes to give the expected product that may be isolated and, if desired further purified by methods known to one skilled in the art.

### Example G.1

### Illustrative Synthesis of 1-cyclopentyl-3-(4-fluorophenyl)-1-[(4-methyl-4-piperidyl)methyl]urea (Compound 86)

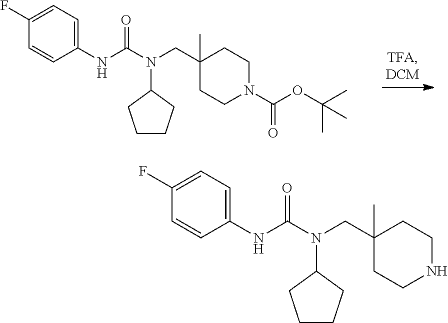

To the solution of tert-butyl 4-[[cyclopentyl-[(4-fluorophenyl)carbamoyl]-amino]methyl]-4-methyl-piperidine-1-carboxylate (40 mg, 1 equiv.) in DCM (2.5 mL), TFA (35 μL, 5 equiv.) was added and the reaction mixture was stirred at room temperature for 30 minutes. Volatiles were removed in vacuo to yield the crude product, which was dissolved in MeOH, applied to a SCX column and eluted with MeOH followed by 2M ammonia in MeOH. Ammonia/MeOH fractions were combined and evaporated to afford the expected product (30 mg). LCMS: MW (calcd): 333.4; MS (ES⁺, m/z): 334.0 [M+H]⁺.

### Method H: General Procedure for Mesylation

Typically, to a solution of an appropriate amine (1 equiv.) in DCM (or any other suitable solvent), DIPEA or other suitable base (2.5 equiv.) and mesyl chloride (1.1 equiv.) are added. The reaction mixture is stirred at room temperature for 45 minutes. The expected compound may be isolated and, if desired, further purified by methods known to one skilled in the art.

### Example H.1

### Illustrative Synthesis of 1-cyclopentyl-3-(4-fluorophenyl)-1-[(4-methyl-1-methylsulfonyl-4-piperidyl)methyl]urea (Compound 113)

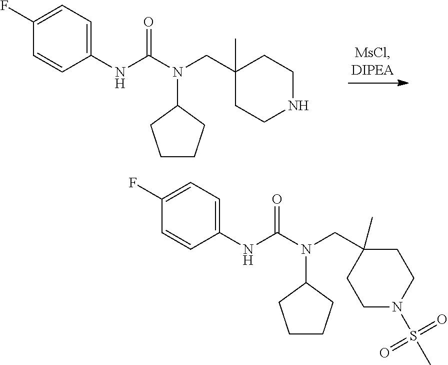

To a solution of 1-cyclopentyl-3-(4-fluorophenyl)-1-[(4-methyl-4-piperidyl)methyl]urea (24 mg, 1 equiv.) and DIPEA (32 μL, 2.5 equiv.) in DCM (1.0 mL) mesyl chloride (6 μL, 1.1 equiv.) was added and the resulting mixture was stirred at RT for 45 minutes. The reaction mixture was transferred to a separatory funnel containing water (5 mL) and extracted with DCM (3×10 mL). Organic layers were combined, then evaporated to dryness to obtain the crude product, which was further purified by preparative LC-MS to afford the expected product (5 mg). LCMS: MW (calcd): 411.5; MS (ES⁺, m/z): 412.18 [M+H]⁺.

### Method I: General Procedure for Acetylation

Typically, to a solution of an appropriate amine (1 equiv.) in THE (or any other suitable solvent) at 0° C., TEA or other suitable base (2.5 equiv.) and acetyl chloride (1.1 equiv.) are added. The reaction mixture is stirred at room temperature for 45 minutes. The expected compound may be isolated and, if desired, further purified by methods known to one skilled in the art.

### Example 1.1

### Illustrative Synthesis of 1-[(1-acetyl-4-methyl-4-piperidyl)methyl]-1-cyclopentyl-3-phenyl-urea (Compound 97)

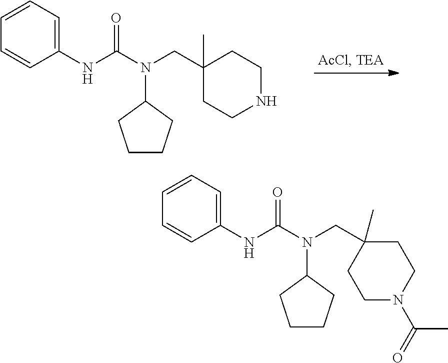

To a solution of 1-cyclopentyl-1-[(4-methyl-4-piperidyl)methyl]-3-phenyl-urea (18 mg, 1 equiv.) and TEA (20 μL, 2.5 equiv.) in THE (1.0 mL) at 0° C. acetyl chloride (4.9 μL, 1.1 equiv.) was added and the resulting mixture was stirred at RT for 45 minutes. The reaction mixture was concentrated, transferred to a separatory funnel containing water (5 mL) and extracted with EtOAc (3×10 mL). Organic layers were evaporated to dryness and the obtained crude product was further purified by flash chromatography on silica gel (eluting with: EtOAc/cyclohexane gradient; 0-50% of EtOAc) to afford the expected product (12 mg). LCMS: MW (calcd): 357.5; MS (ES⁺, m/z): 358.24 [M+H]⁺.

### Method J: General Procedure for Methylation

Typically, to a solution of an appropriate urea (1 equiv.) in DMF (or any other suitable solvent), Cs₂CO₃ or other suitable base (1.5 equiv.) and methyl iodide (1.1 equiv.) are added. The reaction mixture is stirred at room temperature for 24 hours. The expected compound may be isolated and, if desired, further purified by methods known to one skilled in the art.

### Example J.1

### Illustrative Synthesis of 1-cyclopentyl-3-methyl-3-phenyl-1-(2-thienylmethyl)urea (Compound 23)

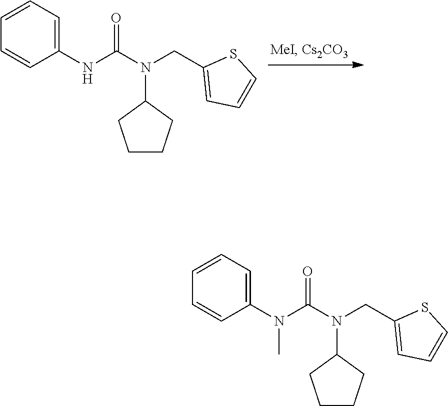 [SMILES: O=C(Nc1ccccc1)N(Cc1cccs1)C1CCCC1 ; CN(C(=O)N(Cc1cccs1)C1CCCC1)c1ccccc1]

To the suspension of 1-cyclopentyl-3-phenyl-1-(2-thienylmethyl)urea (50 mg, 1 equiv.) and Cs₂CO₃ (81.3 mg, 1.5 equiv.) in DMF (1.0 mL), methyl iodide (12 μL, 1.1 equiv.) was added and the resulting mixture was stirred at RT for 16 hours. The reaction mixture was diluted with EtOAc (5 mL) and washed with brine (3×10 mL). Organic layer was dried over sodium sulphate, filtered and evaporated to dryness. The obtained crude product was further purified by preparative LC-MS to afford the expected product (21 mg). LCMS: MW (calcd): 314.4; MS (ES⁺, m/z): 315.70 [M+H]⁺.

### Method K: General Procedure for Negishi Coupling

Typically, to a suspension of zinc (9.3 equiv.) in THE (or any other suitable solvent) an appropriate alkyl iodide (6.6 equiv.) is added. The reaction mixture is heated at 80° C. for 90 minutes. The reaction temperature is lowered and aryl iodide (1 equiv.), palladium catalyst (0.05 equiv.), such as Pd(OAc)₂ or any other suitable catalyst, and ligand (0.1 equiv.), such as CPhos or any other suitable ligand, are added. The reaction mixture is stirred at 60° C. for 2 hours and then at room temperature for 16 hours. The expected compound may be isolated and, if desired, further purified by methods known to one skilled in the art.

### Example K.1

### Illustrative Synthesis of tert-butyl 3-[4-[[cyclopentyl-[(5-methyl-2-furyl)methyl]-carbamoyl]amino]phenyl]azetidine-1-carboxylate (Compound 159)

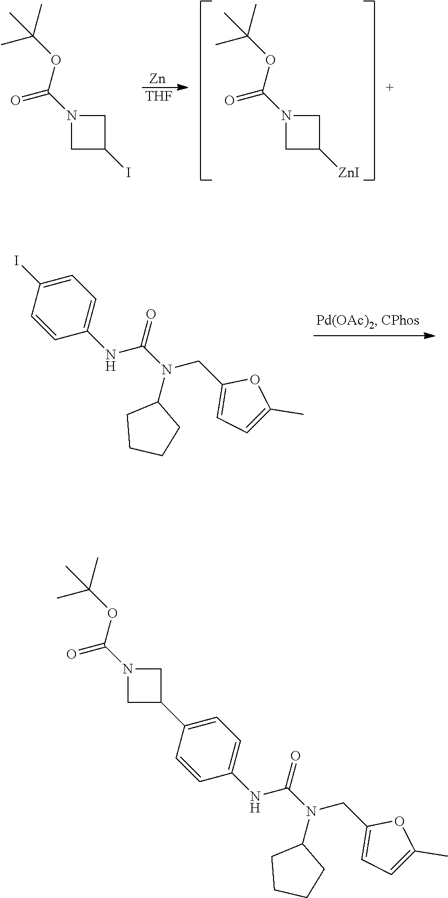

HCl-activated Zn (90.1 mg, 9.3 equiv.) was suspended in THE (0.5 mL) under argon atmosphere. A solution of 1-Boc-3-iodoazetidine (300 mg, 6.6 equiv.) in THE (0.5 mL) was added and the reaction mixture was heated at 80° C. for 90 minutes. The reaction temperature was lowered and 1-cyclopentyl-3-(4-iodophenyl)-1-[(5-methyl-2-furyl)methyl]urea (63 mg, 1 equiv.), Pd(OAc)₂ (1.6 mg, 0.05 equiv.) and CPhos (10 mg, 0.1 equiv.) were added. The reaction mixture was heated at 60° C. for 2 hours and then at room temperature for 16 hours. The reaction mixture was diluted with DCM (10 mL) and filtered through a celite pad. Collected organic fraction was transferred to a separatory funnel and washed with sat. aq. sol. NaHCO₃ (10 mL). Organic layer was dried, concentrated and purified by flash chromatography on silica gel (eluting with: EtOAc/cyclohexane gradient; 0-20% of EtOAc) to afford the expected product (36 mg).

### Method L: General Procedure for Nitro Reduction

Typically, to a solution of an appropriate nitro compound (1.0 equiv.), Pd/C 10% (on carbon) (0.01 equiv.) and in a suitable solvent, such as MeOH at room temperature is added. The reaction mixture is stirred under hydrogen atmosphere at room temperature for 4 hours. The expected compound may be isolated and, if desired, further purified by methods known to one skilled in the art.

### Example L.1

### Illustrative Synthesis of [1-(4-aminophenyl)triazol-4-yl]methanol

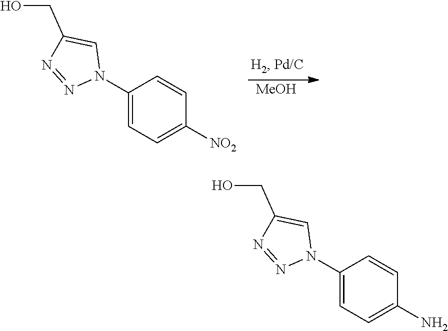

To a solution of [1-(4-nitrophenyl)triazol-4-yl]methanol (500 mg, 1 equiv.) in MeOH (45.0 mL), Pd/C (10% on carbon) (36.0 mg, 0.01 equiv.) at room temperature was added. The resulting mixture was stirred under H₂ atmosphere for 4 hours. The reaction mixture filtered through Celite. Obtained filtrate was concentrated to afford the expected product (422 mg). LCMS: MW (calcd): 190.20; MS (ES⁺, m/z): 191.09 [M+H]⁺.

### Example L.2

### Illustrative Synthesis of 4-(4-methyltriazol-1-yl)aniline

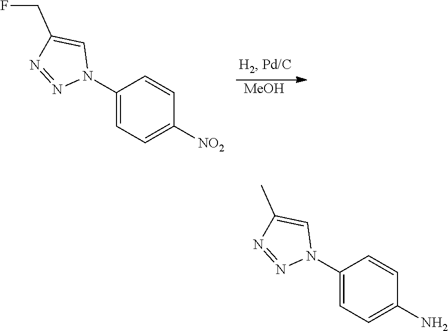 [SMILES: "MeOH" ; O=[N+]([O-])c1ccc(-n2cc(CF)nn2)cc1 ; Cc1cn(-c2ccc(N)cc2)nn1]

To a solution of 4-(fluoromethyl)-1-(4-nitrophenyl)triazole (7 mg, 1 equiv.) in MeOH (2.0 mL), Pd/C (10% on carbon) (5 mg, 0.15 equiv.) at room temperature was added. The resulting mixture was stirred under H₂ atmosphere for 18 hours. The reaction mixture was filtered through syringe filter (PTFE, 0.45 m). Obtained filtrate was concentrated to afford the expected product (3.5 mg). LCMS: MW (calcd): 174.20; MS (ES⁺, m/z): 175.06 [M+H]⁺.

### Method M: General Procedure for Silyl Protection

Typically, to a solution of an appropriate compound (1.0 equiv.), DMAP (0.3 equiv.) and a base (1.5 equiv.), such as TEA in a suitable solvent, such as DCM at 0° C., a solution of tert-butyl-chloro-dimethyl-silane (1.5 equiv.) in a suitable solvent, such as DCM is added. The reaction mixture is stirred at 0° C. for 10 minutes and then at room temperature for 2.5-16 hours. The expected compound may be isolated and, if desired, further purified by methods known to one skilled in the art.

### Example M.1

### Illustrative Synthesis of 4-[4-[[tert-butyl(dimethyl)silyl]oxymethyl]triazol-1-yl]aniline

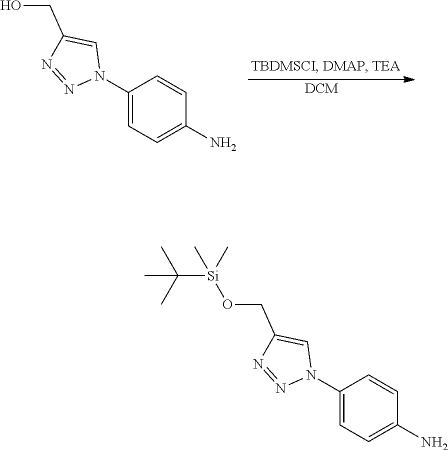

To a solution of [1-(4-aminophenyl)triazol-4-yl]methanol (50 mg, 1 equiv.), DMAP (9.6 mg, 0.3 equiv.) and TEA (54 μL, 1.5 equiv.) in DCM (0.65 mL) at 0° C. TBDMSCl (59 mg, 1.5 equiv.) was added. The resulting mixture was stirred at 0° C. for 10 minutes, and then at room temperature for 16 hours. The reaction mixture was transferred to a separatory funnel containing sat. aq. sol. NaHCO₃ (5 mL) and extracted with DCM (3×5 mL). Organic layers were combined, dried, concentrated and purified by flash chromatography on silica gel (eluting with: EtOAc/cyclohexane gradient; 0-30% of EtOAc) to afford the expected product (72 mg). LCMS: MW (calcd): 304.46; MS (ES⁺, m/z): 305.62 [M+H]⁺.

### Method N: General Procedure for Silyl Deprotection

Typically, to a solution of tert-butyldimethyl silyl protected compound (1.0 equiv.) in an appropriate solvent, such as THE at 0° C., TBAF×H₂O (1 equiv.) is added. The reaction mixture is stirred at 0° C. for 3 hours. The expected compound may be isolated and, if desired, further purified by methods known to one skilled in the art.

### Example N.1

### Illustrative Synthesis of 1-cyclopentyl-3-[4-[4-(hydroxymethyl)triazol-1-yl]phenyl]-1-[(5-methyl-2-furyl)methyl]urea (Compound 164)

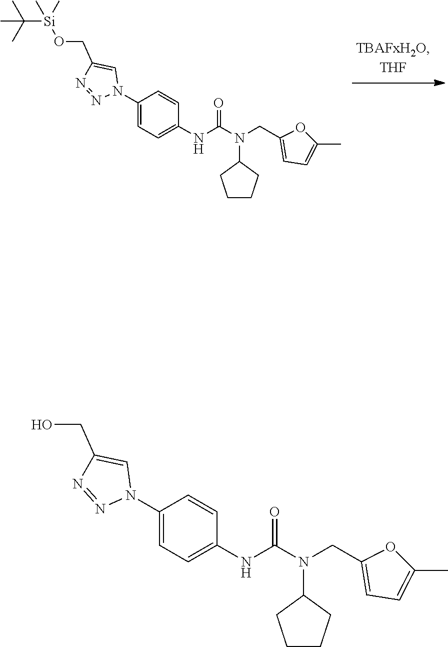 [SMILES: Cc1ccc(CN(C(=O)Nc2ccc(-n3cc(CO[Si](C)(C)C(C)(C)C)nn3)cc2)C2CCCC2)o1 ; Cc1ccc(CN(C(=O)Nc2ccc(-n3cc(CO)nn3)cc2)C2CCCC2)o1]

To a solution of 3-[4-[4-[[tert-butyl(dimethyl)silyl]oxymethyl]triazol-1-yl]phenyl]-1-cyclopentyl-1-[(5-methyl-2-furyl)methyl]urea (8 mg, 1 equiv.) in THF (0.5 mL) at 0° C. TBAF×H₂O (5 mg, 1 equiv.) was added. The resulting mixture was stirred at 0° C. After completion, the reaction mixture was concentrated and purified by a column chromatography (φ 0.5 cm, h-SiO₂ 8 cm, eluent: DCM/MeOH=20/1). Collected fractions were combined and evaporated till dryness to afford the expected product (3 mg). LCMS: MW (calcd): 395.45; MS (ES⁺, m/z): 396.76 [M+H]⁺.

### Method O: General Procedure for Benzyl Deprotection

Typically, to a solution of O-benzyl protected compound (1.0 equiv.) in the mixture of solvents ethanol/ethyl-acetate (1:1), Pearlman's catalyst (20% wt. % loading activated) was added. The reaction mixture was hydrogenated in a Parr apparatus at 3 bar at ambient temperature for 17 hours. The expected compound may be isolated and, if desired, further purified by methods known to one skilled in the art.

### Example O.1

### Illustrative Synthesis of 1-cyclopentyl-3-(4-hydroxyphenyl)-1-[(5-methyl-2-furyl)methyl]urea (Compound 292)

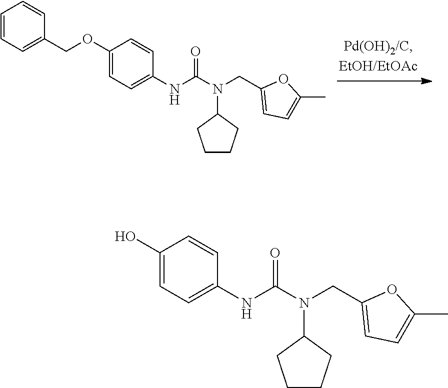 [SMILES: Cc1ccc(CN(C(=O)Nc2ccc(OCc3ccccc3)cc2)C2CCCC2)o1 ; Cc1ccc(CN(C(=O)Nc2ccc(O)cc2)C2CCCC2)o1]

To a solution of 3-(4-benzyloxyphenyl)-1-cyclopentyl-1-[(5-methyl-2-furyl)methyl]urea (70 mg, 1 equiv.) in mixture of 96% EtOH/EtOAc (1:1) (30 mL), 20% wt. % Pd(OH)₂/C (39 mg, 1.7 equiv) was added. The reaction mixture was hydrogenated in a Parr apparatus at 3 bar at ambient temperature for 17 hours. The resulting suspension was filtered and concentrated in vacuo. Obtained crude product was purified by flash chromatography on silica gel (eluting with: DCM/2.5% MeOH in DCM gradient; 0-10% of 2.5% MeOH in DCM) to afford the expected product (9.6 mg). LCMS: MW (calcd): 314.38; MS (ES⁺, m/z): 315.07 [M+H]⁺.

### Method P: General Procedure for the Base-Catalysed Conversion of Nitriles to Amides by Hydrogen Peroxide

Typically, to a solution of nitrile compound (1.0 equiv.) in DMSO, or any other suitable solvent, a base (30 equiv.), such as potassium carbonate, and hydrogen peroxide (95-115 equiv.) are added. The reaction mixture is stirred at RT for 16 hours. The expected compound may be isolated and, if desired, further purified by methods known to one skilled in the art.

### Example P.1

### Illustrative Synthesis of 4-[[cyclopentyl-[(5-methyl-2-furyl)methyl]carbamoyl]-amino]benzamide (Compound 117)

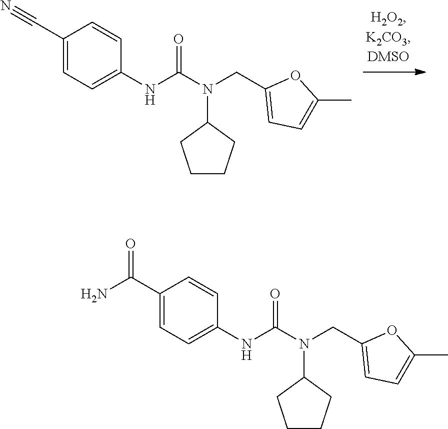 [SMILES: Cc1ccc(CN(C(=O)Nc2ccc(C#N)cc2)C2CCCC2)o1 ; Cc1ccc(CN(C(=O)Nc2ccc(C(N)=O)cc2)C2CCCC2)o1]

To a suspension of 3-(4-cyanophenyl)-1-cyclopentyl-1-[(5-methyl-2-furyl)methyl]urea (50 mg, 1 equiv.) and K₂CO₃ (639 mg, 30 equiv.) in DMSO (2.5 mL) 30 wt % H₂O₂ in water (1.5 mL, 95 equiv.) was added. The resulting mixture was stirred at room temperature for 16 hours. The reaction mixture was transferred to a separatory funnel containing water (10 mL) and extracted with DCM (3×10 mL). Organic layers were dried, evaporated in vacuo and crystalized from diethyl ether to afford the expected product (51 mg). LCMS: MW (calcd): 341.40; MS (ES⁺, m/z): 342.19 [M+H]⁺.

### Method O: General Procedure for Phenyl Carbamate Synthesis

Typically, to a solution or a suspension of an appropriate amine (1 equiv.) and a base (1.1 equiv.), such as pyridine in a suitable solvent, such as DCM or CH₃CN, phenyl chloroformate (1-1.05 equiv.) is added at 0° C. The resulting mixture is stirred at 0° C. or at room temperature for 2-16 hours. The expected phenyl carbamate may be isolated and, if desired, further purified by methods known to one skilled in the art or used as a crude product in the following reaction step.

### Example Q.1

### Illustrative Synthesis of phenyl N-(5-ethynyl-2-pyridyl)carbamate

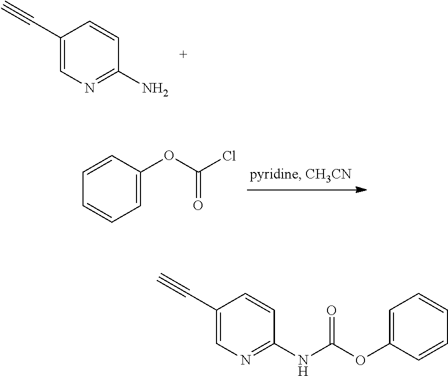

To a stirred solution of 5-ethynylpyridine-2-amine (50 mg, 1 equiv.) and pyridine (37 μL, 1.1 equiv.) in dry CH₃CN (1 mL) at 0° C. was added phenyl chloroformate (53 μL, 1 equiv.). A brown precipitate appeared, which was filtered, washed with cold acetonitrile and dried under vacuum overnight to afford the expected product (84 mg). LCMS: MW (calcd): 238.07; MS (ES⁺, m/z): 239.63 [M+H]⁺.

### Method R: General Procedure for Isopropenyl Carbamate Synthesis

Typically, to a solution or a suspension of an appropriate amine (1 equiv.) and a base such as aqueous saturated solution of NaHCO₃ with a suitable solvent, such as EtOAc, isopropenyl chloroformate (1-1.1 equiv.) is added at room temperature. The resulting mixture is stirred at room temperature for 1 hour. The expected phenyl isopropenyl carbamate may be isolated and, if desired, further purified by methods known to one skilled in the art or used as a crude product in the following reaction step.

### Example R.1

Illustrative Synthesis of isopropenyl N-[4-[2-[tert-butyl(dimethyl)silyl]oxyethynyl]phenyl]carbamate

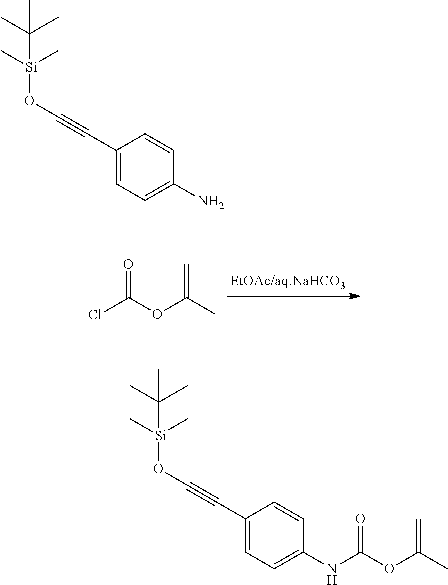

To a stirred solution of 4-[2-[tert-butyl(dimethyl)silyl]oxyethynyl]aniline (200 mg, 1.0 equiv.) in EtOAc (6.98 mL), saturated aqueous solution of NaHCO₃ (6.98 mL) and isopropenyl chloroformate (0.092 ml, 1.1 equiv.) were added. The reaction mixture was stirred at room temperature for 1 hour. Organic layer was separated, aqueous layer was extracted with EtOAc (3×15 mL). Combined organic extracts were dried over Na₂SO₄, filtered and evaporated in vacuo to yield expected product (278 mg). LCMS: MW (calcd): 345.51; MS (ES⁻, m/z): 344.14 [M−H]+.

### Method S: General Procedure for Copper-Catalysed C-N Coupling of Amides in the Presence of Caesium Fluoride

Typically, to a solution or a suspension of an appropriate aryl iodide (1.0 equiv.), CsF (2.5 equiv.) and a copper catalyst, such as CuI, in a suitable solvent, such as dioxane, an amide (1.2 equiv.), such as 2-pyrrolidinone, and a ligand (0.1 equiv.), such as 1,2-bis(methylamino)ethane, were added. The resulting mixture is stirred at room temperature for 3 hours. The expected product may be isolated and, if desired, further purified by methods known to one skilled in the art or used as a crude product in the following reaction step.

### Example S.1

### Illustrative Synthesis of 1-(4-aminophenyl)pyrrolidin-2-one

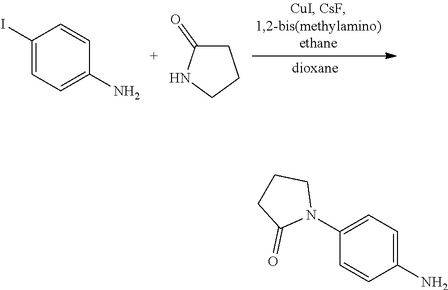 [SMILES: Nc1ccc(I)cc1 ; O=C1CCCN1 ; Nc1ccc(N2CCCC2=O)cc1]

Into a reaction tube were placed CsF (190 mg, 2.5 equiv.), CuI (4.8 mg, 0.05 equiv.) and 4-iodoaniline (110 mg, 1 equiv.). The tube was evacuated and back-filled with argon (3×). The solids were dissolved in 1,4-dioxane (0.5 mL) and 2-pyrrolidinone (45 μL, 1.2 equiv.) and 1,2-bis(methylamino)ethane (5.4 μL, 0.1 equiv.) were added. The reaction mixture was stirred at room temperature for 3 hours. The reaction mixture was diluted with EtOAc (5 mL), transferred to a separatory funnel containing sat. aq. sol. NH₄Cl (5 mL) and was extracted with EtOAc (2×5 mL). The combined organics were dried over Na₂SO₄ (anhyd.), filtered and concentrated. Obtained crude product was pooled with crude product from another reaction that was done in the same way and with the same amount of the starting material and reagents. The combined crude product was purified by flash chromatography on silica gel (eluting with: MeOH/DCM gradient; 0-5% of MeOH) to afford the expected product (62 mg). ¹H NMR (400 MHz, CDCl₃): δ=7.32 (d, J=8.9 Hz, 2H), 6.66 (d, J=8.9 Hz, 2H), 3.78 (t, J=7.0 Hz, 2H), 2.55 (t, J=7.8 Hz, 2H), 2.11 (quint, J=7.6 Hz, 2H) ppm.

### Method T: General Procedure for Stille Coupling

Typically, to a solution or a suspension of an appropriate aryl bromide (1.0 equiv.) and a palladium catalyst (0.05 equiv.), such as Pd(PPh₃)₄ or any other suitable catalyst, in a suitable solvent, such as toluene, tributyl-propynylstannane (1.2 equiv.) is added. The resulting mixture is heated at 100° C. for 16 hours. The expected product may be isolated and, if desired, further purified by methods known to one skilled in the art or used as a crude product in the following reaction step.

### Example T.1

### Illustrative Synthesis of 4-prop-1-ynylaniline

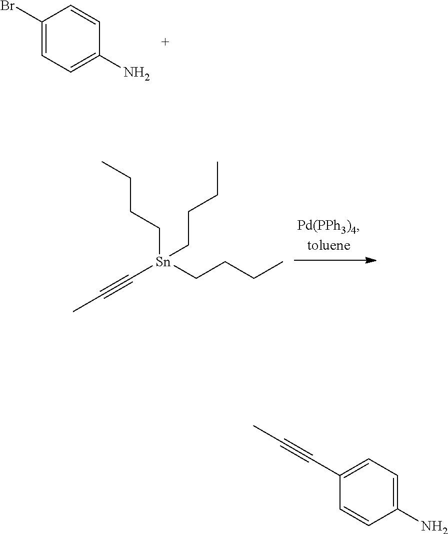

To a solution of 4-bromoaniline (150 mg, 1 equiv.) and Pd(PPh₃)₄ (50 mg, 0.05 equiv.) in toluene (8.7 mL) under argon atmosphere, tributyl-propynylstannane (344 mg, 1.2 equiv.) was added. The reaction mixture was heated at 100° C. for 16 hours. The reaction was cooled to room temperature, transferred to a separatory funnel containing sat. aq. sol. NaHCO₃ (100 mL) and extracted with EtOAc (3×50 mL). The combined organic layers were dried over Na₂SO₄ (anhyd.), filtered and concentrated. The crude product was purified by flash chromatography on silica gel (eluting with: EtOAc/cyclohexane gradient; 0-25% of EtOAc) to afford the expected product as a 1:1 mixture with starting 4-bromoaniline (60 mg), and was used in the following reaction without further purification. LCMS: MW (calcd): 131.17; MS (ES⁺, m/z): 131.41 [M+H]⁺.

### Method U: General Procedure for Substitution of Hydroxyl Group with Fluorine

Typically, to a solution or a suspension of an appropriate alkyl hydroxide (1.0 equiv.) in dry DCM, Deoxo-Fluor reagent (13 equiv) is added under argon atmosphere. The resulting mixture was stirred at −40° C. for 72 hours. The expected product may be isolated and, if desired, further purified by methods known to one skilled in the art or used as a crude product in the following reaction step.

### Example U.1

### Illustrative Synthesis of 4-(fluoromethyl)-1-(4-nitrophenyl)triazole

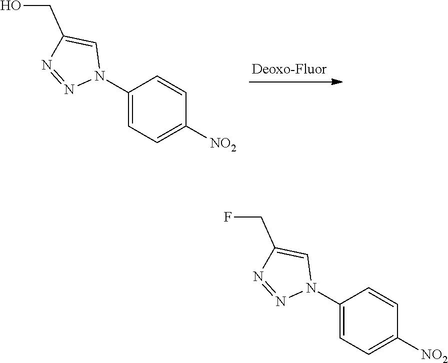 [SMILES: O=[N+]([O-])c1ccc(-n2cc(CO)nn2)cc1 ; O=[N+]([O-])c1ccc(-n2cc(CF)nn2)cc1]

To a solution of [1-(4-nitrophenyl) triazol-4-yl]methanol (50 mg, 1 equiv.) in dry DCM (2.5 mL), previously cooled to −40° C., under argon atmosphere, Deoxo-Fluor (50% solution in THF) (0.625 mL, 12.94 equiv.) was added. The reaction mixture was stirred at −40° C. for 72 hours under argon atmosphere. To the reaction mixture, water (15 mL) was added and resulting suspension was extracted with EtOAc (3×15 mL). Combined organic layers were filtered through phase separator and evaporated in vacuo to dryness. The obtained crude product was further purified by preparative LC-MS to afford the expected product (9.2 mg). LCMS: MW (calcd): 222.18; MS (ES⁺, m/z): 223.06 [M+H]⁺.

### Method V: General Procedure for Azide Formation from Amine

Typically, to a cooled solution or a suspension of an appropriate amine (1.0 equiv.) in aqueous solution of HCl acid, a solution of sodium nitrite (1.05 equiv.) in water is added dropwise. Reaction mixture was stirred for 10 minutes, followed by addition of sodium azide (1.03 equiv.). The resulting mixture was stirred for 10 minutes, followed by addition of EtOAc. The expected product may be isolated and, if desired, further purified by methods known to one skilled in the art or used as a crude product in the following reaction step.

### Example V.1

### Illustrative Synthesis of 1-azido-2-methoxy-4-nitro-benzene

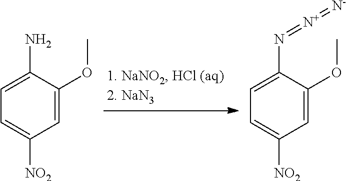

To an ice cooled solution of 2-methoxy-4-nitro-aniline (500 mg, 1 equiv.) in 2M aqueous solution of HCl (10 mL), a solution of sodium nitrite (215 mg, 1.05 equiv.) in water (0.9 mL) was added dropwise during 10 minutes. Reaction mixture was continued to stir on ice for additional 10 minutes, followed by addition of sodium azide (200 mg, 1.03 equiv.). The reaction mixture was stirred for 10 minutes, EtOAc (30 mL) was added, and stirring was continued for 10 minutes. Aqueous layer was separated and extracted with EtOAc (2×10 mL). Combined organic layers were filtered through phase separator and evaporated in vacuo to afford the expected product (536 mg). ¹H NMR (300 MHz, DMSO-d6): δ=7.91-7.76 (m, 2H), 7.32-7.24 (m, 1H), 3.96 (s, 3H) ppm.

### Method W: General Procedure for Synthesis of 1,2,3-triazoles

In the mixture of suitable solvents (typically DMF/water), an appropriate azide (1.0 equiv), alkyne (1.0 equiv) in the presence of catalyst (typically CuSO₄×5H₂O, 0.8 equiv) and sodium ascorbate (1.35 equiv.) were added. Resulting mixture was exposed to microwave irradiation at 150° C. for 2 minutes. The expected product may be isolated and, if desired, further purified by methods known to one skilled in the art or used as a crude product in the following reaction step.

### Example W.1 Illustrative Synthesis of [1-(2-methoxy-4-nitro-phenyl)triazol-4-yl]methanol

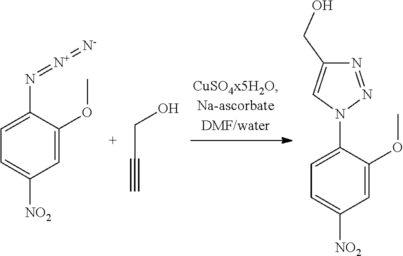

To the mixture of DMF (1.95 mL) and water (0.65 mL), 1-azido-2-methoxy-4-nitro-benzene (50 mg, 1.0 equiv), prop-2-yn-1-ol (0.015 mL, 1.0 equiv.), CuSO₄×5H₂O (50 mg, 0.78 equiv.) and sodium ascorbate (69 mg, 1.35 equiv.) were added. To the reaction mixture, water (10 mL) and brine (10 mL) were added. Resulting solution was extracted with DCM (3×15 mL). Combined organic extracts were washed with brine, filtered through phase separator and evaporated in vacuo to afford the expected product (924 mg). LCMS: MW (calcd): 250.21; MS (ES⁺, m/z): 251.08 [M+H]⁺.

Table with Representative Compounds of Formula (I):

| | | MS | MS | | | | |
|---|---|---|---|---|---|---|---|
| Cpd | | (m/z, | (m/z, | | Starting | | |
| # | Structure | ES+) | ES−) | δ NMR Data | Int/Cpd# | Sequence of Methods | Chemical Name |
| 1 | 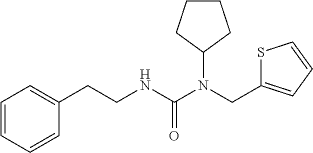 [SMILES: O=C(NCCc1ccccc1)N(Cc1cccs1)C1CCCC1] | 329.8 | | 1H NMR (300 MHz, DMSO-d6): δ = 7.32 (dd, J = 1.7, 4.5 Hz, 1H), 7.29-7.21 (m, 2H), 7.19-7.12 (m, 3H), 6.91-6.87 (m, 2H), 6.36 (t, J = 5.3 Hz, 1H), 4.49 (s, 2H), 4.24- 4.12 (m, 1H), 3.27-3.19 (m, 2H), 2.70 (t, J = 7.2 Hz, 2H), 1.72-1.53 (m, 4H), 1.51-1.37 (m, 4H) ppm | N-(2- thienyl- methyl)- cyclopentan- amine | General method D1 using 2- isocyanatoethyl- benzene | 1-cyclopentyl-3- (2-phenylethyl)- 1-(2- thienylmethyl) urea |
| 2 | 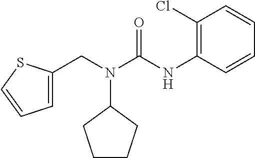 [SMILES: O=C(Nc1ccccc1Cl)N(Cc1cccs1)C1CCCC1] | 335.7 | | 1H NMR (300 MHz, DMSO-d6): δ = 7.74 (s, 1H), 7.66 (dd, J = 1.5, 8.0 Hz, 1H), 7.41 (d, J = 1.4 Hz, 1H), 7.39 (dd, J = 3.2, 1.2 Hz, 1H) 7.26 (td, J = 7.7, 1.5 Hz, 1H), 7.11- 7.04 (m, 2H), 6.98-6.94 (m, 1H), 4.66 (s, 2H), 4.51-4.41 (m, 1H), 1.88-1.76 (m, 2H), 1.69-1.44 (m, 6H) ppm | N-(2- thienyl- methyl)- cyclopentan- amine | General method D1 using 1-chloro-2- isocyanato- benzene | 3-(2- chlorophenyl)-1- cyclopentyl-1- (2- thienylmethyl) urea |
| 3 | 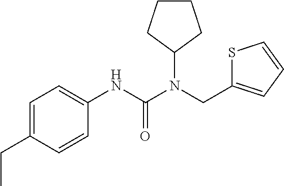 [SMILES: CCc1ccc(NC(=O)N(Cc2cccs2)C2CCCC2)cc1] | 329.8 | | 1H NMR (300 MHz, DMSO-d6): δ = 8.24 (s, 1H), 7.36-7.32 (m, 2H), 7.30 (s, 1H), 7.05 (d, J = 8.4 Hz, 2H), 7.00-6.96 (m, 1H), 6.94- 6.98 (m, 1H), 4.65 (s, 2H), 4.46- 4.36 (m, 1H), 2.56 (m, 2H), 1.82- 1.69 (m, 2H), 1.67-1.42 (m, 6H), 1.13 (t, J = 7.4 Hz, 3H) ppm | N-(2- thienyl- methyl)- cyclopentan- amine | General method D1 using 1-ethyl-4- isocyanato- benzene | 1-cyclopentyl-3- (4-ethylphenyl)- 1-(2- thienylmethyl) urea |
| 4 | 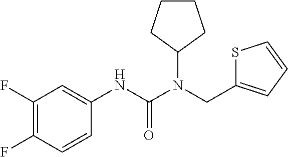 [SMILES: O=C(Nc1ccc(F)c(F)c1)N(Cc1cccs1)C1CCCC1] | 337.7 | | 1H NMR (300 MHz, DMSO-d6): δ = 8.58 (s, 1H), 7.65-7.57 (m, 1H), 7.34 (dd, J = 5.0, 1.2 Hz, 1H), 7.31-7.18 (m, 2H), 7.01-6.97 (m, 1H), 6.94-6.90 (m, 1H), 4.65 (s, 2H), 4.45-4.34 (m, 1H), 1.37-1.85 (m, 8H) ppm | N-(2- thienyl- methyl)- cyclopentan- amine | General method D1 using 1,2-difluoro-4- isocyanato- benzene | 1-cyclopentyl-3- (3,4- difluorophenyl)- 1-(2- thienylmethyl) urea |
| 5 | 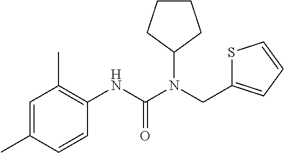 [SMILES: Cc1ccc(NC(=O)N(Cc2cccs2)C2CCCC2)c(C)c1] | 329.7 | | 1H NMR (300 MHz, DMSO-d6): δ = 7.67 (s, 1H), 7.36 (dd, J = 5.0, 1.2 Hz, 1H), 7.07 (d, J = 8.0 Hz, 1H), 7.02-6.97 (m, 1H), 6.95-6.87 (m, 3H), 4.63 (s, 2H), 4.47-4.36 (m, 1H), 2.22 (s, 3H), 2.01 (s, 3H), 1.84-1.71 (m, 2H), 1.67-1.42 (m, 6H) ppm | N-(2- thienyl- methyl)- cyclopentan- amine | General method D1 using 1-isocyanato-2,4- dimethylbenzene | 1-cyclopentyl-3- (2,4- dimethylphenyl)- 1-(2- thienylmethyl) urea |
| 6 | 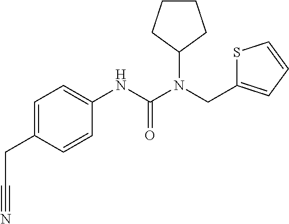 [SMILES: N#CCc1ccc(NC(=O)N(Cc2cccs2)C2CCCC2)cc1] | 340.7 | | 1H NMR (300 MHz, DMSO-d6): δ = 8.42 (s, 1H), 7.45 (d, J = 8.5 Hz, 2H), 7.33 (dd, J = 4.7, 1.7 Hz, 1H), 7.19 (d, J = 8.5 Hz, 2H), 7.00- 6.95 (m, 1H), 6.93-6.89 (m, 1H), 4.66 (s, 2H), 4.48-4.36 (m, 1H), 3.92 (s, 2H), 1.82-1.69 (m, 2H), 1.68-1.43 (m, 6H) ppm | N-(2- thienyl- methyl)- cyclopentan- amine | General method D1 using 2-(4- isocyanatophenyl) acetonitrile | 3-[4- (cyanomethyl) phenyl]-1- cyclopentyl-1- (2- thienylmethyl) urea |
| 7 | 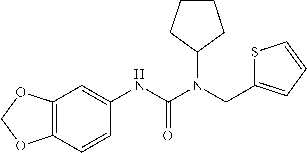 [SMILES: O=C(Nc1ccc2c(c1)OCO2)N(Cc1cccs1)C1CCCC1] | 345.7 | | 1H NMR (300 MHz, DMSO-d6): δ = 8.23 (s, 1H), 7.33 (dd, J = 5.0, 1.3 Hz, 1H), 7.10 (d, J = 1.4 Hz, 1H), 7.02-6.97 (m, 1H), 6.94-6.89 (m, 1H), 6.81-6.75 (m, 2H), 5.93 (s, 2H), 4.63 (s, 2H), 4.44-4.34 (m, 1H), 1.81-1.69 (m, 2H), 1.68- 1.42 (m, 6H) ppm | N-(2- thienyl- methyl)- cyclopentan- amine | General method D1 using 5-isocyanato-1,3- benzodioxole | 3-(1,3- benzodioxol-5- cyclopentyl-1- (2- thienylmethyl) urea |
| 8 | 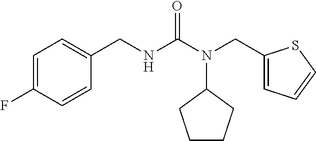 [SMILES: O=C(NCc1ccc(F)cc1)N(Cc1cccs1)C1CCCC1] | 333.7 | | 1H NMR (300 MHz, DMSO-d6): δ = 7.36-7.25 (m, 2H), 7.08-6.84 (m, 6H), 4.55 (s, 2H), 4.32-4.21 (m, 3H), 1.78-1.68 (m, 2H), 1.67- 1.55 (m, 2H), 1.54-1.39 (m, 4H) ppm | N-(2- thienyl- methyl)- cyclopentan- amine | General method D1 using 1-fluoro-4- (isocyanatomethyl) benzene | 1-cyclopentyl- [(4- fluorophenyl) methyl]-1-(2- thienylmethyl) urea |
| 9 | 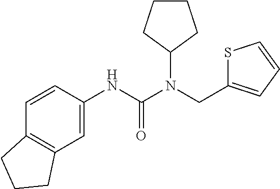 [SMILES: O=C(Nc1ccc2c(c1)CCC2)N(Cc1cccs1)C1CCCC1] | 341.8 | | 1H NMR (300 MHz, DMSO-d6): δ = 8.19 (s, 1H), 7.35-7.30 (m, 2H), 7.14-7.09 (m, 1H), 7.04 (d, J = 8.4 Hz, 1H), 7.00-6.96 (m, 1H), 6.94-6.89 (m, 1H), 4.64 (s, 2H), 4.47-4.36 (m, 1H), 2.77 (q, J = 7.3 Hz, 4H), 2.02-1.92 (m, 2H), 1.80- 1.69 (m, 2H), 1.67-1.42 (m, 6H) ppm | N-(2- thienyl- methyl)- cyclopentan- amine | General method D1 using 5- isocyanatoindane | 1-cyclopentyl-3- indan-5-yl-1-(2- thienylmethyl) urea |
| 10 | 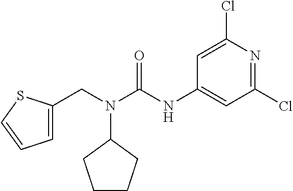 [SMILES: O=C(Nc1cc(Cl)nc(Cl)c1)N(Cc1cccs1)C1CCCC1] | 370.7 | | 1H NMR (300 MHz, DMSO-d6): δ = 9.27 (s, 1H), 7.63 (s, 2H), 7.36 (dd, J = 5.1, 1.1 Hz, 1H), 7.01-6.98 (m, 1H), 6.94-6.89 (m, 1H), 4.67 (s, 2H), 4.37 (quin, J = 8.0 Hz, 1H), 1.84-1.71 (m, 2H), 1.70-1.43 (m, 6H) ppm | N-(2- thienyl- methyl)- cyclopentan- amine | General method D1 using 2,6-dichloro-4- isocyanato-pyridine | 1-cyclopentyl-3- (2,6-dichloro-4- pyridyl)-1-(2- thienylmethyl) urea |
| 11 | 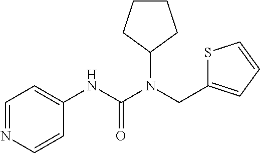 [SMILES: O=C(Nc1ccncc1)N(Cc1cccs1)C1CCCC1] | 302.6 | | 1H NMR (300 MHz, DMSO-d6): δ = 8.80 (s, 1H), 8.29 (d, J = 6.5 Hz, 2H), 7.46 (d, J = 6.2 Hz, 2H), 7.34 (dd, J = 1.1, 5.0 Hz, 1H), 7.01-6.98 (m, 1H), 6.92 (dd, J = 3.6, 5.0 Hz, 1H), 4.68 (s, 2H), 4.50-4.37 (m, 1H), 1.84-1.70 (m, 2H), 1.70-1.42 (m, 6H) ppm | N-(2- thienyl- methyl)- cyclopentan- amine | General method D1 using 4- isocyanatopyridine | 1-cyclopentyl-3- (4-pyridyl)-1-(2- thienylmethyl) urea |
| 12 | 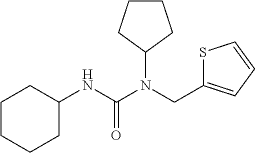 [SMILES: O=C(NC1CCCCC1)N(Cc1cccs1)C1CCCC1] | 307.7 | | 1H NMR (300 MHz, DMSO-d6): δ = 7.32 (dd, J = 4.8, 1.5 Hz, 1H), 6.95-6.88 (m, 2H), 5.82 (d, J = 7.9 Hz, 1H), 4.49 (s, 2H), 4.32-4.21 (m, 1H), 3.49-3.36 (m, 1H), 1.72- 1.36 (m, 13H), 1.27-0.98 (m, 5H) ppm | N-(2- thienyl- methyl)- cyclopentan- amine | General method D1 using isocyanatocyclo- hexane | 3-cyclohexyl-1- cyclopentyl-1- (2- thienylmethyl) urea |
| 13 | 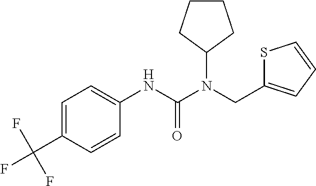 [SMILES: O=C(Nc1ccc(C(F)(F)F)cc1)N(Cc1cccs1)C1CCCC1] | 369.7 | | 1H NMR (300 MHz, DMSO-d6): δ = 8.76 (s, 1H), 7.68 (d, J = 8.0 Hz, 2H), 7.57 (d, J = 8.0 Hz, 2H), 7.34 (dd, J = 5.0, 1.4 Hz, 1H), 7.02- 6.97 (m, 1H), 6.94-6.90 (m, 1H), 4.69 (s, 2H), 4.52-4.36 (m, 1H), 1.84-1.71 (m, 2H), 1.70-1.44 (m, 6H) ppm | N-(2- thienyl- methyl)- cyclopentan- amine | General method D1 using 1-isocyanato-4- (trifluoromethyl) benzene | 1-cyclopentyl)- (2- thienylmethyl)- 3-[4- (trifluoromethyl) phenyl]urea |
| 14 | 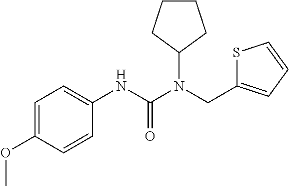 [SMILES: COc1ccc(NC(=O)N(Cc2cccs2)C2CCCC2)cc1] | 331.7 | | 1H NMR (500 MHz, DMSO-d6): δ = 8.19 (s, 1H), 7.27-7.38 (m, 3H), 6.96-7.03 (m, 1H), 6.91-6.95 (m, 1H), 6.81 (d, J = 8.85 Hz, 2H), 4.64 (s, 2H), 4.35-4.48 (m, 1H), 3.70 (s, 3H), 1.80-1.70 (m, 2H), 1.70-1.60 (m, 2H), 1.42-1.59 (m, 4H) ppm | N-(2- thienyl- methyl)- cyclopentan- amine | General method D1 using 1-isocyanato-4- methoxybenzene | 1-cyclopentyl-3- (4- methoxyphenyl)- 1-(2- thienylmethyl) urea |
| 15 | 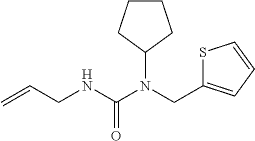 [SMILES: C=CCNC(=O)N(Cc1cccs1)C1CCCC1] | 265.7 | | 1H NMR (500 MHz, DMSO-d6): δ = 7.32 (dd, J = 4.9, 1.2 Hz, 1H), 6.95-6.90 (m, 2H), 6.47 (t, J = 5.8 Hz, 1H), 5.85-5.75 (m, 1H), 5.06- 4.95 (m, 2H), 4.52 (s, 2H), 4.29- 4.21 (m, 1H), 3.68 (t, J = 5.2 Hz, 2H), 1.75-1.65 (m, 2H), 1.64-1.54 (m, 2H), 1.51-1.46 (m, 4H) ppm | N-(2- thienyl- methyl)- cyclopentan- amine | General method D1 using 3-isocyanato- prop-1-ene | 3-allyl-1- cyclopentyl-1- (2- thienylmethyl) urea |
| 16 | 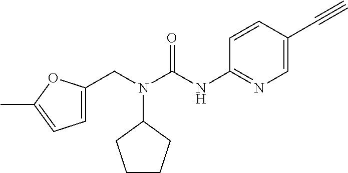 [SMILES: C#Cc1ccc(NC(=O)N(Cc2ccc(C)o2)C2CCCC2)nc1] | 324.7 | | 1HNMR (300 MHz, DMSO-d6): δ = 8.89 (br.s., 1H), 8.32 (d, J = 1.8 Hz, 1H), 7.83-7.74 (m, 2H), 6.12 (d, J = 3.1 Hz, 1H), 5.96-5.94 (m, 1H), 4.55-4.45 (m, 1H), 4.47 (br.s., 2H), 4.24 (s, 1H), 2.19 (s, 3H), 1.78-1.40 (m, 8H) ppm | 5- methylfuran- 2- carbaldehyde | General method A using cyclopentanamine, AcOH and NaBH₃CN; method Q for carbamate synthesis; method D5 using phenyl N-(5-ethynyl-2- pyridyl)carbamate | 1-cyclopentyl-3- (5-ethynyl-2- pyridyl)-1-[(5- methyl-2- furyl)methyl] urea |
| 17 | 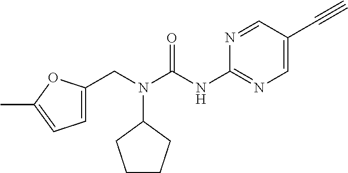 [SMILES: C#Cc1cnc(NC(=O)N(Cc2ccc(C)o2)C2CCCC2)nc1] | 325.7 | | 1HNMR (300 MHz, DMSO-d6): δ = 9.59 (s, 1H), 8.63 (s, 2H), 6.07 (d, J = 2.1 Hz, 1H), 5.93 (br.s., 1H), 4.47-4.32 (m, 4H), 2.17 (s, 3H), 1.75-1.44 (m, 8H) ppm | 5- methylfuran- 2- carbaldehyde | General method A using cyclopentanamine, AcOH and NaBH₃CN; method Q for carbamate synthesis; method D5 using phenyl N-(5- ethynylpyrimidin-2- yl)carbamate and TEA | 1-cyclopentyl-3- (5-ethynyl- pyrimidin- 2-yl)-1-[(5- methyl-2- furyl)methyl] urea |
| 18 | 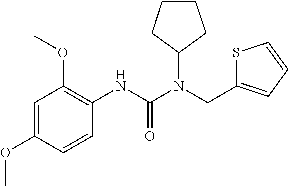 [SMILES: COc1ccc(NC(=O)N(Cc2cccs2)C2CCCC2)c(OC)c1] | 361.8 | | 1H NMR (300 MHz, DMSO-d6): δ = 7.59 (d, J = 9.2 Hz, 1H), 7.43 (dd, J = 4.9, 1.4 Hz, 1H), 7.18 (s, 1H), 7.08-7.06 (m, 1H), 7.00 (dd, J = 5.1, 3.5 Hz, 1H), 6.52 (d, J = 2.6 Hz, 1H), 6.42 (dd, J = 8.8, 2.6 Hz, 1H), 4.60 (s, 2H), 4.55-4.42 (m, 1H), 3.70 (s, 3H), 3.67 (s, 3H), 1.88-1.73 (m, 2H), 1.72-1.60 (m, 2H), 1.58-1.44 (m, 4H) ppm | N-(2- thienyl- methyl)- cyclopentan- amine | General method D1 using 1-isocyanato-2,4- dimethoxybenzene | 1-cyclopentyl-3- (2,4- dimethoxy- phenyl)-1-(2- thienylmethyl)- urea |
| 19 | 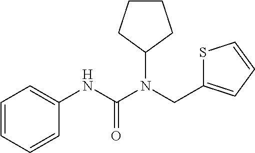 [SMILES: O=C(Nc1ccccc1)N(Cc1cccs1)C1CCCC1] | 301.7 | | 1H NMR (300 MHz, DMSO-d6): δ = 8.33 (s, 1H), 7.42 (d, J = 7.7 Hz, 2H), 7.34 (dd, J = 5.1, 1.2 Hz, 1H), 7.21 (t, J = 7.9 Hz, 2H), 7.01- 6.98 (m, 1H), 6.95-6.89 (m, 2H), 4.66 (s, 2H), 4.50-6.37 (m, 1H), 1.82-1.70 (m, 2H), 1.68-1.41 (m, 6H) ppm | N-(2- thienyl- methyl)- cyclopentan- amine | General method D1 using phenyl isocyanate | 1-cyclopentyl-3- phenyl-1-(2- thienylmethyl) urea |
| 20 | 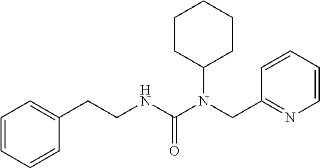 [SMILES: O=C(NCCc1ccccc1)N(Cc1ccccn1)C1CCCCC1] | 338.8 | | 1H NMR (300 MHz, DMSO-d6): δ = 8.43-8.38 (m, 1H), 7.70 (td, J = 7.6, 1.7 Hz, 1H), 7.29-7.21 (m, 2H), 7.21-7.13 (m, 5H), 6.65 (t, J = 5.6 Hz, 1H), 4.37 (s, 2H), 3.92- 3.80 (bs, 1H), 3.30-3.22 (m, 2H), 2.71 (t, J = 7.3 Hz, 2H), 1.70-1.59 (m, 2H), 1.57-1.42 (m, 3H), 1.34- 1.14 (m, 4H), 1.08-0.92 (m, 1H) ppm | N-(2- pyridyl- methyl) cyclohexan amine | General method D1 using 2- isocyanatoethyl- benzene | 1-cyclohexyl-3- (2-phenylethyl)- 1-(2- pyridylmethyl) urea |
| 21 | 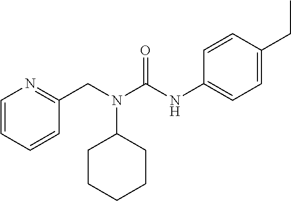 [SMILES: CCc1ccc(NC(=O)N(Cc2ccccn2)C2CCCCC2)cc1] | 338.8 | | 1H NMR (300 MHz, DMSO-d6): δ = 9.08 (s, 1H), 8.55 (d, J = 4.2 Hz, 1H), 7.78 (dt, J = 6.8, 1.7 Hz, 1H), 7.41-7.25 (m, 4H), 7.05 (d, J = 8.4 Hz, 2H), 4.54 (s, 2H), 4.12- 3.98 (m, 1H), 2.55-2.48 (m, 2H), 1.74-1.64 (m, 2H), 1.61-1.49 (m, 3H), 1.48-1.19 (m, 4H), 1.18-0.98 (m, 4H) ppm | N-(2- pyridyl- methyl) cyclohexan amine | General method D1 using 1-ethyl-4- isocyanatobenzene | 1-cyclohexyl-3- (4-ethylphenyl)- 1-(2- pyridylmethyl) urea |
| 22 | 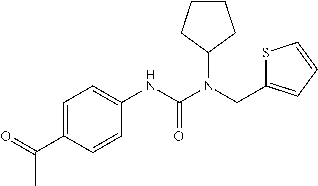 [SMILES: CC(=O)c1ccc(NC(=O)N(Cc2cccs2)C2CCCC2)cc1] | 343.8 | | 1H NMR (300 MHz, DMSO-d6): δ = 8.75 (s, 1H), 7.85 (d, J = 8.8 Hz, 2H), 7.60 (d, J = 8.8 Hz, 2H), 7.34 (dd, J = 5.1, 1.2 Hz, 1H), 7.02-6.98 (m, 1H), 6.92 (dd, J = 3.5, 5.1 Hz, 1H), 4.69 (s, 2H), 4.54-4.35 (m, 1H), 1.86-1.71 (m, 2H), 1.70-1.40 (m, 6H) ppm | N-(2- thienyl- methyl)- cyclopentan- amine | General method D1 using 1-(4- isocyanatophenyl) ethanone | 3-(4- acetylphenyl)-1- cyclopentyl-1- (2- thienylmethyl) urea |
| 23 | 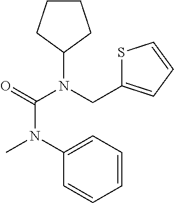 [SMILES: CN(C(=O)N(Cc1cccs1)C1CCCC1)c1ccccc1] | 315.7 | | 1H NMR (300 MHz, DMSO-d6): δ = 7.38 (d, J = 4.7 Hz, 1H), 7.32 (t, J = 7.4 Hz, 2H), 7.14-7.01 (m, 3H), 6.98-6.88 (m, 2H), 4.30 (s, 2H), 4.08-3.96 (m, 1H), 3.01 (s, 3H), 1.61-1.46 (m, 2H), 1.45-1.18 (m, 6H) ppm | N-(2- thienyl- methyl)- cyclopentan- amine | General method D1 using phenyl isocyanate, method J using MeI and Cs₂CO₃ | 1-cyclopentyl-3- methyl-3- phenyl-1-(2- thienylmethyl) urea |
| 24 | 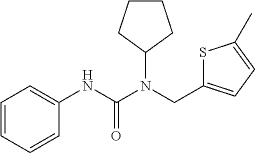 [SMILES: Cc1ccc(CN(C(=O)Nc2ccccc2)C2CCCC2)s1] | 315.7 | | 1H NMR (300 MHz, DMSO-d6): δ = 8.28 (s, 1H), 7.43 (d, J = 7.7 Hz, 2H), 7.21 (t, J = 7.9 Hz, 2H), 6.91 (t, J = 6.4 Hz, 1H), 6.76 (d, J = 3.3 Hz, 1H), 6.58 (dd, J = 3.3, 1.1 Hz, 1H), 4.57 (s, 2H), 4.48- 4.36 (m, 1H), 2.34 (s, 3H), 1.83- 1.69 (m, 2H), 1.68-1.41 (m, 6H) ppm | 5- methylthio- phene-2- carbaldehyde | General method A using cyclopentanamine, AcOH and NaBH₄, method D1 using phenyl isocyanate | 1-cyclopentyl-1- [(5-methyl-2- thienyl)methyl]- 3-phenyl-urea |
| 25 | 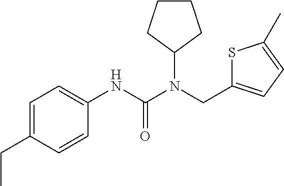 [SMILES: CCc1ccc(NC(=O)N(Cc2ccc(C)s2)C2CCCC2)cc1] | 343.8 | | 1H NMR (300 MHz, DMSO-d6): δ = 8.19 (s, 1H), 7.32 (d, J = 8.4 Hz, 2H), 7.05 (d, J = 8.4 Hz, 2H), 6.76 (d, J = 3.34 Hz, 1H), 6.58 (dd, J = 3.3, 1.1 Hz, 1H), 4.55 (s, 2H), 4.47-4.34 (m, 1H), 2.56-2.48 (m, 2H), 2.34 (s, 3H), 1.80-1.68 (m, 2H), 1.67-1.41 (m, 6H), 1.13 (t, J = 7.6 Hz, 3H) ppm | 5- methylthio- phene-2- carbaldehyde | General method A using cyclopentanamine, AcOH and NaBH₄, method D1 using 1- ethyl-4- isocyanatobenzene | 1-cyclopentyl)- (4-ethylphenyl- 1-[(5-methyl- thienyl)methyl] urea |
| 26 | 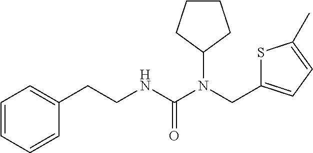 [SMILES: Cc1ccc(CN(C(=O)NCCc2ccccc2)C2CCCC2)s1] | 343.8 | | 1H NMR (300 MHz, DMSO-d6): δ = 7.29-7.22 (m, 2H), 7.20-7.12 (m, 3H), 6.65 (d, J = 3.7 Hz, 1H), 6.55 (dd, J = 3.1, 1.1 Hz, 1H), 6.30 (t, J = 5.4 Hz, 1H), 4.39 (s, 2H), 4.23-4.11 (m, 1H), 3.28-3.16 (m, 2H), 2.70 (t, J = 7.2 Hz, 2H), 2.35 (s, 3H), 1.74-1.52 (m, 4H), 1.51- 1.35 (m, 4H) ppm | 5- methylthio- phene-2- carbaldehyde | General method A using cyclopentanamine, AcOH and NaBH₄, method D1 using 2- isocyanatoethyl- benzene | 1-cyclopentyl-1- [(5-methyl-2- thienyl)methyl]- 3-(2- phenylethyl)urea |
| 27 | 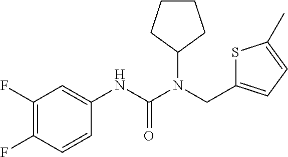 [SMILES: Cc1ccc(CN(C(=O)Nc2ccc(F)c(F)c2)C2CCCC2)s1] | 351.7 | | 1H NMR (300 MHz, DMSO-d6): δ = 8.54 (s, 1H), 7.66-7.56 (m, 1H), 7.34-7.18 (m, 2H), 6.76 (d, J = 3.3 Hz, 1H), 6.58 (dd, J = 3.3, 1.1 Hz, 1H), 4.56 (s, 2H), 4.44- 4.30 (m, 1H), 2.34 (s, 3H), 1.83- 1.69 (m, 2H), 1.68-1.42 (m, 6H) ppm | 5- methylthio- phene-2- carbaldehyde | General method A using cyclopentanamine, AcOH and NaBH₄, method D1 using 1,2- difluoro-4- isocyanatobenzene | 1-cyclopentyl-3- (3,4- difluorophenyl)- 1-[(5-methyl- thienyl)methyl] urea |
| 28 | 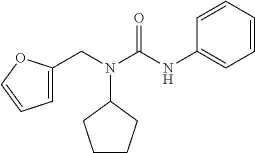 [SMILES: O=C(Nc1ccccc1)N(Cc1ccco1)C1CCCC1] | 285.7 | | 1H NMR (300 MHz, DMSO-d6): δ = 8.22 (s, 1H), 7.55 (d, J = 0.9 Hz, 1H), 7.43 (d, J = 7.7 Hz, 2H), 7.21 (t, J = 7.7 Hz, 2H), 6.92 (t, J = 7.7 Hz, 1H), 6.39-6.34 (m, 1H), 6.23 (d, J = 3.0 Hz, 1H), 4.49 (s, 2H), 4.47-4.37 (m, 1H), 1.82-1.68 (m, 2H), 1.66-1.39 (m, 6H) ppm | furan-2- carbaldehyde | General method A using cyclopentanamine, AcOH and NaBH₄, method D1 using phenyl isocyanate | 1-cyclopentyl-1- (2-furylmethyl)- 3-phenyl-urea |
| 29 | 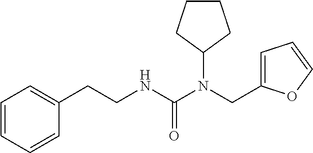 [SMILES: O=C(NCCc1ccccc1)N(Cc1ccco1)C1CCCC1] | 313.8 | | 1H NMR (300 MHz, DMSO-d6): δ = 7.51 (m, 1H), 7.31-7.22 (m, 2H), 7.21-7.13 (m, 3H), 6.36-6.28 (m, 2H), 6.11 (d, J = 3.1 Hz, 1H), 4.30 (s, 2H), 4.27-4.16 (m, 1H), 3.27-3.18 (m, 2H), 2.69 (t, J = 7.0 Hz, 2H), 1.72-1.50 (m, 4H), 1.49- 1.32 (m, 4H) ppm | furan-2- carbaldehyde | General method A using cyclopentanamine, AcOH and NaBH₄, method D1 using 2- isocyanatoethyl- benzene | 1-cyclopentyl-1- (2-furylmethyl)- 3-(2- phenylethyl)urea |
| 30 | 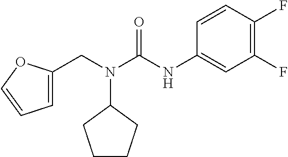 [SMILES: O=C(Nc1ccc(F)c(F)c1)N(Cc1ccco1)C1CCCC1] | 321.7 | | 1H NMR (300 MHz, DMSO-d6): δ = 8.53 (s, 1H), 7.72-7.49 (m, 2H), 7.38-7.15 (m, 2H), 6.37 (bs, 1H), 6.22 (bs, 1H), 4.48 (bs, 2H), 4.44-4.30 (m, 1H), 1.85-1.38 (m, 8H) ppm | furan-2- carbaldehyde | General method A using cyclopentanamine, AcOH and NaBH₄, method D1 using 1,2- difluoro-4- isocyanatobenzene | 1-cyclopentyl-1- (3,4- difluorophenyl)- 1-(2- furylmethyl)urea |
| 31 | 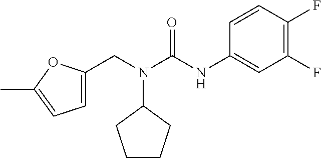 [SMILES: Cc1ccc(CN(C(=O)Nc2ccc(F)c(F)c2)C2CCCC2)o1] | 335.8 | | 1H NMR (300 MHz, DMSO-d6): δ = 8.48 (s, 1H), 7.70-7.54 (m, 1H), 7.34-7.15 (m, 2H), 6.08 (d, J = 3.1 Hz, 1H), 5.95 (d, J = 2.1 Hz, 1H), 4.50-4.32 (m, 3H), 2.20 (s, 3H), 1.81-1.69 (m, 2H), 1.68-1.41 (m, 6H) ppm | 5- methylfuran- 2- carbaldehyde | General method A using cyclopentanamine, AcOH and NaBH₄, method D1 using 1,2- difluoro-4- isocyanatobenzene | 1-cyclopentyl-3- (3,4- difluorophenyl)- 1-[(5-methyl-2- furyl)methyl] urea |
| 32 | 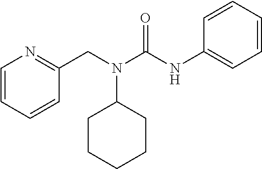 [SMILES: O=C(Nc1ccccc1)N(Cc1ccccn1)C1CCCCC1] | 310.7 | | 1H NMR (300 MHz, DMSO-d6): δ = 9.22 (s, 1H), 8.57 (d, J = 4.9 Hz, 1H), 7.79 (dt, J = 7.8, 1.6 Hz, 1H), 7.45-7.36 (m, 3H), 7.32-7.18 (m, 3H), 6.92 (t, J = 7.1 Hz, 1H), 4.55 (s, 2H), 4.13-3.96 (m, 1H), 1.77-1.64 (m, 2H), 1.61-1.50 (m, 3H), 1.49-1.19 (m, 4H), 1.16-0.97 (m, 1H) ppm | N-(2- pyridyl- methyl) cyclohexan amine | General method D1 using phenyl isocyanate | 1-cyclohexyl-3- phenyl-1-(2- pyridylmethyl) urea |
| 33 | 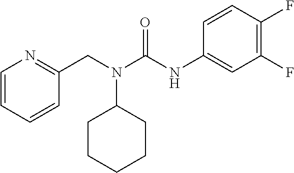 [SMILES: O=C(Nc1ccc(F)c(F)c1)N(Cc1ccccn1)C1CCCCC1] | 346.7 | | 1H NMR (300 MHz, DMSO-d6): δ = 9.30 (s, 1H), 8.55 (d, J = 4.2 Hz, 1H), 7.78 (dt, J = 7.7, 1.5 Hz, 1H), 7.67-54 (m, 1H), 7.40-7.33 (m, 1H), 7.32-7.22 (m, 2H), 7.20- 7.12 (m, 1H), 4.56 (s, 2H), 4.13- 3.97 (m, 1H), 1.75-1.62 (m, 2H), 1.61-1.49 (m, 3H), 1.48-1.18 (m, 4H), 1.15-0.96 (m, 1H) ppm | N-(2- pyridyl- methyl) cyclohexan amine | General method D1 using 1,2-difluoro-4- isocyanatobenzene | 1-cyclohexyl-3- (3,4- difluorophenyl)- 1-(2- pyridylmethyl) urea |
| 34 | 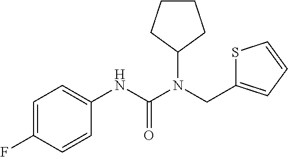 [SMILES: O=C(Nc1ccc(F)cc1)N(Cc1cccs1)C1CCCC1] | 319.7 | | 1H NMR (300 MHz, DMSO-d6): δ = 8.39 (s, 1H), 7.47-7.39 (m, 2H), 7.33 (dd, J = 5.2. 1.2 Hz, 1H), 7.05 (t, J = 8.8 Hz, 2H), 7.00-6.96 (m, 1H), 6.94-6.89 (m, 1H), 4.65 (s, 2H), 4.48-4.32 (m, 1H), 1.83- 1.69 (m, 2H), 1.68-1.41 (m, 6H) ppm | N-(2- thienyl- methyl)- cyclopentan- amine | General method D1 using 4- fluorophenylisocyanate | 1-cyclopentyl-3- (4- fluorophenyl)-1- (2- thienylmethyl) urea |
| 35 | 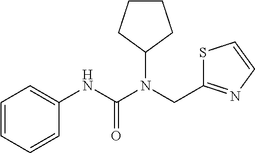 [SMILES: O=C(Nc1ccccc1)N(Cc1nccs1)C1CCCC1] | 302.7 | | 1H NMR (300 MHz, DMSO-d6): δ = 8.65 (s, 1H), 7.71 (d, J = 4.0 Hz, 1H), 7.60 (d, J = 7.60 Hz, 1H), 7.43 (d, J = 8.2 Hz, 2H), 7.23 (t, J = 8.2 Hz, 2H), 6.98-6.90 (m, 1H), 4.74 (s, 2H), 4.58-4.43 (m, 1H), 1.88-1.72 (m, 2H), 1.71-1.60 (m, 2H), 1.59-1.41 (m, 4H), ppm | thiazole-2- carbaldehyde | General method A using cyclopentanamine, AcOH and NaBH₄, method D1 using phenyl isocyanate | 1-cyclopentyl-3- phenyl-1- (thiazol-2- ylmethyl)urea |
| 36 | 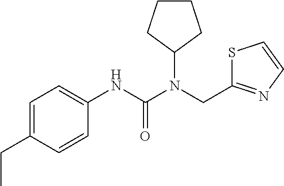 [SMILES: CCc1ccc(NC(=O)N(Cc2nccs2)C2CCCC2)cc1] | 330.7 | | 1H NMR (300 MHz, DMSO-d6): δ = 8.52 (s, 1H), 7.70 (d, J = 3.2 Hz, 1H), 7.59 (d, J = 3.2 Hz, 1H), 7.33 (d, J = 8.4 Hz, 2H), 7.06 (d, J = 8.4 Hz, 2H), 4.73 (s, 2H), 4.56- 4.40 (m, 1H), 2.58-2.48 (m, 2H), 1.86-1.70 (m, 2H), 1.69-1.85 (m, 2H), 1.57-1.41 (m, 4H), 1.13 (t, J = 7.6 Hz, 3H) ppm | thiazole-2- carbaldehyde | General method A using cyclopentanamine, AcOH and NaBH₄, method D1 using 1- ethyl-4- isocyanatobenzene | l-cyclopentyl-3- (4-ethylphenyl)- 1-(thiazol-2- ylmethyl)urea |
| 37 | 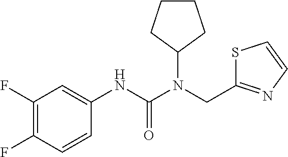 [SMILES: O=C(Nc1ccc(F)c(F)c1)N(Cc1nccs1)C1CCCC1] | 338.7 | | 1H NMR (300 MHz, DMSO-d6): δ = 8.80 (s, 1H), 7.71 (d, J = 3.3 Hz, 1H), 7.66-7.54 (m, 2H), 7.36- 7.18 (m, 2H), 4.74 (s, 2H), 4.56- 4.38 (m, 1H), 1.86-1.72 (m, 2H), 1.71-1.59 (m, 2H), 1.58-1.48 (m, 4H) ppm | thiazole-2- carbaldehyde | General method A using cyclopentanamine, AcOH and NaBH₄, method D1 using 1,2- difluoro-4- isocyanatobenzene | 1-cyclopentyl-3- (3,4- difluorophenyl)- 1-(thiazol-2- ylmethyl)urea |
| 38 | 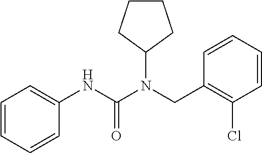 [SMILES: O=C(Nc1ccccc1)N(Cc1ccccc1Cl)C1CCCC1] | 329.8 | | 1H NMR (300 MHz, DMSO-d6): δ = 8.44 (s, 1H), 7.47-7.40 (m, 3H), 7.36-7.16 (m, 5H), 6.96-6.89 (m, 1H), 4.65-4.52 (m, 1H), 4.50 (s, 2H), 1.87-1.70 (m, 2H), 1.68- 1.44 (m, 4H), 1.43-1.26 (m, 2H) ppm | 2- chlorobenz- aldehyde | General method A using cyclopentanamine, AcOH and NaBH₄, method D1 using phenyl isocyanate | 1-[(2- chlorophenyl) methyl]-1- cyclopentyl-3- phenyl-urea |
| 39 | 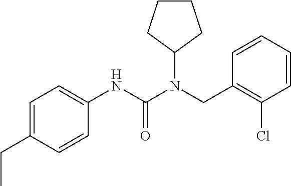 [SMILES: CCc1ccc(NC(=O)N(Cc2ccccc2Cl)C2CCCC2)cc1] | 357.8 | | 1H NMR (300 MHz, DMSO-d6): δ = 8.36 (s, 1H), 7.42 (dd, J = 7.8, 1.6 Hz, 1H), 7.36-7.16 (m, 5H), 7.05 (d, J = 8.4 Hz, 2H), 4.65-4.52 (m, 1H), 4.50 (s, 2H), 2.57-2.51 (m, 2H), 1.88-1.69 (m, 2H), 1.67- 1.42 (m, 4H), 1.41-1.26 (m, 2H), 1.13 (t, J = 7.6 Hz, 3H) ppm | 2- chlorobenz- aldehyde | General method A using cyclopentanamine, AcOH and NaBH₄, method D1 using 1- ethyl-4- isocyanatobenzene | 1-[(2- chlorophenyl) methyl]-1- cyclopentyl-3- (4- ethylphenyl)urea |
| 40 | 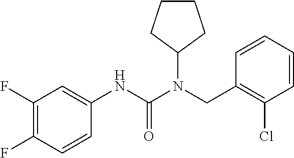 [SMILES: O=C(Nc1ccc(F)c(F)c1)N(Cc1ccccc1Cl)C1CCCC1] | 365.7 | | 1H NMR (300 MHz, DMSO-d6): δ = 8.65 (s, 1H), 7.61 (ddd, J = 13.9, 7.6, 2.0 Hz, 1H), 7.43 (dd, J = 7.6, 1.5 Hz, 1H), 7.36-7.14 (m, 5H), 4.61-4.46 (m, 3H), 1.85-1.71 (m, 2H), 1.67-1.27 (m, 6H) ppm | 2- chlorobenz- aldehyde | General method A using cyclopentanamine, AcOH and NaBH₄, method D1 using 1,2- difluoro-4- isocyanatobenzene | 1-[(2- chlorophenyl) methyl]-1- cyclopentyl-3- (3,4- difluorophenyl) urea |
| 41 | 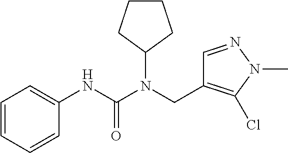 [SMILES: Cn1ncc(CN(C(=O)Nc2ccccc2)C2CCCC2)c1Cl] | 333.7 | | 1H NMR (300 MHz, DMSO-d6): δ = 8.33 (s, 1H), 7.21 (t, J = 7.4 Hz, 2H), 7.37 (s, 1H), 7.26-7.16 (m, 2H), 6.92 (t, J = 7.4 Hz, 1H), 4.48- 4.30 (m, 1H), 4.24 (s, 2H), 3.74 (s, 3H), 1.86-1.70 (m, 2H), 1.69- 1.50 (m, 2H), 1.49-1.23 (m, 4H) ppm | 5-chloro-1- methyl- pyrazole-4- carbaldehyde | General method A using cyclopentanamine, AcOH and NaBH₄, method D1 using phenyl isocyanate | 1-[(5-chloro-1- methyl-pyrazol- 4-yl)methyl]-1- cyclopentyl-3- phenyl-urea |
| 42 | 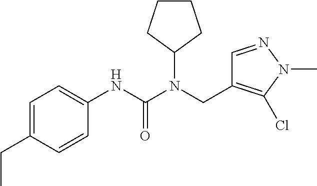 [SMILES: CCc1ccc(NC(=O)N(Cc2cnn(C)c2Cl)C2CCCC2)cc1] | 361.8 | | 1H NMR (300 MHz, DMSO-d6): δ = 8.25 (s, 1H), 7.26-7.41 (m, 3H), 7.05 (d, J = 8.4 Hz, 3H), 4.47- 4.29 (m, 1H), 4.23 (s, 2H), 3.74 (s, 3H), 2.52-2.48 (m, 2H), 1.82- 1.69 (m, 2H), 1.68-1.50 (m, 2H), 1.67-1.24 (m, 4H), 1.13 (t, J = 7.6 Hz, 3H) ppm | 5-chloro-1- methyl- pyrazole-4- carbaldehyde | General method A using cyclopentanamine, AcOH and NaBH₄, method D1 using 1- ethyl-4- isocyanatobenzene | 1-[(5-chloro-1- methyl-pyrazol- 4-yl)methyl]-1- cyclopentyl-3- (4- ethylphenyl)urea |
| 43 | 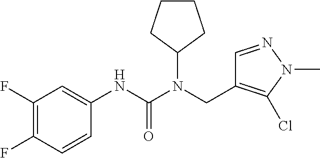 [SMILES: Cn1ncc(CN(C(=O)Nc2ccc(F)c(F)c2)C2CCCC2)c1Cl] | 369.7 | | 1H NMR (300 MHz, DMSO-d6): δ = 8.56 (s, 1H), 7.69-7.50 (m, 1H), 7.37 (s, 1H), 7.35-7.11 (m, 2H), 4.44-4.27 (m, 1H), 4.23 (s, 2H), 3.74 (s, 3H), 1.86-1.70 (m, 2H), 1.69-1.55 (m, 2H), 1.55-1.39 (m, 4H) ppm | 5-chloro-1- methyl- pyrazole-4- carbaldehyde | General method A using cyclopentanamine, AcOH and NaBH₄, method D1 using 1,2- difluoro-4- isocyanatobenzene | 1-[(5-chloro-1- methyl-pyrazol- 4-yl)methyl]-1- cyclopentyl-3- (3,4- difluorophenyl) urea |
| 44 | 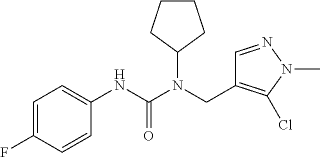 [SMILES: Cn1ncc(CN(C(=O)Nc2ccc(F)cc2)C2CCCC2)c1Cl] | 351.7 | | 1H NMR (300 MHz, DMSO-d6): δ = 8.39 (s, 1H), 7.49-7.38 (m, 2H), 7.37 (s, 1H), 7.05 (t, J = 9.0 Hz, 2H), 4.40-4.34 (m, 1H), 4.23 (s, 2H), 3.74 (s, 3H), 1.76 (m, 2H), 1.64 (m, 2H), 1.56-1.38 (m, 4H) ppm | 5-chloro-1- methyl- pyrazole-4- carbaldehyde | General method A using cyclopentanamine, AcOH and NaBH₄, method D1 using 4- fluorophenyl- isocyanate | 1-[(5-chloro-1- methyl-pyrazol- 4-yl)methyl]-1- cyclopentyl-3- (4- fluorophenyl) urea |
| 45 | 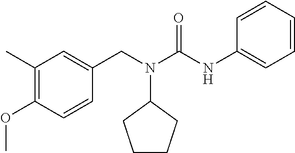 [SMILES: COc1ccc(CN(C(=O)Nc2ccccc2)C2CCCC2)cc1C] | 339.8 | | 1H NMR (300 MHz, DMSO-d6): δ = 8.16 (s, 1H), 7.39 (d, J = 7.5 Hz, 2H), 7.19 (t, J = 7.7 Hz, 2H), 6.98-7.05 (m, 2H), 6.77-6.96 (m, 2H), 4.45-4.58 (m, 1H), 4.43 (s, 2H), 3.73 (s, 3H), 2.10 (s, 3H), 1.64-1.80 (m, 2H), 1.53-1.66 (m, 2H), 1.37-1.53 (m, 4H) ppm | 4-methoxy- 3-methyl- benzaldehyde | General method A using cyclopentanamine, AcOH and NaBH₄, method D1 using phenyl isocyanate | 1-cyclopentyl-1- [(4-methoxy-3- methyl- phenyl)methyl]- 3-phenyl-urea |
| 46 | 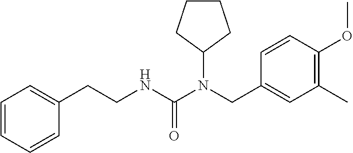 [SMILES: COc1ccc(CN(C(=O)NCCc2ccccc2)C2CCCC2)cc1C] | 367.8 | | 1H NMR (300 MHz, DMSO-d6): δ = 7.29-7.05 (m, 5H), 6.95-6.88 (m, 2H), 6.85-6.78 (m, 1H), 6.12 (t, J = 5.7 Hz, 1H), 4.40-4.26 (m, 1H), 4.24 (s, 2H), 3.74 (s, 3H), 3.28-3.15 (m, 2H), 2.67 (m, 2H), 2.10 (s, 3H), 1.70-1.47 (m, 4H), 1.49-1.22 (m, 4H) ppm | 4-methoxy- 3-methyl- benzaldehyde | General method A using cyclopentanamine, AcOH and NaBH₄, method D1 using 1- ethyl-4- isocyanatobenzene | 1-cyclopentyl-1- [(4-methoxy-3- methyl- phenyl)methyl]- 3-(2- phenylethyl)urea |
| 47 | 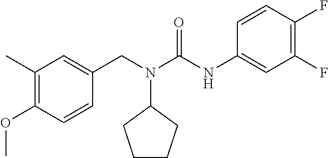 [SMILES: COc1ccc(CN(C(=O)Nc2ccc(F)c(F)c2)C2CCCC2)cc1C] | 375.8 | | 1H NMR (300 MHz, DMSO-d6): δ = 8.44 (s, 1H), 7.71-7.52 (m, 1H), 7.33-7.12 (m, 2H), 7.03-6.94 (m, 2H), 6.88-6.81 (m, 1H), 4.53- 4.37 (m, 3H), 3.73 (s, 3H), 2.10 (s, 3H), 1.79-1.66 (m, 2H), 1.65- 1.54 (m, 2H), 1.54-1.35 (m, 4H) ppm | 4-methoxy- 3-methyl- benzaldehyde | General method A using cyclopentanamine, AcOH and NaBH₄, method D1 using 1,2- difluoro-4- isocyanatobenzene | 1-cyclopentyl-3- (3,4- difluorophenyl)- 1-[(4-methoxy- 3-methyl- phenyl)methyl] urea |
| 48 | 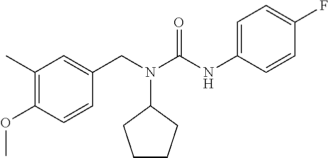 [SMILES: COc1ccc(CN(C(=O)Nc2ccc(F)cc2)C2CCCC2)cc1C] | 357.8 | | 1H NMR (300 MHz, DMSO-d6): δ = 8.25 (s, 1H), 7.48-7.33 (m, 2H), 7.09-6.93 (m, 4H), 6.91-6.78 (m, 1H), 4.55-4.44 (m, 1H), 4.42 (s, 2H), 3.73 (s, 3H), 2.10 (s, 3H), 1.79-1.66 (m, 2H), 1.64-1.54 (m, 2H), 1.53-1.36 (m, 4H) ppm | 4-methoxy- 3-methyl- benzaldehyde | General method A using cyclopentanamine, AcOH and NaBH₄, method D1 using 4- fluorophenyl- isocyanate | 1-cyclopentyl-3- (4- fluorophenyl)-1- [(4-methoxy-3- methyl- phenyl)methyl] urea |
| 49 | 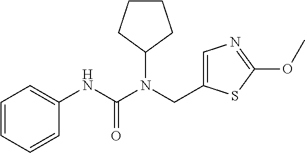 [SMILES: COc1ncc(CN(C(=O)Nc2ccccc2)C2CCCC2)s1] | 332.7 | | 1H NMR (300 MHz, DMSO-d6): δ = 8.39 (s, 1H), 7.43 (d, J = 7.7 Hz, 2H), 7.29-7.17 (t, J = 7.7 Hz, 2H), 7.09 (s, 1H), 6.99-6.86 (m, 1H), 4.47 (s, 2H), 4.43-4.27 (m, 1H), 3.93 (s, 3H), 1.84-1.41 (m, 8H) ppm | 5- methoxy- thiazole-2- carbaldehyde | General method A using cyclopentanamine, AcOH and NaBH₄, method D1 using phenyl isocyanate | 1-cyclopentyl-1- [(2- methoxythiazol- 5-yl)methyl]- phenyl-urea |
| 50 | 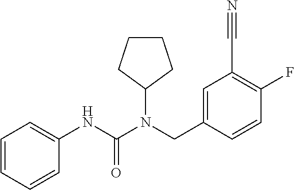 [SMILES: N#Cc1cc(CN(C(=O)Nc2ccccc2)C2CCCC2)ccc1F] | 338.7 | | 1H NMR (300 MHz, DMSO-d6): δ = 8.39 (s, 1H), 7.78-7.67 (m, 1H), 7.68-7.54 (m, 1H), 7.54-7.38 (m, 3H), 7.21 (t, J = 7.4 Hz, 2H), 7.01-6.83 (m, 1H), 4.51-4.45 (m, 3H), 1.87-1.69 (m, 2H), 1.69-1.51 (m, 2H), 1.55-1.30 (m, 4H) ppm | 2-fluoro-5- formyl- benzonitrile | General method A using cyclopentanamine, AcOH and NaBH₄, method D1 using phenyl isocyanate | 1-[(3-cyano-4- fluoro- phenyl)methyl]- 1-cyclopentyl-3- phenyl-urea |
| 51 | 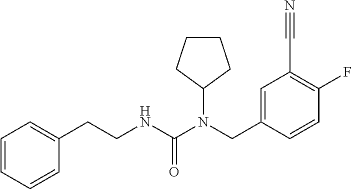 [SMILES: N#Cc1cc(CN(C(=O)NCCc2ccccc2)C2CCCC2)ccc1F] | 366.8 | | 1H NMR (300 MHz, DMSO-d6): δ = 7.60 (dd, J = 1.9, 6.1 Hz, 1H), 7.55-7.49 (m, 1H), 7.48-7.39 (m, 1H), 7.28-7.21 (m, 2H), 7.21-7.09 (m, 2H), 6.50-6.38 (m, 1H), 4.35 (s, 2H), 4.32-4.17 (m, 1H), 3.30- 3.19 (m, 2H), 2.76-2.64 (m, 2H), 1.71-1.49 (m, 4H), 1.50-1.36 (m, 2H), 1.34-1.17 (m, 2H) ppm | 2-fluoro-5- formyl- benzonitrile | General method A using cyclopentanamine, AcOH and NaBH₄, method D1 using 2- isocyanatoethyl- benzene | 1-[(3-cyano-4- fluoro- phenyl)methyl]- 1-cyclopentyl-3- (2- phenylethyl)urea |
| 52 | 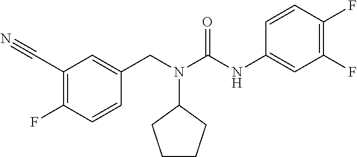 [SMILES: N#Cc1cc(CN(C(=O)Nc2ccc(F)c(F)c2)C2CCCC2)ccc1F] | 374.7 | | 1H NMR (300 MHz, DMSO-d6): δ = 8.61 (s, 1H), 7.72 (d, J = 6.3 Hz, 1H), 7.68-7.53 (m, 2H), 7.52- 7.43 (m, 1H), 7.36-7.15 (m, 2H), 4.51 (s, 2H), 4.49-4.35 (m, 1H), 1.84-1.69 (m, 2H), 1.69-1.57 (m, 2H), 1.56-1.27 (m, 4H) ppm | 2-fluoro-5- formyl- benzonitrile | General method A using cyclopentanamine, AcOH and NaBH₄, method D1 using 1,2- difluoro-4- isocyanatobenzene | 1-[(3-cyano-4- fluoro- phenyl)methyl]- 1-cyclopentyl-3- (3,4- difluorophenyl) urea |
| 53 | 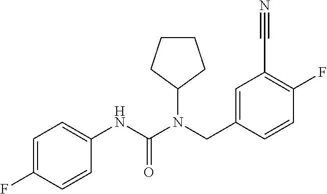 [SMILES: N#Cc1cc(CN(C(=O)Nc2ccc(F)cc2)C2CCCC2)ccc1F] | 356.7 | | 1H NMR (300 MHz, DMSO-d6): δ = 8.44 (s, 1H), 7.80-7.67 (m, 1H), 7.67-7.55 (m, 1H), 7.54-7.35 (m, 3H), 7.12-6.97 (m, 2H), 4.50 (s, 2H), 4.50-4.36 (m, 1H), 1.87- 1.69 (m, 2H), 1.69-1.56 (m, 2H), 1.56-1.29 (m, 4H) ppm | 2-fluoro-5- formyl- benzonitrile | General method A using cyclopentanamine, AcOH and NaBH₄, method D1 using 4- fluorophenyl- isocyanate | 1-[(3-cyano-4- fluoro- phenyl)methyl]- 1-cyclopentyl-3- (4- fluorophenyl) urea |
| 54 | 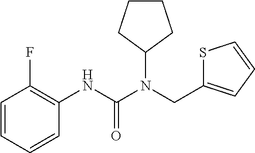 [SMILES: O=C(Nc1ccccc1F)N(Cc1cccs1)C1CCCC1] | 319.7 | | 1H NMR (300 MHz, DMSO-d6): δ = 8.01 (s, 1H), 7.53-7.48 (m, 1H), 7.37 (dd, J = 5.1, 1.2 Hz, 1H), 7.21-7.04 (m, 3H),7.04-6.88 (m, 2H), 4.65 (s, 2H), 4.48-4.34 (m, 1H), 1.83-1.72 (m, 2H), 1.69-1.38 (m, 6H) ppm | N-(2- thienyl- methyl)- cyclopentan- amine | General method D1 using 2- fluorophenylisocyanate | 1-cyclopentyl-3- (2- fluorophenyl)-1- (2- thienylmethyl) urea |
| 55 | 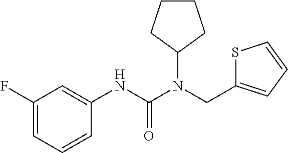 [SMILES: O=C(Nc1cccc(F)c1)N(Cc1cccs1)C1CCCC1] | 319.6 | | 1H NMR (300 MHz, DMSO-d6): δ = 8.56 (s, 1H), 7.51-7.37 (m, 1H), 7.34 (dd, J = 5.1, 1.2 Hz, 1H), 7.28-7.16 (m, 2H), 7.01-6.96 (m, 1H), 6.92 (dd, J = 5.1, 3.5 Hz, 1H), 6.81-6.66 (m, 1H), 4.66 (s, 2H), 4.51-4.31 (m, 1H), 1.84-1.70 (m, 2H), 1.69-1.42 (m, 6H) ppm | N-(2- thienyl- methyl)- cyclopentan- amine | General method D1 using 3- fluorophenylisocyanate | 1-cyclopentyl-3- (3- fluorophenyl)-1- (2- thienylmethyl) urea |
| 56 | 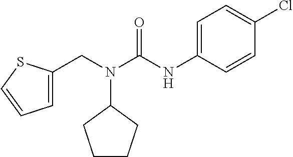 [SMILES: O=C(Nc1ccc(Cl)cc1)N(Cc1cccs1)C1CCCC1] | 335.6 | | 1H NMR (300 MHz, DMSO-d6): δ = 8.49 (s, 1H), 7.52-7.41 (m, 2H), 7.33 (dd, J = 5.1, 1.2 Hz, 1H), 7.30-7.19 (m, 2H), 6.99-6.96 (m, 1H), 6.92 (dd, J = 5.1, 3.5 Hz, 1H), 4.65 (s, 2H), 4.49-4.43 (m, 1H), 1.82-1.68 (m, 2H), 1.69-1.39 (m, 6H) ppm | N-(2- thienyl- methyl)- cyclopentan- amine | General method D1 using 2- chlorophenylisocyanate | 3-(4- chlorophenyl)-1- cyclopentyl-1- (2- thienylmethyl) urea |
| 57 | 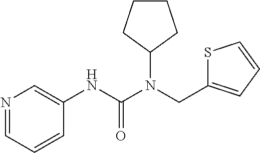 [SMILES: O=C(Nc1cccnc1)N(Cc1cccs1)C1CCCC1] | 302.7 | | 1H NMR (300 MHz, DMSO-d6): δ = 8.64-8.57 (m, 1H), 8.14 (dd, J = 4.8, 1.4 Hz, 1H), 7.89-7.83 (m, 1H), 7.34 (dd, J = 5.1, 1.2 Hz, 1H), 7.26-7.21 (m, 1H), 7.02-6.97 (m, 1H), 6.95-6.89 (m, 1H), 4.68 (s, 1H), 4.51-4.35 (m, 1H), 1.84-1.72 (m, 2H), 1.70-1.42 (m, 2H), 1.69- 1.39 (m, 6H) ppm | N-(2- thienyl- methyl)- cyclopentan- amine | General method D2 using 3- aminopyridine | 1-cyclopentyl-1- (3-pyridyl)-1- thienylmethyl- urea |
| 58 | 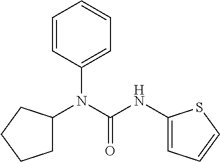 [SMILES: O=C(Nc1cccs1)N(c1ccccc1)C1CCCC1] | 287.6 | | 1H NMR (300 MHz, DMSO-d6): δ = 8.60 (s, 1H), 7.55-7.35 (m, 3H), 7.28-7.12 (m, 2H), 6.74 (dd, J = 5.4, 1.1 Hz, 1H), 6.73-6.62 (m, 1H), 6.51 (dd, J = 3.7, 1.5 Hz, 1H), 4.69-4.54 (m, 1H), 1.88-1.70 (m, 2H), 1.53-1.34 (m, 4H), 1.31-1.14 (m, 2H) ppm | N- cyclopentyl- aniline | General method D1 using 2-thienyl isocyanate | 1-cyclopentyl-1- phenyl-3-(2- thienyl)urea |
| 59 | 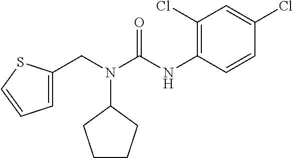 [SMILES: O=C(Nc1ccc(Cl)cc1Cl)N(Cc1cccs1)C1CCCC1] | 369.6 | | 1H NMR (400 MHz, DMSO-d6): δ = 8.66 (s, 1H), 7.83-7.82 (m, 1H), 7.49-7.44 (m, 2H), 7.34 (dd, J = 3.6, 1.0 Hz, 1H), 7.00-6.96 (m, 1H), 6.92 (dd, J = 5.1, 3.6 Hz, 1H), 4.66 (s, 2H), 4.44-4.34 (m, 1H), 1.83-1.70 (m, 2H), 1.69-1.61 (m, 2H), 1.60-1.45 (m, 4H) ppm | N-(2- thienyl- methyl)- cyclopentan- amine | General method D1 using 2,4-dichloro-1- isocyanatobenzene | 1-cyclopentyl-3- (2,4- dichlorophenyl)- 1-(2- thienylmethyl) urea |
| 60 | 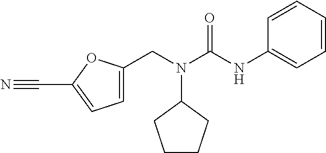 [SMILES: N#Cc1ccc(CN(C(=O)Nc2ccccc2)C2CCCC2)o1] | 310.6 | | 1H NMR (400 MHz, DMSO-d6): δ = 8.41 (s, 1H), 7.52 (d, J = 3.6 Hz, 1H), 7.44-7.40 (m, 2H), 7.24- 7.18 (m, 2H), 6.95-6.91 (m, 1H), 6.49 (d, J = 3.7 Hz, 1H), 4.54 (s, 2H), 4.51-4.42 (m, 1H), 1.86-1.73 (m, 2H), 1.71-1.58 (m, 2H), 1.56- 1.41 (m, 4H) ppm | 5- formylfuran- 2-carbonitrile | General method A using cyclopentanamine, AcOH and NaBH₄, method D1 using phenyl isocyanate | 1-[(5-cyano-2- furyl)methyl] cyclopentyl- phenyl-urea |
| 61 | 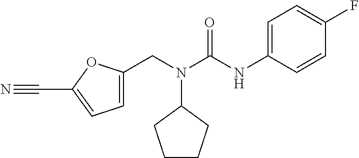 [SMILES: N#Cc1ccc(CN(C(=O)Nc2ccc(F)cc2)C2CCCC2)o1] | 328.6 | | 1H NMR (400 MHz, DMSO-d6): δ = 8.46 (s, 1H), 7.51 (d, J = 3.7 Hz, 1H), 7.44-7.39 (m, 2H), 7.05 (t, J = 8.9 Hz, 2H), 6.49 (d, J = 3.7 Hz, 1H), 4.53 (s, 2H), 4.49-4.39 (m, 1H), 1.86-1.73 (m, 2H), 1.71- 1.57 (m, 2H), 1.56-1.40 (m, 4H) ppm | 5- formylfuran- 2-carbonitrile | General method A using cyclopentanamine, AcOH and NaBH₄, method D1 using 4- fluorophenyl- isocyanate | 1-[(5-cyano-2- furyl)methyl]-1- cyclopentyl-3- (4- fluorophenyl) urea |
| 62 | 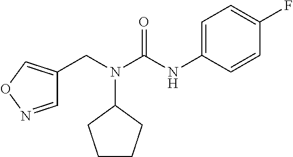 [SMILES: O=C(Nc1ccc(F)cc1)N(Cc1cnoc1)C1CCCC1] | 304.6 | | 1H NMR (400 MHz, DMSO-d6): δ = 8.79 (d, J = 1.6 Hz, 1H), 8.46 (s, 1H), 7.46-7.41 (m, 2H), 7.09- 7.02 (m, 2H), 6.41 (d, J = 1.6 Hz, 1H), 4.54 (s, 2H), 4.48-4.39 (m, 1H), 1.83-1.71 (m, 2H), 1.68-1.58 (m, 2H), 1.55-1.43 (m, 4H) ppm | isoxazole-4- carbaldehyde | General method A using cyclopentanamine, AcOH and NaBH₄, method D1 using 4- fluorophenyl- isocyanate | 1-cyclopentyl-3 (4- fluorophenyl)-1- (isoxazol-4- ylmethyl)urea |
| 63 | 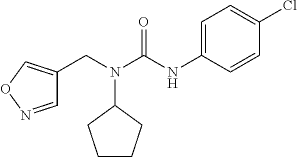 [SMILES: O=C(Nc1ccc(Cl)cc1)N(Cc1cnoc1)C1CCCC1] | 320.6 | | 1H NMR (400 MHz, DMSO-d6): δ = 8.79 (d, J = 1.6 Hz, 1H), 8.54 (s, 1H), 7.48 (d, J = 9.0 Hz, 2H), 7.27 (d, J = 9.0 Hz, 2H), 6.41 (d, J = 1.6 Hz, 1H), 4.55 (s, 2H), 4.49- 4.39 (m, 1H), 1.84-1.71 (m, 2H), 1.69-1.57 (m, 2H), 1.56-1.43 (m, 4H) ppm | isoxazole-4- carbaldehyde | General method A using cyclopentanamine, AcOH and NaBH₄, method D1 using 4- chlorophenyl- isocyanate | 3-(4- chlorophenyl)-1- cyclopentyl- (isoxazol-4- ylmethyl)urea |
| 64 | 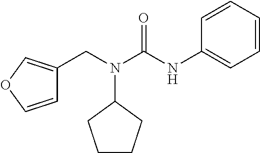 [SMILES: O=C(Nc1ccccc1)N(Cc1ccoc1)C1CCCC1] | 285.6 | | 1H NMR (400 MHz, DMSO-d6): δ = 8.19 (s, 1H), 7.55 (t, J = 1.4 Hz, 1H), 7.53-7.51 (m, 1H), 7.44-7.40 (m, 2H), 7.23-7.17 (m, 2H), 6.96- 6.88 (m, 1H), 6.39 (dd, J = 1.7, 0.9 Hz, 1H), 4.49-4.38 (m, 1H), 4.31 (s, 2H), 1.81-1.70 (m, 2H), 1.67- 1.59 (m, 2H), 1.57-1.43 (m, 4H) ppm | furane-3- carbaldehyde | General method A using cyclopentanamine, AcOH and NaBH₄, method D1 using phenyl isocyanate | 1-cyclopentyl-1- (3-furylmethyl)- 3-phenyl-urea |
| 65 | 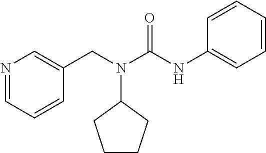 [SMILES: O=C(Nc1ccccc1)N(Cc1cccnc1)C1CCCC1] | 296.7 | | 1H NMR (300 MHz, DMSO-d6): δ = 8.46 (d, J = 1.6 Hz, 1H), 8.40 (dd, J = 4.5, 1.8 Hz, 1H), 8.38 (s, 1H), 7.63-7.58 (m, 1H), 7.45-7.39 (m, 2H), 7.35-7.29 (m, 1H), 7.24- 7.16 (m, 2H), 6.95-6.88 (m, 1H), 4.59-4.46 (m, 3H), 1.84-1.70 (m, 2H), 1.69-1.56 (m, 2H), 1.55- 1.36 (m, 4H) ppm | pyridine-3- carbaldehyde | General method A using cyclopentanamine, AcOH and NaBH₄, method D1 using phenyl isocyanate | 1-cyclopentyl-3- phenyl-1-(3- pyridylmethyl) urea |
| 66 | 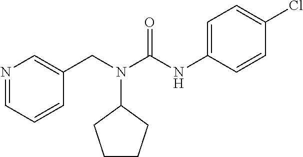 [SMILES: O=C(Nc1ccc(Cl)cc1)N(Cc1cccnc1)C1CCCC1] | 330.6 | | 1H NMR (300 MHz, DMSO-d6): δ = 8.52 (s, 1H), 8.47-8.43 (m, 1H), 8.40 (dd, J = 4.9, 1.6 Hz, 1H), 7.62-7.57 (m, 1H), 7.49-7.42 (m, 2H), 7.37-7.29 (m, 1H), 7.28-7.20 (m, 2H), 4.57-4.43 (m, 3H), 1.84- 1.69 (m, 2H), 1.68-1.55 (m, 2H), 1.54-1.35 (m, 4H) ppm | pyridine-3- carbaldehyde | General method A using cyclopentanamine, AcOH and NaBH₄, method D1 using 4- chlorophenyl- isocyanate | 3-(4- chlorophenyl)-1- cyclopentyl-1- (3- pyridylmethyl) urea |
| 67 | 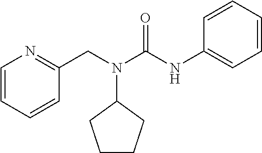 [SMILES: O=C(Nc1ccccc1)N(Cc1ccccn1)C1CCCC1] | 296.7 | | 1H NMR (300 MHz, DMSO-d6): δ = 9.01 (s, 1H), 8.58-8.52 (m, 1H), 7.78 (td, J = 7.6, 2.2 Hz, 1H), 7.46-7.39 (m, 2H), 7.36-7.16 (m, 4H), 6.95-6.87 (m, 1H), 4.60-4.46 (m, 3H), 1.80-1.55 (m, 4H), 1.54- 1.37 (m, 4H) ppm | pyridine-2- carbaldehyde | General method A using cyclopentanamine, AcOH and NaBH₄, method D1 using phenyl isocyanate | 1-cyclopentyl-3- phenyl-1-(2- pyridylmethyl) urea |
| 68 | 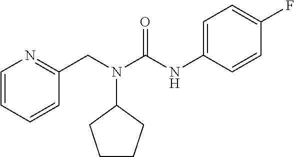 [SMILES: O=C(Nc1ccc(F)cc1)N(Cc1ccccn1)C1CCCC1] | 314.6 | | 1H NMR (300 MHz, DMSO-d6): δ = 8.96 (s, 1H), 8.57-8.51 (m, 1H), 7.77 (td, J = 7.7, 1.8 Hz, 1H), 7.47-7.37 (m, 2H), 7.35-7.23 (m, 2H), 7.11-6.99 (m, 2H), 4.60-4.44 (m, 3H), 1.79-1.55 (m, 4H), 1.54- 1.37 (m, 4H) ppm | pyridine-2- carbaldehyde | General method A using cyclopentanamine, AcOH and NaBH₄, method D1 using 4- flurophenyl- isocyanate | 1-cyclopentyl- (4- fluorophenyl)- (2- pyridylmethyl) urea |
| 69 | 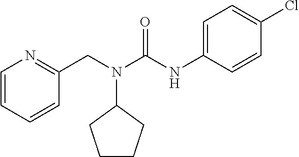 [SMILES: O=C(Nc1ccc(Cl)cc1)N(Cc1ccccn1)C1CCCC1] | 330.6 | | 1H NMR (300 MHz, DMSO-d6): δ = 9.09 (s, 1H), 8.57-8.51 (m, 1H), 7.77 (td, J = 7.7, 1.8 Hz, 1H), 7.51-7.43 (m, 2H), 7.35-7.22 (m, 4H), 4.58-4.44 (m, 3H), 1.80-1.55 (m, 4H), 1.54-1.37 (m, 4H) ppm | pyridine-2- carbaldehyde | General method A using cyclopentanamine, AcOH and NaBH₄, method D1 using 4- chlorophenyl- isocyanate | 3-(4- chlorophenyl)-1- cyclopentyl-1- (2- pyridylmethyl) urea |
| 70 | 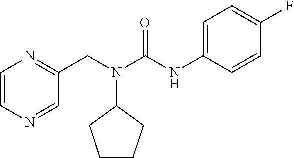 [SMILES: O=C(Nc1ccc(F)cc1)N(Cc1cnccn1)C1CCCC1] | 315.6 | | 1H NMR (300 MHz, DMSO-d6): δ = 8.60-8.55 (m, 3H), 8.49 (d, J = 2.4 Hz, 1H), 7.46-7.37 (m, 2H), 7.04 (t, J = 8.8 Hz, 2H), 4.60 (s, 2H), 4.57-4.43 (m, 1H), 1.87-1.70 (m, 2H), 1.69-1.56 (m, 2H), 1.55- 1.37 (m, 4H) ppm | pyridine-2- carbaldehyde | General method A using cyclopentanamine, AcOH and NaBH₄, method D1 using 4- fluorophenyl- isocyanate | 1-cyclopentyl-3- (4- fluorophenyl)-1- (pyrazin-2- ylmethyl)urea |
| 71 | 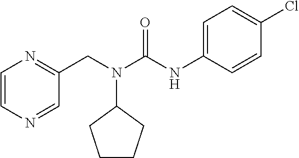 [SMILES: O=C(Nc1ccc(Cl)cc1)N(Cc1cnccn1)C1CCCC1] | 331.6 | | 1H NMR (300 MHz, DMSO-d6): δ = 8.67 (s, 1H), 8.59-8.55 (m, 2H), 8.50 (d, J = 2.5 Hz, 1H), 7.46 (d, J = 8.9 Hz, 2H), 7.25 (d, J = 8.9 Hz, 2H), 4.60 (s, 2H), 4.59-4.45 (m, 1H), 1.87-1.68 (m, 2H), 1.67- 1.56 (m, 2H), 1.55-1.38 (m, 4H) ppm | pyridine-2- carbaldehyde | General method A using cyclopentanamine, AcOH and NaBH₄, method D1 using 4- chlorophenyl- isocyanate | 3-(4- chlorophenyl)-1- cyclopentyl- (pyrazin-2- ylmethyl)urea |
| 72 | 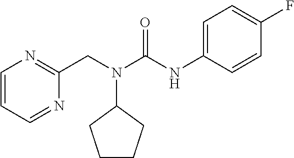 [SMILES: O=C(Nc1ccc(F)cc1)N(Cc1ncccn1)C1CCCC1] | 315.6 | | 1H NMR (300 MHz, DMSO-d6): δ = 8.77 (d, J = 4.9 Hz, 2H), 8.62 (s, 1H), 7.45-7.35 (m, 3H), 7.07-6.98 (m, 2H), 4.64 (s, 2H), 4.61-4.46 (m, 1H), 1.82-1.66 (m, 2H), 1.66- 1.54 (m, 2H), 1.53-1.35 (m, 4H) ppm | pyridine-2- carbaldehyde | General method A using cyclopentanamine, AcOH and NaBH₄, method D1 using 4- fluorophenyl- isocyanate | 1-cyclopentyl-3- (4- fluorophenyl)-1- (pyrimidin-2- ylmethyl)urea |
| 73 | 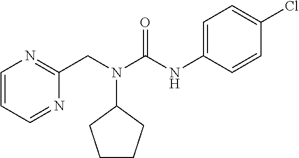 [SMILES: O=C(Nc1ccc(Cl)cc1)N(Cc1ncccn1)C1CCCC1] | 331.6 | | 1H NMR (300 MHz, DMSO-d6): δ = 8.77 (d, J = 4.9 Hz, 2H), 8.71 (s, 1H), 7.45 (d, J = 8.9 Hz, 2H), 7.38 (t, J = 1.8 Hz, 1H), 7.23 (d, J = 8.9 Hz, 2H), 4.65 (s, 2H), 4.61-4.48 (m, 1H), 1.83-1.67 (m, 2H), 1.65- 1.34 (m, 6H) ppm | pyridine-2- carbaldehyde | General method A using cyclopentanamine, AcOH and NaBH₄, method D1 using 4- chlorophenyl- isocyanate | 3-(4- chlorophenyl)-1- cyclopentyl-1- (pyrimidin-2- ylmethyl)urea |
| 74 | 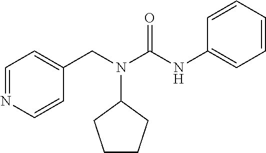 [SMILES: O=C(Nc1ccccc1)N(Cc1ccncc1)C1CCCC1] | 296.7 | | 1H NMR (300 MHz, DMSO-d6): δ = 8.49-8.44 (m, 2H), 8.38 (s, 1H), 7.46-7.38 (m, 2H), 7.25-7.15 (m, 4H), 6.96-6.88 (m, 1H), 4.63- 4.49 (m, 3H), 1.84-1.69 (m, 2H), 1.68-1.55 (m, 2H), 1.54-1.27 (m, 4H) ppm | pyridine-4- carbaldehyde | General method A using cyclopentanamine, AcOH and NaBH₄, method D1 using phenyl isocyanate | 1-cyclopentyl-1- phenyl-1-(4- pyridylmethyl) urea |
| 75 | 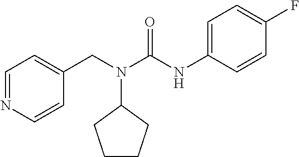 [SMILES: O=C(Nc1ccc(F)cc1)N(Cc1ccncc1)C1CCCC1] | 314.6 | | 1H NMR (300 MHz, DMSO-d6): δ = 8.49-8.44 (m, 2H), 8.43 (s, 1H), 7.42 (dd, J = 9.2, 5.1 Hz, 2H), 7.25-7.18 (m, 2H), 7.04 (t, J = 8.9 Hz, 2H), 4.61-4.46 (m, 3H), 1.86- 1.67 (m, 2H), 1.66-1.54 (m, 2H), 1.53-1.25 (m, 4H) ppm | pyridine-4- carbaldehyde | General method A using cyclopentanamine, AcOH and NaBH₄, method D1 using 4- fluorophenyl- isocyanate | 1-cyclopentyl-1- (4- fluorophenyl)-1- (4- pyridylmethyl) urea |
| 76 | 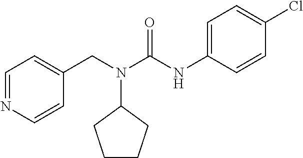 [SMILES: O=C(Nc1ccc(Cl)cc1)N(Cc1ccncc1)C1CCCC1] | 330.6 | | 1H NMR (300 MHz, DMSO-d6): δ = 8.52 (s, 1H), 8.49-8.44 (m, 2H), 7.50-7.43 (m, 2H), 7.29-7.18 (m, 4H), 4.62-4.45 (m, 3H), 1.88- 1.68 (m, 2H), 1.56-1.54 (m, 2H), 1.53-1.29 (m, 4H) ppm | pyridine-4- carbaldehyde | General method A using cyclopentanamine, AcOH and NaBH₄, method D1 using 4- chlorophenyl- isocyanate | 3-(4- chlorophenyl)-1- cyclopentyl-1- (4- pyridylmethyl) urea |
| 77 | 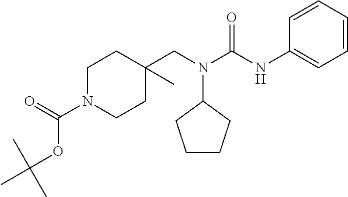 [SMILES: CC1(CN(C(=O)Nc2ccccc2)C2CCCC2)CCN(C(=O)OC(C)(C)C)CC1] | 416.8 | | 1H NMR (500 MHz, DMSO-d6): δ = 8.22 (s, 1H), 7.39 (d, J = 7.6 Hz, 2H), 7.21 (t, J = 4.4, 2H), 6.91 (t, J = 4.3 Hz, 1H), 3.90-3.76 (m, 1H), 3.72-3.57 (m, 2H), 3.21 (s, 2H), 3.06-2.58 (m, 2H), 1.84-1.65 (m, 6H), 1.51-1.39 (m, 4H), 1.37 (s, 9H), 1.30-1.19 (m, 2H), 0.95 (s, 3H) ppm | tert-butyl 4- formyl-4- methyl- piperidine-1- carboxylate | General method A using cyclopentanamine and NaBH₄, method D1 using phenyl isocyanate | tert-butyl 4- [[cyclopentyl (phenylcarbamoyl) amino]methyl]-4- methyl- piperidine-1- carboxylate |
| 78 | 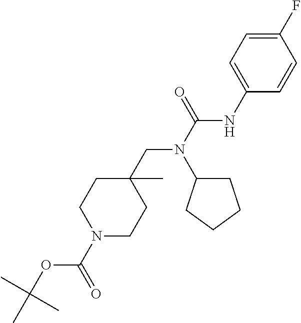 [SMILES: CC1(CN(C(=O)Nc2ccc(F)cc2)C2CCCC2)CCN(C(=O)OC(C)(C)C)CC1] | 434.8 | | 1H NMR (500 MHz, DMSO-d6): δ = 8.25 (s, 1H), 7.43-7.36 (m, 2H), 7.05 (t, J = 5.6 Hz, 2H), 3.86- 3.76 (m, 1H), 3.69-3.58 (m, 2H), 3.36-3.42 (m, 1H), 3.20 (s, 2H), 3.05-2.86 (bs, 2H), 1.82-1.65 (m, 6H), 1.49-1.39 (m, 4H), 1.37 (s, 9H), 1.28-1.20 (m, 2H), 0.95 (s, 3H) ppm | tert-butyl 4- formyl-4- methyl- piperidine-1- carboxylate | General method A using cyclopentanamine and NaBH₄, method D1 using 4- fluorophenyl- isocyanate | tert-butyl 4- [[cyclopentyl- (phenylcarbamoyl) amino]methyl]-4- methyl- piperidine-1- carboxylate |
| 79 | 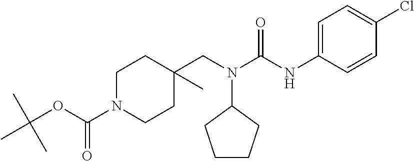 [SMILES: CC1(CN(C(=O)Nc2ccc(Cl)cc2)C2CCCC2)CCN(C(=O)OC(C)(C)C)CC1] | 450.7 | | 1H NMR (500 MHz, DMSO-d6): δ = 8.35 (s, 1H), 7.44 (d, J = 8.9 Hz, 2H), 7.26 (d, J = 8.9 Hz, 2H), 3.85-3.76 (m, 1H), 3.70-3.57 (m, 2H), 3.21 (s, 2H), 3.08-2.85 (bs, 1H), 1.83-1.66 (m, 6H), 1.50-1.40 (m, 4H), 1.39 (s, 9H), 1.28-1.19 (m, 2H), 0.94 (s, 3H) ppm | tert-butyl 4- formyl-4- methyl- piperidine-1- carboxylate | General method A using cyclopentanamine and NaBH₄, method D1 using 4- chlorophenyl- isocyanate | tert-butyl 4-[[(4- chlorophenyl) carbamoyl- cyclopentyl- amino]methyl]- 4-methyl- piperidine-1- carboxylate |
| 80 | 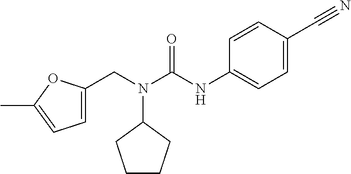 [SMILES: Cc1ccc(CN(C(=O)Nc2ccc(C#N)cc2)C2CCCC2)o1] | 324.6 | | 1H NMR (300 MHz, DMSO-d6): δ = 8.76 (s, 1H), 7.65 (s, 4H), 6.08 (d, J = 3.0 Hz, 1H), 5.97-5.93 (m, 1H), 4.50-4.36 (m, 3H), 2.19 (s, 3H), 1.84-1.70 (m, 2H), 1.69-1.40 (m, 6H) ppm | 5- methylfuran- 2- carbaldehyde | General method A using cyclopentanamine, AcOH and NaBH₄, method D1 using 4- isocyanato- benzonitrile | 3-(4- cyanophenyl)- cyclopentyl- [(5-methyl-2- furyl)methyl] urea |
| 81 | 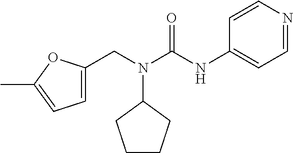 [SMILES: Cc1ccc(CN(C(=O)Nc2ccncc2)C2CCCC2)o1] | 300.1 | | 1H NMR (300 MHz, DMSO-d6): δ = 8.70 (s, 1H), 8.30-8.25 (m, 2H), 7.48-7.42 (m, 2H), 6.08 (d, J = 3.1 Hz, 1H), 5.96-5.90 (m, 1H), 4.48-4.36 (m, 3H), 2.19 (s, 3H), 1.85-1.71 (m, 2H), 1.70-1.40 (m, 6H) ppm | 5- methylfuran- 2- carbaldehyde | General method A using cyclopentanamine, AcOH and NaBH₄, method D1 using 4- isocyanato- pyridine | 1-cyclopentyl-1- [(5-methyl-2- furyl)methyl]-3- (4-pyridyl)urea |
| 82 | 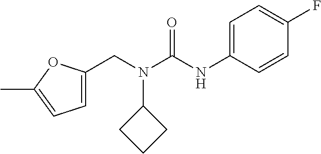 [SMILES: Cc1ccc(CN(C(=O)Nc2ccc(F)cc2)C2CCC2)o1] | 303 | | 1H NMR (300 MHz, DMSO-d6): δ = 8.27 (s, 1H), 7.57-7.28 (m, 2H), 7.05 (t, J = 8.5 Hz, 2H), 6.06 (s, 1H), 5.96 (s, 1H), 4.51 (s, 2H), 4.46-4.24 (m, 1H), 2.20 (s, 3H), 2.17-1.99 (m, 4H), 1.69-1.37 (m, 2H) ppm | 5- methylfuran- 2- carbaldehyde | General method A using cyclobutanamine, AcOH and NaBH₄, method D1 using 4- fluorophenyl- isocyanate | 1-cyclobutyl-3- (4- fluorophenyl)-1- [(5-methyl-2- furyl)methyl]urea |
| 83 | 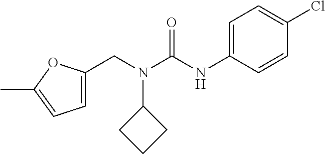 [SMILES: Cc1ccc(CN(C(=O)Nc2ccc(Cl)cc2)C2CCC2)o1] | 319.6 | | 1H NMR (300 MHz, DMSO-d6): δ = 8.37 (s, 1H), 7.47 (d, J = 8.9 Hz, 2H), 7.26 (d, J = 8.9 Hz, 2H), 6.06 (d, J = 3.0 Hz, 1H), 5.98-5.92 (m, 1H), 4.51 (s,2H), 4.45-4.31 (m, 1H), 2.20 (s, 3H), 2.16-1.99 (m, 4H), 1.68-1.42 (m, 2H) ppm | 5- methylfuran- 2- carbaldehyde | General method A using cyclobutanamine, AcOH and NaBH₄, method D1 using 4- chlorophenyl- isocyanate | 3-(4- chlorophenyl)-1- cyclobutyl-1- methyl-2- furyl)methyl]urea |
| 84 | 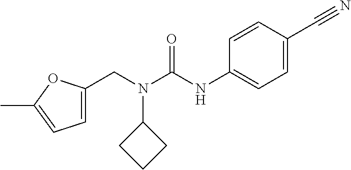 [SMILES: Cc1ccc(CN(C(=O)Nc2ccc(C#N)cc2)C2CCC2)o1] | 310.6 | | 1H NMR (300 MHz, DMSO-d6): δ = 8.74 (s, 1H), 7.70-7.61 (m, 4H), 6.07 (d, J = 3.1 Hz, 1H), 5.97- 5.93 (m, 1H), 4.53 (s, 2H), 4.45- 4.29 (m, 1H), 2.19 (s, 3H), 2.17- 2.01 (m, 4H), 1.68-1.46 (m, 2H) ppm | 5- methylfuran- 2- carbaldehyde | General method A using cyclobutanamine, AcOH and NaBH₄, method D1 using 4- isocyanato- benzonitrile | 3-(4- cyanophenyl)-1- cyclobutyl-1-[(5- methyl-2- furyl)methyl]urea |
| 85 | 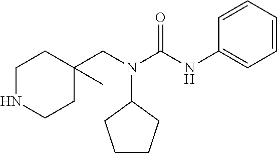 [SMILES: CC1(CN(C(=O)Nc2ccccc2)C2CCCC2)CCNCC1] | 316.1 | | 1H NMR (500 MHz, DMSO-d6): δ = 8.16 (s, 1H), 7.40 (d, J = 7.7 Hz, 2H), 7.20 (t, J = 7.7 Hz, 2H), 6.90 (t, J = 4.4 Hz, 1H), 3.86-3.75 (m, 1H), 3.19 (s, 2H), 2.73-2.66 (m, 2H), 2.65-2.56 (m, 2H), 1.88- 1.65 (m, 6H), 1.51-1.35 (m, 4H), 1.27-1.13 (m, 3H), 0.92 (s, 3H) ppm | tert-butyl 4- formyl-4- methyl- piperidine-1- carboxylate | General method A using cyclopentanamine and NaBH₄, method D1 using phenyl isocyanate, method G using TFA | 1-cyclopentyl-1- [(4-methyl-4- piperidyl)methyl]- 3-phenyl-urea |
| 86 | 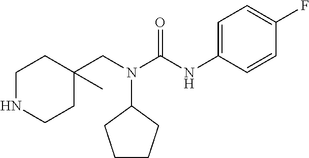 [SMILES: CC1(CN(C(=O)Nc2ccc(F)cc2)C2CCCC2)CCNCC1] | 334 | | 1H NMR (500 MHz, DMSO-d6): δ = 8.19 (s, 1H), 7.40 (dd, J = 9.0, 4.9 Hz, 2H), 7.04 (t, J = 9.0 Hz, 2H), 3.82-3.72 (m, 1H), 3.17 (s, 2H), 2.73-2.64 (m, 2H), 2.63-2.55 (m, 2H), 1.87-1.64 (m, 6H), 1.50- 1.34 (m, 4H), 1.24-1.14 (m, 2H), 0.92 (s, 3H) ppm | tert-butyl 4- formyl-4- methyl- piperidine-1- carboxylate | General method A using cyclopentanamine and NaBH₄, method D1 using 4- fluorophenyl- isocyanate, method G using TFA | 1-cyclopentyl-3- (4- fluorophenyl)- [(4-methyl-4- piperidyl)methyl] urea |
| 87 | 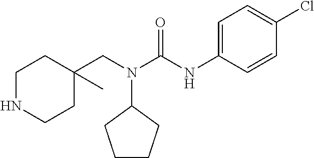 [SMILES: CC1(CN(C(=O)Nc2ccc(Cl)cc2)C2CCCC2)CCNCC1] | 350 | | 1H NMR (500 MHz, DMSO-d6): δ = 8.30 (s, 1H), 7.44 (d, J = 8.9 Hz, 2H), 7.25 (d, J = 8.9 Hz, 2H), 3.83-3.70 (m, 1H), 3.19 (s, 2H), 2.73-2.65 (m, 2H), 2.64-2.55 (m, 2H), 1.88-1.63 (m, 6H), 1.50-1.34 (m, 4H), 1.26-1.14 (m, 2H), 0.91 (s, 3H) ppm | tert-butyl 4- formyl-4- methyl- piperidine-1- carboxylate | General method A using cyclopentanamine and NaBH₄, method D1 using 4- chlorophenyl- isocyanate, method G using TFA | 3-(4- chlorophenyl)-1- cyclopentyl-1- [(4-methyl-4- piperidyl)methyl] urea |
| 88 | 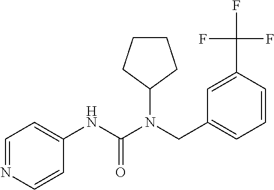 [SMILES: O=C(Nc1ccncc1)N(Cc1cccc(C(F)(F)F)c1)C1CCCC1] | 364.2 | | 1H NMR (300 MHz, DMSO-d6): δ = 8.84 (s, 1H), 8.31-8.26 (m, 2H), 7.62-7.49 (m, 4H), 7.48-43 (m, 2H), 4.63 (s, 2H), 4.59-4.47 (m, 1H), 1.85-1.69 (m, 2H), 1.68- 1.55 (m, 2H), 1.54-1.37 (m, 4H) ppm | 3- (trifluoro- methyl) benzaldehyde | General method A using cyclopentanamine, AcOH and NaBH₄, method D1 using 4- isocyanatopyridine | 1-cyclopentyl-3- (4-pyridyl)-1- [[3- (trifluoromethyl) phenyl]methyl] urea |
| 89 | 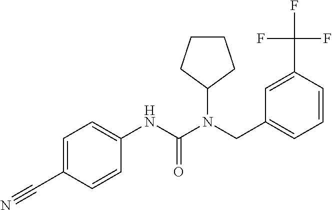 [SMILES: N#Cc1ccc(NC(=O)N(Cc2cccc(C(F)(F)F)c2)C2CCCC2)cc1] | 388.2 | | 1H NMR (300 MHz, DMSO-d6): δ = 8.89 (s, 1H), 7.69-7.7.61 (m, 4H), 7.59-7.48 (m, 4H), 4.63 (s, 2H), 4.59-4.45 (m, 1H), 1.86-1.69 (m, 2H), 1.68-1.55 (m, 2H), 1.54- 1.38 (m, 4H) ppm | 3- (trifluoro- methyl) benzaldehyde | General method A using cyclopentanamine, AcOH and NaBH₄, method D1 using 4- isocyanatobenzonitrile | 3-(4- cyanophenyl)-1- cyclopentyl-1- [[3- (trifluoromethyl) phenyl]methyl] urea |
| 90 | 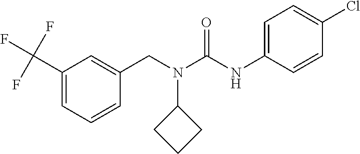 [SMILES: O=C(Nc1ccc(Cl)cc1)N(Cc1cccc(C(F)(F)F)c1)C1CCC1] | 383.1 | | 1H NMR (300 MHz, DMSO-d6): δ = 8.47 (s, 1H), 7.62-7.42 (m, 6H), 7.30-7.23 (m, 2H), 4.73 (s, 2H), 4.65-4.49 (m, 1H), 2.16-1.91 (m, 4H), 1.62-1.40 (m, 2H) ppm | 3- (trifluoro- methyl) benzaldehyde | General method A using cyclobutanamine, AcOH and NaBH₄, method D1 using 4- chlorophenyl- isocyanate | 3-(4- chlorophenyl)- cyclobutyl-1-[[3- (trifluoromethyl) phenyl]methyl] urea |
| 91 | 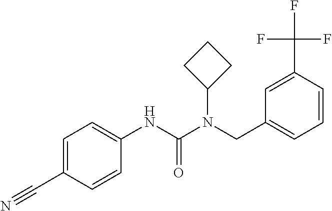 [SMILES: N#Cc1ccc(NC(=O)N(Cc2cccc(C(F)(F)F)c2)C2CCC2)cc1] | 374 | | 1H NMR (300 MHz, DMSO-d6): δ = 8.83 (s, 1H), 7.70-7.61 (m, 4H), 7.59-7.46 (m, 4H), 4.75 (s, 2H), 4.66-4.50 (m, 1H), 2.15-1.96 (m, 4H), 1.63-1.45 (m, 2H) ppm | 3- (trifluoro- methyl) benzaldehyde | General method A using cyclobutanamine, AcOH and NaBH₄, method D1 using 4- isocyanato- benzonitrile | 3-(4- cyanophenyl)-1- cyclobutyl-1-[[3- (trifluoromethyl) phenyl]methyl] urea |
| 92 | 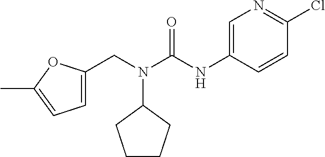 [SMILES: Cc1ccc(CN(C(=O)Nc2ccc(Cl)nc2)C2CCCC2)o1] | 334.1 | | 1H NMR (300 MHz, DMSO-d6): δ = 8.64 (s, 1H), 8.47 (d, J = 2.4 Hz, 1H), 7.93 (d, J = 3.0 Hz, 1H), 7.36 (d, J = 8.7 Hz, 1H), 6.09 (d, J = 3.0 Hz, 1H), 5.97-5.92 (m, 1H), 4.49-4.30 (m, 3H), 2.20 (s, 3H), 1.83-1.70 (m, 2H), 1.69-1.43 (m, 6H) ppm | 5- methylfuran- 2- carbaldehyde | General method A using cyclopentanamine, AcOH and NaBH₄, method D2 using 6- chloropyridin-3- amine | 3-(6-chloro-3- pyridyl)-1- cyclopentyl-1- [(5-methyl-2- furyl)methyl]urea |
| 93 | 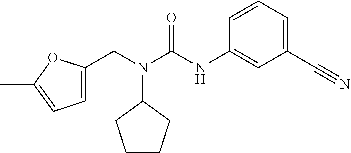 [SMILES: Cc1ccc(CN(C(=O)Nc2cccc(C#N)c2)C2CCCC2)o1] | 324.1 | | 1H NMR (300 MHz, DMSO-d6): δ = 8.63 (s, 1H), 7.93 (t, J = 1.7 Hz, 1H), 7.77-7.70 (m, 1H), 7.47-7.33 (m, 2H), 6.09 (d, J = 3.1 Hz, 1H), 5.99-5.92 (m, 1H), 4.51-4.32 (m, 3H), 2.21 (s, 3H), 1.86-1.70 (m, 2H), 1.69-1.39 (m, 6H) ppm | 5- methylfuran- 2- carbaldehyde | General method A using cyclopentanamine, AcOH and NaBH₄, method D1 using 3- isocyanato- benzonitrile | 3-(3- cyanophenyl)-1- cyclopentyl-1- [(5-methyl-2- furyl)methyl]urea |
| 94 | 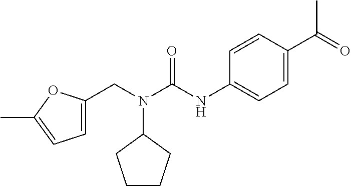 [SMILES: CC(=O)c1ccc(NC(=O)N(Cc2ccc(C)o2)C2CCCC2)cc1] | 341.2 | | 1H NMR (300 MHz, DMSO-d6): δ = 8.66 (s, 1H), 7.84 (d, J = 8.9 Hz, 2H), 7.59 (d, J = 8.9 Hz, 2H), 6.09 (d, J = 3.1 Hz, 1H), 5.98-5.93 (m, 1H), 4.54-4.34 (m, 3H), 2.20 (m, 3H), 1.83-1.71 (m, 2H), 1.70- 1.43 (m, 6H) ppm | 5- methylfuran- 2- carbaldehyde | General method A using cyclopentanamine, AcOH and NaBH₄, method D1 using 1-(4- isocyanatophenyl) ethanone | 3-(4- acetylphenyl)-1- cyclopentyl-1- [(5-methyl-2- furyl)methyl]urea |
| 95 | 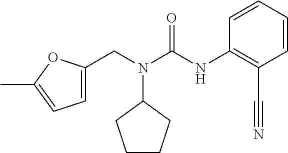 [SMILES: Cc1ccc(CN(C(=O)Nc2ccccc2C#N)C2CCCC2)o1] | 324.1 | | 1H NMR (300 MHz, DMSO-d6): δ = 8.57 (s, 1H), 7.71 (dd, J = 7.6, 1.3 Hz, 1H), 7.63-7.56 (m, 1H), 7.48-7.42 (m, 1H), 7.22 (dt, J = 7.6, 1.2 Hz, 1H), 6.16 (d, J = 3.0 Hz, 1H), 5.98-5.94 (m, 1H), 4.44 (s, 2H), 4.42-4.31 (m, 1H), 2.22 (s, 3H), 1.84-1.72 (m, 2H), 1.69-1.42 (m, 6H) ppm | 5- methylfuran- 2- carbaldehyde | General method A using cyclopentanamine, AcOH and NaBH₄, method D1 using 2- isocyanato- benzonitrile | 3-(2- cyanophenyl)-1- cyclopentyl- [(5-methyl-2- furyl)methyl]urea |
| 96 | 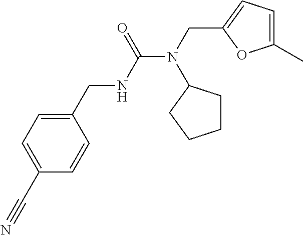 [SMILES: Cc1ccc(CN(C(=O)NCc2ccc(C#N)cc2)C2CCCC2)o1] | 338.1 | | 1H NMR (300 MHz, DMSO-d6): δ = 7.80-7.72 (m, 2H), 7.42-7.34 (m, 2H), 6.99 (t, J = 6.0 Hz, 1H), 6.02 (d, J = 3.0 Hz, 1H), 5.97-5.93 (m, 1H), 4.36-4.22 (m, 5H), 2.21 (s, 3H), 1.77-1.54 (m, 4H), 1.53- 1.36 (m, 4H) ppm | 5- methylfuran- 2- carbaldehyde | General method A using cyclopentanamine, AcOH and NaBH₄, method D2 using 4- (aminomethyl) benzonitrile | 3-[(4- cyanophenyl) methyl]-1- cyclopentyl-1- [(5-methyl-2- furyl)methyl]urea |
| 97 | 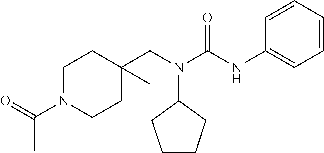 [SMILES: CC(=O)N1CCC(C)(CN(C(=O)Nc2ccccc2)C2CCCC2)CC1] | 358.2 | | 1H NMR (300 MHz, DMSO-d6): δ = 8.24 (s, 1H), 7.42-7.34 (m, 2H), 7.20 (t, J = 8.0 Hz, 2H), 6.94- 6.87 (m, 1H), 3.99-3.77 (m, 2H), 3.64-3.50 (m, 1H), 3.26-3.11 (m, 3H), 2.94-2.78 (m, 1H), 1.94 (s, 3H), 1.84-1.62 (m, 6H), 1.52-1.19 (m, 6H), 0.96 (s, 3H) ppm | tert-butyl 4- formyl-4- methyl- piperidine-1- carboxylate | General method A using cyclopentanamine and NaBH₄, method D1 using phenyl isocyanate, method G using TFA, method I using AcCl | 1-[(1-acetyl-4- methyl-4- piperidyl)methyl]- 1-cyclopentyl- 3-phenyl-urea |
| 98 | 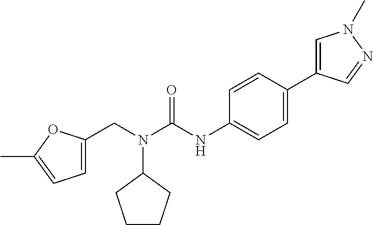 [SMILES: Cc1ccc(CN(C(=O)Nc2ccc(-c3cnn(C)c3)cc2)C2CCCC2)o1] | 379.1 | | 1H NMR (500 MHz, CDCl3): δ = 7.69 (s, 1H), 7.54 (s, 1H), 7.37 (d, J = 8.5 Hz, 2H), 7.32 (d, J = 8.5 Hz, 2H), 6.97 (bs, 1H), 6.23 (d, J = 2.5 Hz, 1H), 5.96 (d, J = 2.5 Hz, 1H), 4.73-4.66 (m, 1H), 4.34 (s, 2H), 3.93 (s, 3H), 2.33 (s, 3H), 1.97- 1.92 (m, 2H), 1.76-1.69 (m, 2H), 1.67-1.55 (m, 4H) ppm | 5- methylfuran- 2- carbaldehyde | General method A using cyclopentanamine, AcOH and NaBH₄, method D1 using 1- bromo-4- isocyanatobenzene, method E using 1- methyl-4-(4,4,5,5- tetramethyl-1,3,2- dioxaborolan-2- yl)pyrazole, Pd(PPh₃)₄ and K₂CO₃ | 1-cyclopentyl-1- [(5-methyl-2- furyl)methyl] [4-(1- methylpyrazol- 4-yl)phenyl]urea |
| 99 | 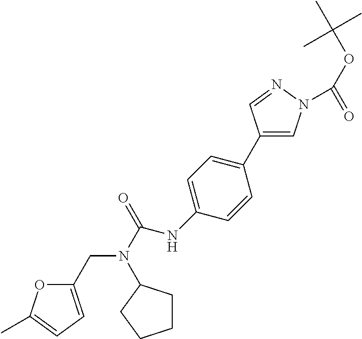 [SMILES: Cc1ccc(CN(C(=O)Nc2ccc(-c3cnn(C(=O)OC(C)(C)C)c3)cc2)C2CCCC2)o1] | 465.3 | | 1H NMR (300 MHz, CDCl3): δ = 8.21 (s, 1H), 7.93 (s, 1H), 7.41 (d, J = 8.7 Hz, 2H), 7.34 (d, J = 8.7 Hz, 2H), 7.01 (s, 1H), 6.18 (d, J = 3.1 Hz, 1H), 5.95 (s, 1H), 4.67 (m, 1H), 4.31 (s, 2H), 2.33 (s, 3H), 2.17-1.85 (m, 2H), 1.79-1.44 (m, 15H) ppm | 5- methylfuran- 2- carbaldehyde | General method A using cyclopentanamine, AcOH and NaBH₄, method D1 using 1- bromo-4- isocyanatobenzene, method E using tert- butyl 4-(4,4,5,5- tetramethyl-1,3,2- dioxaborolan-2- yl)pyrazole-1- carboxylate, Pd(PPh₃)₄ and K₂CO₃ | tert-butyl 4-[4- [[cyclopentyl- [(5-methyl-2- furyl)methyl] carbamoyl]amino] phenyl]pyrazole- 1-carboxylate |
| 100 | 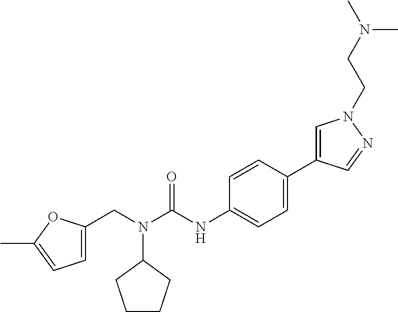 | 436.2 | | 1H NMR (300 MHz, CDCl3): δ = 7.73 (s, 1H), 7.68 (s, 1H), 7.38 (d, J = 8.6 Hz, 2H), 7.32 (d, J = 8.6 Hz, 2H), 6.96 (s, 1H), 6.20 (d, J = 2.9 Hz, 1H), 5.97 (d, J = 2.0 Hz, 1H), 4.69 (p, J = 8.4, 8.0 Hz, 1H), 4.34 (s, 2H), 4.30 (t, J = 6.4 Hz, 2H), 2.89 (bs, 2H), 2.31 (s, 3H), 2.30 (s, 3H), 2.92 (s, 3H), 1.96-1.87 (m, 2H), 1.74-1.62 (m, 2H), 1.62- 1.48 (m, 4H) ppm | 5- methylfuran- 2- carbaldehyde | General method A using cyclopentanamine, AcOH and NaBH₄, method D1 using 1- bromo-4- isocyanatobenzene, method E using N,N- dimethyl-2-[4-(4,4,5,5- tetramethyl-1,3,2- dioxaborolan-2- yl)pyrazole-1- yl]ethanamine, Pd(PPh₃)₄ and K₂CO₃ | 1-cyclopentyl-3- [4-[1-[2- (dimethylamino) ethyl]pyrazol- yl]phenyl]-1-[(5- methyl-2- furyl)methyl]urea |
| 101 | 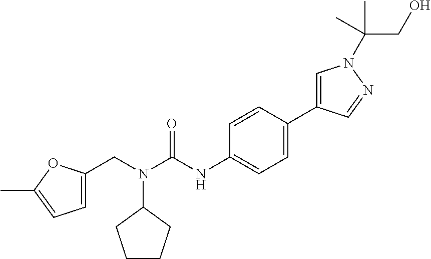 [SMILES: Cc1ccc(CN(C(=O)Nc2ccc(-c3cnn(C(C)(C)CO)c3)cc2)C2CCCC2)o1] | 437.2 | | 1H NMR (400 MHz, CDCl3): δ = 7.70 (s, 1H), 7.67 (s, 1H), 7.36 (d, J = 8.6 Hz, 2H), 7.30 (d, J = 8.6 Hz, 2H), 6.95 (s, 1H), 6.18, (d, J = 3.2 Hz, 1H), 5.95-5.92 (m, 1H), 4.71-4.64 (m, 1H), 4.31 (s, 2H), 3.8 (s,2H), 3.82-3.7 (bs, 1H), 2.31 (s, 3H), 1.94-1.88 (m, 2H), 1.76-1.66 (m, 2H), 1.65-1.52 (m, 10H) ppm | 5- methylfuran- 2- carbaldehyde | General method A using cyclopentanamine, AcOH and NaBH₄, method D1 using 1- bromo-4- isocyanatobenzene, method E using 2- methyl-2-[4-(4,4,5,5- tetramethyl-1,3,2- dioxaborolan-2- yl)pyrazole-1-yl]propan- 1-ol, Pd(PPh₃)₄ and K₂CO₃ | 1-cyclopentyl-3- [4-[1-(2- hydroxy-1,1- dimethyl- ethyl)pyrazol-4- yl]phenyl]-1-[(5- methyl-2- furyl)methyl]urea |
| 102 | 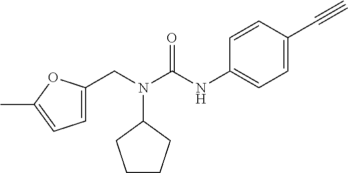 [SMILES: C#Cc1ccc(NC(=O)N(Cc2ccc(C)o2)C2CCCC2)cc1] | 323.2 | | 1H NMR (400 MHz, CDCl3): δ = 7.33 (d, J = 8.7 Hz, 2H), 7.23 (d, J = 8.7 Hz, 2H), 7.00 (bs, 1H), 6.11 (d, J = 2.6 Hz, 1H), 5.90 (dd, J = 2.6, 1.2 Hz, 1H), 4.64-4.56 (m, 1H), 4.25 (s, 2H), 2.94 (s, 1H), 2.26 (s, 3H), 1.89-1.83 (m, 2H), 1.66- 1.61 (m, 2H), 1.59-1.46 (m, 4H) ppm | 5- methylfuran- 2- carbaldehyde | General method A using cyclopentanamine, AcOH and NaBH₄, method D2 using 4- ethynylaniline | 1-cyclopentyl-3- (4- ethynylphenyl)- 1-[(5-methyl- furyl)methyl]urea |
| 103 | 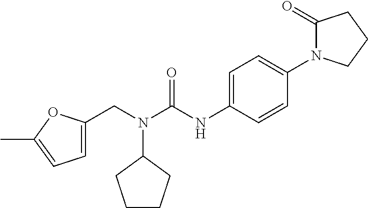 [SMILES: Cc1ccc(CN(C(=O)Nc2ccc(N3CCCC3=O)cc2)C2CCCC2)o1] | 382.2 | | 1H NMR (400 MHz, CDCl3): δ = 7.42 (d, J = 8.5 Hz, 2H), 7.19 (d, J = 8.5 Hz, 2H), 6.88 (bs, 1H), 6.10 (d, J = 2.6 Hz, 1H), 5.86 (dd, J = 2.6, 1.2 Hz, 1H), 4.62-4.54 (m, 1H), 4.27 (s, 2H), 3.74 (t, J = 7.6 Hz, 2H), 2.50 (t, J = 7.6 Hz, 2H), 2.22 (s, 3H), 2.09-2.01 (m, 2H), 1.86- 1.80 (m, 2H), 1.63-1.59 (m, 2H), 1.55-1.43 (m, 4H) ppm | 5- methylfuran- 2- carbaldehyde | General method A using cyclopentanamine, AcOH and NaBH₄, method S using 4- iodoaniline and 2- pyrrolidinone; method D2 | 1-cyclopentyl-1- [(5-methyl-2- furyl)methyl]-3- [4-(2- oxopyrrolidin-1- yl)phenyl]urea |
| 104 | 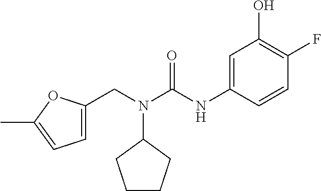 [SMILES: Cc1ccc(CN(C(=O)Nc2ccc(F)c(O)c2)C2CCCC2)o1] | 333.7 | | 1H NMR (400 MHz, CDCl3): δ = 7.27-7.23 (m, 1H), 6.91-6.86 (m, 2H), 6.75-6.60 (m, 1H), 6.91- 6.86 (m, 1H), 6.27 (d, J = 3.2 Hz, 1H), 5.96-5.93 (m, 1H), 4.69- 4.60 (m, 1H), 4.29 (s, 2H), 2.30 (s, 3H), 1.96-1.86 (m, 2H), 1.75- 1,64 (m, 2H), 1.63-1.47 (m, 4H) ppm | 5- methylfuran- 2- carbaldehyde | General method A using cyclopentanamine, AcOH and NaBH₄; method M using tert- butyl-chloro-dimethyl silane; method D2, method N using TBAFxH₂O | 1-cyclopentyl-3- (4-fluoro-3- hydroxy- phenyl)-1-[(5- methyl-2- furyl)methyl]urea |
| 105 | 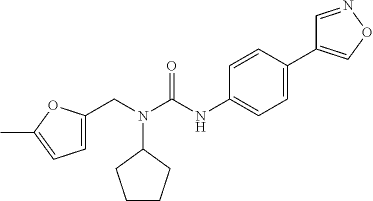 [SMILES: Cc1ccc(CN(C(=O)Nc2ccc(-c3cnoc3)cc2)C2CCCC2)o1] | 366.8 | | 1H NMR (500 MHz, CDCl3): δ = 8.50 (s, 1H), 8.40 (s, 1H), 7.43- 7.36 (m, 4H), 6.99 (s, 1H), 6.09 (d, J = 2.0 Hz, 1H), 5.86 (d, J = 2.0 Hz, 1H), 4.61-4.54 (m, 1H) 4.23 (s, 2H), 2.22 (s, 3H), 1.90-1.78 (m, 2H), 1.68-1.56 (m, 2H), 1.55- 1.43 (m, 4H) ppm | 5- methylfuran- 2- carbaldehyde | General method A using cyclopentanamine, AcOH and NaBH₄, method D1 using 1- bromo-4- isocyanatobenzene, method E using 4- (4,4,5,5-tetramethyl- 1,3,2-dioxaborolan-2- yl)isoxazole, Pd(PPh₃)₄ and K₂CO₃ | 1-cyclopentyl-3- (4-isoxazol-4- ylphenyl)-1-[(5- methyl-2- furyl)methyl]urea |
| 106 | 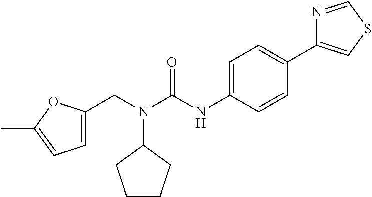 [SMILES: Cc1ccc(CN(C(=O)Nc2ccc(-c3cscn3)cc2)C2CCCC2)o1] | 382.7 | | 1H NMR (500 MHz, CDCl3): δ = 8.71 (s, 1H), 8.00 (s, 1H), 7.48 (d, J = 8.2 Hz, 2H), 7.38 (d, J = 8.2 Hz, 2H), 7.10 (s, 1H), 6.23 (d, J = 2.0 Hz, 1H), 5.97 (d, J = 2.0 Hz, 1H), 4.73-4.66 (m, 1H), 4.34 (s, 2H), 2.34 (s, 3H), 1.97-1.89 (m, 2H), 1.79-1.68 (m, 2H), 1.67-1.55 (m, 4H) ppm | 5- methylfuran- 2- carbaldehyde | General method A using cyclopentanamine, AcOH and NaBH₄, method D1 using 1- bromo-4- isocyanatobenzene, method E using 4- (4,4,5,5-tetramethyl- 1,3,2-dioxaborolan-2- yl)thiazole, Pd(PPh₃)₄ and K₂CO₃ | 1-cyclopentyl-1- [(5-methyl-2- furyl)methyl]-3- (4-thiazol-4- ylphenyl)urea |
| 107 | 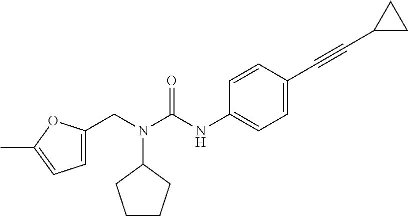 [SMILES: Cc1ccc(CN(C(=O)Nc2ccc(C#CC3CC3)cc2)C2CCCC2)o1] | 363.8 | | 1H NMR (500 MHz, CDCl3): δ = 7.28 (d, J = 7.2 Hz, 2H), 7.24 (d, J = 7.2 Hz, 2H), 6.99 (s, 1H), 6.18 (d, J = 3.0 Hz, 1H), 5.96 (d, J = 3.0 Hz, 1H), 4.70-4.63 (m, 1H), 4.31 (s, 2H), 2.32 (s, 3H), 1.97-1.90 (m, 2H), 1.74-1.67 (m, 2H), 1.63- 1.53 (m, 4H), 1.46-1.41 (m, 1H), 0.89-0.82 (m, 2H), 0.80-0.77 (m, 2H) ppm | 5- methylfuran- 2- carbaldehyde | General method A using cyclopentanamine, AcOH and NaBH₄, method D1 using 1- iodo-4- isocyanatobenzene, method F using 4- ethynylcyclopropane, 1,3,2-dioxaborolan-2- Pd(PPh₃)₂Cl₂, CuI and TEA | 1-cyclopentyl-3- [4-(2- cyclopropyl- ethynyl)phenyl]-1- [(5-methyl-2- furyl)methyl]urea |
| 108 | 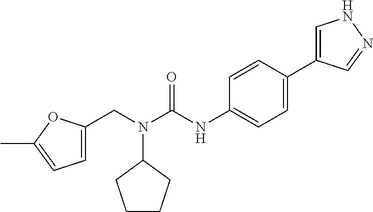 [SMILES: Cc1ccc(CN(C(=O)Nc2ccc(-c3cn[nH]c3)cc2)C2CCCC2)o1] | 365.8 | | 1H NMR (400 MHz, DMSO-d6): δ = 12.85-12.72 (bs, 1H), 8.23 (s, 1H), 8.02-7.88 (bs, 2H), 7.46- 7.40 (m, 4H), 6.12-6.08 (d, J = 3.6 Hz, 1H), 5.97-5.95 (m, 1H), 4.48-4.40 (m, 3H), 2.21 (s, 3H), 1.82-1.72 (m, 2H), 1.68-1.60 (m, 2H), 1.59-1.44 (m, 4H) ppm | 5- methylfuran- 2- carbaldehyde | General method A using cyclopentanamine, AcOH and NaBH₄, method D1 using 1- bromo-4- isocyanatobenzene, method E using 4- (4,4,5,5-tetramethyl- 1,3,2-dioxaborolan-2- yl)-1H-pyrazole, Pd(PPh₃)₄ and K₂CO₃ | 1-cyclopentyl-1- [(5-methyl-2- furyl)methyl]-3- [4-(1H-pyrazol- 4-yl)phenyl]urea |
| 109 | 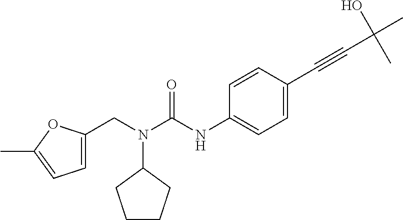 [SMILES: Cc1ccc(CN(C(=O)Nc2ccc(C#CC(C)(C)O)cc2)C2CCCC2)o1] | 381.8 | | 1H NMR (500 MHz, CDCl3): δ = 7.32 (d, J = 7.4 Hz, 2H), 7.29 (d, J = 7.4 Hz, 2H), 7.04 (s, 1H), 6.18 (d, J = 3.0 Hz, 1H), 5.97 (d, J = 3.0 Hz, 1H), 4.70-4.63 (m, 1H), 4.32 (s, 2H), 2.33 (s, 3H), 2.11-1.88 (m, 3H), 1.74-1.67 (m, 2H), 1.66- 1.51 (m, 10H) ppm | 5- methylfuran- 2- carbaldehyde | General method A using cyclopentanamine, AcOH and NaBH₄, method D1 using 1- iodo-4- isocyanatobenzene, method F using 2- methylbut-3-yn-2-ol, Pd(PPh₃)₂Cl₂, CuI and TEA | l-cyclopentyl-3- [4-(3-hydroxy-3- methyl-but-1- ynyl)phenyl]-1- [(5-methyl-2- furyl)methyl]urea |
| 110 | 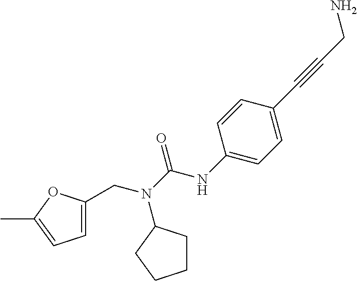 [SMILES: Cc1ccc(CN(C(=O)Nc2ccc(C#CCN)cc2)C2CCCC2)o1] | 352.7 | | 1H NMR (500 MHz, DMSO-d6): δ = 8.40 (s, 1H), 7.44 (d, J = 8.5 Hz, 2H), 7.24 (d, J = 8.5 Hz, 2H), 6.11 (d, J = 2.9 Hz, 1H), 5.96 (d, J = 2.9 Hz, 1H), 4.49-4.38 (m, 3H), 3.55-3.42 (bs, 2H), 2.20 (s, 3H), 1.82-1.70 (m, 2H), 1.69-1.60 (m, 2H), 1.58-1.44 (m, 4H) ppm | 5- methylfuran- 2- carbaldehyde | General method A using cyclopentanamine, AcOH and NaBH₄, method D1 using 1- iodo-4- isocyanatobenzene, method F using prop- 2-yn-1-amine, Pd(PPh₃)₂Cl₂, CuI and TEA | 3-[4-(3- aminoprop-1- ynyl)phenyl]-1- cyclopentyl-1- [(5-methyl-2- furyl)methyl]urea |
| 111 | 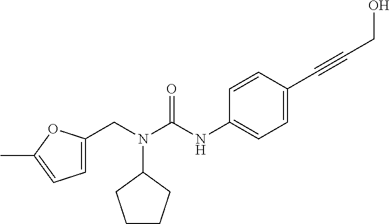 [SMILES: Cc1ccc(CN(C(=O)Nc2ccc(C#CCO)cc2)C2CCCC2)o1] | 353.7 | | 1H NMR (500 MHz, DMSO-d6): δ = 8.43 (s, 1H), 7.46 (d, J = 8.5 Hz, 2H), 7.28 (d, J = 8.5 Hz, 2H), 6.11 (d, J = 2.0 Hz, 1H), 5.96 (d, J = 2.0 Hz, 1H), 5.26 (t, J = 5.7 Hz, 1H), 4.48-4.39 (m, 3H), 4.27 (d, J = 5.7 Hz, 2H), 2.21 (s, 3H), 1.83- 1.71 (m, 2H), 1.70-1.61 (m, 2H), 1.59-1.44 (m, 4H) ppm | 5- methylfuran- 2- carbaldehyde | General method A using cyclopentanamine, AcOH and NaBH₄, method D1 using 1- iodo-4- isocyanatobenzene, method F using prop- 2-yn-1-ol, Pd(PPh₃)₂Cl₂, CuI and TEA | 1-cyclopentyl-3- [4-(3- hydroxyprop-1- ynyl)phenyl]-1- [(5-methyl-2- furyl)methyl]urea |
| 112 | 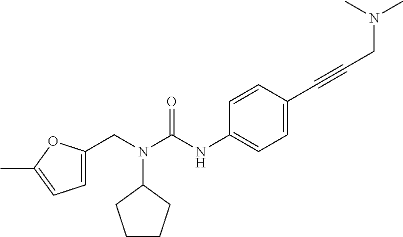 | 380.8 | | 1H NMR (500 MHz, DMSO-d6): δ = 8.42 (s, 1H), 7.46 (d, J = 8.5 Hz, 2H), 7.26 (d, J = 8.5 Hz, 2H), 6.08 (d, J = 2.0 Hz, 1H), 5.96 (d, J = 2.0 Hz, 1H), 4.48-4.40 (m, 3H), 3.42 (s, 2H), 2.24 (s, 6H), 2.21 (s, 3H), 1.84-1.70 (m, 2H), 1.69- 1.61 (m, 2H), 1.59-1.46 (m, 4H) ppm | 5- methylfuran- 2- carbaldehyde | General method A using cyclopentanamine, AcOH and NaBH₄, method D1 using 1- iodo-4- isocyanatobenzene, method F using N,N- dimethylprop-2-yn-1- amine, Pd(PPh₃)₂Cl₂, CuI and TEA | 1-cyclopentyl-3- [4-[3- (dimethylamino) prop-1- ynyl]phenyl]-1- [(5-methyl-2- furyl)methyl]urea |
| 113 | 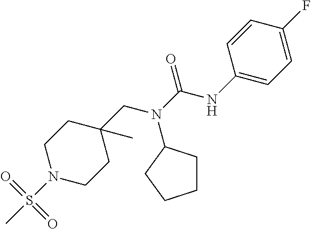 [SMILES: CC1(CN(C(=O)Nc2ccc(F)cc2)C2CCCC2)CCN(S(C)(=O)=O)CC1] | 412.2 | | 1H NMR (300 MHz, DMSO-d6): δ = 8.31 (s, 1H), 7.46-7.33 (m, 2H), 7.04 (t, J = 8.9 Hz, 2H), 3.92- 3.77 (m, 1H), 3.21 (s, 2H), 2.99- 2.79 (m, 5H), 1.85-1.62 (m, 7H), 1.61-1.31 (m, 7H), 0.94 (s, 3H) ppm | tert-butyl 4- formyl-4- methyl- piperidine-1- carboxylate | General method A using cyclopentanamine and NaBH₄, method D1 using 4- fluorophenylisocyanate, method F using TFA, method H using MsCl | 1-cyclopentyl- (4- fluorophenyl)-1- [(4-methyl-1- methylsulfonyl- 4- piperidyl)methyl] urea |
| 114 | 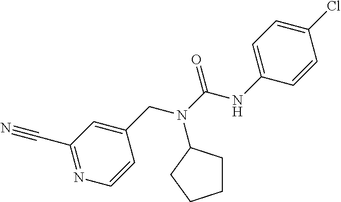 [SMILES: N#Cc1cc(CN(C(=O)Nc2ccc(Cl)cc2)C2CCCC2)ccn1] | 355.1 | | 1H NMR (300 MHz, DMSO-d6): δ = 8.66 (d, J = 5.8 Hz, 1H), 8.59 (s, 1H), 7.86 (s, 1H), 7.55 (dd, J = 5.1, 1.4 Hz, 1H), 7.52-7.44 (m, 2H), 7.32-7.22 (m, 2H), 4.61-4.44 (m, 3H), 1.92-1.71 (m, 2H), 1.69- 1.44 (m, 4H), 1.43-1.24 (m, 2H) ppm | 4- formyl- pyridine-2- carbonitrile | General method A using cyclopentanamine and NaBH₄, method D1 using 4- chlorophenylisocyanate | 3-(4- chlorophenyl)-1- [(2-cyano-4- pyridyl)methyl]- 1-cyclopentyl- urea |
| 115 | 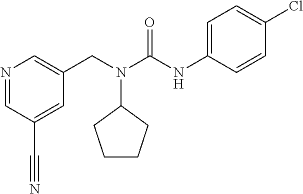 [SMILES: N#Cc1cncc(CN(C(=O)Nc2ccc(Cl)cc2)C2CCCC2)c1] | 355.1 | | 1H NMR (300 MHz, DMSO-d6): δ = 8.87 (d, J = 1.9 Hz, 1H), 8.73 (d, J = 2.0 Hz, 1H), 8.61 (s, 1H), 8.10-8.06 (m, 1H), 7.48 (d, J = 8.9 Hz, 2H), 7.26 (d, J = 8.9 Hz, 2H), 4.56 (s, 2H), 4.53-4.42 (m, 1H), 1.89-1.73 (m, 2H), 1.72-1.57 (m, 2H), 1.56-1.31 (m, 4H) ppm | 5- formyl- pyridine-2- carbonitrile | General method A using cyclopentanamine and NaBH₄, method D1 using 4- chlorophenylisocyanate | 3-(4- chlorophenyl)-1- [(5-cyano-3- pyridyl)methyl]- 1-cyclopentyl- urea |
| 116 | 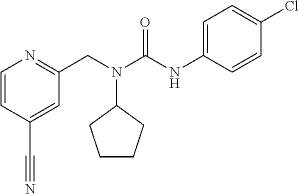 [SMILES: N#Cc1ccnc(CN(C(=O)Nc2ccc(Cl)cc2)C2CCCC2)c1] | 355.1 | | 1H NMR (300 MHz, DMSO-d6): δ = 8.80-8.74 (m, 1H), 8.69 (s, 1H), 7.69 (s, 2H), 7.75-766 (m, 2H), 7.48 (d, J = 8.9 Hz, 2H), 7.26 (d, J = 8.9 Hz, 2H), 4.59 (s, 2H), 4.57-4.43 (m, 1H), 1.89-1.70 (m, 2H), 1.69-1.56 (m, 2H), 1.55-1.32 (m, 4H) ppm | 2-- formyl- pyridine-4- carbonitrile | General method A using cyclopentanamine and NaBH₄, method D1 using 4- chlorophenylisocyanate | 3-(4- chlorophenyl)-1- [(4-cyano-2- pyridyl)methyl]- 1-cyclopentyl- urea |
| 117 | 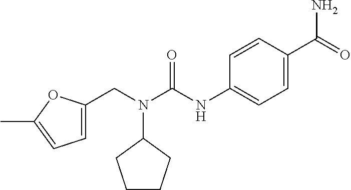 [SMILES: Cc1ccc(CN(C(=O)Nc2ccc(C(N)=O)cc2)C2CCCC2)o1] | 342.2 | | 1H NMR (300 MHz, DMSO-d6): δ = 8.49 (s, 1H), 7.74 (d, J = 8.8 Hz, 3H), 7.50 (d, J = 8.8 Hz, 2H), 7.17-7.07 (bs, 1H), 6.09 (d, J = 3.0 Hz, 1H), 5.98- 5.92 (m, 1H), 4.52- 4.36 (m, 3H), 2.20 (s, 3H), 1.86- 1.71 (m, 2H), 1.70-1.41 (m, 6H) ppm | 5- methylfuran- 2- carbaldehyde | General method A using cyclopentanamine, AcOH and NaBH₄, method D1 using 4- isocyanatobenzonitrile, method P using H₂O₂ and K₂CO₃ | 4-[[cyclopentyl- [(5-methyl-2- furyl)methyl]car- bamoyl]amino- benzamide |
| 118 | 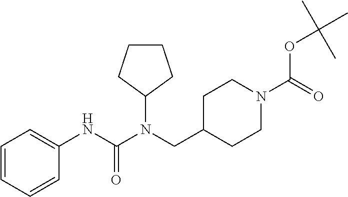 [SMILES: CC(C)(C)OC(=O)N1CCC(CN(C(=O)Nc2ccccc2)C2CCCC2)CC1] | 402.3 | | 1H NMR (300 MHz, DMSO-d6): δ = 8.18 (s, 1H), 7.42 (dd, J = 8.7, 1.2 Hz, 2H), 7.20 (t, J = 7.9 Hz, 2H), 6.96-6.87 (m, 1H), 4.26-4.10 (m, 1H), 4.00-3.85 (m, 2H), 3.11 (d, J = 7.1 Hz, 2H), 2.69-2.54 (m, 2H), 1.86-1.42 (m, 11H), 1.37 (s, 9H), 1.10-0.92 (m, 2H) ppm | tert-butyl 4- formyl- piperidine-1- carboxylate | General method A using cyclopentanamine and NaBH₄, method D1 using phenyl isocyanate | tert-butyl 4- [[cyclopentyl (phenylcarbamoyl) amino]methyl] piperidine-1- carboxylate |
| 119 | 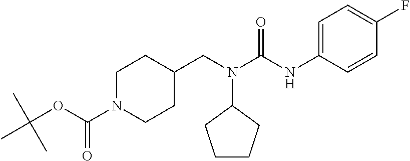 [SMILES: CC(C)(C)OC(=O)N1CCC(CN(C(=O)Nc2ccc(F)cc2)C2CCCC2)CC1] | 420.3 | | 1H NMR (300 MHz, DMSO-d6): δ = 8.22 (s, 1H), 7.47-7.35 (m, 2H), 7.10-6.98 (m, 2H), 4.22-4.06 (m, 1H), 4.00-3.86 (m, 2H), 3.14- 3.08 (m, 2H), 2.70-2.52 (m, 2H), 1.85-1.42 (m, 11H), 1.37 (s, 9H), 1.10-0.91 (m, 2H) ppm | tert-butyl 4- formyl- piperidine-1- carboxylate | General method A using cyclopentanamine and NaBH₄, method D1 using 4- fluorophenylisocyanate | tert-butyl 4- [[cyclopentyl- [(4- fluorophenyl) carbamoyl]amino] methyl]piperine- 1-carboxylate |
| 120 | 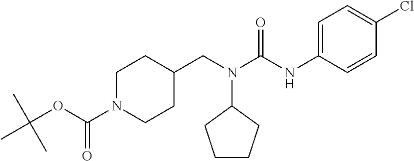 [SMILES: CC(C)(C)OC(=O)N1CCC(CN(C(=O)Nc2ccc(Cl)cc2)C2CCCC2)CC1] | 436.2 | | 1H NMR (300 MHz, DMSO-d6): δ = 8.35 (s, 1H), 7.44 (d, J = 8.9 Hz, 2H), 7.26 (d, J = 8.9 Hz, 2H), 4.12-4.07 (m, 1H), 3.99-3.83 (m, 2H), 3.16-3.06 (m, 2H), 2.70-2.53 (m, 2H), 1.84-1.41 (m, 11H), 1.37 (s, 9H), 1.09-0.92 (m, 2H) ppm | tert-butyl 4- formyl- piperidine-1- carboxylate | General method A using cyclopentanamine and NaBH₄, method D1 using 4- chlorophenylisocyanate | tert-butyl 4-[[(4- chlorophenyl) carbamoyl- cyclopentyl- amino]methyl] piperidine-1- carboxylate |
| 121 | 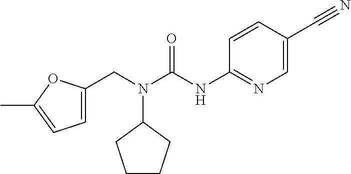 [SMILES: Cc1ccc(CN(C(=O)Nc2ccc(C#N)cn2)C2CCCC2)o1] | 325.2 | | 1H NMR (300 MHz, DMSO-d6): δ = 9.38 (s, 1H), 8.67 (dd, J = 2.4, 0.8 Hz, 1H), 8.12-8.06 (m, 1H), 7.93-7.88 (m, 1H), 6.11 (d, J = 3.1 Hz, 1H), 5.98-5.94 (m, 1H), 4.56- 4.38 (m, 3H), 2.18 (s, 3H), 1.84- 1.69 (m, 2H), 1.68-1.40 (m, 6H) ppm | 5- methylfuran- 2- carbaldehyde | General method A using cyclopentanamine, AcOH and NaBH₄, method D2 using 6- aminopyridine-3- carbonitrile | 3-(5-cyano-2- pyridyl)-1- cyclopentyl-1- [(5-methyl-2- furyl)methyl]urea |
| 122 | 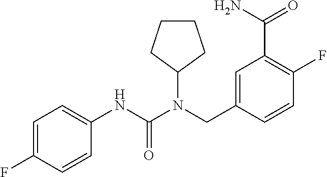 [SMILES: NC(=O)c1cc(CN(C(=O)Nc2ccc(F)cc2)C2CCCC2)ccc1F] | 374.7 | | 1H NMR (300 MHz, DMSO-d6): δ = 8.39 (s, 1H), 7.62 (bs, 2H), 7.58-7.49 (m, 1H), 7.47-7.38 (m, 2H), 7.37-7.29 (m, 1H), 7.26-7.15 (m, 1H), 7.04 (t, J = 9.0 Hz, 2H), 4.63-4.32 (m, 3H), 1.82-1.67 (m, 2H), 1.66-1.33 (m, 6H) ppm | 2-fluoro-5- formyl- benzonitrile | General method A using cyclopentanamine, AcOH and NaBH₄, method D1 using 4- fluorophenylisocyanate, method P using H₂O₂ and K₂CO₃ | 5-[[cyclopentyl- [(4- fluorophenyl) carbamoyl]amino] methyl]-2- fluoro- benzamide |
| 123 | 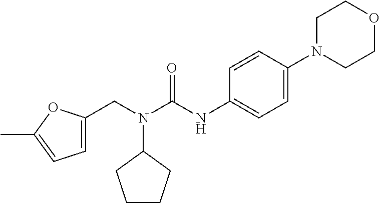 [SMILES: Cc1ccc(CN(C(=O)Nc2ccc(N3CCOCC3)cc2)C2CCCC2)o1] | 384.8 | | 1H NMR (300 MHz, DMSO-d6): δ = 8.03 (s, 1H), 7.26 (d, J = 8.9 Hz, 2H), 6.82 (d, J = 8.9 Hz, 2H), 6.07 (d, J = 3.3 Hz, 1H), 5.97-5.93 (m, 1H), 4.45-4.32 (m, 3H), 3.77- 3.63 (m, 4H), 3.03-2.92 (m, 4H), 2.20 (s, 3H), 1.83-1.67 (m, 2H), 1.66-1.40 (m, 6H) ppm | 5- methylfuran- 2- carbaldehyde | General method A using cyclopentanamine, AcOH and NaBH₄, method D4 using 4- morpholinoaniline | 1-cyclopentyl-1- [(5-methyl-2- furyl)methyl]-3- (4- morpholino- phenyl)urea |
| 124 | 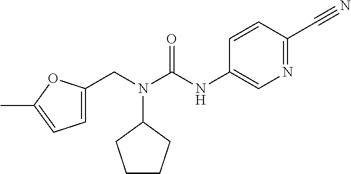 [SMILES: Cc1ccc(CN(C(=O)Nc2ccc(C#N)nc2)C2CCCC2)o1] | 325.7 | | 1H NMR (300 MHz, DMSO-d6): δ = 9.03 (s, 1H), 8.79 (d, J = 2.4 Hz, 1H), 8.11 (dd, J = 8.6, 2.4 Hz, 1H), 7.86 (d, J = 8.6 Hz, 1H), 6.10 (d, J = 3.3 Hz, 1H), 5.96-5.93 (m, 1H), 4.53-4.37 (m, 3H), 2.19 (s, 3H), 1.84-1.71 (m, 2H), 1.70-1.43 (m, 6H) ppm | 5- methylfuran- 2- carbaldehyde | General method A using cyclopentanamine, AcOH and NaBH₄, method D4 using 5- aminopyridine-2- carbonitrile | 3-(6-cyano-3- pyridyl)-1- cyclopentyl- [(5-methyl-2- furyl)methyl]urea |
| 125 | 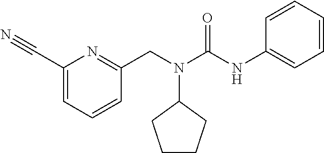 [SMILES: N#Cc1cccc(CN(C(=O)Nc2ccccc2)C2CCCC2)n1] | 321.7 | | 1H NMR (300 MHz, DMSO-d6): δ = 8.52 (s, 1H), 8.01 (t, J = 7.8 Hz, 1H), 7.91 (d, J = 7.6 Hz, 1H), 7.60 (d, J = 7.8 Hz, 1H), 7.41 (d, J = 7.8 Hz, 2H), 7.21 (t, J = 7.6 Hz, 2H), 6.92 (t, J = 7.6 Hz, 1H), 4.70-4.47 (m, 3H), 1.87-1.70 (m, 2H), 1.69- 1.33 (m, 6H) ppm | 6- formyl- pyridine-2- carbonitrile | General method A using cyclopentanamine and NaBH₄, method D1 using phenyl isocyanate | 1-[(6-cyano-2- pyridyl)methyl]- 1-cyclopentyl-3- phenyl-urea |
| 126 | 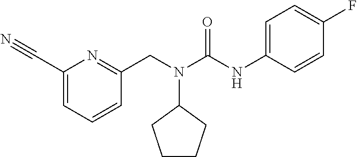 [SMILES: N#Cc1cccc(CN(C(=O)Nc2ccc(F)cc2)C2CCCC2)n1] | 339.7 | | 1H NMR (300 MHz, DMSO-d6): δ = 8.55 (s, 1H), 8.01 (t, J = 7.8 Hz, 1H), 7.91 (d, J = 6.5 Hz, 1H), 7.58 (d, J = 8.0 Hz, 1H), 7.42 (dd, J = 9.0, 5.1 Hz, 2H), 7.05 (t, J = 8.8 Hz, 2H), 4.59 (s, 2H), 4.65-4.44 (m, 3H), 1.85-1.70 (m, 2H), 1.69-1.56 (m, 2H), 1.55-1.31 (m, 4H) ppm | 6- formyl- pyridine-2- carbonitrile | General method A using cyclopentanamine and NaBH₄, method D1 using 4- fluorophenylisocyanate | 1-[(6-cyano-2- pyridyl)methyl]- 1-cyclopentyl-3- (4- fluorophenyl)urea |
| 127 | 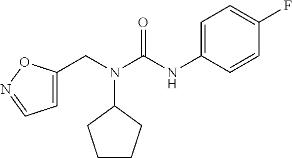 [SMILES: O=C(Nc1ccc(F)cc1)N(Cc1ccno1)C1CCCC1] | 304.7 | | 1H NMR (300 MHz, DMSO-d6): δ = 8.49 (s, 1H), 8.44 (d, J = 1.8 Hz, 1H), 7.48-7.37 (m, 2H), 7.10- 7.00 (m, 2H), 6.28-6.6.24 (m, 1H), 4.62 (s, 2H), 4.52-4.39 (m, 1H), 1.89-1.72 (m, 2H), 1.71-1.57 (m, 2H), 1.56-1.38 (m, 4H) ppm | isoxazole-5- carbaldehyde | General method A using cyclopentanamine and NaBH₄, method D1 using 4- fluorophenylisocyanate | 1-cyclopentyl-2- (4- fluorophenyl)-1- (isoxazol-5- ylmethyl)urea |
| 128 | 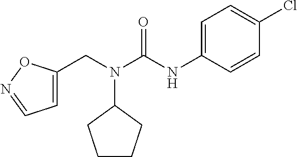 [SMILES: O=C(Nc1ccc(Cl)cc1)N(Cc1ccno1)C1CCCC1] | 320.7 | | 1H NMR (300 MHz, DMSO-d6): δ = 8.58 (s, 1H), 8.44 (d, J = 1.6 Hz, 1H), 7.47 (d, J = 9.0 Hz, 2H), 7.26 (d, J = 9.0 Hz, 2H), 6.27 (d, J = 1.6 Hz, 1H), 4.63 (s, 2H), 4.53- 4.39 (m, 1H), 1.88-1.72 (m, 2H), 1.71-1.58 (m, 2H), 1.57-1.38 (m, 4H) ppm | isoxazole-5- carbaldehyde | General method A using cyclopentanamine and NaBH₄, method D1 using 4- chlorophenylisocyanate | 3-(4- chlorophenyl)-1- cyclopentyl-1- (isoxazol-5- ylmethyl)urea |
| 129 | 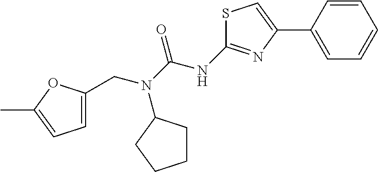 [SMILES: Cc1ccc(CN(C(=O)Nc2nc(-c3ccccc3)cs2)C2CCCC2)o1] | 382.7 | | 1H NMR (300 MHz, DMSO-d6): δ = 10.81 (s, 1H), 7.87 (d, J = 7.3 Hz, 2H), 7.45-7.35 (m, 3H), 7.32- 7.24 (m, 1H), 6.11-6.07 (m, 1H), 5.97-5.94 (m, 1H), 4.58-4.40 (m, 3H), 2.21 (s, 3H), 1.82-1.69 (m, 2H), 1.68-1.41 (m, 6H) ppm | 5- methylfuran- 2- carbaldehyde | General method A using cyclopentanamine, AcOH and NaBH₄, method D4 using 4- phenylthiazol-2 amine | 1-cyclopentyl-1- [(5-methyl-2- furyl)methyl]-3- (4- phenylthiazol-2- yl)urea |
| 130 | 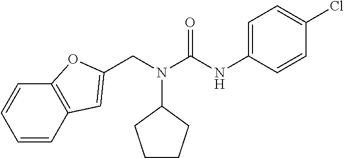 [SMILES: O=C(Nc1ccc(Cl)cc1)N(Cc1cc2ccccc2o1)C1CCCC1] | 369.7 | | 1H NMR (300 MHz, DMSO-d6): δ = 8.55 (s, 1H), 7.59-7.45 (m, 4H), 7.29-7.14 (m, 4H), 6.67 (s, 1H), 4.65 (s, 2H), 4.58-4.43 (m, 1H), 1.91-1.73 (m, 2H), 1.72-1.43 (m, 6H) ppm | benzofuran- 2- carbaldehyde | General method A using cyclopentanamine and NaBH₄, method D1 using 4- chlorophenylisocyanate | 1-(benzofuran-2- ylmethyl)-3-(4- chlorophenyl)-1- cyclopentyl-urea |
| 131 | 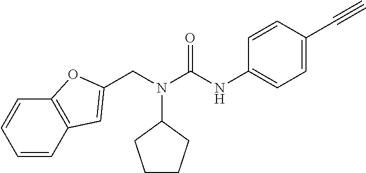 [SMILES: C#Cc1ccc(NC(=O)N(Cc2cc3ccccc3o2)C2CCCC2)cc1] | 359.7 | | 1H NMR (300 MHz, DMSO-d6): δ = 8.61 (s, 1H), 7.59-7.44 (m, 4H), 7.32 (d, J = 8.7 Hz, 2H), 7.27- 7.13 (m, 2H), 6.67 (s, 1H), 4.66 (s, 2H), 4.58-4.44 (m, 1H), 4.00 (s, 1H), 1.91-1.73 (m, 2H), 1.72- 1.42 (m, 6H) ppm | benzofuran- 2- carbaldehyde | General method A using cyclopentanamine and NaBH₄, method D4 using 4-ethynylaniline | 1-(benzofuran-2- ylmethyl)-1- cyclopentyl-3- (4- ethynylphenyl) urea |
| 132 | 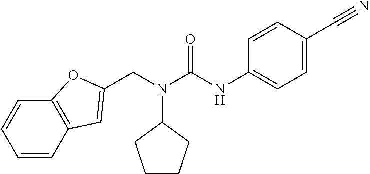 [SMILES: N#Cc1ccc(NC(=O)N(Cc2cc3ccccc3o2)C2CCCC2)cc1] | 359.7 | | 1H NMR (300 MHz, DMSO-d6): δ = 8.90 (m, 1H), 7.72-7.63 (m, 4H), 7.57-7.47 (m, 2H), 7.27-7.15 (m, 2H), 6.68 (m, 1H), 4.69 (s, 2H), 4.60-4.42 (m, 1H), 1.90-1.74 (m, 2H), 1.73-1.44 (m, 6H) ppm | benzofuran- 2- carbaldehyde | General method A using cyclopentanamine and NaBH₄, method D1 using 4- isocyanato- benzonitrile | 1-(benzofuran-2- ylmethyl)-3-(4- cyanophenyl)-1- cyclopentyl-urea |
| 133 | 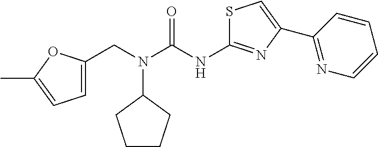 [SMILES: Cc1ccc(CN(C(=O)Nc2nc(-c3ccccn3)cs2)C2CCCC2)o1] | 383.7 | | 1H NMR. (300 MHz, DMSO-d6): δ = 10.87 (s, 1H), 8.57 (d, J = 4.70 Hz, 1H), 7.94 (d, J = 7.5 Hz, 1H), 7.85 (dd, J = 7.5, 1.7 Hz, 1H), 7.67 (s, 1H), 7.33-7.25 (m, 1H), 6.10 (d, J = 2.9 Hz, 1H), 5.99-5.93 (m, 1H), 4.63-4.37 (m, 3H), 2.21 (s, 3H), 1.88-1.69 (m, 2H), 1.68-1.42 (m, 6H) ppm | 5- methylfuran- 2- carbaldehyde | General method A using cyclopentanamine, AcOH and NaBH₄, method D3 using 4-(2- pyridyl)thiazol-2-amine | 1-cyclopentyl-1- [(5-methyl-2- furyl)methyl]- [4-(2- pyridyl)thiazol- 2-yl]urea |
| 134 | 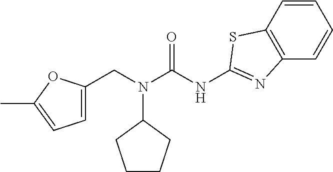 [SMILES: Cc1ccc(CN(C(=O)Nc2nc3ccccc3s2)C2CCCC2)o1] | 356.7 | | 1H NMR (300 MHz, DMSO-d6): δ = 12.15-12.73 (m, 1H), 10.70- 11.37 (m, 1H), 7.47-7.96 (m, 1H), 7.27-7.44 (m, 1H), 7.06-7.26 (m, 1H), 6.01-6.19 (m, 1H), 5.96 (br. s., 1H), 4.54 (m, 3H), 2.20 (s, 3H), 1.33-1.93 (m, 8H) ppm | 5- methylfuran- 2- carbaldehyde | General method A using cyclopentanamine, AcOH and NaBH₄, method D3 using 1,3- benzothiazol-2-amine | 3-(1,3- benzothiazol-2- yl)-1- cyclopentyl-1- [(5-methyl-2- furyl)methyl]urea |
| 135 | 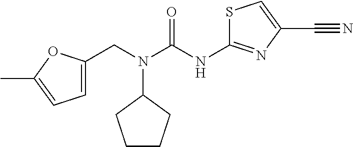 [SMILES: Cc1ccc(CN(C(=O)Nc2nc(C#N)cs2)C2CCCC2)o1] | 331.7 | | 1H NMR (300 MHz, DMSO-d6): δ = 11.28 (s, 1H), 8.19 (s, 1H), 6.09 (d, J = 2.9 Hz, 1H), 5.98-5.94 (m, 1H), 4.49 (s, 2H), 4.46-4.34 (m, 1H), 2.20 (s, 3H), 1.82-1.68 (m, 2H), 1.67-1.40 (m, 6H) ppm | 5- methylfuran- 2- carbaldehyde | General method A using cyclopentanamine, AcOH and NaBH₄, method D3 using 2- aminothiazole-4- carbonitrile | 3-(4- cyanothiazol-2- yl)-1- cyclopentyl-1- [(5-methyl-2- furyl)methyl]urea |
| 136 | 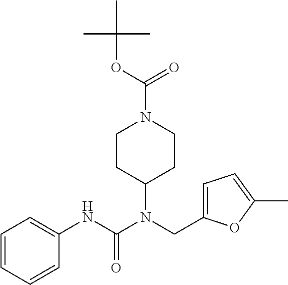 [SMILES: Cc1ccc(CN(C(=O)Nc2ccccc2)C2CCN(C(=O)OC(C)(C)C)CC2)o1] | 414.8 | | 1H NMR (300 MHz, DMSO-d6): δ = 8.27 (s, 1H), 7.47-7.39 (m, 2H), 7.28-7.17 (m, 2H), 6.98-6.90 (m, 1H), 6.13 (d, J = 2.95 Hz, 1H), 5.99-5.94 (m, 1H), 4.47 (bs, 2H), 4.23-4.10 (m, 1H), 4.00 (d, J = 13.18 Hz, 2H), 2.93-2.61 (m, 2H), 2.21 (d, J = 0.70 Hz, 3H), 1.64- 1.51 (m, 4H), 1.40 (s, 9H) ppm | 5- methylfuran- 2- carbaldehyde | General method A using tert-butyl 4- aminopiperidine-1- carboxylate, AcOH and NaBH₄, method D1 using phenyl isocyanate | tert-butyl 4-[(5- methyl-2- furyl)methyl- (phenylcarba- moyl)amino] piperdine-1- carboxylate |
| 137 | 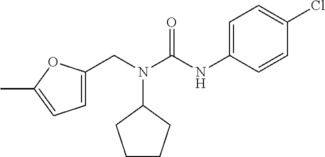 [SMILES: Cc1ccc(CN(C(=O)Nc2ccc(Cl)cc2)C2CCCC2)o1] | 333.7 | | 1H NMR (400 MHz, DMSO-d6): δ = 8.39 (s, 1H), 7.47 (d, J = 9.0 Hz, 2H), 7.25 (d, J = 9.0 Hz, 2H), 6.08 (d, J = 3.0 Hz, 1H), 5.96-5.94 (m, 1H), 4.42 (s, 2H), 4.44-4.36 (m, 1H), 2.19 (s, 3H), 1.79-1.69 (m, 2H), 1.67-1.60 (m, 2H), 1.59- 1.43 (m, 4H) ppm | 5- methylfuran- 2- carbaldehyde | General method A using cyclopentanamine, AcOH and NaBH₄, method D1 using 4- chlorophenylisocyanate | 3-(4- chlorophenyl)-1- cyclopentyl-1- [(5-methyl-2- furyl)methyl]urea |
| 138 | 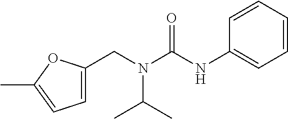 [SMILES: Cc1ccc(CN(C(=O)Nc2ccccc2)C(C)C)o1] | 273.7 | | 1H NMR (400 MHz, DMSO-d6): δ = 8.18 (s, 1H), 7.46-7.41 (m, 2H), 7.23-7.17 (m, 2H), 6.93-6.89 (m, 1H), 6.12 (d, J = 3.0 Hz, 1H), 5.97-5.94 (m, 1H), 4.43 (s, 2H), 4.46-4.34 (m, 1H), 2.20 (s, 3H), 1.10 (d, J = 6.8 Hz, 6H) ppm | 5- methylfuran- 2- carbaldehyde | General method A using propan-2-amine, AcOH and NaBH₄, method D1 using phenyl isocyanate | 1-isopropyl-1- [(5-methyl-2- furyl)methyl]-3- phenyl-urea |
| 139 | 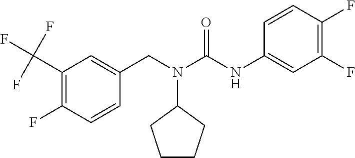 [SMILES: O=C(Nc1ccc(F)c(F)c1)N(Cc1ccc(F)c(C(F)(F)F)c1)C1CCCC1] | 417.7 | | 1H NMR (400 MHz, DMSO-d6): δ = 8.61 (s, 1H), 7.62-7.54 (m, 3H), 7.49-7.42 (m, 1H), 7.32-7.22 (m, 1H), 7.22-7.18 (m, 1H), 4.55 (s, 2H), 4.50-4.44 (m, 1H), 1.80- 1.70 (m, 2H), 1.68-1.58 (m, 2H), 1.54-1.36 (m, 4H) ppm | 4-fluoro-3- trifluoro- methyl- benzaldehyde | General method A using cyclopentanamine, AcOH and NaBH₄, method D1 using 1,2- difluoro-4- isocyanatobenzene | 1-cyclopentyl-3- (3,4- difluorophenyl)- 1-[[4-fluoro- (trifluoromethyl) phenyl]methyl] urea |
| 140 | 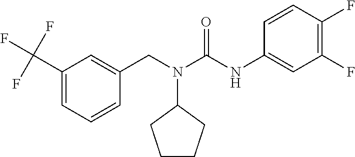 [SMILES: O=C(Nc1ccc(F)c(F)c1)N(Cc1cccc(C(F)(F)F)c1)C1CCCC1] | 399.7 | | 1H NMR (400 MHz, DMSO- d6):δ = 8.60 (s, 1H), 7.62-7.49 (m, 5H), 7.32-7.22 (m, 1H), 7.22- 7.18 (m, 1H), 4.60 (s, 2H), 4.54- 4.46 (m, 1H), 1.80-1.70 (m, 2H), 1.68-1.58 (m, 2H), 1.54-1.36 (m, 4H) ppm | 3- trifluoro- methyl- benzaldehyde | General method A using cyclopentanamine, AcOH and NaBH₄, method D1 using 1,2- difluoro-4- isocyanatobenzene | 1-cyclopentyl-3- (3,4- difluorophenyl)- 1-[[3- (trifluoromethyl) phenyl]methyl] urea |
| 141 | 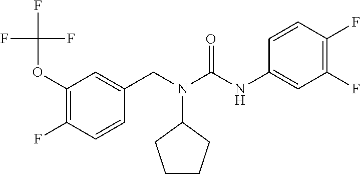 [SMILES: O=C(Nc1ccc(F)c(F)c1)N(Cc1ccc(F)c(OC(F)(F)F)c1)C1CCCC1] | 433.6 | | 1H NMR (400 MHz, DMSO- d6):δ = 8.60 (s, 1H), 7.63-7.55 (m, 1H), 7.48-7.41 (m, 1H), 7.38- 7.33 (m, 1H), 7.33-7.23 (m, 2H), 7.23-7.17 (m, 1H), 4.52 (s, 2H), 4.50-4.43 (m, 1H), 1.80-1.70 (m, 2H), 1.68-1.56 (m, 2H), 1.54-1.36 (m, 4H) ppm | 4-fluoro-3- trifluoro- methyl- benzaldehyde | General method A using cyclopentanamine, AcOH and NaBH₄, method D1 using 1,2- difluoro-4- isocyanatobenzene | 1-cyclopentyl-3- (3,4- difluorophenyl)- 1-[[4-fluoro-3- (trifluoromethoxy) phenyl]methyl] urea |
| 142 | 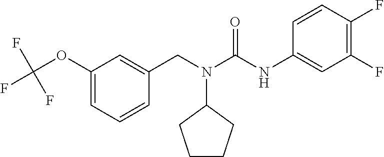 [SMILES: O=C(Nc1ccc(F)c(F)c1)N(Cc1cccc(OC(F)(F)F)c1)C1CCCC1] | 415.7 | | 1H NMR (400 MHz, DMSO-d6): δ = 8.59 (s, 1H), 7.62-7.55 (m, 1H), 7.47-7.42 (m, 1H), 7.31-7.23 (m, 2H), 7.22-7.15 (m, 3H), 4.56 (s, 2H), 4.53-4.46 (m, 1H), 1.80- 1.70 (m, 2H), 1.68-1.56 (m, 2H), 1.54-1.36 (m, 4H) ppm | 3- trifluoro- methoxy- benzaldehyde | General method A using cyclopentanamine, AcOH and NaBH₄, method D1 using 1,2- difluoro-4- isocyanatobenzene | 1-cyclopentyl-3- (3,4- difluorophenyl)- 1-[[3- (trifluoromethoxy) phenyl]methyl] urea |
| 143 | 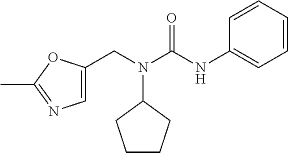 [SMILES: Cc1ncc(CN(C(=O)Nc2ccccc2)C2CCCC2)o1] | 300.6 | | 1H NMR (400 MHz, DMSO-d6): δ = 8.32 (s, 1H), 7.45-7.41 (m, 2H), 7.24-7.18 (m, 2H), 6.95-6.90 (m, 1H), 6.79 (s, 1H), 4.48 (s, 2H), 4.46-4.38 (m, 1H), 2.34 (s, 3H), 1.84-1.74 (m, 2H), 1.70-1.60 (m, 2H), 1.58-1.46 (m, 4H) ppm | 2-methyl- oxazole-5- carbaldehyde | General method A using cyclopentanamine, AcOH and NaBH₄, method D1 using phenyl isocyanate | 1-cyclopentyl-1- [(2- methyloxazol-5- yl)methyl]-3- phenyl-urea |
| 144 | 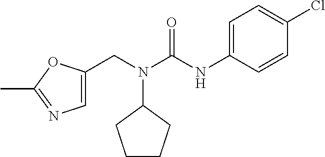 [SMILES: Cc1ncc(CN(C(=O)Nc2ccc(Cl)cc2)C2CCCC2)o1] | 334.6 | | 1H NMR (400 MHz, DMSO-d6): δ = 8.47 (s, 1H), 7.47 (d, J = 8.9 Hz, 2H), 7.26 (d, J = 8.9 Hz, 2H), 6.79 (s, 1H), 4.48 (s, 2H), 4.46- 4.34 (m, 1H), 2.34 (s, 3H), 1.84- 1.74 (m, 2H), 1.70-1.60 (m, 2H), 1.58-1.46 (m, 4H) ppm | 2-methyl- oxazole-5- carbaldehyde | General method A using cyclopentanamine, AcOH and NaBH₄, method D1 using 4- chlorophenylisocyanate | 3-(4- chlorophenyl)-1- cyclopentyl-1- [(2- methyloxazol-5 yl)methyl]urea |
| 145 | 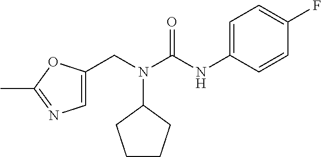 [SMILES: Cc1ncc(CN(C(=O)Nc2ccc(F)cc2)C2CCCC2)o1] | 318.6 | | 1H NMR (400 MHz, DMSO-d6): δ = 8.37 (s, 1H), 7.46-7.40 (m, 2H), 7.08-7.02 (m, 2H), 6.79 (s, 1H), 4.47 (s, 2H), 4.44-4.36 (m, 1H), 2.34 (s, 3H), 1.84-1.74 (m, 2H), 1.70-1.60 (m, 2H), 1.58-1.44 (m, 4H) ppm | 2-methyl- oxazole-5- carbaldehyde | General method A using cyclopentanamine, AcOH and NaBH₄, method D1 using 4- fluorophenylisocyanate | 1-cyclopentyl-3- (4- fluorophenyl)-1- [(2- methyloxazol- yl)methyl]urea |
| 146 | 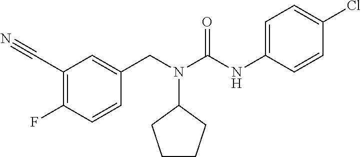 [SMILES: N#Cc1cc(CN(C(=O)Nc2ccc(Cl)cc2)C2CCCC2)ccc1F] | 372.6 | | 1H NMR (300 MHz, DMSO-d6): δ = 8.53 (s, 1H), 7.72 (dd, J = 2.1, 6.3 Hz, 1H), 7.66-7.58 (m, 1H), 7.52-7.43 (m, 3H), 7.30-7.23 (m, 2H), 4.50 (s, 2H), 4.52-4.43 (m, 1H), 1.84-1.70 (m, 2H), 1.68-1.58 (m, 2H), 1.56-1.32 (m, 4H) ppm | 2-fluoro-5- formyl- benzonitrile | General method A using cyclopentanamine, AcOH and NaBH₄, method D1 using 4- chlorophenylisocyanate | 3-(4- chlorophenyl)- [(3-cyano-4- fluoro- phenyl)methyl]- 1-cyclopentyl- urea |
| 147 | 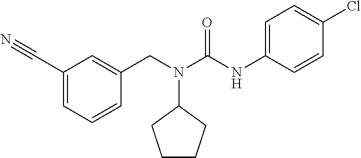 [SMILES: N#Cc1cccc(CN(C(=O)Nc2ccc(Cl)cc2)C2CCCC2)c1] | 354.6 | | 1H NMR (300 MHz, DMSO- d6):δ = 8.53 (s, 1H), 7.68 (m, 1H), 7.64-7.62 (m, 1H), 7.58-7.50 (m, 2H), 7.47 (d, J = 9.0 Hz, 2H), 7.26 (d, J = 9.0 Hz, 2H), 4.55 (s, 2H), 4.55-4.45 (m, 1H), 1.82-1.72 (m, 2H), 1.66-1.56 (m, 2H), 1.54-1.34 (m, 4H) ppm | 3- formylbenzo nitrile | General method A using cyclopentanamine, AcOH and NaBH₄, method D1 using 4- chlorophenylisocyanate | 3-(4- chlorophenyl)-1- [(3- cyanophenyl) methyl]-1- cyclopentyl-urea |
| 148 | 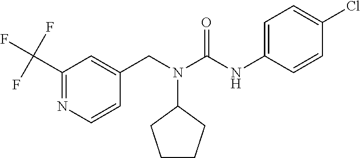 [SMILES: O=C(Nc1ccc(Cl)cc1)N(Cc1ccnc(C(F)(F)F)c1)C1CCCC1] | 398.1 | | 1H NMR (400 MHz, DMSO-d6): δ = 8.67 (d, J = 5.0 Hz, 1H), 8.60 (s, 1H), 7.71 (s, 1H), 7.52 (d, J = 5.0 Hz, 1H), 7.47 (d, J = 9.0 Hz, 2H), 7.26 (d, J = 9.0 Hz, 2H), 4.62 (s, 2H), 4.58-4.50 (m, 1H), 1.84- 1.74 (m, 2H), 1.68-1.58 (m, 2H), 1.56-1.44 (m, 2H), 1.44-1.32 (m, 2H) ppm | 2- (trifluoro- methyl) pyridine-4- carbaldehyde | General method A using cyclopentanamine, AcOH and NaBH₄, method D1 using 4- chlorophenylisocyanate | 3-(4- chlorophenyl)-1- cyclopentyl-1- [[2- (trifluoromethyl)- 4- pyridyl]methyl] urea |
| 149 | 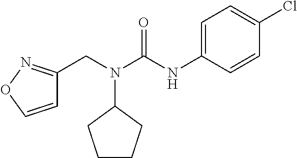 [SMILES: O=C(Nc1ccc(Cl)cc1)N(Cc1ccon1)C1CCCC1] | 320.1 | | 1H NMR (400 MHz, DMSO-d6): δ = 8.8 (d, J = 1.7 Hz, 1H), 8.56 (s, 1H), 7.49 (d, J = 9.0 Hz, 2H), 7.27 (d, J = 9.0 Hz, 2H), 6.43 (d, J = 1.7 Hz, 1H), 4.56 (s, 2H), 4.50-4.40 (m, 1H), 1.84-1.74 (m, 2H), 1.72- 1.60 (m, 2H), 1.56-1.44 (m, 4H) ppm | isoxazole-3- carbaldehyde | General method A using cyclopentanamine, AcOH and NaBH₄, method D1 using 4- chlorophenylisocyanate | 3-(4- chlorophenyl)-1- cyclopentyl-1- (isoxazol-3- ylmethyl)urea |
| 150 | 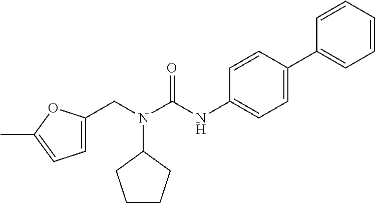 [SMILES: Cc1ccc(CN(C(=O)Nc2ccc(-c3ccccc3)cc2)C2CCCC2)o1] | 375.8 | | 1H NMR (300 MHz, DMSO-d6): δ = 8.36 (s, 1H), 7.61 (d, J = 7.4 Hz, 2H), 7.54 (s, 4H), 7.41 (t, J = 7.3 Hz, 2H), 7.29 (t, J = 7.2 Hz, 1H), 6.10 (d, J = 3.0 Hz, 1H), 5.98- 5.94 (m, 1H), 4.45 (s, 2H), 4.50- 4.40 (m, 1H), 2.21 (s, 3H), 1.84- 1.72 (m, 2H), 1.70-1.44 (m, 6H) ppm | 5- methylfuran- 2- carbaldehyde | General method A using cyclopentanamine, AcOH and NaBH₄, method D3 using 4- phenylaniline | 1-cyclopentyl-1- [(5-methyl-2- furyl)methyl]-3- (4- phenylphenyl) urea |
| 151 | 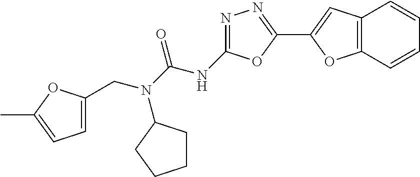 [SMILES: Cc1ccc(CN(C(=O)Nc2nnc(-c3cc4ccccc4o3)o2)C2CCCC2)o1] | 407.8 | | 1H NMR (300 MHz, DMSO-d6): δ = 7.79 (d, J = 7.6 Hz, 1H), 7.74 (d, J = 8.3 Hz, 1H), 7.69 (s, 1H), 7.49 (t, J = 7.5 Hz, 1H), 7.37 (t, J = 7.4 Hz, 1H), 6.07-6.11 (m, 1H), 5.95-5.99 (m, 1H), 4.38-4.52 (m, 3H), 2.21 (s, 3H), 1.43-1.80 (m, 8H) ppm | 5- methylfuran- 2- carbaldehyde | General method A using cyclopentanamine, AcOH and NaBH₄, method D1 using 5- (benzofuran-2-yl)- 1,3,4-oxadiazole-2- amine | 3-[5- (benzofuran-2- yl)-1,3,4- oxadiazol-2-yl- 1-cyclopentyl- [(5-methyl-2- furyl)methyl]urea |
| 152 | 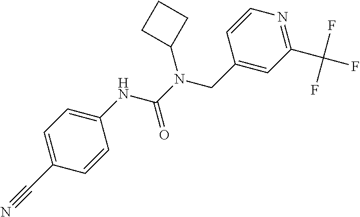 [SMILES: N#Cc1ccc(NC(=O)N(Cc2ccnc(C(F)(F)F)c2)C2CCC2)cc1] | 375.7 | | 1H NMR (300 MHz, DMSO-d6): δ = 8.88 (s, 1H), 8.68 (d, J = 5.1 Hz, 1H), 7.71-7.63 (m, 5H), 7.50 (d, J = 5.1 Hz, 1H), 4.79 (s, 2H), 4.68- 4.60 (m, 1H), 2.12-1.94 (m, 4H), 1.59-1.51 (m, 2H) ppm | 2- (trifluoro- methyl) pyridine-4- carbaldehyde | General method A using cyclopentanamine, AcOH and NaBH₄, method D1 using 4- isocyanatobenzonitrile | 3-(4- cyanophenyl)- cyclobutyl-1-[[2- (trifluoromethyl)- 4- pyridyl]methyl] urea |
| 153 | 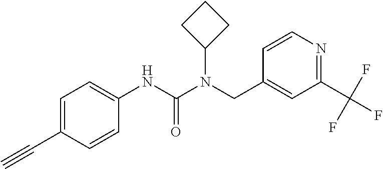 [SMILES: C#Cc1ccc(NC(=O)N(Cc2ccnc(C(F)(F)F)c2)C2CCC2)cc1] | 374.8 | | 1H NMR (500 MHz, DMSO-d6): δ = 8.69 (d, J = 4.0 Hz, 1H), 8.61 (s, 1H), 7.71 (m, 1H), 7.51 (d, J = 4.0 Hz, 1H), 7.47 (d, J = 8.0 Hz, 2H), 7.34 (d, J = 8.0 Hz, 2H), 4.79 (s, 2H), 4.71-4.58 (m, 1H), 4.02 (s, 1H), 2.18-2.06 (m, 2H), 2.04-1.94 (m, 2H), 1.63-1.47 (m, 2H) ppm | 2- (trifluoro- methyl) pyridine-4- carbaldehyde | General method A using cyclopentanamine, AcOH and NaBH₄, method D2 using 4- ethynylaniline | 1-cyclobutyl-3- (4- ethynylphenyl)- 1-[[2- (trifluoromethyl)- 4- pyridyl]methyl] urea |
| 154 | 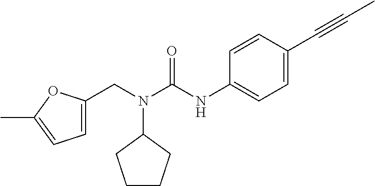 [SMILES: CC#Cc1ccc(NC(=O)N(Cc2ccc(C)o2)C2CCCC2)cc1] | 337.7 | | 1H NMR (500 MHz, DMSO-d6): δ = 8.38 (s, 1H), 7.42 (d, J = 8.5 Hz, 2H), 7.22 (d, J = 8.5 Hz, 2H), 6.09 (bs, 1H), 5.96 (bs, 1H), 4.47- 4.38 (m, 3H), 2.21 (s, 3H), 2.00 (s, 3H), 1.82-1.70 (m, 2H), 1.69- 1.60 (m, 2H), 1.59-1.44 (m, 4H) ppm | 5- methylfuran- 2- carbaldehyde | General method A using cyclopentanamine, AcOH and NaBH₄, method T using 4- bromoaniline, tributyl- propynylstannane and Pd(PPh₃)₄, method D2 using 4-prop-1- ynylaniline | 1-cyclobutyl-3- (4-prop-1- ynylphenyl)-1- [[2- (trifluoromethyl)- 4- pyridyl]methyl] urea |
| 155 | 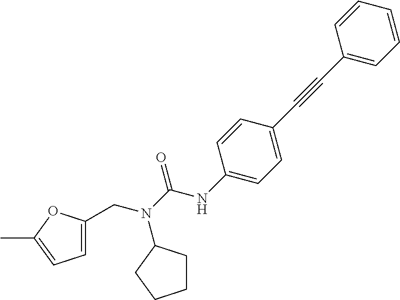 [SMILES: Cc1ccc(CN(C(=O)Nc2ccc(C#Cc3ccccc3)cc2)C2CCCC2)o1] | 399.8 | | 1H NMR (500 MHz, DMSO-d6): δ = 8.49 (s, 1H), 7.56-7.48 (m, 4H), 7.43-7.32 (m, 5H), 6.11 (bs, 1H), 5.96 (bs, 1H), 4.48-4.38 (m, 3H), 3.86 (s, 2H), 2.21 (s, 3H), 1.81-1.71 (m, 2H), 1.70-1.60 (m, 2H), 1.58-1.42 (m, 4H) ppm | 5- methylfuran- 2- carbaldehyde | General method A using cyclopentanamine, AcOH and NaBH₄, method D1 using 1- iodo-4- isocyanatobenzene, method F using ethynylbenzene, Pd(PPh₃)₂Cl₂, CuI and TEA | 1-cyclopentyl-1- [(5-methyl-2- furyl)methyl]-3- [4-(2- phenylethynyl) phenyl]urea |
| 156 | 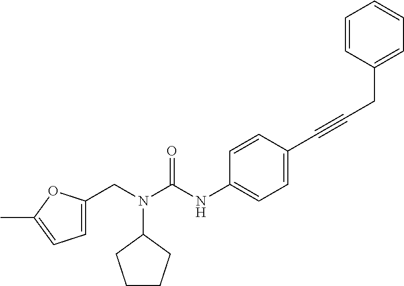 [SMILES: Cc1ccc(CN(C(=O)Nc2ccc(C#CCc3ccccc3)cc2)C2CCCC2)o1] | 413.8 | | 1H NMR (500 MHz, DMSO-d6): δ = 8.41 (s, 1H), 7.47-7.24 (m, 9H), 6.12 (bs, 1H), 5.96 (bs, 1H), 4.49-4.39 (m, 3H), 3.86 (s, 2H), 2.21 (s, 3H), 1.81-1.71 (m, 2H), 1.70-1.60 (m, 2H), 1.58-1.42 (m, 4H), ppm | 5- methylfuran- 2- carbaldehyde | General method A using cyclopentanamine, AcOH and NaBH₄, method D1 using 1- iodo-4- isocyanatobenzene, method F using prop-2- ynylbenzene, Pd(PPh₃)₂Cl₂, CuI and TEA | 1-cyclopentyl-1- [(5-methyl-2- furyl)methyl]-3- [4-(3- phenylprop-1- ynyl)phenyl]urea |
| 157 | 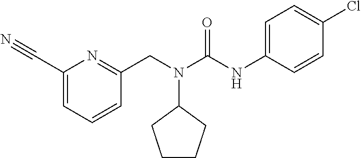 [SMILES: N#Cc1cccc(CN(C(=O)Nc2ccc(Cl)cc2)C2CCCC2)n1] | 355.6 | | 1H NMR (300 MHz, DMSO-d6): δ = 8.64 (s, 1H), 8.01 (d, J = 7.5 Hz, 1H), 7.90 (d, J = 7.5 Hz, 1H), 7.59 (d, J = 7.5 Hz, 1H), 7.46 (d, J = 8.0 Hz, 2H), 7.26 (d, J = 8.0 Hz, 2H), 4.61 (s, 2H), 4.61-4.49 (m, 1H), 1.86-1.72 (m, 2H), 1.70- 1.56 (m, 2H), 1.55-1.38 (m, 4H), ppm | 6- formyl- pyridine-2- carbonitrile | General method A using cyclopentanamine, AcOH and NaBH₄, method D1 using 4- chlorophenylisocyanate | 3-(4- chlorophenyl)- [(6-cyano-2- pyridyl)methyl- 1-cyclopentyl] urea |
| 158 | 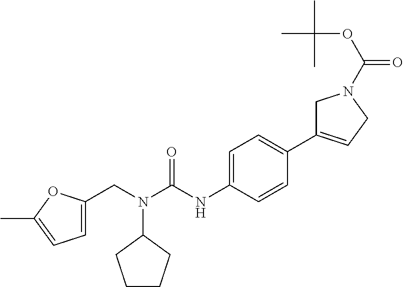 [SMILES: Cc1ccc(CN(C(=O)Nc2ccc(C3=CCN(C(=O)OC(C)(C)C)C3)cc2)C2CCCC2)o1] | 466.8 | | 1H NMR (500 MHz, DMSO-d6): δ = 8.35 (s, 1H), 7.46 (d, J = 8.5 Hz, 2H), 7.35-7.31 (m, 2H), 6.20 (d, J = 18 Hz, 1H), 6.11 (bs, 1H), 5.96 (bs, 1H), 4.48-4.41 (m, 3H), 4.38 - 4.34 (m, 2H), 4.19-4.15 (m, 2H), 2.21 (s, 3H), 1.82-1.71 (m, 2H), 1.70-1.62 (m, 2H), 1.60-1.48 (m, 4H), 1.47-1.40 (m, 9H) ppm | 5- methylfuran- 2- carbaldehyde | General method A using cyclopentanamine, AcOH and NaBH₄, method D1 using 1- bromo-4- isocyanatobenzene, method E using tert- butyl 3-(4,4,5,5- tetramethyl-1,3,2- dioxaborolan-2-yl)-2,5- dihydropyrrole-1- carboxylate, Pd(PPh₃)₂ and K₂CO₃ | tert-butyl 3-[4- [[cyclopentyl- [(5-methyl-2- furyl)methyl]car- bamoyl]amino] phenyl]-2,5- dihydropyrrole- 1-carboxylate |
| 159 | 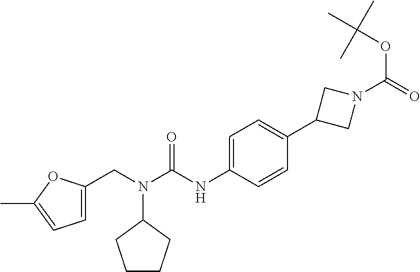 [SMILES: Cc1ccc(CN(C(=O)Nc2ccc(C3CN(C(=O)OC(C)(C)C)C3)cc2)C2CCCC2)o1] | 0 | | 1H NMR (500 MHz, DMSO-d6): δ = 8.26 (s, 1H), 7.42 (d, J = 8.5 Hz, 2H), 7.18 (d, J = 8.5 Hz, 2H), 6.09 (d, J = 2.0 Hz, 1H), 5.95 (d, J = 2.0 Hz, 1H), 4.48-4.41 (m, 3H), 4.38-4.34 (m, 2H), 4.27- 4.17 (m, 2H), 3.83-3.76 (m, 2H), 3.73-3.64 (m, 1H), 2.21 (s, 3H), 1.80-“1.71 (m, 2H), 1.70-“1.60 (m, 2H), 1.58-“ 1.48 (m, 4H), 1.40 (s, 9H) ppm | 5- methylfuran- 2- carbaldehyde | General method A using cyclopentanamine, AcOH and NaBH₄, method D1 using 1- iodo-4- isocyanatobenzene, method K using 1-Boc- 3-iodoazetidine, Zn, Pd(OAc)₂ and Cphos | tert-butyl 3-[4- [[cyclopentyl- [(5-methyl-2- furyl)methyl]car- bamoyl]amino] phenyl]azetidine- 1-carboxylate |
| 160 | 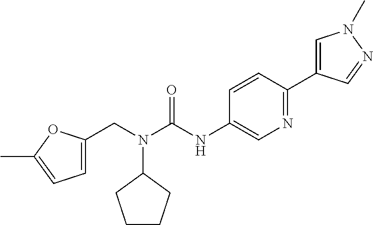 [SMILES: Cc1ccc(CN(C(=O)Nc2ccc(-c3cnn(C)c3)nc2)C2CCCC2)o1] | 380.8 | | 1H NMR (500 MHz, DMSO-d6): δ = 8.54 (d, J = 2.5 Hz, 1H), 8.47 (s, 1H), 8.16 (s, 1H), 7.89 (s, 1H), 7.84 (dd, J = 8.5, 2.5 Hz, 1H), 7.51 (d, J = 8.5 Hz, 1H), 6.12 (d, J = 2.0 Hz, 1H), 5.96 (d, J = 2.0 Hz, 1H), 4.48-4.41 (m, 3H), 3.86 (s, 3H) 2.22 (s, 3H), 1.84-“1.73 (m, 2H), 1.72-“1.62 (m, 2H), 1.60-“1.40 (m, 4H) ppm | 5- methylfuran- 2- carbaldehyde | General method A using cyclopentanamine, AcOH and NaBH₄, method D2 using 6- chloropyridin-3-amine, method E using 1- methyl-4-(4,4,5,5- tetramethyl-1,3,2- dioxaborolan-2- yl)pyrazole, X-Phos-Pd- G1, XPhos and K₂CO₃ | 1-cyclopentyl-1- [(5-methyl-2- furyl)methyl]-3- [6-(1- methylpyrazol- 4-yl)-3- pyridyl]urea |
| 161 | 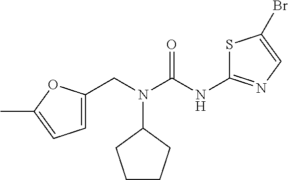 [SMILES: Cc1ccc(CN(C(=O)Nc2ncc(Br)s2)C2CCCC2)o1] | 384.6, 386.7 | | 1H NMR (300 MHz, DMSO-d6): δ = 10.75 (bs, 1H), 7.41 (s, 1H), 6.08 (d, J = 3.0 Hz, 1H), 5.95 (d, J = 2.5 Hz, 1H), 4.48 (s, 2H), 4.45- 4.35 (m, 1H), 2.20 (s, 3H), 1.80- 1.43 (m, 8H) ppm | 5- methylfuran- 2- carbaldehyde | General method A using cyclopentanamine, AcOH and NaBH₄, method Q for phenyl carbamate synthesis; method D5 using phenyl N-(5- bromothiazol-2- yl)carbamate | 3-(5- bromothiazol-2- yl)-1- cyclopentyl-1- [(5-methyl-2- furyl)methyl]urea |
| 162 | 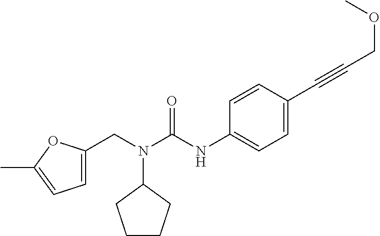 [SMILES: COCC#Cc1ccc(NC(=O)N(Cc2ccc(C)o2)C2CCCC2)cc1] | 367.7 | | 1H NMR (400 MHz, DMSO-d6): δ = 8.44 (s, 1H), 7.46 (d, J = 8.6 Hz, 2H), 7.31 (d, J = 8.6 Hz, 2H), 6.11 (d, J = 2.7 Hz, 1H), 5.95-5.94 (m, 1H), 4.46-4.38 (m, 3H), 4.28 (s, 2H), 3.30 (s, 3H), 2.20 (s, 3H), 1.80-1.70 (m, 2H), 1.69-1.59 (m, 2H), 1.59-1.45 (m, 4H) ppm | 5- methylfuran- 2- carbaldehyde | General method A using cyclopentanamine, AcOH and NaBH₄, method D1 using 1- iodo-4- isocyanatobenzene, method F using 3- methoxyprop-1-yne, Pd(PPh₃)₂Cl₂, CuI and TEA | 1-cyclopentyl-3- [4-(3- methoxyprop-1- ynyl)phenyl]-1- [(5-methyl-2- furyl)methyl]urea |
| 163 | 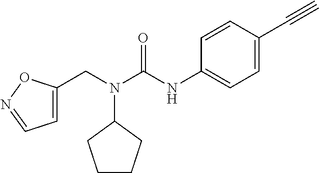 [SMILES: C#Cc1ccc(NC(=O)N(Cc2ccno2)C2CCCC2)cc1] | 310.7 | | 1H NMR (400 MHz, DMSO-d6): δ = 8.63 (s, 1H), 8.44 (d, J = 2.0 Hz, 1H), 7.46 (d, J = 8.6 Hz, 2H), 7.32 (d, J = 8.6 Hz, 2H), 6.11 (d, J = 2.0 Hz, 1H), 4.63 (s, 2H), 4.52- 4.45 (m, 1H), 4.03 (s, 1H), 1.86- 1.74 (m, 2H), 1.71-1.61 (m, 2H), 1.57-1.45 (m, 4H) ppm | isoxazole-5- carbaldehyde | General method A using cyclopentanamine, AcOH and NaBH₄, method D2 using 4- ethynylaniline | 1-cyclopentyl-3- (4- ethynylphenyl)- 1-(isoxazol-5- ylmethyl)urea |
| 164 | 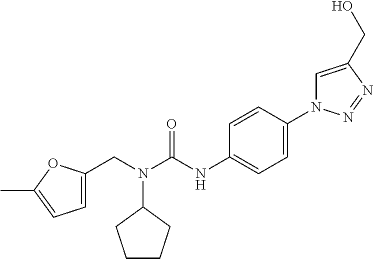 [SMILES: Cc1ccc(CN(C(=O)Nc2ccc(-n3cc(CO)nn3)cc2)C2CCCC2)o1] | 396.8 | | 1H NMR (500 Hz, CDCl3): δ = 8.55 (s, 2H), 7.74 (d, J = 8.8 Hz, 2H), 7.65 (d, J = 8.8 Hz, 2H), 6.08 (d, J = 2.0 Hz, 1H), 5.97 (d, J = 2.0 Hz, 1H), 5.29 (t, J = 5.5 Hz, 1H), 4.59 (d, J = 5.5 Hz, 2H), 4.49- “4.24 (m, 3H), 2.22 (s, 3H), 1.84- “1.73 (m, 2H), 1.72-“1.63 (m, 2H), 1.62-“1.46 (m, 4H) ppm | 5- methylfuran- 2- carbaldehyde | General method A using cyclopentanamine, AcOH and NaBH₄; method M using tert- butyl-chloro-dimethyl silane; method D2, method N using TBAFxH₂O | 1-cyclopentyl-3- [4-[4- (hydroxymethyl) triazol-1- yl]phenyl]-1-[(5- methyl-2- furyl)methyl]urea |
| 165 | 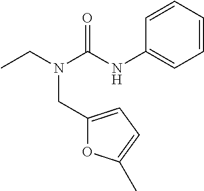 [SMILES: CCN(Cc1ccc(C)o1)C(=O)Nc1ccccc1] | 259.7 | | 1H NMR (300 MHz, DMSO-d6): δ = 8.28 (s, 1H), 7.46 (d, J = 8.2 Hz, 2H), 7.22 (t, J = 7.8 Hz, 2H), 6.92 (t, J = 7.3 Hz, 1H), 6.18 (d, J = 2.8 Hz, 1H), 6.00-5.96 (m, 1H), 4.47 (s, 2H), 3.40-3.26 (m, 2H), 2.21 (s, 3H), 1.03 (t, J = 6.9 Hz, 3H) ppm | 5- methylfuran- 2- carbaldehyde | General method A using ethanaminexHCl, AcOH and NaBH₄, method D1 using phenyl isocyanate | 1-ethyl-1-[(5- methyl-2- furyl)methyl]-3- phenyl-urea |
| 166 | 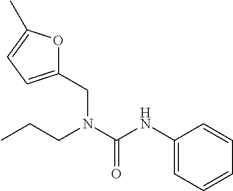 [SMILES: CCCN(Cc1ccc(C)o1)C(=O)Nc1ccccc1] | 273.7 | | 1H NMR (300 MHz, DMSO-d6): δ = 8.27 (s, 1H), 7.45 (d, J = 8.2 Hz, 2H), 7.22 (t, J = 7.8 Hz, 2H), 6.92 (t, J = 7.4 Hz, 1H), 6.18 (d, J = 2.8 Hz, 1H), 6.00-5.96 (m, 1H), 4.47 (s, 2H), 3.25 (t, J = 7.3 Hz, 2H), 2.21 (s, 3H), 1.55-1.40 (m, 2H), 0.81 (t, J = 7.3 Hz, 3H) ppm | 5- methylfuran- 2- carbaldehyde | General method A using ethanaminexHCl, AcOH and NaBH₄, method D1 using phenyl isocyanate | 1-[(5-methyl-2- furyl)methyl]-3- phenyl-1-propyl- urea |
| 167 | 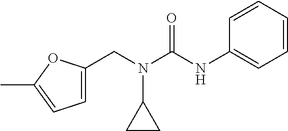 [SMILES: Cc1ccc(CN(C(=O)Nc2ccccc2)C2CC2)o1] | 271.7 | | 1H NMR (300 MHz, DMSO-d6): δ = 8.25 (s, 1H), 7.51 (d, J = 8.2 Hz, 2H), 7.23 (t, J = 7.8 Hz, 2H), 6.95 (t, J = 7.3 Hz, 1H), 6.10 (d, J = 2.8 Hz, 1H), 6.00-5.95 (m, 1H), 4.41 (s, 2H), 2.62-2.52 (m, 1H), 2.21 (s, 3H), 0.94-0.84 (m, 2H), 0.88-0.68 (m, 2H) ppm | 5- methylfuran- 2- carbaldehyde | General method A using cyclopropanamine, AcOH and NaBH₄, method D1 using phenyl isocyanate | 1-cyclopropyl-1- [(5-methyl-2- furyl)methyl]-3- phenyl-urea |
| 168 | 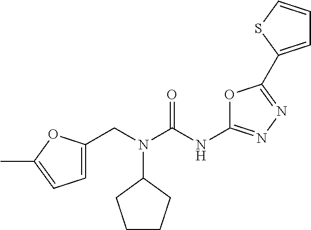 [SMILES: Cc1ccc(CN(C(=O)Nc2nnc(-c3cccs3)o2)C2CCCC2)o1] | 373.7 | | 1H NMR (300 MHz, DMSO-d6): δ = 7.89 (d, J = 5.0 Hz, 1H), 7.71 (d, J = 3.5 Hz, 1H), 7.25 (t, J = 4.8 Hz, 1H), 6.10-6.04 (m, 1H), 5.99- 5.92 (m, 1H), 4.60-4.20 (m, 3H), 2.21 (s, 3H), 1.80-1.40 (m, 8H) ppm | 5- methylfuran- 2- carbaldehyde | General method A using cyclopentanamine, AcOH and NaBH₄, method D3 using 5-(2- thienyl)-1,3,4- oxadiazole-2-amine | 1-cyclopentyl-1- [(5-methyl-2- furyl)methyl]-3- [5-(2-thienyl)- 1,3,4-oxadiazol- 2-yl]urea |
| 169 | 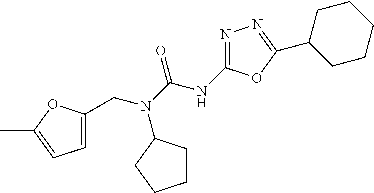 [SMILES: Cc1ccc(CN(C(=O)Nc2nnc(C3CCCCC3)o2)C2CCCC2)o1] | 373.9 | | 1H NMR (600 MHz, DMSO-d6): δ = 6.10-6.00 (bs, 1H), 5.95 (s, 1H), 4.56-4.26 (m, 3H), 2.90-2.70 (m, 1H), 2.21 (s, 3H), 1.97-1.90 (m, 2H), 1.78-1.66 (m, 3H), 1.66- 1.58 (m, 3H), 1.58-1.40 (m, 6H), 1.40-1.30 (m, 2H), 1.28-1.20 (m, 2H) ppm | 5- methylfuran- 2- carbaldehyde | General method A using cyclopentanamine, AcOH and NaBH₄, method D3 using 5- cyclohexyl-1,3,4- oxadiazole-2-amine | 3-(5-cyclohexyl- 1,3,4-oxadiazol- 2-yl)-1- cyclopentyl-1- [(5-methyl-2- furyl)methyl]urea |
| 170 | 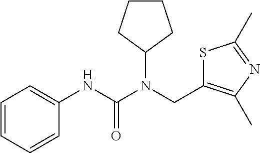 [SMILES: Cc1nc(C)c(CN(C(=O)Nc2ccccc2)C2CCCC2)s1] | 330.8 | | 1H NMR (500 MHz, CDCl3): δ = 7.30-7.25 (m, 4H), 7.05-7.00 (m, 1H), 6.37 (s, 1H), 4.54-4.45 (m, 1H), 4.49 (s, 2H), 2.64 (s, 3H), 2.43 (s, 3H), 2.04-1.96 (m, 2H), 1.80-1.72 (m, 2H), 1.69-1.62 (m, 2H), 1.62-1.52 (m, 2H) ppm | 2,4- dimethyl- thiazole-5- carbaldehyde | General method A using cyclopentanamine, AcOH and NaBH₄, method D1 using phenyl isocyanate | 1-cyclopentyl-1- [(2,4- dimethylthiazol- 5-yl)methyl]-3- phenyl-urea |
| 171 | 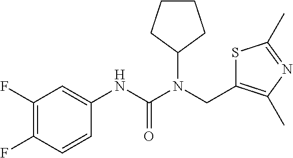 [SMILES: Cc1nc(C)c(CN(C(=O)Nc2ccc(F)c(F)c2)C2CCCC2)s1] | 366.8 | | 1H NMR (500 MHz, CDCl3): δ = 7.44-7.35 (m, 1H), 7.06-6.99 (m, 1H), 6.82-6.77 (m, 1H), 6.34 (s, 1H), 4.50-4.41 (m, 1H), 4.47 (s, 2H), 2.64 (s, 3H), 2.42 (s, 3H), 2.04-1.96 (m, 2H), 1.80-1.72 (m, 2H), 1.69-1.62 (m, 2H), 1.62-1.52 (m, 2H) ppm | 2,4- dimethyl- thiazole-5- carbaldehyde | General method A using cyclopentanamine, AcOH and NaBH₄, method D1 using 1,2- difluoro-4- isocyanatobenzene | 1-cyclopentyl-1- [(2,4- dimethylthiazol- 5-yl)methyl]-3- (3,4- difluorophenyl)- urea |
| 172 | 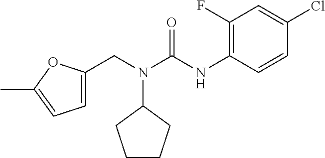 [SMILES: Cc1ccc(CN(C(=O)Nc2ccc(Cl)cc2F)C2CCCC2)o1] | 351.8 | | 1H NMR (500 MHz, DMSO-d6): δ = 8.05 (s, 1H), 7.58-7.54 (m, 1H), 7.41-7.38 (m, 1H), 7.20-7.18 (m, 1H), 6.14 (d, J = 2.9, 1H), 5.99- 5.98 (m, 1H), 4.44-4.38 (m, 1H), 4.42 (s, 2H) 2.25 (s, 3H), 1.79- 1.73 (m, 2H), 1.67-1.62 (m, 2H), 1.59-1.52 (m, 2H), 1.52-1.45 (m, 2H) ppm | 5- methylfuran- 2- carbaldehyde | General method A using cyclopentanamine, AcOH and NaBH₄, method D3 using 4- chloro-2-fluoro aniline | 3-(4-chloro- fluoro-phenyl)- 1-cyclopentyl-1- [(5-methyl-2- furyl)methyl]urea |
| 173 | 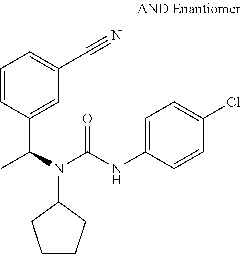 [SMILES: C[C@@H](c1cccc(C#N)c1)N(C(=O)Nc1ccc(Cl)cc1)C1CCCC1] | 368.8 | | 1H NMR (600 MHz, DMSO-d6): δ = 8.50 (s, 1H), 7.75 (s, 1H), 7.72 (d, J = 7.7 Hz, 1H), 7.66 (d, J = 8.0 Hz, 1H), 7.57 (t, J = 7.8 Hz, 1H), 7.47 (d, J = 8.9 Hz, 2H), 7.26 (s, J = 8.9 Hz, 2H), 5.03-4.96 (m, 1H), 3.76-3.68 (m, 1H), 1.89-1.82 (m, 1H), 1.82-1.62 (m, 4H), 1.66 (d, J = 6.8 Hz, 3H), 1.49-1.41 (m, 2H), 1.41-1.34 (m, 1H) ppm | 3- acetyl- benzonitrile | General method A using cyclopentanamine, AcOH and NaCNBH₃, method D1 using 4- chlorophenylisocyanate | 3-(4- chlorophenyl)-1- [1-(3- cyanophenyl) ethyl]-1- cyclopentyl-urea |
| 174 | 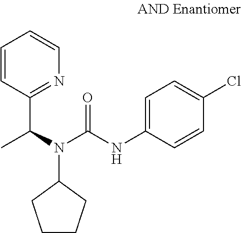 [SMILES: C[C@@H](c1ccccn1)N(C(=O)Nc1ccc(Cl)cc1)C1CCCC1] | 344.8 | | 1H NMR (300 MHz, DMSO-d6): δ = 9.60 (s, 1H), 8.63-8.58 (m, 1H), 7.81 (dt, J = 1.8, 7.8 Hz, 1H), 7.51-7.42 (m, 3H), 7.35-7.29 (m, 1H), 7.26 (d, J = 8.9 Hz, 2H), 4.97-4.86 (m, 1H), 4.10-3.95 (m, 1H), 1.84-1.64 (m, 5H), 1.62 (d, J = 6.9 Hz, 3H), 1.52-1.34 (m, 3H) ppm | 2- acetyl- pyridine | General method A using cyclopentanamine, AcOH and NaCNBH₃, method D1 using 4- chlorophenylisocyanate | 3-(4- chlorophenyl)-1- cyclopentyl-1- [1-(2- pyridyl)ethyl]urea |
| 175 | 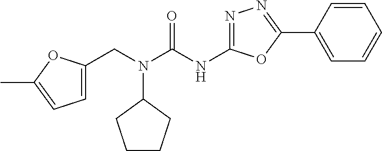 [SMILES: Cc1ccc(CN(C(=O)Nc2nnc(-c3ccccc3)o2)C2CCCC2)o1] | 367.8 | | 1H NMR (400 MHz, DMSO-d6): δ = 7.89-7.87 (m, 2H), 7.62-7.54 (m, 3H), 6.08-6.06 (m, 1H), 5.96- 5.95 (m, 1H), 4.48-4.42 (m, 1H), 4.46 (s, 2H), 2.20 (s, 3H), 1.79- 1.68 (m, 2H), 1.68-1.60 (m, 2H), 1.60-1.52 (m, 2H), 1.52-1.45 (m, 2H) ppm | 5- methylfuran- 2- carbaldehyde | General method A using cyclopentanamine, AcOH and NaBH₄, method D3 using 5- phenyl-1,3,4- oxadiazole-2-amine | 1-cyclopentyl-1- [(5-methyl-2- furyl)methyl]-3- (5-phenyl-1,3,4- oxadiazol-2- yl)urea |
| 176 | 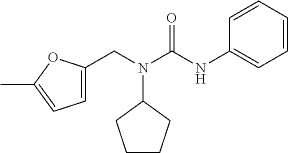 [SMILES: Cc1ccc(CN(C(=O)Nc2ccccc2)C2CCCC2)o1] | 299.8 | | 1H NMR (300 MHz, DMSO-d6): δ 8.23 (s, 1H), 7.42 (d, J = 8.7 Hz, 2H), 7.20 (t, J = 7.9 Hz, 2H), 6.91 (t, J = 7.3 Hz, 1H), 6.09 (d, J = 2.9 Hz, 1H), 5.95 (dd, J = 1.0, 3.0 Hz, 1H), 4.50-4.36 (m, 1H), 4.42 (s, 2H), 2.20 (s, 3H), 1.82-1.68 (m, 2H), 1.68-1.58 (m, 2H), 1.58-1.42 (m, 4H) ppm | 5- methylfuran- 2- carbaldehyde | General method A using cyclopentanamine, AcOH and NaBH₄, method D1 using phenyl isocyanate | 1-cyclopentyl-1- [(5-methyl-2- furyl)methyl]-3- phenyl-urea |
| 177 | 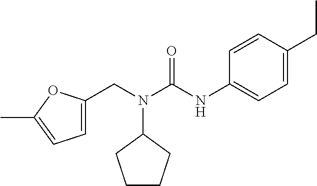 [SMILES: CCc1ccc(NC(=O)N(Cc2ccc(C)o2)C2CCCC2)cc1] | 327.8 | | 1H NMR (600 MHz, DMSO-d6): δ = 8.15 (s, 1H), 7.32 (d, J = 8.5 Hz, 2H), 7.05 (d, J = 8.5 Hz, 2H), 6.09 (d, J = 3.0 Hz, 1H), 5.96 (dd, J = 1.0, 3.0 Hz, 1H), 4.47-4.40 (m, 1H), 4.42 (s, 2H), 2.55-2.48 (m, 2H), 2.21 (s, 3H), 1.79-1.72 (m, 2H), 1.70-1.60 (m, 2H), 1.58-1.46 (m, 4H), 1.14 (t, J = 7.6 Hz, 3H) ppm | 5- methylfuran- 2- carbaldehyde | General method A using cyclopentanamine, AcOH and NaBH₄, method D1 using 1- ethyl-4- isocyanatobenzene | 1-cyclopentyl-3- (4-ethylphenyl)- 1-[(5-methyl-2- furyl)methyl]urea |
| 178 | 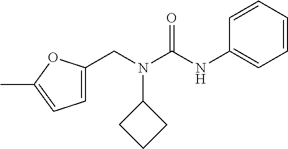 [SMILES: Cc1ccc(CN(C(=O)Nc2ccccc2)C2CCC2)o1] | 285.8 | | 1H NMR (300 MHz, DMSO-d6): δ = 8.20 (s, 1H), 7.42 (d, J = 8.8 Hz, 2H), 7.21 (t, J = 7.8 Hz, 2H), 6.91 (t, J = 7.3 Hz, 1H), 6.06 (d, J = 3.0 Hz, 1H), 5.96 (dd, J = 1.0, 3.0 Hz, 1H), 4.51 (s, 2H), 4.46-4.32 (m, 1H), 2.20 (s, 3H), 2.17-2.01 (m, 4H), 1.66-1.44 (m, 2H) ppm | 5- methylfuran- 2- carbaldehyde | General method A using cyclobutanamine, AcOH and NaBH₄, method D1 using phenyl isocyanate | 1-cyclobutyl-1- [(5-methyl-2- furyl)methyl]-3- phenyl-urea |
| 179 | 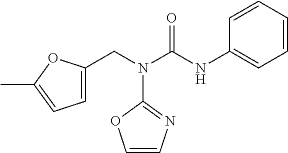 [SMILES: Cc1ccc(CN(C(=O)Nc2ccccc2)c2ncco2)o1] | 298.8 | | 1H NMR (500 MHz, DMSO-d6): δ = 11.32 (s, 1H), 8.00 (s, 1H), 7.52 (d, J = 7.6 Hz, 2H), 7.35 (t, J = 7.6 Hz, 2H), 7.25 (s, 1H), 7.10 (t, J = 7.3 Hz, 1H), 6.20 (d, J = 3.0 Hz, 1H), 5.99 (dd, J = 3.0, 0.8 Hz, 1H), 5.06 (s, 2H), 2.19 (s, 3H) ppm | 5- methylfuran- 2- carbaldehyde | General method A using oxazol-2-amine, AcOH and NaCNBH₃, method D1 using phenyl isocyanate | 1-[(5-methyl-2- furyl)methyl]-1- oxazol-2-yl-3- phenyl-urea |
| 180 | 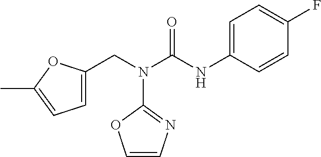 [SMILES: Cc1ccc(CN(C(=O)Nc2ccc(F)cc2)c2ncco2)o1] | 316.8 | | 1H NMR (500 MHz, DMSO-d6): δ = 11.26 (s, 1H), 8.00 (s, 1H), 7.55-7.53 (m, 2H), 7.24 (s, 1H), 7.21-7.17 (m, 2H), 6.20 (d, J = 2.8 Hz, 1H), 5.99-5.98 (m, 1H), 5.05 (s, 2H), 2.19 (s, 3H) ppm | 5- methylfuran- 2- carbaldehyde | General method A using oxazol-2-amine, AcOH and NaCNBH₃, method D1 using 4- fluorophenylisocyanate | 3-(4- fluorophenyl)-1- [(5-methyl-2- furyl)methyl]-1- oxazol-2-yl-urea |
| 181 | 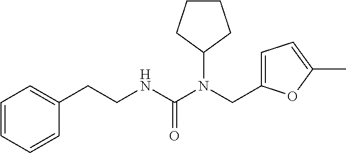 [SMILES: Cc1ccc(CN(C(=O)NCCc2ccccc2)C2CCCC2)o1] | 327.9 | | 1H NMR (500 MHz, DMSO-d6): δ 7.28-7.25 (m, 2H), 7.19-7.16 (m, 3H), 6.25 (t, J = 5.4 Hz, 1H), 5.97-5.96 (m, 1H), 5.94-5.93 (m, 1H), 4.28-4.21 (m. 3H), 3.26-3.21 (m, 2H), 2.70 (t, J = 7.7 Hz, 2H), 2.20 (s, 3H), 1.68-162 (m, 2H), 1.62-1.56 (m, 2H), 1.47-1.40 (m, 4H) ppm | 5- methylfuran- 2- carbaldehyde | General method A using cyclopentanamine, AcOH and NaBH₄, method D1 using 2- isocyanatoethylbenzene | 1-cyclopentyl-1- [(5-methyl-2- furyl)methyl]-3- (2- phenylethyl)urea |
| 182 | 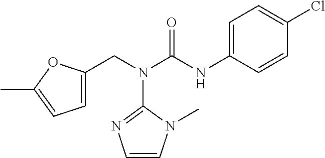 [SMILES: Cc1ccc(CN(C(=O)Nc2ccc(Cl)cc2)c2nccn2C)o1] | 345.7 | | 1H NMR (400 MHz, DMSO-d6): δ = 8.48 (bs, 1H), 7.46 (d, J = 9.0 Hz, 2H), 7.29 (d, J = 9.0 Hz, 2H), 7.07 (d, J = 1.4 Hz, 1H), 6.84 (d, J = 1.4 Hz, 1H), 4.73 (s, 2H), 3.21 (s, 3H), 2.18 (s, 3H) ppm | 5- methylfuran- 2- carbaldehyde | General method A using 1-methyl-2- aminoimidazolexHCl, Ti(iPrO)₄, TEA and NaBH₄, method D1 using 4- chlorophenylisocyanate | 3-(4- chlorophenyl)-1- [(5-methyl-2- furyl)methyl]-1- (1- methylimidazol- 2-yl)urea |
| 183 | 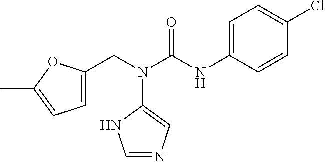 [SMILES: Cc1ccc(CN(C(=O)Nc2ccc(Cl)cc2)c2cnc[nH]2)o1] | 331.7 | | 1H NMR (400 MHz, DMSO-d6, 353 K): δ = 11.65 (bs, 1H), 7.50 (d, J = 8.6 Hz, 2H), 7.31 (d, J = 9.0 Hz, 2H), 6.88 (bs, 2H), 6.10 (d, J = 2.9 Hz, 1H), 5.94-5.91 (m, 1H), 5.06 (s, 2H), 2.18 (s, 3H) ppm | 5- methylfuran- 2- carbaldehyde | General method A using 2-aminoimidazole hemisulfate, Ti(iPrO)₄, TEA and NaBH₄, method D1 using 4- chlorophenylisocyanate | 3-(4- chlorophenyl)-1- (1H-imidazol-5- yl)-1-[(5-methyl- 2- furyl)methyl]urea |
| 184 | 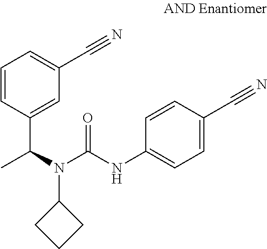 [SMILES: C[C@@H](c1cccc(C#N)c1)N(C(=O)Nc1ccc(C#N)cc1)C1CCC1] | 345.8 | | 1H NMR (600 MHz, DMSO-d6): δ = 8.74 (s, 1H), 7.73 (s, 1H), 7.71 (d, J = 7.8 Hz, 1H), 7.67-7.64 (m, 3H), 7.63-7.60 (m, 2H), 7.55 (t, J = 7.2 Hz, 1H), 5.04-5.00 (m, 1H), 4.27-4.21 (m, 1H), 2.32-2.22 (m, 2H), 2.17-2.08 (m, 2H), 1.68 (d, J = 6.8 Hz, 3H), 1.64-1.59 (m, 1H), 1.56-1.48 (m, 1H) ppm | 3- acetyl- benzonitrile | General method A using cyclobutanamine, AcOH and NaCNBH₃, method D1 using 4- cyanophenylisocyanate | 3-(4- cyanophenyl)-1- [1-(3- cyanophenyl) ethyl]-1- cyclobutyl-urea |
| 185 | 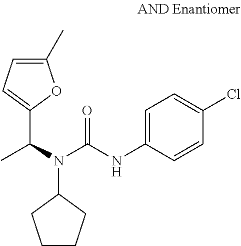 [SMILES: Cc1ccc([C@H](C)N(C(=O)Nc2ccc(Cl)cc2)C2CCCC2)o1] | 347.7 | | 1H NMR (500 MHz, DMSO-d6): δ = 8.34 (s, 1H), 7.51-7.47 (m, 2H), 7.27-7.24 (m, 2H), 6.22 (d, J = 2.9 Hz, 1H), 6.03-5.99 (m, 1H), 5.09-5.05 (m, 1H), 3.62- 3.57 (m, 1H), 2.22 (s, 3H), 1.94- 1.82 (m, 2H), 1.74-1.60 (m, 3H), 1.47 (d, J = 6.6 Hz, 3H), 1.46- 1.22 (m, 3H) ppm | 1-(5-methyl- 2- furyl) ethanone | General method A using cyclopentanamine, AcOH and NaCNBH₃, method D1 using 4- cyanophenylisocyanate | 3-(4- chlorophenyl)-1- cyclopentyl-1- [1-(5-methyl-2- furyl)ethyl]urea |
| 186 | 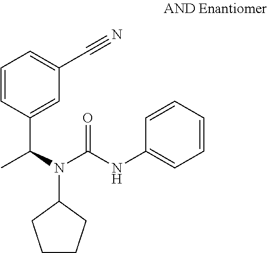 [SMILES: C[C@@H](c1cccc(C#N)c1)N(C(=O)Nc1ccccc1)C1CCCC1] | 334.8 | | 1H NMR (500 MHz, DMSO-d6): δ = 8.31 (s, 1H), 7.75 (s, 1H), 7.71 (d, J = 7.5 Hz, 1H), 7.67 (d, J = 8.0 Hz, 1H), 7.57 (t, J = 8.0 Hz, 1H), 7.41 (d, J = 8.5 Hz, 2H), 7.21 (t, J = 7.5 Hz, 2H), 6.92 (t, J = 7.5 Hz, 1H), 5.02-4.98 (m, 1H), 3.76-3.71 (m, 1H), 1.89-1.84 (m, 1H), 1.82-1.67 (m, 4H), 1.65 (d, J = 7.0 Hz, 3H), 1.49-1.36 (m, 3H) ppm | 3- acetyl- benzonitrile | General method A using cyclopentanamine, AcOH and NaCNBH₃, method D1 using phenylisocyanate | 1-[1-(3- cyanophenyl) ethyl]-1- cyclopentyl-3- phenyl-urea |
| 187 | 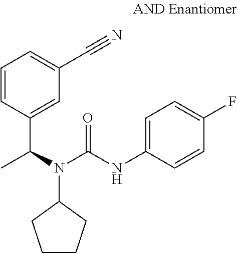 [SMILES: C[C@@H](c1cccc(C#N)c1)N(C(=O)Nc1ccc(F)cc1)C1CCCC1] | 352.8 | | 1H NMR (500 MHz, DMSO-d6): δ = 8.40 (s, 1H), 7.75 (s, 1H), 7.71 (d, J = 7.5 Hz, 1H), 7.66 (d, J = 8.0 Hz, 1H), 7.56 (t, J = 8.0 Hz, 1H), 7.44-7.40 (m, 2H), 7.07- 7.02 (m, 2H), 5.00-4.96 (m, 1H), 3.78-3.74 (m, 1H), 1.87-1.68 (m, 5H), 1.65 (d, J = 7.0 Hz, 3H), 1.49-1.33 (m, 3H) ppm | 3- acetyl- benzonitrile | General method A using cyclopentanamine, AcOH and NaCNBH₃, method D1 using 4- fluorophenylisocyanate | 1-[1-(3- cyanophenyl) ethyl]-1- cyclopentyl-3- (4- fluorophenyl)urea |
| 188 | 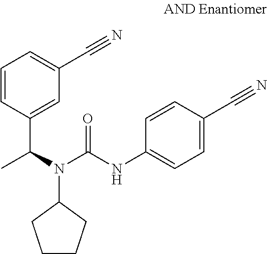 [SMILES: C[C@@H](c1cccc(C#N)c1)N(C(=O)Nc1ccc(C#N)cc1)C1CCCC1] | 359.8 | | 1H NMR (500 MHz, DMSO-d6): δ = 8.88 (s, 1H), 7.76 (s, 1H), 7.72 (d, J = 7.5 Hz, 1H), 7.67-7.63 (m, 5H), 7.57 (t, J = 8.0 Hz, 1H), 5.06-4.99 (m, 1H), 3.78-3.68 (m, 1H), 1.91-1.69 (m, 5H), 1.66 (d, J = 7.0 Hz, 3H), 1.47-1.33 (m, 3H) ppm | 3- acetyl- benzonitrile | General method A using cyclopentanamine, AcOH and NaCNBH₃, method D1 using 4- cyanophenylisocyanate | 3-(4- cyanophenyl)-1- [1-(3- cyanophenyl) ethyl]-1- cyclopentyl-urea |
| 189 | 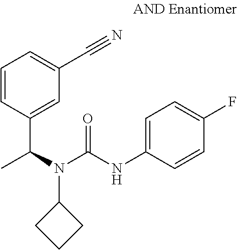 [SMILES: C[C@@H](c1cccc(C#N)c1)N(C(=O)Nc1ccc(F)cc1)C1CCC1] | 338.8 | | 1H NMR (600 MHz, DMSO-d6): δ = 8.26 (s, 1H), 7.72 (s, 1H), 7.71 (d, J = 7.2 Hz, 1H), 7.65-7.64 (m, 1H), 7.55 (t, J = 7.8 Hz, 1H), 7.43-7.39 (m, 2H), 7.06-7.02 (m, 2H), 5.04-5.00 (m, 1H), 4.23- 4.17 (m, 1H), 2.31-2.21 (m, 2H), 2.16-2.07 (m, 2H), 1.66 (d, J = 7.2 Hz, 3H), 1.63-1.57 (m, 1H), 1.55-1.47 (m, 1H) ppm | 3- acetyl- benzonitrile | General method A using cyclobutanamine, AcOH and NaCNBH₃, method D1 using fluorophenylisocyanate | 1-[1-(3- cyanophenyl) ethyl]-1- cyclobutyl- 3-(4- fluorophenyl)urea |
| 190 | 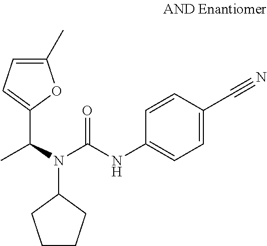 [SMILES: Cc1ccc([C@H](C)N(C(=O)Nc2ccc(C#N)cc2)C2CCCC2)o1] | 338.7 | | 1H NMR (500 MHz, DMSO-d6): δ = 8.75 (s, 1H), 7.66 (s, 4H), 6.25 (d, J = 2.8 Hz, 1H), 6.04-6.00 (m, 1H), 5.13-5.09 (m, 1H), 3.62- 3.54 (m, 1H), 2.21 (s, 3H), 1.93- 1.82 (m, 2H), 1.74-1.63 (m, 3H), 1.48 (d, J = 6.5 Hz, 3H), 1.45- 1.23 (m, 3H) ppm | 1-(5-methyl- 2- furyl) ethanone | General method A using cyclopentanamine, AcOH and NaCNBH₃, method D1 using 4- cyanophenylisocyanate | 3-(4- cyanophenyl)-1- cyclopentyl-1- [1-(5-methyl-2- furyl)ethyl]urea |
| 191 | 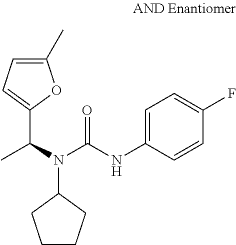 [SMILES: Cc1ccc([C@H](C)N(C(=O)Nc2ccc(F)cc2)C2CCCC2)o1] | 331.7 | | 1H NMR (500 MHz, DMSO-d6): δ = 8.23 (s, 1H), 7.45-7.42 (m, 2H), 7.07-7.02 (m, 2H), 6.21 (d, J = 2.8 Hz, 1H), 6.04-6.00 (m, 1H), 5.09-5.05 (m, 1H), 3.63- 3.56 (m, 1H), 2.22 (s, 3H), 1.93- 1.85 (m, 2H), 1.72-1.61 (m, 3H), 1.48 (d, J = 6.8 Hz, 3H), 1.43- 1.28 (m, 3H) ppm | 1-(5-methyl- 2- furyl) ethanone | General method A using cyclopentaneamine, AcOH and NaCNBH₃, method D1 using 4- fluorophenylisocyanate | 1-cyclopentyl-3- (4- fluorophenyl)-1- [1-(5-methyl-2- furyl)ethyl]urea |
| 192 | 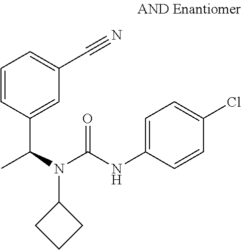 [SMILES: C[C@@H](c1cccc(C#N)c1)N(C(=O)Nc1ccc(Cl)cc1)C1CCC1] | 354.8 | | 1H NMR (600 MHz, DMSO-d6): δ = 8.36 (s, 1H), 7.72 (s, 1H), 7.70 (d, J = 7.2 Hz, 1H), 7.64 (d, J = 7.8 Hz, 1H), 7.55 (t, J = 7.2 Hz, 1H), 7.45-7.44 (m, 2H), 7.32- 7.24 (m, 2H), 5.04-5.00 (m, 1H), 4.24-4.19 (m, 1H), 2.31-2.21 (m, 2H), 2.16-2.07 (m, 2H), 1.66 (d, J = 6.7 Hz, 3H), 1.63-1.58 (m, 1H), 1.55-1.49 (m, 1H) ppm | 3- acetyl- benzonitrile | General method A using cyclobutanamine, AcOH and NaCNBH₃, method D1 using 4- chlorophenylisocyanate | 3-(4- chlorophenyl)-1- [1-(3- cyanophenyl) ethyl]-1- cyclobutyl-urea |
| 193 | 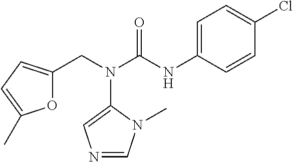 [SMILES: Cc1ccc(CN(C(=O)Nc2ccc(Cl)cc2)c2cncn2C)o1] | 345.7 | | 1H NMR (600 MHz, DMSO-d6): δ = 8.25 (s, 1H), 7.61 (s, 1H), 7.48- 7.46 (m, 2H), 7.29-7.27 (m, 2H), 7.73 (d, J = 0.6 Hz, 1H), 6.06 (d, J = 3.2 Hz, 1H), 5.97-5.94 (m, 1H), 4.67 (s, 2H), 3.27 (s, 3H), 2.22 (s, 3H) ppm | 5- methylfuran- 2- carbaldehyde | General method A using 1-methyl-1H- imidazol-5-amine hydrochloride, Ti(iPrO)₄ and NaBH₄, method D1 using 4- chlorophenylisocyanate | 3-(4- chlorophenyl)-1- [(5-methyl-2- furyl)methyl]-1- (3- methylimidazol- 4-yl)urea |
| 194 | 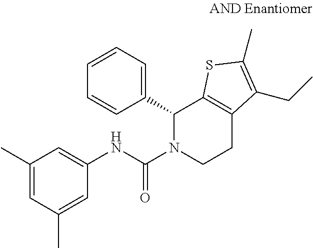 [SMILES: CCc1c(C)sc2c1CCN(C(=O)Nc1cc(C)cc(C)c1)[C@@H]2c1ccccc1] | | | | | | N-(3,5- dimethylphenyl)- 3-ethyl-2- methyl-7- phenyl-5,7- dihydro-4H- thieno[2,3- c]pyridine-6- carboxamide |
| 195 | 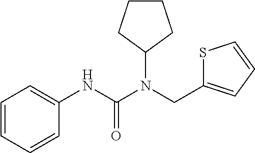 [SMILES: O=C(Nc1ccccc1)N(Cc1cccs1)C1CCCC1] | | | | | | 1-cyclopentyl-3- phenyl-1-(2- thienylmethyl) urea |
| 196 | 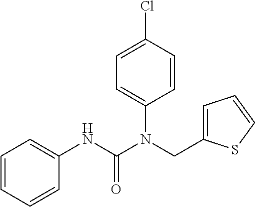 [SMILES: O=C(Nc1ccccc1)N(Cc1cccs1)c1ccc(Cl)cc1] | | | | | | 1-(4- chlorophenyl)-3- phenyl-1-(2- thienylmethyl) urea |
| 197 | 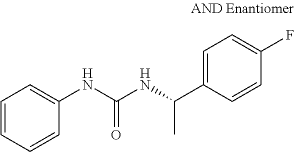 [SMILES: C[C@H](NC(=O)Nc1ccccc1)c1ccc(F)cc1] | | | | | | 1-[1-(4- fluorophenyl) ethyl]-3-phenyl- urea |
| 198 | 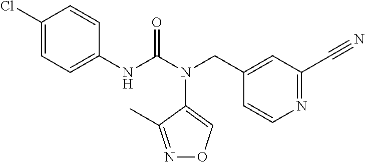 [SMILES: Cc1nocc1N(Cc1ccnc(C#N)c1)C(=O)Nc1ccc(Cl)cc1] | | 365.99 | 1H NMR (300 MHz, DMSO-d6) δ: 9.05 (s, 1H), 8.70 (d, J = 4.9 Hz, 1H), 8.63 (s, 1H), 7.93 (br.s., 1H), 7.66-7.61 (m, 1H), 7.52-7.44 (m, 2H), 7.33-7.25 (m, 2H), 4.81 (s, 2H), 2.12 (s, 3H) ppm. | 4- formyl- pyridine-2- carbonitrile | General method A using 3- methylisooxazol-4- amine hydrochloride, TEA, molecular sieves and NaBH₄, method D1 using 4- chlorophenylisocyanate | 3-(4- chlorophenyl)-1- [(2-cyano-4- pyridyl)methyl]- 1-(3- methylisoxazol- 4-yl)urea |
| 199 | 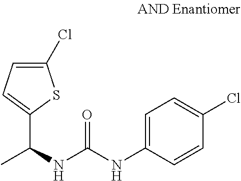 [SMILES: C[C@H](NC(=O)Nc1ccc(Cl)cc1)c1ccc(Cl)s1] | | | | | | 1-(4- chlorophenyl)-3- [1-(5-chloro-2- thienyl)ethyl]urea |
| 200 | 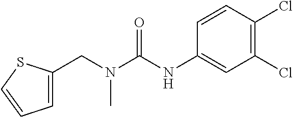 [SMILES: CN(Cc1cccs1)C(=O)Nc1ccc(Cl)c(Cl)c1] | | | | | | 3-(3,4- dichlorophenyl)- 1-methyl-1-(2- thienylmethyl) urea |
| 201 | 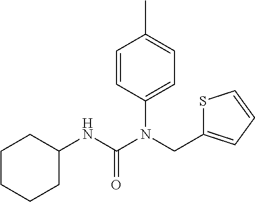 [SMILES: Cc1ccc(N(Cc2cccs2)C(=O)NC2CCCCC2)cc1] | | | | | | 3-cyclohexyl-1- (p-tolyl)-1-(2- thienylmethyl) urea |
| 202 | 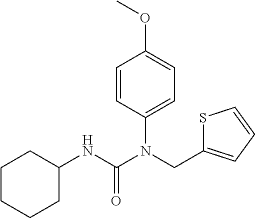 [SMILES: COc1ccc(N(Cc2cccs2)C(=O)NC2CCCCC2)cc1] | | | | | | 3-cyclohexyl- (4- methoxyphenyl)- 1-(2- thienylmethyl) urea |
| 203 | 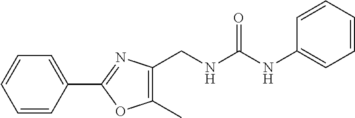 [SMILES: Cc1oc(-c2ccccc2)nc1CNC(=O)Nc1ccccc1] | | | | | | 1-[(5-methyl-2- phenyl-oxazoI-4- yl)methyl]-3- phenyl-urea |
| 204 | 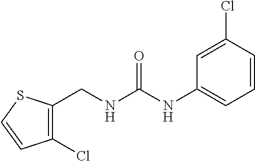 [SMILES: O=C(NCc1sccc1Cl)Nc1cccc(Cl)c1] | | | | | | 1-(3- chlorophenyl)- [(3-chloro-2- thienyl)methyl] urea |
| 205 | 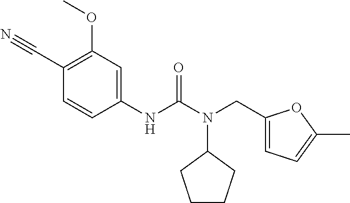 [SMILES: COc1cc(NC(=O)N(Cc2ccc(C)o2)C2CCCC2)ccc1C#N] | 354.25 | | 1H NMR (300 MHz, DMSO-d6) δ: 8.75 (s, 1H), 7.52 (d, J = 8.5 Hz, 1H), 7.48-7.44 (m, 1H), 7.22 (dd, J = 8.5 Hz, 1.6 Hz, 1H), 6.09 (d, J = 2.9 Hz, 1H), 5.98-5.93 (m, 1H), 4.51-4.36 (m, 1H), 4.46 (s, 2H), 3.83 (s, 3H), 2.20 (s, 3H), 1.85-1.41 (m, 8H). ppm. | 5- methylfuran- 2- carbaldehyde | General method A using cyclopentanamine and NaBH₄, method R using isopropenyl chloroformate, method D6 | 3-(4-cyano-3- methoxy- phenyl)-1- cyclopentyl-1- [(5-methyl-2- furyl)methyl]urea |
| 206 | 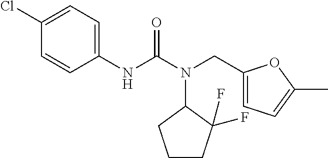 [SMILES: Cc1ccc(CN(C(=O)Nc2ccc(Cl)cc2)C2CCCC2(F)F)o1] | 369.7 | | 1H NMR (300 MHz, DMSO-d6) δ: 8.57 (s, 1H), 7.52-7.41 (m, 2H), 7.33-7.23 (m, 2H), 6.12 (d, J = 2.8 Hz, 1H), 5.99-5.91 (m, 1H), 4.97- 4.75 (m, 2H), 4.31 (d, J = 17.7 Hz, 1H), 2.19 (br.s., 3H), 2.16- 1.88 (m, 4H), 1.87-1.73 (m, 1H), 1.72-1.55 (m, 1H) ppm | 5- methylfuran- 2- carbaldehyde | General method A using 2,2- difluorocyclopentane-1- amine hydrochloride, TEA and NaBH₄, method D1 using 4- chlorophenylisocyanate | 3-(4- chlorophenyl)-1- (2,2- difluorocyclo- pentyl)-1-[(5- methyl-2- furyl)methyl]urea |
| 207 | 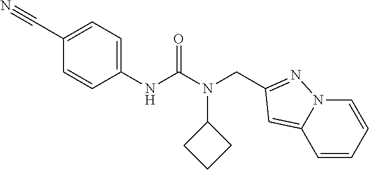 [SMILES: N#Cc1ccc(NC(=O)N(Cc2cc3ccccn3n2)C2CCC2)cc1] | 346.8 | | 1HNMR (300 MHz, CDCl3) δ: 8.90 (br. s, 1H), 8.50-8.43 (m, 1H), 7.60-7.50 (m, 5H), 7.23-7.15 (m, 1H), 6.84 (td, J = 6.8, 1.3 Hz, 1H), 6.47 (s, 1H), 4.78-4.65 (m, 1H), 4.63 (s, 2H), 2.36-2.14 (m, 4H), 1.81-1.65 (m, 2H) ppm | pyrazolo(1,5- a)pyridine-2- carbaldehyde | General method A using cyclobutanamine and NaBH₄, method D1 using 4- cyanophenylisocyanate | 3-(4- cyanophenyl)-1- cyclobutyl- (pyrazolo[1,2- a]pyridin-2- ylmethyl)urea |
| 208 | 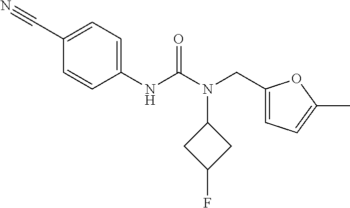 [SMILES: Cc1ccc(CN(C(=O)Nc2ccc(C#N)cc2)C2CC(F)C2)o1] | 328.8 | | 1H NMR (300 MHz, DMSO-d6) δ: 8.90 (s, 1H), 7.76-7.59 (m, 4H), 6.12 (d, J = 2.9 Hz, 1H), 5.99-5.95 (m, 1H), 4.97-4.66 (m, 1H), 4.62- 4.53 (m, 2H), 3.96-3.77 (m, 1H), 2.78-2.59 (m, 2H), 2.42-2.20 (m, 2H), 2.19 (s, 3H) ppm | 5- methylfuran- 2- carbaldehyde | General method A using 3- fluorocyclobutan-1- amine hydrochloride, TEA and NaBH₄, method D1 using 4- chlorophenylisocyanate | 3-(4- cyanophenyl)- (3- fluorocyclobutyl)- 1-[(5-methyl- 2- furyl)methyl]urea |
| 209 | 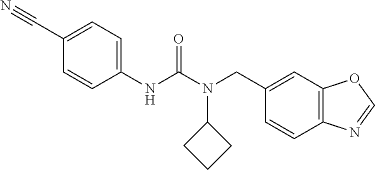 [SMILES: N#Cc1ccc(NC(=O)N(Cc2ccc3ncoc3c2)C2CCC2)cc1] | 347.7 | | 1HNMR (600 MHz, CDCl3) δ: 8.11 (s, 1H), 7.79 (d, J = 8.1 Hz, 1H), 7.56-7.50 (m, 3H), 7.43-7.39 (m, 2H), 7.32 (d, J = 8.3 Hz, 1H), 6.57 (br. s, 1H), 4.76 (s, 2H), 4.58-4.50 (m, 1H), 2.32-2.26 (m, 2H), 2.24-2.15 (m, 2H), 1.80-1.69 (m, 2H) ppm | 1,3- benzoxazole- 6- carbaldehyde | General method A using cyclobutanamine, magnesium perchlorate and NaBH₄, method D1 using 4- cyanophenylisocyanate | 1-(1,3- benzoxazol-6- ylmethyl)-3-(4- cyanophenyl)-1- cyclobutyl-urea |
| 210 | 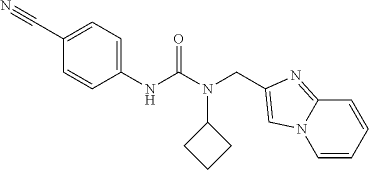 [SMILES: N#Cc1ccc(NC(=O)N(Cc2cn3ccccc3n2)C2CCC2)cc1] | 346.7 | | 1HNMR (300 MHz, DMSO-d6) δ: 9.84 (br. s, 1H), 8.52 (d, J = 6.8 Hz, 1H), 7.87 (s, 1H), 7.69 (s, 4H), 7.58 (d, J = 9.0 Hz, 1H), 7.31-7.20 (m, 1H), 6.96-6.85 (m, 1H), 4.67 (s, 2H), 4.63-4.49 (m, 1H), 2.25-2.00 (m, 4H), 1.72-1.46 (m, 2H) ppm | imidazo(1,2- a)pyridine-2- carbaldehyde | General method A using cyclobutanamine, acetic acid and NaBH₄, method D1 using 4- cyanophenylisocyanate | 3-(4- cyanophenyl)-1- cyclobutyl-1- (imidazo[1,2- a]pyridin-2- ylmethyl)urea |
| 211 | 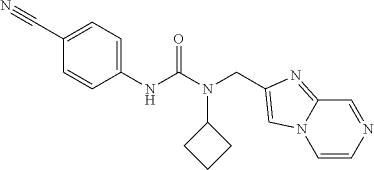 [SMILES: N#Cc1ccc(NC(=O)N(Cc2cn3ccncc3n2)C2CCC2)cc1] | 347.7 | | 1HNMR (300 MHz, DMSO-d6) δ: 9.16 (br. s, 1H), 9.03 (d, J = 0.8 Hz, 1H), 8.55 (dd, J = 4.5 Hz, 1.4 Hz, 1H), 8.01 (s, 1H), 7.87 (d, J = 4.4 Hz, 1H), 7.68 (s, 4H), 4.78 (s, 2H), 4.56 (m, 1H), 2.22-2.02 (m, 4H), 1.68-1.47 (m, 2H) ppm. | imidazo(1,2- a)pyridine-2- carbaldehyde | General method A using cyclobutanamine, and NaBH₄, method D1 using 4- cyanophenylisocyanate | 3-(4- cyanophenyl)- cyclobutyl-1- (imidazo[1,2- a]pyrazin-2- ylmethyl)urea |
| 212 | 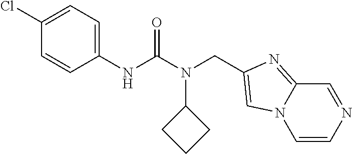 [SMILES: O=C(Nc1ccc(Cl)cc1)N(Cc1cn2ccncc2n1)C1CCC1] | 356.7 | | 1HNMR (300 MHz, DMSO-d6) δ: 9.02 (d, J = 0.8 Hz, 1H), 8.77 (s, 1H), 8.55 (dd, J = 4.5 Hz, 1.5 Hz, 1H), 7.99 (s, 1H), 7.86 (d, J = 4.4 Hz, 1H), 7.56-7.47 (m, 2H), 7.32-7.23 (m, 2H), 4.76 (s, 2H), 4.56 (m, 1H), 2.21-2.02 (m, 4H), 1.70-1.46 (m, 2H) ppm. | imidazo(1,2- a)pyridine-2- carbaldehyde | General method A using cyclobutanamine, and NaBH₄, method D1 using 4- cyanophenylisocyanate | 3-(4- chlorophenyl)-1- cyclobutyl-1- (imidazo[1,2- a]pyrazin-2- ylmethyl)urea |
| 213 | 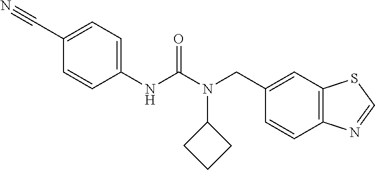 [SMILES: N#Cc1ccc(NC(=O)N(Cc2ccc3ncsc3c2)C2CCC2)cc1] | | 361.5 | 1HNMR (300 MHz, CDCl3) δ : 9.00 (s, 1H), 8.13 (d, J = 8.5 Hz, 1H), 7.90-7.86 (m, 1H), 7.55-7.49 (m, 2H), 7.47-7.39 (m, 3H), 6.49 (br. s, 1H), 4.78 (s, 2H), 4.59-4.44 (m, 1H), 2.36-2.24 (m, 2H), 2.24- 2.13 (m, 2H), 1.81-1.67 (2H) ppm. | 1,3- benzothiazole- 6- carbaldehyde | General method A cyclobutanamine, magnesium perchlorate and NaBH₄, method D1 using 4- cyanophenylisocyanate | 1-(1,3- benzothiazol-6- ylmethyl)-3-(4- cyanophenyl)- cyclobutyl-urea |
| 214 | 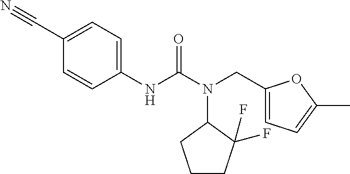 [SMILES: Cc1ccc(CN(C(=O)Nc2ccc(C#N)cc2)C2CCCC2(F)F)o1] | 360.7 | | 1H NMR (300 MHz, DMSO-d6) δ: 8.93 (s, 1H), 7.78-7.55 (m, 4H), 6.11 (d, J = 2.9 Hz, 1H), 5.91-5.91 (m, 1H), 4.96-4.75 (m, 2H), 4.34 (d, J = 17.8 Hz, 1H), 2.17 (br.s., 3H), 2.23-1.91 (m, 4H), 1.88-1.74 (m, 1H), 1.73-1.58 (m, 1H) ppm | 5- methylfuran- 2- carbaldehyde | General method A using 2,2- difluorocyclopentane-1- amine hydrochloride, TEA and NaBH₄, method D1 using 4- cyanophenylisocyanate | 3-(4- cyanophenyl)- (2,2- difluorocyclo- pentyl)-1-[(5- methyl-2- furyl)methyl]urea |
| 215 | 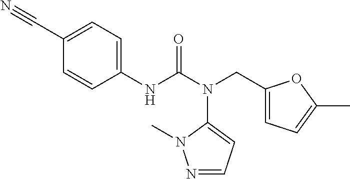 [SMILES: Cc1ccc(CN(C(=O)Nc2ccc(C#N)cc2)c2ccnn2C)o1] | 336.8 | | 1H NMR (300 MHz, DMSO-d6): δ = 8.71 (bs, 1H), 7.68 (s, 4H), 7.42 (d, J = 2.0 Hz, 1H), 6.11 (d, J = 2.0 Hz, 1H), 6.08 (d, J = 3.0 Hz, 1H), 5.95 (dd, J = 3.0, 1.0 Hz, 1H), 4.72 (s, 2H), 3.45 (s, 3H), 2.20 (s, 3H) ppm. | 5- methylfuran- 2- carbaldehyde | General method A using 1-methyl-1H- pyrazol-5-amine, Na₂SO₄ and NaBH₄, method D1 using 4- cyanophenylisocyanate | 3-(4- cyanophenyl)-1- [(5-methyl-2- furyl)methyl]-1- (2- methylpyrazol- 3-yl)urea |
| 216 | 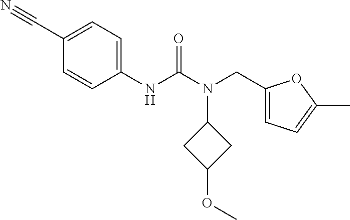 [SMILES: COC1CC(N(Cc2ccc(C)o2)C(=O)Nc2ccc(C#N)cc2)C1] | 340.7 | | 1H NMR (300 MHz, DMSO-d6) δ: 8.76-8.80 (m, 1H), 7.70-7.60 (m, 4H), 6.10-6.06 (m, 1H), 5.97- 5.93 (m, 1H), 4.65-4.55 (m, 1H), 4.55-4.50 (m, 2H), 3.88-3.48 (m, 1 H), 3.16-3.10 (m, 3H), 2.37- 2.13 (m, 3H), 2.18 (s, 3H), 1.98- 1.82 (m, 1H) ppm | 5- methylfuran- 2- carbaldehyde | General method A using 3- methoxycyclobutan-1- amine, TEA and NaBH₄, method D1 using 4- cyanophenylisocyanate | 3-(4- cyanophenyl)-1- (3- methoxycyclo- butyl)-1-[(5- methyl-2- furyl)methyl]urea |
| 217 | 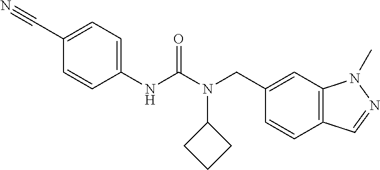 [SMILES: Cn1ncc2ccc(CN(C(=O)Nc3ccc(C#N)cc3)C3CCC3)cc21] | 360.8 | | 1HNMR (300 MHz, CDCl3) δ: 7.98 (d, J = 0.9 Hz, 1H), 7.74 (dd, J = 8.3 Hz, 0.6 Hz, 1H), 7.53-7.47 (m, 2H), 7.39-7.33 (m, 2H), 7.27 (br. s, 1H), 7.08 (dd, J = 8.3 Hz, 1.4 Hz, 1H), 6.49 (br. s, 1H), 4.77 (s, 2H), 4.72-4.58 (m, 1H), 4.06 (s, 3H), 2.36-2.24 (m, 2H), 2.24- 2.12 (m, 2H), 1.80-1.65 (m, 2H) ppm | 1- methyl- indazole-6- carbaldehyde | General method A using molecular sieves, cyclobutanamine and NaBH₄, method D1 using 4- cyanophenylisocyanate | 3-(4- cyanophenyl)-1- cyclobutyl-1-[(1- methylindazol-6- yl)methyl]urea |
| 218 | 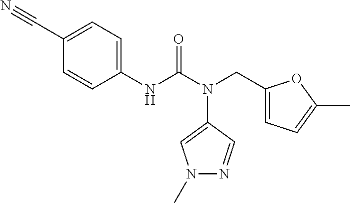 [SMILES: Cc1ccc(CN(C(=O)Nc2ccc(C#N)cc2)c2cnn(C)c2)o1] | 336.7 | | 1HNMR (300 MHz, CDCl3) δ: 7.49-7.43 (m, 2H), 7.43-7.37 (m, 2H), 7.34 (s, 1H), 7.20 (s, 1H), 6.71 (s, 1H), 6.02 (d, J = 3.0 Hz, 1H), 5.84-5.80 (m, 1H), 4.64 (s, 2H), 3.87 (s, 3H), 2.21 (s, 3H) ppm. | 5- methylfuran- 2- carbaldehyde | General method A using 1-methylpyrazol- 4-amine dihydrochloride, magnesium perchlorate, TEA and NaBH₄, method D1 using 4- cyanophenyl-isocyanate | 3-(4- cyanophenyl)-1- [(5-methyl-2- fiiryl)methyl]-1- (1- methylpyrazol- 4-yl)urea |
| 219 | 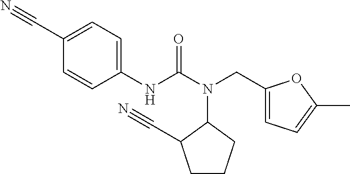 [SMILES: Cc1ccc(CN(C(=O)Nc2ccc(C#N)cc2)C2CCCC2C#N)o1] | 349.8 | | 1H NMR (300 MHz, DMSO-d6) δ: 8.99-8.93 (m, 1H), 7.75-7.61 (m, 4H), 6.23-6.16 (m, 1H), 6.01- 5.94 (m, 1H), 4.94-4.62 (m, 1H), 4.62-4.43 (m, 2H), 3.49-3.15 (m, 1H), 2.20 (s, 3H), 2.17-1.87 (m, 2H), 1.85-1.60 (m, 4H) ppm | 5- methylfuran- 2- carbaldehyde | General method A using 2- aminocyclopentane-1- carbonitrile, TEA and NaBH₄, method D1 using 4- cyanophenylisocyanate | 1-(2- cyanocyclo- pentyl)-3-(4- cyanophenyl)- [(5-methyl-2- furyl)methyl]urea |
| 220 | 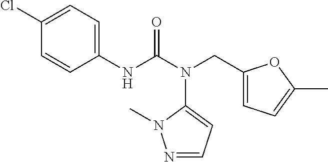 [SMILES: Cc1ccc(CN(C(=O)Nc2ccc(Cl)cc2)c2ccnn2C)o1] | 345.8 | | 1H NMR (300 MHz, DMSO-d6): δ = 8.37 (bs, 1H), 7.47 (d, J = 9.0 Hz, 2H), 7.41 (d, J = 2.0 Hz, 1H), 7.28 (d, J = 8.9 Hz, 2H), 6.09 (d, J = 2.0 Hz, 1H), 6.06 (d, J = 3.0 Hz, 1H), 5.95 (dd, J = 3.0, 1.0 Hz, 1H), 4.70 (s, 2H), 3.45 (s, 3H), 2.20 (s, 3H) ppm. | 5- methylfuran- 2- carbaldehyde | General method A using 1-methyl-1H- pyrazol-5 amine, Na₂SO₄ and NaBH₄, method D1 using 4- chlorophenylisocyanate | 3-(4- chlorophenyl)-1- [(5-methyl-2- furyl)methyl]-1- (2- methylpyrazol- 3-yl)urea |
| 221 | 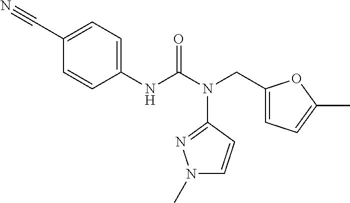 [SMILES: Cc1ccc(CN(C(=O)Nc2ccc(C#N)cc2)c2ccn(C)n2)o1] | 336.7 | | 1HNMR (300 MHz, CDCl3) δ: 11.13 (s, 1H), 7.71-7.62 (m, 2H), 7.60-7.52 (m, 2H), 7.31 (d, J = 2.3 Hz, 1H), 6.16 (d, J = 3.1 Hz, 1H), 6.10 (d, J = 2.6 Hz, 1H), 5.89-5.86 (m, 1H), 4.94 (s, 2H), 3.89 (s, 3H), 2.24 (s, 3H) ppm. | 5- methylfuran- 2- carbaldehyde | General method A using 1-methylpyrazol- 3-amine, magnesium perchlorate and NaBH₄, method D1 using 4- cyanophenyl-isocyanate | 3-(4- cyanophenyl)-1- [(5-methyl-2- furyl)methyl]-1- (1- methylpyrazol- 3-yl)urea |
| 222 | 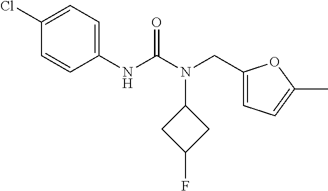 [SMILES: Cc1ccc(CN(C(=O)Nc2ccc(Cl)cc2)C2CC(F)C2)o1] | 337.7 | | 1H NMR (300 MHz, DMSO-d6) δ: 8.53 (d, J = 4.7 Hz, 1H), 7.51- 7.42 (m, 2H), 7.30-7.23 (m, 2H), 6.10 (d, J = 3.0 Hz, 1H), 5.99-5.94 (m, 1H), 5.28-4.65 (m, 1H), 4.53 (d, J = 7.0 Hz, 2H), 3.92-3.78 (m, 1H), 2.73-2.51 (m, 2H), 2.44-2.21 (m, 2H), 2.19 (s, 3H) ppm | 5- methylfuran- 2- carbaldehyde | General method A using 3- fluorocyclobutan-1- amine hydrochloride, TEA and NaBH₄, method D1 using 4- chlorophenyl- isocyanate | 3-(4- chlorophenyl)-1- (3- fluorocyclobutyl)- 1-[(5-methyl- 2- furyl)methyl]urea |
| 223 | 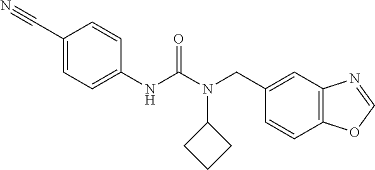 [SMILES: N#Cc1ccc(NC(=O)N(Cc2ccc3ocnc3c2)C2CCC2)cc1] | 347.7 | | 1HNMR (600 MHz, CDCl3) δ: 8.13 (s, 1H), 7.73 (s, 1H), 7.59 (d, J = 8.5 Hz, 1H), 7.53-7.50 (m, 2H), 7.42-7.39 (m, 2H), 7.38-7.35 (m, 1H), 6.61 (br. s., 1H), 4.74 (s, 2H), 4.51 (m, 1H), 2.33-2.25 (m, 2H), 2.25-2.15 (m, 2H), 1.82-1.67 (m, 2H) ppm. | 1,3- benzoxazole- 6- carbaldehyde | General method A using cyclobutanamine, magnesium perchlorate and NaBH₄, method D1 using 4- cyanophenylisocyanate | 1-(1,3- benzoxazol-5- ylmethyl)-3-(4- cyanophenyl)-1- cyclobutyl-urea |
| 224 | 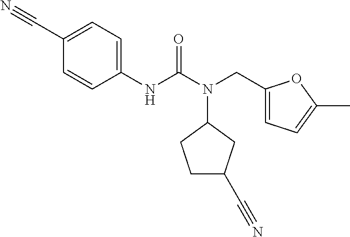 [SMILES: Cc1ccc(CN(C(=O)Nc2ccc(C#N)cc2)C2CCC(C#N)C2)o1] | 349.7 | | 1H NMR (300 MHz, DMSO) δ: 8.83 (br.s., 1H), 7.71-7.61 (m, 4H), 6.14 (d, J = 3.2 Hz, 1H), 5.99-5.96 (m, 1H), 4.55 (br.s., 2H), 4.51-4.37 (m, 1H), 3.05-2.91 (m, 1H), 2.22 (br.s., 3H), 2.20- 2.12 (m, 1H), 2.04-1.72 (m, 5H) ppm | 5- methylfuran- 2- carbaldehyde | General method A using 3- aminocyclopentane-1- carbonitrile, TEA and NaBH₄, method D1 using 4- cyanophenylisocyanate | 1-(3- cyanocyclo- pentyl)-3-(4- cyanophenyl)-1- [(5-methyl-2- furyl)methyl]urea |
| 225 | 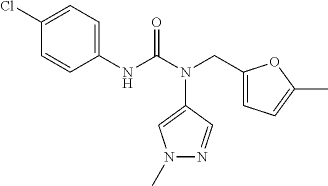 [SMILES: Cc1ccc(CN(C(=O)Nc2ccc(Cl)cc2)c2cnn(C)c2)o1] | 345.7 | | 1HNMR (300 MHz, CDCl3) δ: 7.39 (s, 1H), 7.33-7.25 (m, 3H), 7.23-7.16 (m, 2H), 6.53 (s, 1H), 6.08 (d, J = 2.9 Hz, 1H), 5.90-5.85 (m, 1H), 4.70 (s, 2H), 3.92 (s, 3H), 2.27 (s, 3H) ppm. | 5- methylfuran- 2- carbaldehyde | General method A using 1- methylimidazol-4- amine dihydrochloride, magnesium perchlorate, TEA and NaBH₄, method D1 using 4- chlorophenylisocyanate | 3-(4- chlorophenyl)-1- [(5-methyl-2- furyl)methyl] (1- methylpyrazol- 4-yl)urea |
| 226 | 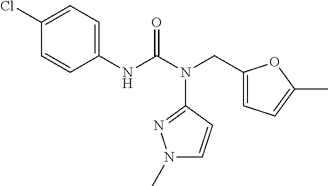 [SMILES: Cc1ccc(CN(C(=O)Nc2ccc(Cl)cc2)c2ccn(C)n2)o1] | 345.7 | | 1HNMR (300 MHz, CDCl3) δ: 10.72 (s, 1H), 7.53-7.45 (m, 2H), 7.30-7.26 (m, 2H), 7.23 (s, 1H), 6.15 (d, J = 3.1 Hz, 1H), 6.07 (d, J = 2.4 Hz, 1H), 5.89-5.83 (m, 1H), 4.94 (s, 2H), 3.86 (s, 3H), 2.24 (s, 3H) ppm. | 5- methylfuran- 2- carbaldehyde | General method A using 1-methypyrazol- 3-amine, magnesium perchlorate and NaBH₄, method D1 using 4- chlorophenylisocyanate | 3-(4- chlorophenyl)-1- [(5-methyl-2- furyl)methyl]-1- (1- methylpyrazol- 3-yl)urea |
| 227 | 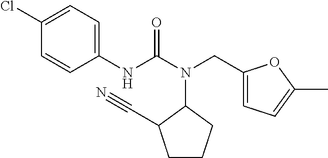 [SMILES: Cc1ccc(CN(C(=O)Nc2ccc(Cl)cc2)C2CCCC2C#N)o1] | 358.7 | | 1H NMR (300 MHz, DMSO) δ: 8.63-8.57 (m, 1H), 7.54-7.44 (m, 2H), 7.32-7.26 (m, 2H), 6.24-6.16 (m, 1H), 6.06-5.95 (m, 1H), 4.92- 4.64 (m, 1H), 4.58-4.40 (m, 2H), 3.48-3.13 (m, 1H), 2.21 (s, 3H), 2.17-1.87 (m, 2H), 1.85-1.60 (m, 4H) ppm. | 5- methylfuran- 2- carbaldehyde | General method A using 2- aminocyclopentane-1- carbonitrile, TEA and NaBH₄, method D1 using 4- chlorophenylisocyanate | 3-(4- chlorophenyl)-1- (2-cyano- cyclopentyl)- 1-[(5-methyl- 2- furyl)methyl]urea |
| 228 | 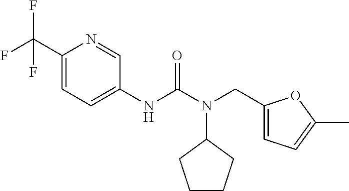 [SMILES: Cc1ccc(CN(C(=O)Nc2ccc(C(F)(F)F)nc2)C2CCCC2)o1] | 368.7 | | 1HNMR (300 MHz, CDCl3) δ: 8.38 (d, J = 2.5 Hz, 1H), 8.22 (dd, J = 8.5 Hz, 2.4 Hz, 1H), 7.59 (d, J = 8.5 Hz, 1H), 7.42 (br. s, 1H), 6.21 (d, J = 3.1 Hz, 1H), 6.00-5.96 (m, 1H), 4.74-4.72 (m, 1H), 4.34 (s, 2H), 2.33 (s, 3H), 2.01-1.88 (m, 2H), 1.80-1.54 (m, 6H) ppm. | 5- methylfuran- 2- carbaldehyde | General method A using cyclopentanamine and NaBH₄, method D2 using 6- (trifluoromethyl)pyridin- 3-amine | 1-cyclopentyl-1- [(5-methyl-2- furyl)methyl]- [6- (trifluoromethyl)- 3-pyridyl]urea |
| 229 | 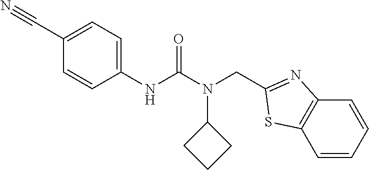 [SMILES: N#Cc1ccc(NC(=O)N(Cc2nc3ccccc3s2)C2CCC2)cc1] | 363.7 | | 1HNMR (300 MHz, CDCl3) δ: 8.57 (br. s, 1H), 8.10-8.03 (m, 1H), 7.93-7.87 (m, 1H), 7.58 (s, 4H), 7.54 (dd, J = 8.1 Hz, 1.1 Hz, 1H), 7.49-7.41 (m, 1H), 4.89 (s, 2H), 4.67-4.52 (m, 1H), 2.41-2.14 (m, 4H), 1.84-1.64 (m, 2H) ppm. | 1,3- benzothiazole- 2- carbaldehyde | General method A using cyclobutanamine and NaBH₄, method D1 using 4- cyanophenylisocyanate | 1-(1,3- benzothiazol-2- ylmethyl)-3-(4- cyanophenyl)-1- cyclobutyl-urea |
| 230 | 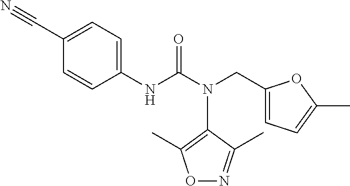 [SMILES: Cc1ccc(CN(C(=O)Nc2ccc(C#N)cc2)c2c(C)noc2C)o1] | | 349.6 | 1HNMR (300 MHz, CDCl3) δ: 7.60-7.49 (m, 4H), 6.68 (br. s, 1H), 6.09 (s, J = 3.0 Hz, 1H), 5.91-5.81 (m, 1H), 4.91-4.71 (m, 1H), 4.56-4.43 (m, 1H), 2.24 (s, 3H), 2.15 (s, 3H), 2.04 (s, 3H) ppm. | 5- methylfuran- 2- carbaldehyde | General method A using 3,3-- dimethylisooxazol-4- amine, magnesium perchlorate and NaBH₄, method D1 using 4- cyanophenylisocyanate | 3-(4- cyanophenyl)-1- (3,5- dimethylisoxazol- 4-yl)-1-[(5- methyl-2- furyl)methyl]urea |
| 231 | 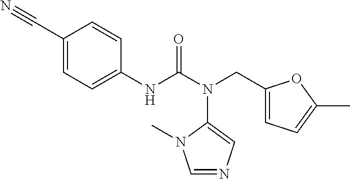 [SMILES: Cc1ccc(CN(C(=O)Nc2ccc(C#N)cc2)c2cncn2C)o1] | 336.7 | | 1HNMR (300 MHz, CDCl3) δ: 7.64 (br. s, 1H), 7.56-7.46 (m, 4H), 7.05 (d, J = 1.5 Hz, 1H), 6.88 (d, J= 1.5 Hz, 1H), 6.11 (d, J = 3.0 Hz, 1H), 5.88-5.83 (m, 1H), 4.88 (s, 2H), 3.36 (s, 3H), 2.22 (s, 3H) ppm | 5- methylfuran- 2- carbaldehyde | General method A using 3- methylimidazol-4- amine hydrochloride, sodium sulfate, TEA and NaBH₄, method D1 using 4-cyanophenyl- isocyanate | 3-(4- cyanophenyl)-1- [(5-methyl-2- furyl)methyl] (3- methylimidazol- 4-yl)urea |
| 232 | 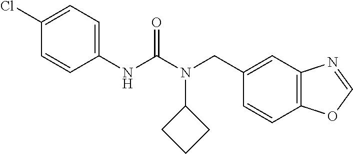 [SMILES: O=C(Nc1ccc(Cl)cc1)N(Cc1ccc2ocnc2c1)C1CCC1] | 347.7 | | 1HNMR (600 MHz, CDCl3) δ: 8.11 (s, 1H), 7.79 (d, J = 8.1 Hz, 1H), 7.56-7.50 (m, 3H), 7.43-7.39 (m, 2H), 7.32 (d, J = 8.3 Hz, 1H), 6.57 (br. s, 1H), 4.76 (s, 2H), 4.50 (m, 1H), 2.32-2.26 (m, 2H), 2.24- 2.15 (m, 2H), 1.80-1.69 (m, 2H) ppm. | 1,3- benzoxazole- 5- carbaldehyde | General method A using cyclobutanamine, magnesium perchlorate and NaBH₄, method D1 using 4- chlorophenylisocyanate | 1-(1,3- benzoxazol-5- ylmethyl)-3-(4- chlorophenyl)-1- cyclobutyl-urea |
| 233 | 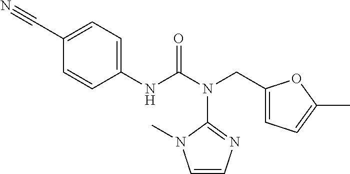 [SMILES: Cc1ccc(CN(C(=O)Nc2ccc(C#N)cc2)c2nccn2C)o1] | 336.9 | | 1H NMR (300 MHz, DMSO-d6): δ = 8.86 (bs, 1H), 7.73-7.64 (m, 4H), 7.09 (d, J = 1.4 Hz, 1H), 6.85 (d, J = 1.4 Hz, 1H), 6.05 (d, J = 3.1 Hz, 1H), 5.93 (dd, J = 3.0, 1.1 Hz, 1H), 4.75 (s, 2H), 3.21 (s, 3H), 2.18 (s, 3H) ppm. | 5- methylfuran- 2- carbaldehyde | General method A using 1- methylimidazol-2- amine hydrochloride, Na₂SO₄, TEA and NaBH₄, method D1 using 4-cyanophenyl- isocyanate | 3-(4- cyanophenyl)-1- [(5-methyl-2- furyl)methyl]-1- (1- methylimidazol- 2-yl)urea |
| 234 | 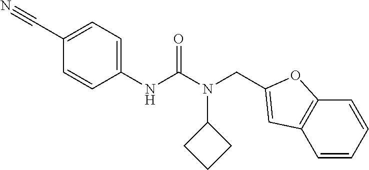 [SMILES: N#Cc1ccc(NC(=O)N(Cc2cc3ccccc3o2)C2CCC2)cc1] | 346.8 | | 1H NMR (300 MHz, DMSO-d6) δ: 8.86 (s, 1H), 7.68 (br.s., 4H), 7.61-7.48 (m, 2H), 7.29-7.15 (m, 2H), 6.68 (d, J = 0.8 Hz, 1H), 4.80 (s, 2H), 4.63-4.39 (m, 1H), 2.25- 2.07 (m, 4H), 1.73-1.49 (m, 2H) ppm | benzofuran- 2- carbaldehyde | General method A using cyclobutanamine, magnesium perchlorate and NaBH₄, method D1 using 4- cyanophenylisocyanate | 1-(benzofuran-2- ylmethyl)-3-(4- cyanophenyl)-1- cyclobutyl-urea |
| 235 | 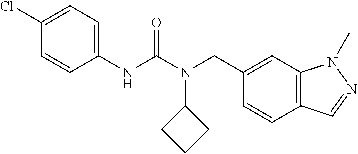 [SMILES: Cn1ncc2ccc(CN(C(=O)Nc3ccc(Cl)cc3)C3CCC3)cc21] | 369.7 | | 1HNMR (300 MHz, CDCl3) δ: 7.97 (d, J = 0.8 Hz, 1H), 7.72 (dd, J = 8.3 Hz, J = 0.4 Hz, 1H), 7.28 (br. s, 1H), 7.19 (s, 4H), 7.08 (dd, J = 8.3 Hz, 1.2 Hz, 1H), 6.28 (br. s, 1H), 4.76 (s, 2H), 4.71-4.58 (m, 1H), 4.06 (s, 3H), 2.36-2.21 (m, 2H), 2.21-2.10 (m, 2H), 1.79-1.61 (m, 2H) ppm | 1- methyl- indazole-6- carbaldehyde | General method A using cyclobutanamine and NaBH₄, method D1 using 4- chlorophenylisocyanate | 3-(4- chlorophenyl)-1- cyclobutyl-1-[(1- methylindazol-6- yl)methyl]urea |
| 236 | 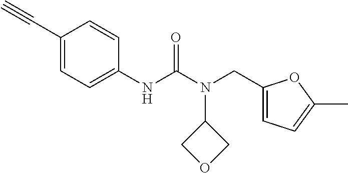 [SMILES: C#Cc1ccc(NC(=O)N(Cc2ccc(C)o2)C2COC2)cc1] | 311.7 | | 1H NMR (300 MHz, DMSO) δ: 8.82 (s, 1H), 7.52-7.41 (m, 2H), 7.39-7.29 (m, 2H), 6.14 (d, J = 3.0 Hz, 1H), 6.00-5.95 (m, 1H), 4.87- 4.73 (m, 1H), 4.61 (br.s, 2H), 4.59 (br.s., 4H), 4.02 (s, 1H), 2.20 (br.s., 3H) ppm | 5- methylfuran- 2- carbaldehyde | General method A using oxetane-3-amine and NaBH₄, method D2 using 4-ethynylaniline | 3-(4- ethynylphenyl)- 1-[(5-methyl-2- furyl)methyl]-1- (oxetan-3- yl)urea |
| 237 | 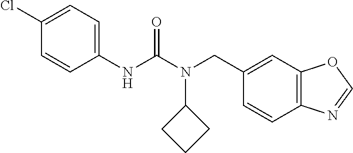 [SMILES: O=C(Nc1ccc(Cl)cc1)N(Cc1ccc2ncoc2c1)C1CCC1] | 356.7 | | 1HNMR (600 MHz, CDCl3) δ: 8.15 (s, 1H), 7.82 (d, J = 8.1 Hz, 1H), 7.62 (s, 1H), 7.38 (d, J = 8.1 Hz, 1H), 7.31-7.21 (m, 4H), 6.62 (br. s, 1H), 4.78 (s, 2H), 4.52 (m, 1H), 2.35-2.28 (m, 2H), 2.28-2.20 (m, 2H), 1.83-1.72 (m, 2H) ppm. | 1,3- benzooxazole- 6- carbaldehyde | General method A using cyclobutanamine, magnesium perchlorate and NaBH₄, method D1 using 4- chlorophenylisocyanate | 1-(1,3- benzoxazol-6- ylmethyl)-3-(4- chlorophenyl)-1- cyclobutyl-urea |
| 238 | 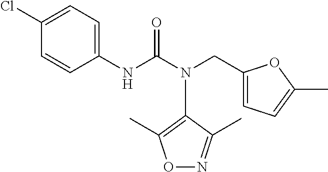 [SMILES: Cc1ccc(CN(C(=O)Nc2ccc(Cl)cc2)c2c(C)noc2C)o1] | 360.5 | | 1HNMR (300 MHz, CDCl3) δ: 7.34-7.37 (m, 2H), 7.25-7.20 (m, 2H), 6.34 (br. s, 1H), 6.08 (d, J = 2.9 Hz, 1H), 5.90-5.84 (m, 1H), 4.81-4.79 (m, 1H), 4.58-4.44 (m, 1H), 2.24 (s, 3H), 2.14 (s, 3H), 2.05 (s, 3H) ppm. | 5- methylfuran- 2- carbaldehyde | General method A using 3,3-- dimethylisooxazol-4- amine, magnesium perchlorate and NaBH₄, method D1 using 4- chlorophenylisocyanate | 3-(4- chlorophenyl)- (3,5- dimethylisoxazol- 4-yl)-1-[(5- methyl-2- furyl)methyl]urea |
| 239 | 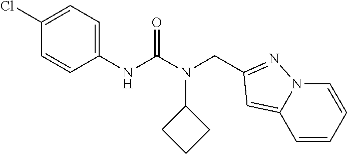 [SMILES: O=C(Nc1ccc(Cl)cc1)N(Cc1cc2ccccn2n1)C1CCC1] | 355.7 | | 1HNMR (300 MHz, CDCl3) δ: 8.45 (dd, J = 7.1 Hz, 0.9 Hz, 1H), 8.19 (br. s, 1H), 7.51 (dt, J = 7.8 Hz, 1.1 Hz, 1H), 7.41-7.34 (m, 2H), 7.24-7.11 (m, 3H), 6.81 (td, J = 6.9 Hz, 1.3 Hz, 1H), 6.46 (s, 1H), 4.77-4.66 (m, 1H), 4.64 (s, 2H), 2.34-2.16 (m, 4H), 1.80-1.63 (m, 2H) ppm. | pyrazolo(1,5- a)pyridine-2- carbaldehyde | General method A using cyclobutanamine and NaBH₄, method D1 using 4- chlorophenylisocyanate | 3-(4- chlorophenyl)-1- cyclobutyl-1- (pyrazolo[1,5- a]pyridin-2- ylmethyl)urea |
| 240 | 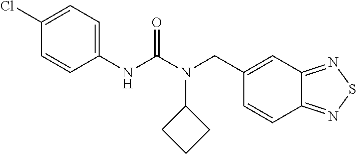 [SMILES: O=C(Nc1ccc(Cl)cc1)N(Cc1ccc2nsnc2c1)C1CCC1] | 373.7 | | 1HNMR (300 MHz, CDCl3) δ: 8.02-7.95 (m, 1H), 7.88-7.83 (m, 1H), 7.55 (dd, J = 9.0 Hz, 1.7 Hz, 1H), 7.33-7.27 (m, 2H), 7.25-7.19 (m, 2H), 6.33 (br. s, 1H), 4.80 (d, J = 0.9 Hz, 2H), 4.48-4.34 (m, 1H), 2.37-2.11 (m, 4H), 1.83-1.66 (m, 2H) ppm. | 2,1,3- benzothia- diazole- 5- carbaldehyde | General method A using cyclobutanamine and NaBH₄, method D1 using 4- chlorophenylisocyanate | 1-(2,1,3- benzothiadiazol- 5-ylmethyl)- (4- chlorophenyl)-1- cyclobutyl-urea |
| 241 | 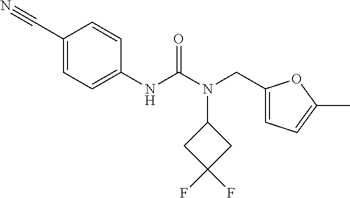 [SMILES: Cc1ccc(CN(C(=O)Nc2ccc(C#N)cc2)C2CC(F)(F)C2)o1] | | 344.7 | 1HNMR (300 MHz, CDCl3) δ: 7.60-7.53 (m, 2H), 7.45-7.35 (m, 2H), 7.32 (br.s., 1H), 6.24 (d, J = 3.0 Hz, 1H), 6.02-5.97 (m, 1H), 4.53-4.42 (m, 1H), 4.37 (s, 2H), 3.06-2.69 (m, 4H), 2.34 (d, J = 0.5 Hz, 3H) ppm. | 5- methylfuran- 2- carbaldehyde | General method A using 3,3- difluorocyclobutane-1- amine, TEA and NaBH₄, method D1 using 4- cyanophenylisocyanate | 3-(4- cyanophenyl) (3,3-difluoro- cyclobutyl)- 1-[(5-methyl- 2- furyl)methyl]urea |
| 242 | 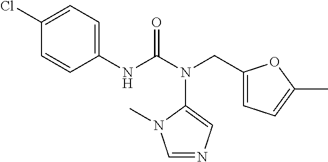 [SMILES: Cc1ccc(CN(C(=O)Nc2ccc(Cl)cc2)c2cncn2C)o1] | 345.8 | | 1H NMR (500 MHz, DMSO-d6) δ : 8.26 (s, 1H), 7.60 (s, 1H), 7.47 (d, J = 8.7 Hz, 2H), 7.28 (d, J = 8.7 Hz, 2H), 6.73 (s, 1H), 6.06 (d, J = 2.8 Hz, 1H), 5.97-5.95 (m, 1H), 4.66 (s, 2H), 3.23 (s, 3H), 2.21 (s, 3H) ppm. | 5- methylfuran- 2- carbaldehyde | General method A using 1-methyl-1H- imidazol-5-amine hydrochloride, Ti(iPrO)₄ and NaBH₄, method D1 using 4- chlorophenylisocyanate | 3-(4- chlorophenyl)-1- [(5-methyl-2- furyl)methyl]-1- (3- methylimidazol- 4-yl)urea |
| 243 | 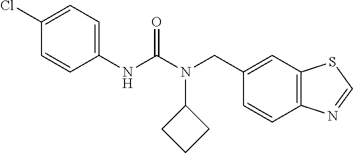 [SMILES: O=C(Nc1ccc(Cl)cc1)N(Cc1ccc2ncsc2c1)C1CCC1] | 372.5 | | 1HNMR (300 MHz, CDCl3) δ : 9.02 (br. s, 1H), 8.12 (d, J = 8.3 Hz, 1H), 7.90 (br. s, 1H), 7.49- 7.40 (m, 1H), 7.26-7.16 (m, 4H), 6.27 (br. s, 1H),4.77(s, 2H), 4.58-4.42 (m, 1H), 2.35-2.22 (m, 2H), 2.22-2.11 (m, 2H), 1.79-1.64 (m, 2H) ppm. | 1,3- benzothiazole- 6- carbaldehyde | General method A cyclobutanamine, magnesium perchlorate and NaBH₄, method D1 using 4- chlorophenylisocyanate | 1-(1,3- benzothiazol-2- ylmethyl)-3-(4- chlorophenyl)-1- cyclobutyl-urea |
| 244 | 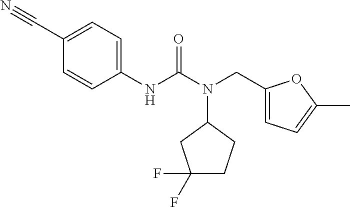 [SMILES: Cc1ccc(CN(C(=O)Nc2ccc(C#N)cc2)C2CCC(F)(F)C2)o1] | 360.7 | | 1H NMR (300 MHz, DMSO) δ: 8.88 (s, 1H), 7.72-7.60 (m, 4H), 6.14 (d, J = 3.0 Hz, 1H), 5.99-5.94 (m, 1H), 4.68-4.57 (m, 1H), 4.55 (s, 2H), 2.40-2.20 (m, 3H), 2.19 (br.s., 3H), 2.04-1.85 (m, 3H) ppm | 5- methylfuran- 2- carbaldehyde | General method A using 3,3- difluorocyclopentane-1- amine hydrochloride, TEA and NaBH₄, method D1 using 4- cyanophenylisocyanate | 3-(4- cyanophenyl)-1- (3,3- difluorocyclo- pentyl)-1-[(5- methyl-2- furyl)methyl]urea |
| 245 | 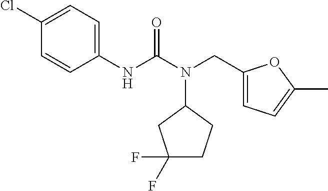 [SMILES: Cc1ccc(CN(C(=O)Nc2ccc(Cl)cc2)C2CCC(F)(F)C2)o1] | 369.7 | | 1H NMR (300 MHz, DMSO) δ: 8.53 (s, 1H), 7.53-7.40 (m, 2H), 7.33-7.22 (m, 2H), 6.14 (d, J = 3.10 Hz, 1H), 5.99-5.95 (m, 1H), 4.65- 4.53 (m, 1H), 4.52 (s, 2H), 2.40-2- 20 (m, 3H), 2.20 (br.s., 3H), 2.08- 1.86 (m, 3H) ppm | 5- methylfuran- 2- carbaldehyde | General method A using 3,3- difluorocyclopentane-1- amine hydrochloride, TEA and NaBH₄, method D1 using 4- cyanophenylisocyanate | 3-(4- chlorophenyl)-1- (3,3- difluorocyclo- pentyl)-1-[(5- methyl-2- furyl)methyl]urea |
| 246 | 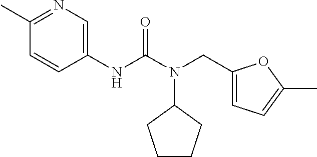 | 314.8 | | 1HNMR (300 MHz, CDCl3) δ: 8.29 (d, J = 2.4 Hz, 1H), 7.95 (dd, J = 8.3 Hz, 2.4 Hz, 1H), 7.18 (br. s, 1H), 7.10 (d, J = 8.4 Hz, 1H), 6.18 (d, J = 3.0 Hz, 1H), 5.97-5.92 (m, 1H), 4.75-4.60 (m, 1H), 4.34 (s, 2H), 2.51 (s, 3H), 2.30 (s, 3H), 2.02-1.85 (m, 2H), 1.80-1.49 (m, 6H) ppm. | 5- methylfuran- 2- carbaldehyde | General method A using cyclopentanamine and NaBH₄, method D2 using 6-methylpyridin- 3-amine | 1-cyclopentyl-1- [(5-methyl-2- furyl)methyl]-2- (6-methyl-3- pyridyl)urea |
| 247 | 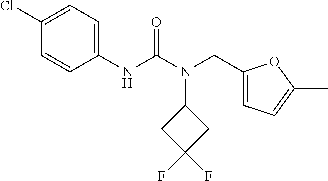 [SMILES: Cc1ccc(CN(C(=O)Nc2ccc(Cl)cc2)C2CC(F)(F)C2)o1] | 355.7 | | 1HNMR (300 MHz, CDCl3) δ: 7.24 (s, 4H), 7.00 (br. s, 1H), 6.21 (d, J = 3.1 Hz, 1H), 6.00-5.95 (m, 1H), 4.52-4.42 (m, 1H), 4.36 (s, 2H), 2.98-2.72 (m, 4H), 2.32 (d, J = 0.5 Hz, 3H) ppm. | 5- methylfuran- 2- carbaldehyde | General method A using 3,3- difluorocyclobutane-1- amine, TEA and NaBH₄, method D1 using 4- cyanophenylisocyanate | 3-(4- chlorophenyl)-1- (3,3-difluoro- cyclobutyl)- 1-[(5-methyl- 2- furyl)methyl]urea |
| 248 | 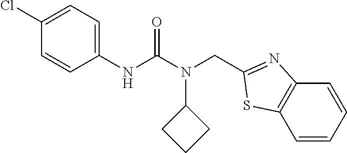 [SMILES: O=C(Nc1ccc(Cl)cc1)N(Cc1nc2ccccc2s1)C1CCC1] | 372.7 | | 1HNMR (300 MHz, CDCl3) δ: 8.04 (s, J = 8.1 Hz, 1H), 7.89 (s, J = 7.8 Hz, 1H), 7.73 (br. s, 1H), 7.56-7.48 (m, 1H), 7.47-7.35 (m, 3H), 7.25-7.20 (m, 2H), 4.91 (s, 2H), 4.59-4.46 (m, 1H), 2.39-2.17 (m, 4H), 1.82-1.65 (m, 2H) ppm. | 1,3- benzothiazole- 2- carbaldehyde | General method A using cyclobutanamine and NaBH₄, method D1 using 4- chlorophenylisocyanate | 1-(1,3- benzothiazol-2- ylmethyl)-3-(4- chlorophenyl)-1- cyclobutyl-urea |
| 249 | 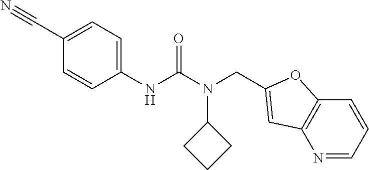 [SMILES: N#Cc1ccc(NC(=O)N(Cc2cc3ncccc3o2)C2CCC2)cc1] | 347.7 | | 1HNMR (300 MHz, CDCl3) δ: 8.52 (dd, J = 5.0 Hz, 1.2 Hz, 1H), 7.87-7.80 (m, 1H), 7.60-7.49 (m, 4H), 7.34-7.26 (m, 1H), 6.95 (d, J = 0.8 Hz, 1H), 6.78 (br. s, 1H), 4.79 (d, J = 0.7 Hz, 2H), 4.42-4.27 (m, 1H), 2.27-2.14 (m, 4H), 1.87- 1.67 (m, 2H) ppm | furo(3,2- b)pyridine-2- carbaldehyde | General method A using cyclobutanamine and NaBH₄, method D1 using 4- cyanophenylisocyanate | 3-(4- cyanophenyl)-1- cyclobutyl- (furo[3,2- b]pyridin-2- ylmethyl)urea |
| 250 | 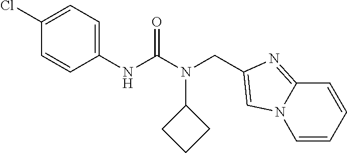 [SMILES: O=C(Nc1ccc(Cl)cc1)N(Cc1cn2ccccc2n1)C1CCC1] | 355.7 | | 1HNMR (300 MHz, DMSO-d6) δ: 9.37 (br. s, 1H), 8.52 (d, J = 6.7 Hz, 1H), 7.85 (s, 1H), 7.61-7.47 (m, 3H), 7.32-7.20 (m, 3H), 6.93- 6.83 (m, 1H), 4.65 (s, 2H), 4.62- 4.50 (m, 1H), 2.23-1.99 (m, 4H), 1.67-1.40 (m, 2H) ppm. | imidazo(1,2- a)pyridine-2- carbaldehyde | General method A using cyclobutanamine and NaBH₄, method D1 using 4- chlorophenylisocyanate | 3-(4- chlorophenyl)-1- cyclobutyl-1- (imidazo[1,2- a]pyridin-2- ylmethyl)urea |
| 251 | 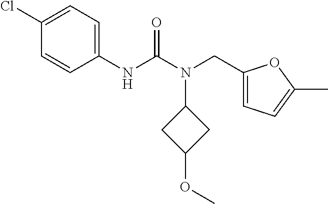 [SMILES: COC1CC(N(Cc2ccc(C)o2)C(=O)Nc2ccc(Cl)cc2)C1] | 349.7 | | 1H NMR (300 MHz, DMSO) δ: 8.45 (d, J = 5.8 Hz, 1H), 7.53-7.41 (m, 2H), 7.37-7.22 (m, 2H), 6.10- 6.04 (m, 1H), 5.96-5.94 (m, 1H), 4.66-3.89 (m, 1H), 4.52 (d, J = 5.2 Hz, 2H), 4.01-3.47 (m, 1H), 3.13 (d, J = 5.8 Hz, 3H), 2.36-2.21 (m, 2H), 2.24-2.15 (m, 1H), 2.21 (s, 3H), 1.97-1.81 (m, 1H) ppm | 5- methylfuran- 2- carbaldehyde | General method A using 3- methoxycyclobutan-1- amine hydrochloride, TEA and NaBH₄, method D1 using 4- chlorophenylisocyanate | 3-(4- chlorophenyl)-1- (3-methoxy- cyclobutyl)- 1-[(5- methyl-2- furyl)methyl]urea |
| 252 | 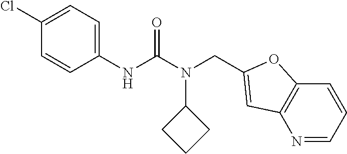 [SMILES: O=C(Nc1ccc(Cl)cc1)N(Cc1cc2ncccc2o1)C1CCC1] | 356.7 | | 1HNMR (300 MHz, CDCl3) δ: 8.51 (dd, J = 4.9 Hz, 1.0 Hz, 1H), 7.75 (dt, J = 7.3 Hz, 1.0 Hz, 1H), 7.36-7.29 (m, 2H), 7.26-7.18 (m, 2H), 6.87 (d, J = 0.8 Hz, 1H), 6.50 (br. s, 1H), 4.77 (d, J = 0.7 Hz, 2H), 4.39-4.25 (m, 1H), 2.41-2.14 (m, 4H), 1.89-1.63 (m, 2H) ppm. | furo(3,2- b)pyridine-2- carbaldehyde | General method A using cyclobutanamine and NaBH₄, method D1 using 4- chlorophenylisocyanate | 3-(4- chlorophenyl)-1- cyclobutyl-1- (furo[3,2- b]pyridin-2- ylmethyl)urea |
| 253 | 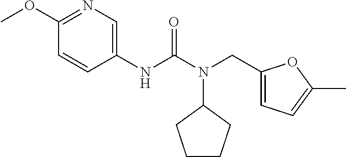 | 330.8 | | 1HNMR (300 MHz, CDCl3) δ : 7.90 (d, J = 2.3 Hz, 1H), 7.80 (dd, J = 8.8 Hz, 2.7 Hz, 1H), 6.84 (br. s, 1H), 6.70 (d, J = 8.7 Hz, 1H), 6.17 (d, J = 3.0 Hz, 1H), 5.97-5.92 (m, 1H), 4.74-4.59 (m, 1H), 4.32 (s, 2H), 3.89 (s, 3H), 2.30 (d, J = 0.5 Hz, 3H), 2.01-1.86 (m, 2H), 1.78-1.48 (m, 6H) ppm. | 5- methylfuran- 2- carbaldehyde | General method A using cyclopentanamine and NaBH₄, method D2 using 6- methoxypyridin-3- amine | 1-cyclopentyl- (6-methoxy-3- pyridyl)-1-[(5- methyl-2- furyl)methyl]urea |
| 254 | 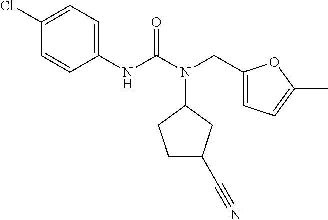 [SMILES: Cc1ccc(CN(C(=O)Nc2ccc(Cl)cc2)C2CCC(C#N)C2)o1] | 358.7 | | 1H NMR (300 MHz, DMSO) δ: 8.46 (br.s., 1H), 7.52-7.42 (m, 2H), 7.32-7.24 (m, 2H), 6.14 (d, J = 2.9 Hz, 1H), 6.00-5.96 (m, 1H), 4.52 (br.s., 2H), 4.49-4.36 (m, 1H), 3.05-2.91 (m, 1H), 2.22 (br.s, 3H), 2.20-2.11 (m, 1H), 2.04-1.72 (m, 5H) ppm | 5- methylfuran- 2- carbaldehyde | General method A using 3- aminocyclopentane-1- carbonitrile, TEA and NaBH₄, method D1 using 4- chlorophenylisocyanate | 3-(4- chlorophenyl)-1- (3- cyanocyclo- pentyl)-1-[(5- methyl-2- furyl)methyl]urea |
| 255 | 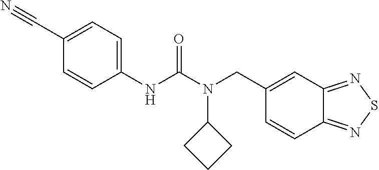 [SMILES: N#Cc1ccc(NC(=O)N(Cc2ccc3nsnc3c2)C2CCC2)cc1] | 364.7 | | 1HNMR (300 MHz, CDCl3) δ/ppm: 8.00 (dd, J = 9.0 Hz, J = 0.3 Hz, 1H), 7.88-7.84 (m, 1H), 7.58-7.51 (m, 3H), 7.50-7.44 (m, 2H), 6.55 (br. s, 1H), 4.80 (s, 2H), 4.50-4.35 (m, 1H), 2.38-2.12 (m, 4H), 1.83-1.66 (m, 2H). | 2,1,3- benzothia- diazole- 5- carbaldehyde | General method A using cyclobutanamine and NaBH₄, method D1 using 4- cyanophenylisocyanate | 1-(2,1,3- benzothiadiazol- 5-ylmethyl)-3- (4- cyanophenyl)- cyclobutyl-urea |
| 256 | 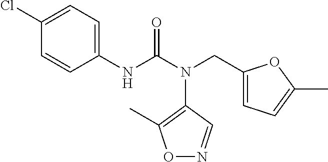 [SMILES: Cc1ccc(CN(C(=O)Nc2ccc(Cl)cc2)c2cnoc2C)o1] | | 344.7 | 1HNMR (300 MHz, CDCl3) δ: 8.08 (s, 1H), 7.33-7.15 (m, 4H), 6.28 (br. s, 1H), 6.13-6.01 (m, 1H), 5.90-5.81 (m, 1H), 4.66 (s, 2H), 2.23 (s, 3H), 2.20 (s, 3H) ppm. | 5- methylfuran- 2- carbaldehyde | General method A using 5-methylisoxazol- 4-amine hydrochloride, TEA and NaBH₄, method D1 using 4- chlorophenylisocyanate | 3-(4- chlorophenyl)-1- [(5-methyl-2- furyl)methyl]-1- (5- methylisoxazol- 4-yl)urea |
| 257 | 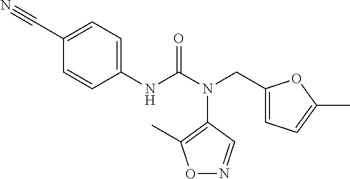 [SMILES: Cc1ccc(CN(C(=O)Nc2ccc(C#N)cc2)c2cnoc2C)o1] | | 335.6 | 1HNMR (300 MHz, DMSO-d6) δ: 8.67 (br. s, 1H), 8.50 (d, J = 0.6 Hz, 1H), 7.69 (s, 4H), 6.10 (d, J = 3.0 Hz, 1H), 5.99-5.94 (m, 1H), 4.67 (s, 2H), 2.21 (d, J = 0.4 Hz, 3H), 2.08 (d, J = 0.4 Hz, 3H) ppm. | 5- methylfuran- 2- carbaldehyde | General method A using 5-methylisoxazol- 4-amine hydrochloride, TEA and NaBH₄, method D1 using 4- cyanophenylisocyanate | 3-(4- cyanophenyl)-1- [(5-methyl-2- furyl)methyl]-1- (5- methylisoxazol- 4-yl)urea |
| 258 | 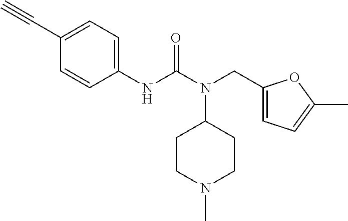 [SMILES: C#Cc1ccc(NC(=O)N(Cc2ccc(C)o2)C2CCN(C)CC2)cc1] | 352.8 | | 1H NMR (300 MHz, DMSO) δ: 8.46 (s, 1H), 7.51-7.43 (m, 2H), 7.38-7.28 (m, 2H), 6.12 (d, J = 3.0 Hz, 1H), 5.98-5.92 (m, 1H), 4.47 (br.s, 2H), 4.00 (s, 1H), 3.99-3.91 (m, 1H), 2.82-2.74 (m, 2H), 2.20 (d, J = 0.7 Hz, 3H), 2.13 (s, 3H), 1.98-1.66 (m, 2H), 1.80-1.64 (m, 2H), 1.58-1.47 (m, 2H) ppm | 5- methylfuran- 2- carbaldehyde | General method A using 4-amino-1- methylpiperidine and NaBH₄, method D2 using 4-ethynylaniline | 3-(4- ethynylphenyl)- 1-[(5-methyl- furyl)methyl]-1- (1-methyl-4- piperidyl)urea |
| 259 | 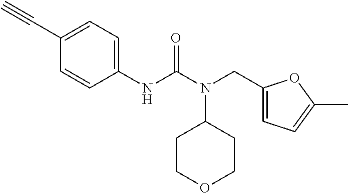 [SMILES: C#Cc1ccc(NC(=O)N(Cc2ccc(C)o2)C2CCOCC2)cc1] | 339.7 | | 1H NMR (300 MHz, DMSO) δ: 8.48 (s, 1H), 7.51-7.44 (m, 2H), 7.37-7.31 (m, 2H), 6.14 (d, J = 3.2 Hz, 1H), 5.99-5.94 (m, 1H), 4.50 (br.s, 2H), 4.30-4.16 (m, 1H), 4.01 (s, 1H), 3.93-3.83 (m, 2H), 3.42- 3.34 (m, 2H), 2.20 (d, J = 0.7 Hz, 3H) 1.84-1.66 (m, 2H), 1.59-1.49 (m, 2H) ppm | 5- methylfuran- 2- carbaldehyde | General method A using 4- aminotetrahydropyran and NaBH₄, method D2 using 4-ethynyl aniline | 3-(4- ethynylphenyl)- 1-[(5-methyl-2- furyl)methyl]-1- tetrahydropyran- 4-yl-urea |
| 260 | 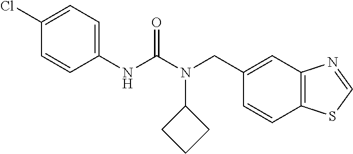 [SMILES: O=C(Nc1ccc(Cl)cc1)N(Cc1ccc2scnc2c1)C1CCC1] | 372.7 | | 1HNMR (300 MHz, CDCl3) δ: 9.02 (s, 1H), 8.05 (d, J = 0.8 Hz, 1H), 7.95 (d, J = 26.1 Hz, 1H), 7.41 (dd, J = 8.3 Hz, 1.4 Hz, 1H), 7.25-7.13 (m, 4H), 6.27 (s, 1H), 4.78 (s, 2H), 4.58-4.44 (m, 1H), 2.34-2.08 (m, 4H), 1.78-1.64 (m, 2H) ppm. | 1,3- benzothiazole- 5- carbaldehyde | General method A using cyclobutanamine and NaBH₄, method D1 using 4- chlorophenylisocyanate | 1-(1,3- benzothiazol-5- ylmethyl)-3-(4- chlorophenyl)-1- cyclobutyl-urea |
| 261 | 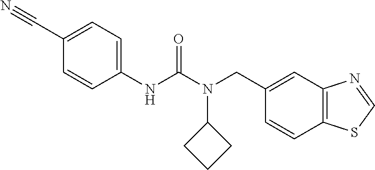 [SMILES: N#Cc1ccc(NC(=O)N(Cc2ccc3scnc3c2)C2CCC2)cc1] | 363.7 | | 1HNMR (300 MHz, CDCl3) δ: 9.03 (s, 1H), 8.04 (d, J = 0.3 Hz, 1H), 7.96 (d, J = 8.3 Hz, 1H), 7.55-7.47 (m, 2H), 7.44-7.36 (m, 3H), 6.50 (s, 1H), 4.79 (s, 2H), 4.61-4.45 (m, 1H), 2.39-2.08 (m, 4H), 1.83-1.65 (m, 2H) ppm. | 1,3- benzothiazole- 5- carbaldehyde | General method A using cyclobutanamine and NaBH₄, method D1 using 4- cyanophenylisocyanate | 1-(1,3- benzothiazol-5- ylmethyl)-3-(4- cyanophenyl)-1- cyclobutyl-urea |
| 262 | 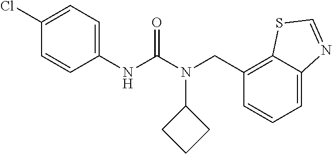 [SMILES: O=C(Nc1ccc(Cl)cc1)N(Cc1cccc2ncsc12)C1CCC1] | 372.6 | | 1HNMR (300 MHz, CDCl3) δ: 9.03 (s, 1H), 8.09 (dd, J = 8.4 Hz, 0.3 Hz, 1H), 7.54 (t, J = 7.8 Hz, 1H), 7.39-7.33 (m, 1H), 7.28-7.18 (m, 4H), 6.28 (br. s, 1H), 4.87 (s, 2H), 4.54-4.38 (m, 1H), 2.29-2.06 (m, 4H), 1.77-1.63 (m, 2H) ppm. | 1,3- benzothiazole- 7- carbaldehyde | General method A using cyclobutanamine and NaBH₄, method D1 using 4- chlorophenylisocyanate | 1-(1,3- benzothiazol-7- ylmethyl)-3-(4- chlorophenyl)-1- cyclobutyl-urea |
| 263 | 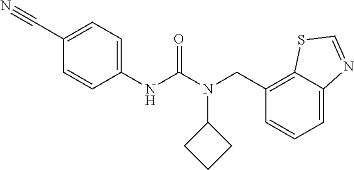 [SMILES: N#Cc1ccc(NC(=O)N(Cc2cccc3ncsc23)C2CCC2)cc1] | 363.7 | | 1HNMR (300 MHz, CDCl3) δ: 9.03 (s, 1H), 8.11 (dd, J = 8.1 Hz, 0.7 Hz, 1H), 7.59-7.49 (m, 3H), 7.45-7.39 (m, 2H), 7.38-7.33 (m, 1H), 6.50 (br. s, 1H), 4.88 (s, 2H), 4.54-4.40 (m, 1H), 2.33-2.07 (m, 4H), 1.81-1.60 (m, 2H) ppm. | 1,3- benzothiazole- 7- carbaldehyde | General method A using cyclobutanamine and NaBH₄, method D1 using 4- cyanophenylisocyanate | 1-(1,3- benzothiazol-7- ylmethyl)-3-(4- cyanophenyl)-1- cyclobutyl-urea |
| 264 | 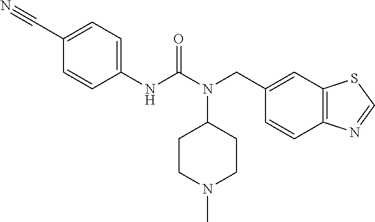 [SMILES: CN1CCC(N(Cc2ccc3ncsc3c2)C(=O)Nc2ccc(C#N)cc2)CC1] | | 404.7 | 1H NMR (300 MHz, DMSO) δ: 9.31 (s, 1H), 8.87 (br.s, 1H), 8.07- 7.96 (m, 2H), 7.65 (s, 4H), 7.44 (dd, J = 8.3 Hz, 1.7 Hz, 1H), 5.75 (s, 1H), 4.74 (br.s, 2H), 4.18-4.02 (m, 2H), 2.77-2.67 (m, 2H), 2.01 (s, 3H), 1.97-1.85 (m, 2H), 1.74- 1.48 (m, 2H) ppm | 1,3- benzothiazole- 6- carbaldehyde | General method A using 4-amino-1- methylpiperidine and NaBH₄, method D1 using 4- cyanophenylisocyanate | 1-(1,3- benzothiazol-6- ylmethyl)-3-(4- cyanophenyl)-1- (1-methyl-4- piperidyl)urea |
| 265 | 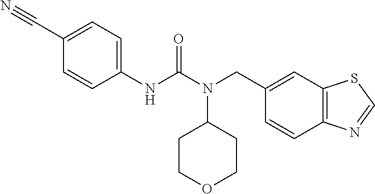 [SMILES: N#Cc1ccc(NC(=O)N(Cc2ccc3ncsc3c2)C2CCOCC2)cc1] | | 391.7 | 1H NMR (300 MHz, DMSO) δ: 9.32 (s, 1H), 8.89 (br.s, 1H), 8.05- 7.99 (m, 2H), 7.66 (s, 4H), 7.44 (dd, J = 8.4 Hz, 1.5 Hz, 1H), 4.76 (br.s., 2H), 4.43-4.29 (m, 1H), 3.88-3.78 (m, 2H), 3.42-3.33 (m, 2H), 1.77-1.61 (m, 2H), 1.61-1.50 (m, 2H) ppm | 1,3- benzothiazole- 6- carbaldehyde | General method A using 4- aminotetrahydropyran and NaBH₄, method D1 using 4-cyanophenyl- isocyanate | 1-(1,3- benzothiazol-6- ylmethyl)-3-(4- cyanophenyl)-1- tetrahydropyran- 4-yl-urea |
| 266 | 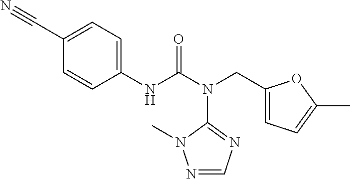 [SMILES: Cc1ccc(CN(C(=O)Nc2ccc(C#N)cc2)c2ncnn2C)o1] | | 335.7 | 1HNMR (300 MHz, DMSO-d6) δ: 9.25 (s, 1H), 7.93 (s, 1H), 7.68- 7.59 (m, 4H), 6.12 (d, J = 2.7 Hz, 1H), 5.99-5.90 (m, 1H), 4.83 (s, 2H), 3.49 (s, 3H), 2.17 (s, 3H) ppm | 5- methylfuran- 2- carbaldehyde | General method A using 2-methyl-1,2,4- triazol-3-amine, acetic acid and NaBH₄, method D1 using 4- cyanophenylisocyanate | 3-(4- cyanophenyl)-1- [(5-methyl-2- furyl)methyl]-1- (2-methyl-1,2,4- triazol-3-yl)urea |
| 267 | 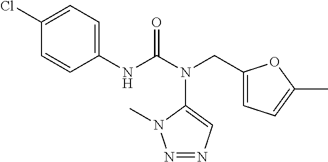 [SMILES: Cc1ccc(CN(C(=O)Nc2ccc(Cl)cc2)c2cnnn2C)o1] | 346.7 | | 1H NMR (300 MHz, DMSO-d6) δ: 8.66 (br.s., 1H), 7.59 (s, 1H), 7.50-7.43 (m, 2H), 7.34-7.28 (m, 2H), 6.11 (d, J = 3.13 Hz, 1H), 5.99-5.95 (m, 1H), 4.76 (br.s., 2H), 3.66 (s, 3H), 2.21 (s, 3H) ppm. | 5- methylfuran- 2- carbaldehyde | General method A using 3-methyl-triazol- 4-amine, acetic acid and NaBH₄, method D1 using 4- chlorophenylisocyanate | 3-(4- chlorophenyl)-1- [(5-methyl-2- furyl)methyl]-1- (3-methyltriazol- 4-yl)urea |
| 268 | 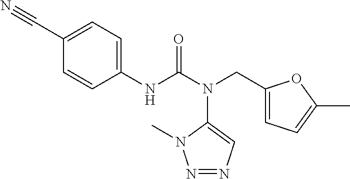 [SMILES: Cc1ccc(CN(C(=O)Nc2ccc(C#N)cc2)c2cnnn2C)o1] | 337.7 | | 1H NMR (300 MHz, DMSO-d6) δ: 8.96 (s, 1H), 7.76-7.64 (m, 4H), 7.60 (s, 1H), 6.13 (d, J = 3.10 Hz, 1H), 5.99-5.95 (m, 1H), 4.78 (br.s., 2H), 3.67 (s, 3H), 2.21 (br.s, 3H) ppm. | 5- methylfuran- 2- carbaldehyde | General method A using 3-methyl-triazol- 4-amine, acetic acid and NaBH₄, method D1 using 4- cyanophenylisocyanate | 3-(4- cyanophenyl)-1- [(5-methyl-2- furyl)methyl]-1- (3-methyltriazol- 4-yl)urea |
| 269 | 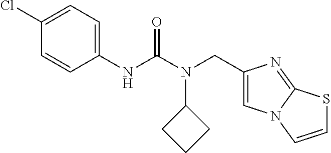 [SMILES: O=C(Nc1ccc(Cl)cc1)N(Cc1cn2ccsc2n1)C1CCC1] | 361.1 | | 1H NMR (300 MHz, DMSO-d6) δ: 8.98 (s, 1H), 7.86 (d, J = 4.5 Hz, 1H), 7.65 (s, 1H), 7.52 7.45 (m, 2H), 7.31-7.24 (m, 2H), 7.23 (d, J = 4.5 Hz, 1H), 4.58-4.43 (m, 1H), 4.53 (s, 2H), 2.26-2.02 (m, 4H), 1.69-1.45 (m, 2H) ppm. | imidazo(2,1- b)thiazole-6- carbaldehyde | General method A using cyclobutylamine, molecular sieves and NaBH₄, method D1 using 4- chlorophenylisocyanate | 3-(4- chlorophenyl)-1- cyclobutyl-1- (imidazo[2,1- b]thiazol-6- ylmethyl)urea |
| 270 | 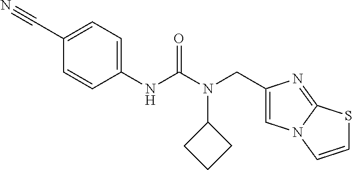 [SMILES: N#Cc1ccc(NC(=O)N(Cc2cn3ccsc3n2)C2CCC2)cc1] | 352.1 | | 1H NMR (300 MHz, DMSO-d6) δ: 9.39 (s, 1H), 7.86 (d, J = 4.4 Hz, 1H), 7.72-7.61 (m, 5H), 7.23 (d, J = 4.5 Hz, 1H), 4.56 (s, 2H), 4.53-4.43 (m, 1H), 2.24-2.03 (m, 4H), 1.70-1.45 (m, 2H) ppm. | imidazo(2,1- b)thiazole-6- carbaldehyde | General method A using cyclobutylamine, molecular sieves and NaBH₄, method D1 using 4- cyanophenylisocyanate | 3-(4- cyanophenyl)-1- cyclobutyl-1- (imidazo[2,1- b]thiazol-6- ylmethyl)urea |
| 271 | 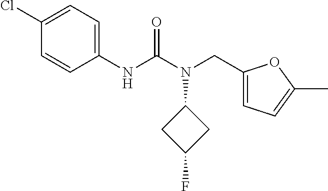 [SMILES: Cc1ccc(CN(C(=O)Nc2ccc(Cl)cc2)[C@H]2C[C@@H](F)C2)o1] | 337.09 | | 1H NMR (300 MHz, DMSO-d6) δ: 8.53 (s, 1H), 7.47 (m , 2H), 7.28 (m , 2H), 6.11 (d, J = 3.0 Hz, 1H), 5.97 (m, 1H), 4.81 (dp, 2JF- H = 56.7 Hz, 3JH-H = 6.7 Hz, 1H), 4.56 (m, 2H), 3.87 (m, 1H), 2.75 2.61 (m, 2H), 2.37-2.16 (m, 5H) ppm. | 5- methylfuran- 2- carbaldehyde | General method A using cis-3- fluorocyclobutan-1- amine hydrochloride, TEA, molecular sieves and NaBH₄, method D1 using 4- chlorophenylisocyanate | 3-(4- chlorophenyl)-1- (3- fluorocyclobutyl)- 1-[(5-methyl- 2- furyl)methyl]urea |
| 272 | 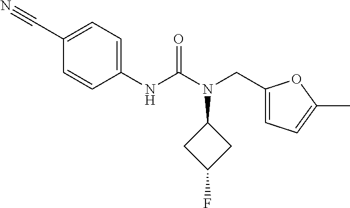 [SMILES: Cc1ccc(CN(C(=O)Nc2ccc(C#N)cc2)[C@H]2C[C@H](F)C2)o1] | 328.09 | | 1H NMR (300 MHz, DMSO-d6) δ: 8.92 (s, 1H), 7.66 (m, 4H), 6.12 (d, J = 3.0 Hz, 1H), 5.97 (m, 1H), 5.16 (dt, J = 57.7, 5.9 Hz, 1H), 4.73 (m, 1H), 4.56 (s, 2H), 2.67- 2.32 (m, 4H), 2.20 (s, 3H) ppm. | 5- methylfuran- 2- carbaldehyde | General method A using trans-3- fluorocyclobutan-1- amine hydrochloride, TEA, molecular sieves and NaBH₄, method D1 using 4- cyanophenylisocyanate | 3-(4- cyanophenyl)- (3- fluorocyclobutyl)- 1-[(5-methyl- 2- furyl)methyl]urea |
| 273 | 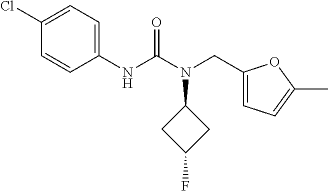 [SMILES: Cc1ccc(CN(C(=O)Nc2ccc(Cl)cc2)[C@H]2C[C@H](F)C2)o1] | 337.09 | | 1H NMR (300 MHz, DMSO-d6) δ: 8.55 (s, 1H), 7.47 (m, 2H), 7.28 (m, 2H), 6.11 (d, J = 2.9 Hz, 1H), 5.97 (m, 1H), 5.15 (dt, J = 57.9, 5.8 Hz, 1H), 4.73 (m, 1H), 4.53 (s, 2H), 2.65-2.31 (m, 4H), 2.21 (s, 3H). ppm. | 5- methylfuran- 2- carbaldehyde | General method A using trans-3- fluorocyclobutan-1- amine hydrochloride, TEA, molecular sieves and NaBH₄, method D1 using 4- chlorophenylisocyanate | 3-(4- chlorophenyl)-1- (3- fluorocyclobutyl)- 1-[(5-methyl- 2- furyl)methyl]urea |
| 274 | 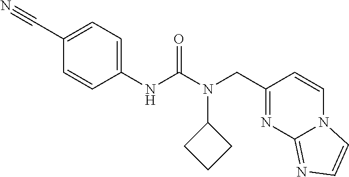 [SMILES: N#Cc1ccc(NC(=O)N(Cc2ccn3ccnc3n2)C2CCC2)cc1] | 347.12 | | 1H NMR (500 MHz, DMSO-d6) δ:8.92 (s, 1H), 8.90 (d, J = 7.0 Hz, 1H), 7.86 (d, J = 1.2 Hz, 1H), 7.67 (m , 4H), 7.64 (d, J = 1.1 Hz, 1H), 6.95 (d, J = 7.0 Hz, 1H), 4.78 (s, 2H), 4.66 (p, J = 8.5 Hz, 1H), 2.18-2.01 (m, 4H), 1.64 1.48 (m, 2H). ppm. | imidazo(1,2- a)pyrimidine- 7- carbaldehyde | General method A using cyclobutylamine, TEA, molecular sieves and NaBH₄, method D1 using 4- cyanophenylisocyanate | 3-(4- cyanophenyl)-1- cyclobutyl-1- (imidazo[1,2- a]pyrimidin- ylmethyl)urea |
| 275 | 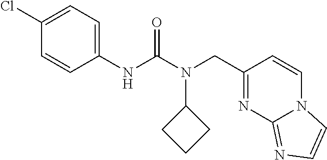 [SMILES: O=C(Nc1ccc(Cl)cc1)N(Cc1ccn2ccnc2n1)C1CCC1] | 356.03 | | 1H NMR (500 MHz, DMSO-d6) δ: 8.89 (d, J = 7.0 Hz, 1H), 8.57 (s, 1H), 7.86 (d, J = 1.2 Hz, 1H), 7.64 (d, J = 1.2 Hz, 1H), 7.50 (m, 2H), 7.27 (m, 2H), 6.93 (d, J = 7.0 Hz, 1H), 4.75 (s, 2H), 4.65 (p, J = 8.5 Hz, 1H), 2.20-1.93 (m, 4H), 1.65 1.48 (m, 2H) ppm. | imidazo(1,2- a)pyrimidine- 7- carbaldehyde | General method A using cyclobutylamine, TEA, molecular sieves and NaBH₄, method D1 using 4- chlorophenylisocyanate | 3-(4- chlorophenyl)-1- cyclobutyl-1- (imidazo[1,2- a]pyrimidin-7- ylmethyl)urea |
| 276 | 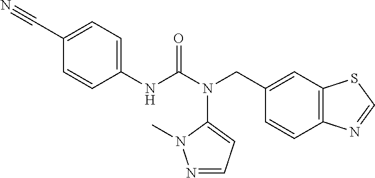 [SMILES: Cn1nccc1N(Cc1ccc2ncsc2c1)C(=O)Nc1ccc(C#N)cc1] | 389.18 | | 1H NMR (600 MHz, DMSO-d6) δ: 10.85 (br. s, 1H), 9.34 (s, 1H), 8.05-8.04 (m, 1H), 8.02 (d, J = 8.4 Hz, 1H), 7.79-7.74 (m, 4H), 7.68 (dd, J = 2.3 Hz, 0.3 Hz, 1H), 7.46 (dd, J = 8.6 Hz, 1.7 Hz, 1H), 6.08 (d, J = 2.4 Hz, 1H), 5.16 (s, 2H), 3.83 (s, 3H) ppm. | 1,3- benzothiazole- 6- carbaldehyde | General method A using 2-methylpyrazol- 3-amine, molecular sieves and NaBH₄, method D1 using 4- cyanophenylisocyanate | 1-(1,3- benzothiazol-6- ylmethyl)-3-(4- cyanophenyl)-1- (2-methyl-1H- pyrazol-3- yl)urea |
| 277 | 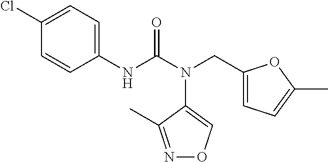 [SMILES: Cc1ccc(CN(C(=O)Nc2ccc(Cl)cc2)c2conc2C)o1] | 346.05 | | 1H NMR (600 MHz, DMSO-d6) δ: 8.88 (s, 1H), 8.45 (s, 1H), 7.48 (m, 2H), 7.29 (m, 2H), 6.11 (d, J = 3.1 Hz, 1H), 5.97 (m, 1H), 4.67 (s, 2H), 2.21 (s, 3H), 1.92 (s, 3H) ppm. | 5- methylfuran- 2- carbaldehyde | General method A using 3-methylisoxazol- 4-amine hydrochloride, TEA and NaBH₄, method D1 using 4- chlorophenylisocyanate | 3-(4- chlorophenyl)-1- [(5-methyl-2- furyl)methyl]-1- (3- methylisoxazol- 4-yl)urea |
| 278 | 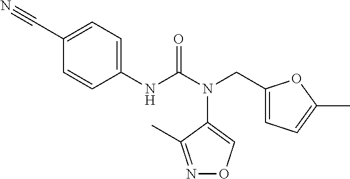 [SMILES: Cc1ccc(CN(C(=O)Nc2ccc(C#N)cc2)c2conc2C)o1] | 339.09 | | 1H NMR (600 MHz, DMSO-d6) δ: 8.91 (d, J = 0.5 Hz, 1H), 8.78 (s, 1H), 7.69 (m, 4H), 6.12 (d, J = 3.0 Hz, 1H), 5.97 (m, 1H), 4.69 (s, 2H), 2.21 (d, J = 0.8 Hz, 3H), 1.92 (d, J = 0.5 Hz, 3H) ppm. | 5- methylfuran- 2- carbaldehyde | General method A using 3-methylisoxazol- 4-amine hydrochloride, TEA and NaBH₄, method D1 using 4- cyanophenylisocyanate | 3-(4- cyanophenyl)-1- [(5-methyl-2- furyl)methyl]-1- (3- methylisoxazol- 4-yl)urea |
| 279 | 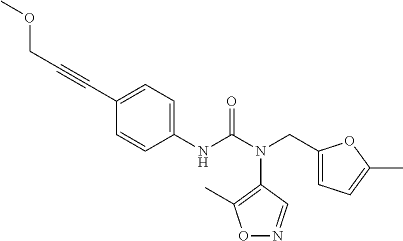 | 380.07 | | 1H NMR (600 MHz, CDCl3) δ: 8.33 (s, 1H), 7.38-7.33 (m, 2H), 7.32-7.28 (m, 2H), 6.35 (br. s, 1H), 6.11 (d, J = 3.0 Hz, 1H), 5.90- 5.87 (m, 1H), 4.70 (s, 2H), 4.29 (s, 2H), 3.43 (s, 3H), 2.29 (s, 3H), 2.10 (s, 3H) ppm. | 5- methylfuran- 2- carbaldehyde | General method A using 5-methylisoxazol- 4-amine hydrochloride, TEA and NaBH₄, method D1 using 1- iodo-4- isocyanatobenzene, method F using 3- methoxyprop-1-yne, Pd(PPh₃)₂Cl₂, CuI and TEA | 3-[4-(3- methoxyprop-1- ynyl)phenyl]-1- [(5-methyl-2- furyl)methyl]-1- (5- methylisoxazol- 4-yl)urea |
| 280 | 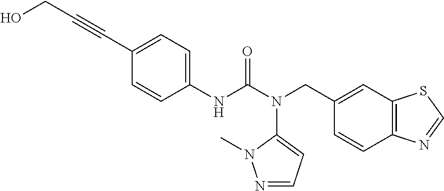 [SMILES: Cn1nccc1N(Cc1ccc2ncsc2c1)C(=O)Nc1ccc(C#CCO)cc1] | 418.07 | | 1H NMR (600 MHz, CDCl3) δ: 10.95 (s, 1H), 8.93 (s, 1H), 8.06 (d, J = 8.6 Hz, 1H), 7.88 (d, J = 0.9 Hz, 1H), 7.57-7.53 (m, 2H), 7.46 (dd, J = 8.4 Hz, 1.7 Hz, 1H), 7.42-7.38 (m, 2H), 7.22 (d, J = 2.4 Hz, 1H), 5.78 (d, J = 2.3 Hz, 1H), 5.22 (s, 2H), 4.49 (s, 2H), 3.87 (s, 3H) ppm. | 1,3- benzothiazole- 6- carbaldehyde | General method A using 2-methylpyrazol- 3-amine and NaBH₄, method D1 using 1- iodo-4- isocyanatobenzene, method F using 3- methoxyprop-1-yne, Pd(PPh₃)₂Cl₂, CuI and TEA | 1-(1,3- benzothiazol-6- ylmethyl)-3-[4- (3-hydroxypropyl- 1-ynyl)phenyl- 1-(2- methylpyrazol- 3-yl)urea |
| 281 | 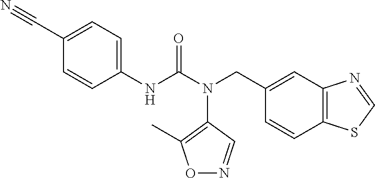 [SMILES: Cc1oncc1N(Cc1ccc2scnc2c1)C(=O)Nc1ccc(C#N)cc1] | 390.06 | | 1H NMR (600 MHz, DMSO-d6) δ: 9.39 (s, 1H), 8.79 (s, 1H), 8.54 (s, 1H), 8.12 (d, J = 8.3 Hz, 1H), 7.93 (s, 1H), 7.73-7.70 (m, 4H), 7.43-7.40 (m, 1H), 4.91 (s, 2H), 2.04 (s, 3H) ppm. | 1,3- benzothiazole- 5- carbaldehyde | General method A using 5-methylisoxazol- 4-amine hydrochloride, TEA and NaBH₄, method D1 using 4- cyanophenylisocyanate | 1-(1,3- benzothiazol-1- ylmethyl)-3-(4- cyanophenyl)-1- (5- methylisoxazol- 4-yl)urea |
| 282 | 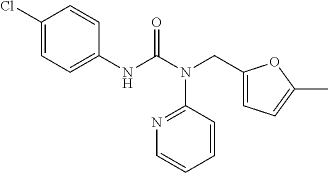 [SMILES: Cc1ccc(CN(C(=O)Nc2ccc(Cl)cc2)c2ccccn2)o1] | 342.01 | | 1H NMR (600 MHz, CDCl3) δ: 12.80 (s, 1H), 8.28-8.24 (m, 1H), 7.68-7.64 (m, 1H), 7.50-7.46 (m, 2H), 7.23 (d, J = 8.6 Hz, 1H), 7.22-7.18 (m, 2H), 6.95 (dd, J = 7.2 Hz, 5.0 Hz, 1H), 6.11 (d, J = 2.9 Hz, 1H), 5.81 (d, J = 2.6 Hz, 1H), 5.06 (s, 2H), 2.18 (s, 3H) | 5- methylfuran- 2- carbaldehyde | General method A using 2-aminopyridine, molecular sieves, p- TsOHxH₂O and NaBH₄, method D1 using 4- chlorophenylisocyanate | 3-(4- chlorophenyl)-1- [(5-methyl-2- furyl)methyl]-1- (2-pyridyl)urea |
| 283 | 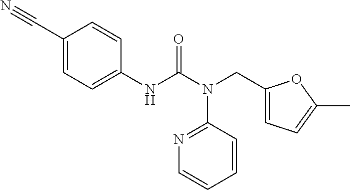 [SMILES: Cc1ccc(CN(C(=O)Nc2ccc(C#N)cc2)c2ccccn2)o1] | 333.03 | | 1H NMR (600 MHz, CDCl3) δ: 13.32 (s, 1H), 8.35 (dd, J = 5.1 Hz, 2.1 Hz, 1H), 7.19-7.74 (m, 1H), 7.74-7.70 (m, 2H), 7.61-7.58 (m, 2H), 7.35 (d, J = 8.7 Hz, 1H), 7.06 (dd, J = 7.2 Hz, 5.1 Hz, 1H), 6.20 (d, J = 3.07 Hz, 1H), 5.91- 5.88 (m, 1H), 5.12 (s, 2H), 2.26 (s, 3H) ppm. | 5- methylfuran- 2- carbaldehyde | General method A using 2-aminopyridine, molecular sieves, p- TsOHxH₂O and NaBH₄, method D1 using 4- cyanophenylisocyanate | 3-(4- cyanophenyl)-1- [(5-methyl-2- furyl)methyl]-1- (2-pyridyl)urea |
| 284 | 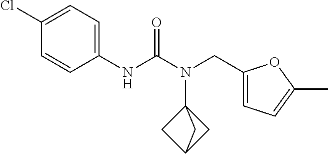 [SMILES: Cc1ccc(CN(C(=O)Nc2ccc(Cl)cc2)C23CC(C2)C3)o1] | 331.12 | | 1H NMR (300 MHz, DMSO-d6) δ: 8.23 (s, 1H), 7.52-7.44 (m, 2H), 7.31-7. 24 (m, 2H), 6.12 (d, J = 2.9 Hz, 1H), 5.99-5.95 (m, 1H), 4.49 (s, 2H), 2.41 (s, 1H), 2.21 (s, 3H), 2.08 (s, 6H) ppm. | 5- methylfuran- 2- carbaldehyde | General method A using bicyclo[1.1.1]pentan-1- amine hydrochloride, TEA and NaBH₄, method D1 using 4- chlorophenylisocyanate | 1-(1- bicyclo[1.1.1] pentanyl)-3-(4- chlorophenyl)-1- [(5-methyl-2- furyl)methyl]urea |
| 285 | 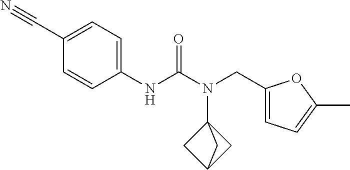 [SMILES: Cc1ccc(CN(C(=O)Nc2ccc(C#N)cc2)C23CC(C2)C3)o1] | 322.09 | | 1H NMR (300 MHz, DMSO-d6) δ: 8.63 (s, 1H), 7.73-7.60 (m, 4H), 6.13 (d, J = 3.0 Hz, 1H), 6.00-5.95 (m, 1H), 4.52 (s, 2H), 2.43 (s, 1H), 2.20 (s, 3H), 2.09 (s, 6H). ppm. | 5- methylfuran- 2- carbaldehyde | General method A using bicyclo[1.1.1]pentan-1- amine hydrochloride, TEA and NaBH₄, method D1 using 4- cyanophenylisocyanate | 1-(1- bicyclo[1.1.1] pentanyl)-3-(4- cyanophenyl)-1- [(5-methyl-2- furyl)methyl]urea |
| 286 | 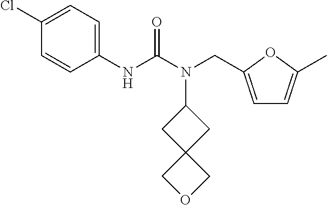 [SMILES: Cc1ccc(CN(C(=O)Nc2ccc(Cl)cc2)C2CC3(COC3)C2)o1] | 361.13 | | 1H NMR (600 MHz, DMSO-d6) δ: 8.39 (s, 1H), 7.46 (m, 2H), 7.27 (m, 2H), 6.06 (d, J = 2.9 Hz, 1H), 5.96 (m, 1H), 4.61 (s, 2H), 4.48 (s, 2H), 4.46 (s, 2H), 4.19 (m, 1H), 2.48 (m, 2H), 2.27 (m, 2H), 2.20 (d, J = 0.7 Hz, 3H) ppm. | 5- methylfuran- 2- carbaldehyde | General method A using 2- oxaspiro[3.3]heptan-6- amine hydrochloride, TEA and NaBH₄, method D1 using 4- chlorophenylisocyanate | 3-(4- chlorophenyl)-1- [(5-methyl-2- furyl)methyl]-1- (2- oxaspiro[3.3] heptan-6-yl)urea |
| 287 | 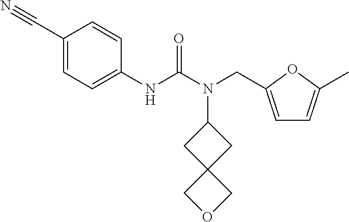 [SMILES: Cc1ccc(CN(C(=O)Nc2ccc(C#N)cc2)C2CC3(COC3)C2)o1] | 352.17 | | 1H NMR (600 MHz, DMSO-d6) δ: 8.76 (s, 1H), 7.68 (m, 2H), 7.63 (m, 2H), 6.08 (d, J = 3.0 Hz, 1H), 5.96 (m, 1H), 4.61 (s, 2H), 4.51 (s, 2H), 4.46 (s, 2H), 4.19 (m, 1H), 2.49 (m, 2H), 2.28 (m, 2H), 2.19 (d, J = 0.7 Hz, 3H) ppm. | 5- methylfuran- 2- carbaldehyde | General method A using 2- oxaspiro[3.3]heptan-6- amine hydrochloride, TEA and NaBH₄, method D1 using 4- cyanophenylisocyanate | 3-(4- cyanophenyl)-1- [(5-methyl-2- furyl)methyl]-1- (2- oxaspiro[3.3] heptan-6-yl)urea |
| 288 | 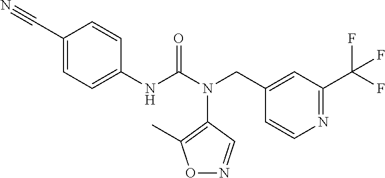 [SMILES: Cc1oncc1N(Cc1ccnc(C(F)(F)F)c1)C(=O)Nc1ccc(C#N)cc1] | 402.11 | | 1H NMR (300 MHz, DMSO-d6) δ: 8.87 (br.s., 1H), 8.72 (d, J = 4.8 Hz, 1H), 8.67 (br.s., 1H), 7.78 (br.s., 1H), 7.71 (s, 4H), 7.68-7.64 (m, 1H), 4.89 (br.s., 2H), 2.22 (s, 3H) ppm. | 4- (chloro- methyl)-2- (trifluoro- methyl) pyridine | General method B.1 using 5- methylisooxazol-4- amine, NaI and DIPEA, method D1 using 4- cyanophenylisocyanate | 3-(4- cyanophenyl)-1- (5- methylisoxazol- 4-yl)-1-[[2- (trifluoromethyl)- 4- pyridyl]methyl] urea |
| 289 | 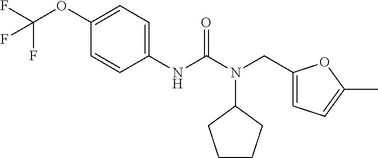 [SMILES: Cc1ccc(CN(C(=O)Nc2ccc(OC(F)(F)F)cc2)C2CCCC2)o1] | 383.15 | | 1H NMR (500 MHz, DMSO-d6) δ: 8.47 (s, 1H), 7.55 (d, J = 9.1 Hz, 2H), 7.22 (d, J = 9.1 Hz, 2H), 6.09 (d, J = 2.8 Hz, 1H), 5.96 (dd, J = 2.8, 0.9 Hz, 1H), 4.47-4.39 (m, 3H), 2.21 (s, 3H), 1.81-1.72 (m, 2H), 1.69-1.61 (m, 2H), 1.59- 1.46 (m, 4H) ppm. | 5- methylfuran- 2- carbaldehyde | General method A using cyclopentanamine and NaBH₄, method D1 using 4- (trifluoromethoxy)- phenyl isocyanate | 1-cyclopentyl-1- [(5-methyl-2- furyl)methyl]-3- [4- (trifluoromethoxy) phenyl]urea |
| 290 | 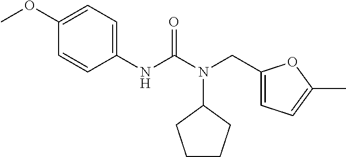 | 329.15 | | 1H NMR (500 MHz, DMSO-d6) δ: 8.09 (s, 1H), 7.31 (d, J = 9.5 Hz, 2H), 6.80 (d, J = 9.5 Hz, 2H), 6.08 (d, J = 3.1 Hz, 1H), 5.97- 5.94 (m, 1H), 4.46-4.38 (m, 3H), 3.70 (s, 3H), 2.21 (s, 3H), 1.80- 1.71 (m, 2H), 1.69-1.60 (m, 2H), 1.58-1.44 (m, 4H) ppm. | 5- methylfuran- 2- carbaldehyde | General method A using cyclopentanamine and NaBH₄, method D1 using 4-methoxyphenyl isocyanate | 1-cyclopentyl-1- (4- methoxyphenyl)- 1-[(5-methyl-2- furyl)methyl]urea |
| 291 | 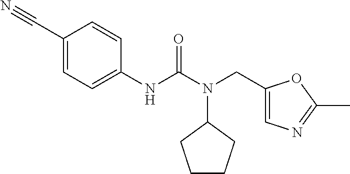 [SMILES: Cc1ncc(CN(C(=O)Nc2ccc(C#N)cc2)C2CCCC2)o1] | 325.14 | | 1H NMR (500 MHz, DMSO-d6) δ: 8.83 (s, 1H), 7.69-7.64 (m, 4H), 6.81 (s, 1H), 4.52 (s, 2H), 4.47- 4.39 (m, 1H), 2.34 (s, 3H), 1.84- 1.75 (m, 2H), 1.71-1.63 (m, 2H), 1.61-1.47 (m, 4H) ppm. | 2- methyl- oxazole-5- carbaldehyde | General method A using cyclopentanamine and NaBH₄, method D1 using 4- cyanophenylisocyanate | 3-(4- cyanophenyl)-1- cyclopentyl-1- [(2- methyloxazol-5- yl)methyl]urea |
| 292 | 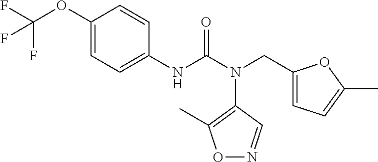 [SMILES: Cc1ccc(CN(C(=O)Nc2ccc(OC(F)(F)F)cc2)c2cnoc2C)o1] | 396.08 | | 1H NMR (400 MHz, DMSO-d6) δ: 8.47 (s, 1H), 8.41 (s, 1H), 7.55 (d, J = 8.9 Hz, 2H), 7.23 (d, J = 8.9 Hz, 2H), 6.07 (d, J = 3.0 Hz, 1H), 5.96-5.94 (m, 1H), 4.65 (s, 2H), 2.20 (s, 3H), 2.07 (s, 3H) ppm. | 5- methylfuran- 2- carbaldehyde | General method A using 5-methylisoxazol- 4-amine hydrochloride, NaOAc and NaCNBH₃, method D1 using 4- cyanophenylisocyanate | 1-[(5-methyl-2- furyl)methyl]-1- (5- methylisoxazol- 4-yl)-3-[4- (trifluoromethoxy) phenyl]urea |
| 293 | 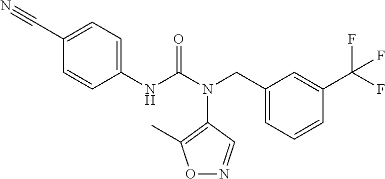 [SMILES: Cc1oncc1N(Cc1cccc(C(F)(F)F)c1)C(=O)Nc1ccc(C#N)cc1] | 401.22 | | 1H NMR (500 MHz, DMSO-d6) δ: 8.79 (s, 1H), 8.58 (s, 1H), 7.71 (s, 4H), 7.67-7.63 (m, 1H), 7.55- 7.26 (m, 3H), 4.84 (s, 2H), 2.05 (s, 3H) ppm. | 3- (trifluoro- methyl) benzaldehyde | General method A using 5-methylisoxazol- 4-amine hydrochloride, NaOAc and NaCNBH₃, method D1 using 4- cyanophenylisocyanate | 3-(4- cyanophenyl)-1- (5- methylisoxazol- 4-yl)-1-[[3- (trifluoromethyl) phenyl]methyl] urea |
| 294 | 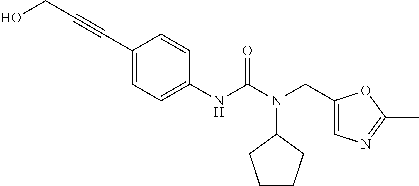 [SMILES: Cc1ncc(CN(C(=O)Nc2ccc(C#CCO)cc2)C2CCCC2)o1] | 354.13 | | 1H NMR (500 MHz, DMSO-d6) δ: 8.50 (s, 1H), 7.47 (d, J = 8.6 Hz, 2H), 7.28 (d, J = 8.6 Hz, 2H), 6.80 (s, 1H), 5.23 (t, J = 5.7 Hz, 1H), 4.40 (s, 2H), 4.45-4.38 (m, 1H), 4.27 (d, J = 5.7 Hz, 2H), 2.34 (s, 3H), 1.84-1.74 (m, 2H), 1.72- 1.62 (m, 2H), 1.60-1.47 (m, 4H) ppm. | 2- methyl- oxazole-5- carbaldehyde | General method A using cyclopentanamine and NaBH₄, method F using 4-iodoaniline, prop-2-yn-1-ol, Pd(PPh₃)₂Cl₂, CuI and TEA, method M using tert-butyl-chloro- dimethyl silane, method R using isopropenyl chloroformate method D6, method N using TBAFxH₂O | 1-cyclopentyl-3- [4-(3- hydroxyprop-1- ynyl)phenyl]-1- [(2- methyloxazol-5- yl)methyl]urea |
| 295 | 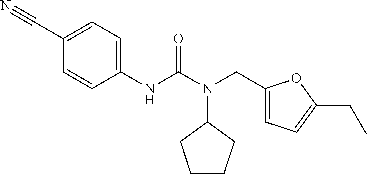 [SMILES: CCc1ccc(CN(C(=O)Nc2ccc(C#N)cc2)C2CCCC2)o1] | 338.2 | | 1H NMR (500 MHz, DMSO-d6) δ: 8.78 (s, 1H), 7.69-7.63 (m, 4H), 6.10 (d, J = 3.0 Hz, 1H), 5.96 (d, J = 3.0 Hz, 1H), 4.48 (s, 2H), 4.46- 4.38 (m, 1H), 2.54-2.50 (m, 2H ), 1.82-1.72 (m, 2H), 1.70-1.62 (m, 2H), 1.60-1.45 (m, 4H), 1.11 (t, J = 7.4 Hz, 3H) ppm. | 5- ethylfuran- 2- carbaldehyde | General method A using cyclopentanamine and NaBH₄, method D1 using 4- cyanophenylisocyanate | 3-(4- cyanophenyl)-1- cyclopentyl- [(5-ethyl-2 furyl)methyl]urea |
| 296 | 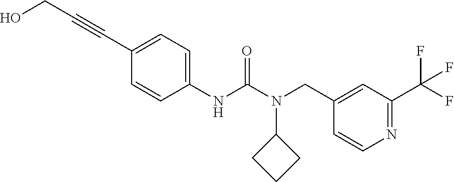 [SMILES: O=C(Nc1ccc(C#CCO)cc1)N(Cc1ccnc(C(F)(F)F)c1)C1CCC1] | 404.08 | | 1H NMR (600 MHz, CDCl3) δ: 8.71 (d, J = 8.7 Hz, 1H), 7.61-7.59 (m, 1H), 7.44-7.42 (m, 1H), 7.42- 7.37 (m, 4H), 6.42 (br.s., 1H), 4.73 (s, 2H), 4.51 (d, J = 6.2 Hz, 2H), 4.33-4.27 (m, 1H), 2.35-2.29 (m, 2H), 2.20-2.12 (m, 2H), 1.86- 1.75 (m, 2H), 1.63 (t, J = 6.0 Hz, 1H) ppm. | 2- (trifluoro- methyl) pyridine-4- carbaldehyde | General method A using cyclobutanamine and NaBH₄, method F using 4-iodoaniline, prop-2-yn-1-ol, Pd(PPh₃)₂Cl₂, CuI and TEA, method M using tert-butyl-chloro- dimethyl silane, method R using isopropenyl chloroformate method | 1-cyclobutyl-3- [4-(3- hydroxyprop-1- ynyl)phenyl]-1- [[2- (trifluoromethyl)- 4- pyridyl]methyl] urea |
| | | | | | | D6, method N using | |
| | | | | | | TBAFxH₂O | |
| 297 | 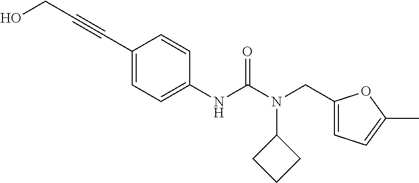 [SMILES: Cc1ccc(CN(C(=O)Nc2ccc(C#CCO)cc2)C2CCC2)o1] | 339.15 | | 1H NMR (500 MHz, DMSO-d6) δ: 8.40 (s, 1H), 7.48-7.43 (m, 2H), 7.30-7.24 (m, 2H), 6.07 (d, J = 2.9 Hz, 1H), 5.97-5.95 (m, 1H), 5.26 (br. s., 1H), 4.52 (s, 2H), 4.38 (m, 1H), 4.26 (s, 2H), 2.20 (s, 3H), 2.16- 2.06 (m, 4H), 1.65-1.49 (m, 2H) ppm. | 5- methylfuran- 2- carbaldehyde | General method A using cyclobutanamine and NaBH₄, method F using 4-iodoaniline, prop-2-yn-1-ol, Pd(PPh₃)₂Cl₂, CuI and TEA, method M using tert-butyl-chloro- dimethyl silane, method R using isopropenyl chloroformate method D6, method N using TBAFxH₂O | 1-cyclobutyl-3- [4-(3- hydroxyprop-1- ynyl)phenyl]-1- [(5-methyl-2- furyl)methyl]urea |
| 298 | 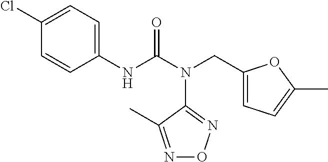 [SMILES: Cc1ccc(CN(C(=O)Nc2ccc(Cl)cc2)c2nonc2C)o1] | | 344.9 | 1H NMR (300 MHz, DMSO-d6) δ: 9.36 (br.s., 1H), 7.53-7.44 (m, 2H), 7.39-7.31 (m, 2H), 6.19 (d, J = 1.6 Hz, 1H), 6.01-5.96 (m, 1H), 4.92 (s, 2H), 2.18 (s, 3H), 2.13 (s, 3H) ppm. | 5- methylfuran- 2- carbaldehyde | General method A using 4-methyl-1,2-5- oxadiazol-3-amine, toluene, MgSO₄ and NaBH₄, method D1 using 4- chlorophenylisocyanate | 3-(4- chlorophenyl)-1- [(5-methyl-2- furyl)methyl]-1- (4-methyl-1,2,5- oxadiazol-3- yl)urea |
| 299 | 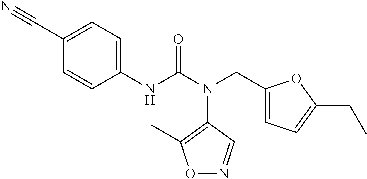 [SMILES: CCc1ccc(CN(C(=O)Nc2ccc(C#N)cc2)c2cnoc2C)o1] | 351.14 | | 1H NMR (600 MHz, DMSO-d6) δ: 8.50 (s, 1H), 7.72-7.67 (m, 4H), 6.12 (d, J = 2.8 Hz, 1H), 5.98 (dt, J = 2.8 Hz, 1.0 Hz, 1H), 4.68 (s, 2H), 2.55 (dq, J = 7.5, 1.0 Hz, 2H), 2.07 (d, 3H), 1.11 (t, J = 7.5 Hz, 3H) ppm. | 5- ethylfuran- 2- carbaldehyde | General method A using 5-methylisoxazol- 4-amine hydrochloride, NaOAc and NaCNBH₃, method D1 using 4- cyanophenylisocyanate | 3-(4- cyanophenyl)-1- [(5-ethyl-2- furyl)methyl]-1- (5- methylisoxazol- 4-yl)urea |
| 300 | 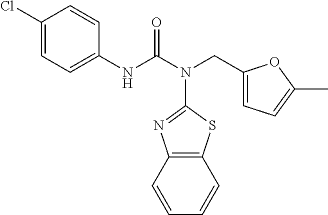 [SMILES: Cc1ccc(CN(C(=O)Nc2ccc(Cl)cc2)c2nc3ccccc3s2)o1] | 398.05 | | 1H NMR (300 MHz, DMSO-d6) δ: 10.0 (s, 1H), 7.93 (d, J = 7.0 Hz, 1H), 7.78 (d, J = 7.7 Hz, 1H), 7.62-7.57 (m, 2H), 7.45-7.38 (m, 3H), 7.31-7.24 (m, 1H), 6.27 (d, J = 2.8 Hz, 1H), 6.00-5.97 (m, 1H), 5.93 (s, 2H), 2.18 (s, 3H) ppm. | 2-chloro-1,3- benzothiazole | General method B.2 using (5-methyl-2- furyl)methanamine and DIPEA, method D1 using 4- chlorophenylisocyanate | 1-(1,3- benzothiazol-2- yl)-3-(4- chlorophenyl)-1- [(5-methyl-2- furyl)methyl]urea |
| 301 | 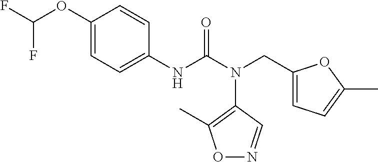 [SMILES: Cc1ccc(CN(C(=O)Nc2ccc(OC(F)F)cc2)c2cnoc2C)o1] | 378.16 | | 1H NMR (300 MHz, DMSO-d6) δ: 8.47 (s, 1H), 8.30 (s, 1H), 7.50- 7.43 (m, 2H), 7.10 (t, J = 74.4 Hz, 1H), 7.09-7.02 (m, 2H), 6.07 (d, J = 3.0 Hz, 1H), 5.97-5.93 (m, 1H), 4.64 (s, 2H), 2.20 (s, 3H), 2.07 (s, 3H) ppm. | 5- methylfuran- 2- carbaldehyde | General method A using 5-methylisoxazol- 4-amine hydrochloride, molecular sieves, TEA and NaBH₄, method D2.2 using 4- (difluoromethoxy) aniline | 3-[4- (difluoromethoxy) phenyl]-1-[(5- methyl-2- furyl)methyl]-1- (5- methylisoxazol- 4-yl)urea |
| 302 | 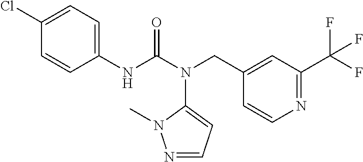 [SMILES: Cn1nccc1N(Cc1ccnc(C(F)(F)F)c1)C(=O)Nc1ccc(Cl)cc1] | 410.11 | | 1H NMR (500 MHz, DMSO-d6) δ: 8.73 (d, J = 8.7 Hz, 1H), 8.59 (s, 1H), 7.75 (s, 1H), 7.65 (d, J = 4.1 Hz, 1H), 7.50-7.46 (m, 2H), 7.42 (d, J = 1.8 Hz, 1H), 7.33-7.28 (m, 2H), 6.12 (d, J = 1.9 Hz, 1H), 4.91 (br.s., 2H), 3.64 (s, 3H) ppm. | 4- (chloro- methyl)-2- (trifluoro- methyl) pyridine | General method B.1 using 2-methylpyrazol- 3-amine, NaI and DIPEA, method D1 using 4- chlorophenylisocyanate | 3-(4- chlorophenyl)-1- (2- methylpyrazol- 3-yl)-1-[[2- (trifluoromethyl)- 4- pyridyl]methyl] urea |
| 303 | 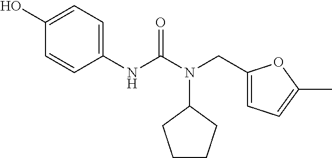 [SMILES: Cc1ccc(CN(C(=O)Nc2ccc(O)cc2)C2CCCC2)o1] | 315.07 | | 1H NMR (600 MHz, DMSO-d6) δ: 8.99 (s, 1H), 7.97 (s, 1H), 7.18- 7.15 (m, 2H), 6.65-6.61 (m, 2H), 6.09 (d, J = 3.1, 1H), 5.98-5.96 (m, 1H), 4.39 (br.s., 2H), 2.63- 2.61 (m, 1H), 2.23-2.21 (m, 3H), 1.79-1.72 (m, 2H), 1.68-1.61 (m, 2H), 1.57-1.48 (m, 4H) ppm. | 5- methylfuran- 2- carbaldehyde | General method A using cyclopentanamine and NaBH₄, method D1 using 1-benzyloxy-4- isocyanato benzene and toluene, method O using Pd(OH)₂/C | 1-cyclopentyl-3- (4- hydroxyphenyl)- 1-[(5-methyl- furyl)methyl]urea |
| 304 | 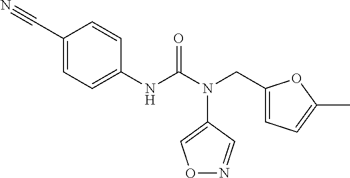 [SMILES: Cc1ccc(CN(C(=O)Nc2ccc(C#N)cc2)c2cnoc2)o1] | 323.05 | | 1H NMR (500 MHz, CDCl3) δ: 8.55 (s, 1H), 8.32 (s, 1H), 7.58- 7.43 (m, 4H), 6.77 (br.s., 1H), 6.17 (d, J = 3.1 Hz, 1H), 5.98-5.93 (m, 1H), 4.71 (s, 2H), 2.28 (s, 3H) ppm. | 5- methylfuran- 2- carbaldehyde | General method A using isooxazol-4- amine, TEA, toluene, molecular sieves and NaBH₄, method D1 using 4- cyanophenylisocyanate | 3-(4- cyanophenyl)-1- isoxazol-4-yl-1- [(5-methyl-2- furyl)methyl]urea |
| 305 | 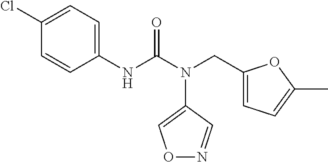 [SMILES: Cc1ccc(CN(C(=O)Nc2ccc(Cl)cc2)c2cnoc2)o1] | 332.07 | | 1H NMR (500 MHz, CDCl3) δ: 8.55 (s, 1H), 8.32 (s, 1H), 7.32- 7.21 (m, 4H), 6.55 (br.s., 1H), 6.19 (d, J = 3.1 Hz, 1H), 5.98-5.93 (m, 1H), 4.71 (s, 2H), 2.28 (s, 3H) ppm. | 5- methylfuran- 2- carbaldehyde | General method A using isooxazoll-4- amine, TEA, toluene, molecular sieves and NaBH₄, method D1 using 4- chlorophenylisocyanate | 3-(4- chlorophenyl)-1- isoxazol-4-yl-1- [(5-methyl-2- furyl)methyl]urea |
| 306 | 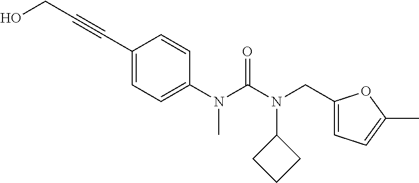 [SMILES: Cc1ccc(CN(C(=O)N(C)c2ccc(C#CCO)cc2)C2CCC2)o1] | 353.21 | | 1H NMR (500 MHz, DMSO-d6) δ: 7.35-7.29 (m, 2H), 7.28-7.05 (m, 2H), 6.08 (d, J = 2.9 Hz, 1H), 5.99-5.96 (m, 1H), 5.28 (t, J = 5.9 Hz, 1H), 4.27 (d, J = 6.1 Hz, 2H), 4.23 (s, 2H), 3.72 (m, 1H), 3.07 (s, 3H), 2.24 (s, 3H), 1.98-1.86 (m, 2H), 1.75-1.66 (m, 2H), 1.52- 1.43 (m, 1H), 1.37-1.27 (m, 1H) ppm. | 5- methylfuran- 2- carbaldehyde | General method A using cyclobutanamine and NaBH₄, method F using 4-iodoaniline, prop-2-yn-1-ol, Pd(PPh₃)₂Cl₂, CuI and TEA, method M using tert-butyl-chloro- dimethyl silane, method R using isopropenyl chloroformate method | 1-cyclobutyl-3- [4-(3- hydroxyprop-1- ynyl)phenyl]-3- methyl-1-[(5- methyl-2- furyl)methyl]urea |
| | | | | | | D6, method J using MeI | |
| | | | | | | and Cs₂CO₃, method N | |
| | | | | | | using TBAFxH₂O | |
| 307 | 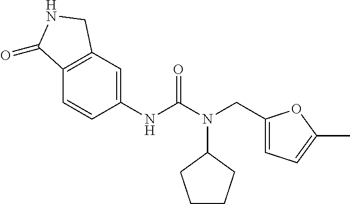 [SMILES: Cc1ccc(CN(C(=O)Nc2ccc3c(c2)CNC3=O)C2CCCC2)o1] | 354.18 | | 1H NMR (300 MHz, DMSO-d6) δ: 8.57 (s, 1H), 8.27 (s, 1H), 7.75 (s, 1H), 7.52-7.43 (m, 2H), 6.10 (d, J = 3.0 Hz, 1H), 5.96 (b.s., 1H), 4.46 (s, 2H), 4.46-4.40 (m, 1H), 4.38 (s, 2H), 2.20 (s, 3H), 1.81- 1.72 (m, 2H), 1.71-1.63 (m, 2H), 1.61-1.44 (m, 4H) ppm. | 5- methylfuran- 2- carbaldehyde | General method A using cyclopentanamine and NaBH₄, method R using isopropenyl chloroformate, method D6 | 1-cyclopentyl) [(5-methyl-2- furyl)methyl]-3- (1- oxoisoindolin-5- yl)urea |
| 308 | 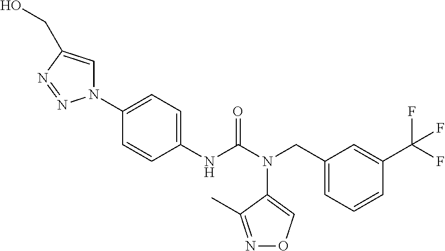 [SMILES: Cc1nocc1N(Cc1cccc(C(F)(F)F)c1)C(=O)Nc1ccc(-n2cc(CO)nn2)cc1] | 473.09 | | 1H NMR (300 MHz, DMSO-d6) δ: 8.95 (s, 1H), 8.69 (s, 1H), 8.56 (s, 1H) 7.77 (d, J = 8.7 Hz, 2H), 7.70-7.54 (m, 6H), 5.28 (t, J = 5.5 Hz, 1H), 4.85 (s, 2H), 4.58 (d, J = 5.5 Hz, 2H), 1.94 (s, 3H) ppm. | 3- (trifluoro- methyl) benzaldehyde | General method A using 3-methylisoxazol- 4-amine hydrochloride, NaOAc and NaCNBH₃, method M using tert- butyl-chloro-dimethyl silane, method D2.2 using 4-[4-[[tert- butyl(dimethyl)silyl] oxymethyl]triazol-1- yl]aniline, method D1 using tert-butyl-((4- isocyanatophenyl) triazol-4- yl)methoxy)dimethyl- silane, method N using TBAFxH₂O | 3-[4-[4- (hydroxymethyl) triazol-1- yl]phenyl]-1-(3- methylisoxazol- 4-yl)-1-[[3- (trifluoromethyl) phenyl]methyl] urea |
| 309 | 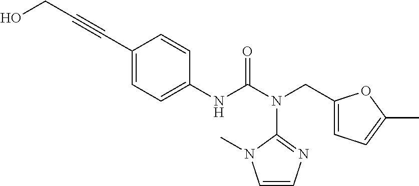 [SMILES: Cc1ccc(CN(C(=O)Nc2ccc(C#CCO)cc2)c2nccn2C)o1] | 365.19 | | 1H NMR (500 MHz, DMSO-d6) δ: 8.53 (s, 1H), 7.49-7.42 (m, 2H), 7.33-7.27 (m, 2H), 7.08 (d, J = 1.2 Hz, 1H), 6.84 (d, J = 1.30 Hz, 1H), 6.04 (d, J = 2.9 Hz, 1H), 5.96-5.91 (m, 1H), 5.27 (t, J = 5.9 Hz, 1H), 4.74 (s, 2H), 4.26 (d, J = 5.7 Hz, 2H), 3.22 (s, 3H), 2.19 (s, 3H) ppm. | 5- methylfuran- 2- carbaldehyde | General method A using 1- methylimidazol-2- amine hydrochloride, Na2SO₄, TEA and NaBH₄, method F using 3-methoxyprop-1-yne, Pd(PPh₃)₂Cl₂, CuI and TEA, method M using tert-butyl-chloro- dimethyl silane, method D2.2 using 4-[3-[tert- | 3-[4-(3- hydroxyprop- ynyl)phenyl]-1- [(5-methyl-2- furyl)methyl]-1- (1- methylimidazol- 2-yl)urea |
| | | | | | | butyl(dimethyl)silyl] | |
| | | | | | | oxyprop-1-ynyl]aniline, | |
| | | | | | | method N using | |
| | | | | | | TBAFxH₂O | |
| 310 | 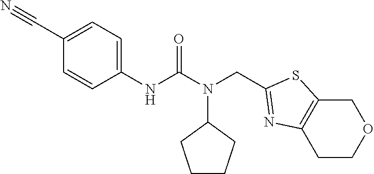 [SMILES: N#Cc1ccc(NC(=O)N(Cc2nc3c(s2)COCC3)C2CCCC2)cc1] | 383.06 | | 1H NMR (500 MHz, DMSO-d6) δ: 9.1 (s, 1H), 7.71-7.65 (m, 4H), 4.71 (m, 4H), 4.55-4.46 (m, 1H), 3.90 (t, J = 3.9 Hz, 2H), 2.78-2.74 (m, 2H), 1.86-1.77 (m, 2H), 1.72- 1.64 (m, 2H), 1.61-1.49 (m, 4H) ppm. | 6,7-dihydro- 4H- pyrano[4,3- d]thiazole-2- carbaldehyde | General method A using cyclopentanamine and NaBH₄ and molecular sieves, method D1 using 4- cyanophenyl isocyanate | 3-(4- cyanophenyl)-1- cyclopentyl-1- (6,7-dihydro-4H- pyrano[4,3- d]thiazol-2- ylmethyl)urea |
| 311 | 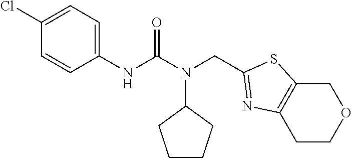 [SMILES: O=C(Nc1ccc(Cl)cc1)N(Cc1nc2c(s1)COCC2)C1CCCC1] | 392.06 | | 1H NMR (500 MHz, DMSO-d6) δ: 8.74 (s, 1H), 7.51-7.46 (m, 2H), 7.31-7.26 (m, 2H), 4.72-4.66 (m, 4H), 4.52-4.44 (m, 1H), 3.90 (t, J = 5.4 Hz, 2H), 2.78-2.73 (m, 2H), 1.85-1.76 (m, 2H), 1.70-1.63 (m, 2H), 1.58-1.47 (m, 4H) ppm. | 6,7-dihydro- 4H- pyrano[4,3- d]thiazole-2- carbaldehyde | General method A using cyclopentanamine and NaBH₄ and molecular sieves, method D1 using 4- chlorophenyl isocyanate | 3-(4- chlorophenyl)-1- cyclopentyl-1- (6,7-dihydro-4H- pyrano[4,3- d]thiazol-2- ylmethyl)urea |
| 312 | 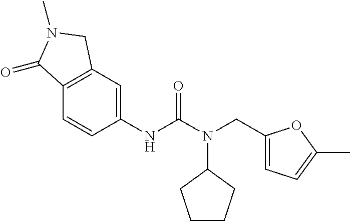 [SMILES: Cc1ccc(CN(C(=O)Nc2ccc3c(c2)CN(C)C3=O)C2CCCC2)o1] | 368.18 | | 1H NMR (500 MHz, DMSO-d6) δ: 8.57 (s, 1H), 7.76 (s, 1H), 7.53- 7.43 (m, 2H), 6.10 (d, J = 3.0 Hz, 1H), 5.98 (b.s., 1H), 4.46 (s, 2H), 4.46-4.40 (m, 1H), 4.38 (s, 2H), 3.03 (s, 3H), 2.20 (s, 3H), 1.82- 1.73 (m, 2H), 1.70-1.62 (m, 2H), 1.62-1.45 (m, 4H) ppm. | 5- methylfuran- 2- carbaldehyde | General method A using cyclopentanamine and NaBH₄, method R using isopropenyl chloroformate, method D6 | 1-cyclopentyl-1- [(5-methyl-2- furyl)methyl]-3- (2-methyl-1- oxo-isoindolin- 5-yl)urea |
| 313 | 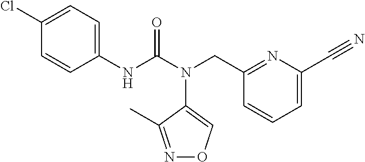 [SMILES: Cc1nocc1N(Cc1cccc(C#N)n1)C(=O)Nc1ccc(Cl)cc1] | 367.98 | | 1H NMR (500 MHz, CDCl3) δ: 8.60 (s, 1H), 7.77 (t, J = 7.8 Hz, 1H), 7.58-7.53 (m, 2H), 7.23-7.14 (m, 4H), 6.56 (br. s, 1H), 4.79 (s, 2H), 2.22 (s, 3H) ppm. | 6-formyl- pyridine-2- carbonitrile | General method A using 3-methylisoxazol- 4-amine hydrochloride, TEA and NaBH₄, method D1 using 4- chlorophenylisocyanate | 3-(4- chlorophenyl)-1- [(6-cyano-2- pyridyl)methyl]- 1-(3- methylisoxazol- 4-yl)urea |
| 314 | 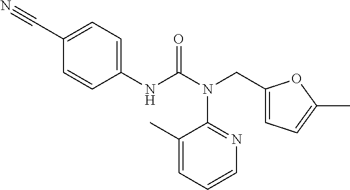 [SMILES: Cc1ccc(CN(C(=O)Nc2ccc(C#N)cc2)c2ncccc2C)o1] | | 345.1 | 1H NMR (500 MHz, DMSO-d6) δ: 8.81 (s, 1H), 8.38 (d, J = 4.6 Hz, 1H), 7.71-7.60 (m, 5H), 7.27 (dd, J = 7.5, 4.6 Hz, 1H), 5.98 (d, J = 2.6 Hz, 1H), 5.89 (b.s., 1H), 4.89 (s, 2H), 2.14 (s, 3H), 1.97 (s, 3H) ppm. | 2-chloro-3- methyl- pyridine | General method C.1 using (5-methyl-2- furyl)methanamine, BINAP, Pd(OAc)₂ and K₂CO₃, method D1 using 4- cyanophenylisocyanate, TEA in toluene | 3-(4- cyanophenyl) [(5-methyl-2- furyl)methyl]-1- (3-methyl-2- pyridyl)urea |
| 315 | 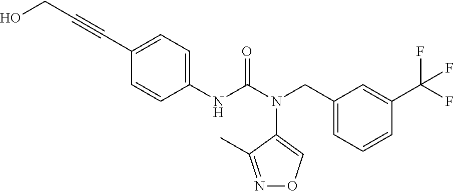 [SMILES: Cc1nocc1N(Cc1cccc(C(F)(F)F)c1)C(=O)Nc1ccc(C#CCO)cc1] | 430.15 | | 1H NMR (300 MHz, DMSO-d6) δ: 8.91 (s, 1H), 8.60 (s, 1H), 7.69- 7.61 (m, 1H), 7.60-7.54 (m, 3H), 7.47 (d, J = 8.3 Hz, 2H), 7.30 (d, J = 8.3 Hz, 2H), 5.26 (t, J = 6.2 Hz, 1H), 4.82 (s, 2H), 4.26 (d, J = 6.2 Hz, 2H), 1.91 (s, 3H) ppm. | 3- (trifluoro- methyl) benzaldehyde | General method A using 3- methylisooxazol-4- amine hydrochloride, NaOAc and NaCNBH₃, method F using 3- methoxyprop-1-yne, Pd(PPh₃)₂Cl₂, CuI and TEA, method M using tert-butyl-chloro- dimethyl silane, method D2.2 using 4-[3-[tert- | 3-[4-(3- hydroxyprop-1- ynyl)phenyl]-1- (3- methylisoxazol- 4-yl)-1-[[3- (trifluoromethyl) phenyl]methyl] urea |
| | | | | | | butyl(dimethyl)silyl] | |
| | | | | | | oxyprop-1-ynyl]aniline, | |
| | | | | | | method N using | |
| | | | | | | TBAFxH₂O | |
| 316 | 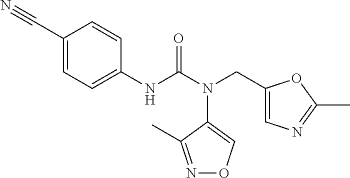 [SMILES: Cc1ncc(CN(C(=O)Nc2ccc(C#N)cc2)c2conc2C)o1] | 338.12 | | 1H NMR (500 MHz, DMSO-d6) δ: 8.99 (s, 1H), 8.81 (s, 1H), 7.74- 7.64 (m, 4H), 6.86 (s, 1H), 4.77 (s, 2H), 2.35 (s, 3H), 1.99 (s, 3H)ppm. | 2-methyl- oxazole-5- carbaldehyde | General method A using 3-methylisoxazol- 4-amine hydrochloride, NaOAc and NaCNBH₃, method D1 using 4- cyanophenylisocyanate | 3-(4- cyanophenyl)- (3- methylisoxazol- 4-yl)-1-[(2- methyloxazol-5- yl)methyl]urea |
| 317 | 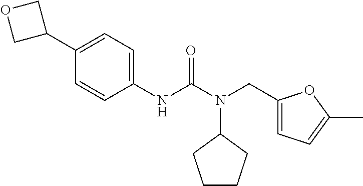 [SMILES: Cc1ccc(CN(C(=O)Nc2ccc(C3COC3)cc2)C2CCCC2)o1] | 355.2 | | 1H NMR (500 MHz, DMSO-d6) δ: 8.25 (s, 1H), 7.46-7.40 (m, 2H), 7.27-7.22 (m, 2H), 6.08 (d, J = 2.8 Hz, 1H), 5.98-5.94 (m, 1H), 4.90 (d, J = 5.80 Hz, 1H), 4.89 (d, J = 5.80 Hz, 1H), 4.57 (t, J = 6.3 Hz, 2H), 4.48-4.38 (m, 1H), 4.43 (s, 2H), 4.16 (m, 1H), 2.21 (s, 3H), 1.81-1.70 (m, 2H), 1.69-1.60 (m, 2H), 1.58-1.44 (m, 4H) ppm. | 5- methylfuran- 2- carbaldehyde | General method A using cyclopentanamine and NaBH₄, method R using isopropenyl chloroformate, method D6 | 1-cyclopentyl-1- [(5-methyl-2- furyl)methyl]-3- [4-(oxetan-3- yl)phenyl]urea |
| 318 | 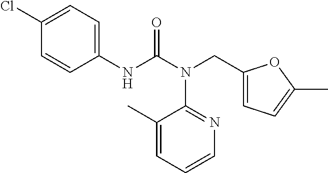 [SMILES: Cc1ccc(CN(C(=O)Nc2ccc(Cl)cc2)c2ncccc2C)o1] | 356.14 | | 1H NMR (500 MHz, DMSO-d6) δ: 8.43 (s, 1H), 8.36 (dd, J = 4.9, 1.4 Hz, 1H), 7.66 (dd, J = 7.4, 1.1 Hz, 1H), 7.48-7.43 (m, 2H), 7.30- 7.23 (m, 3H), 5.96 (d, J = 2.6 Hz, 1H), 5.90-5.86 (m, 1H), 4.88 (s, 2H), 2.14 (s, 3H), 1.98 (s, 3H)ppm. | 2-chloro-3- methyl- pyridine | General method C.1 using (5-methyl-2- furyl)methanamine, BINAP, Pd(OAc)₂ and K₂CO₃, method D1 using 4- chlorophenylisocyanate, TEA in toluene | 3-(4- chlorophenyl)-1- [(5-methyl-2- furyl)methyl]-1- (3-methyl-2- pyridyl)urea |
| 319 | 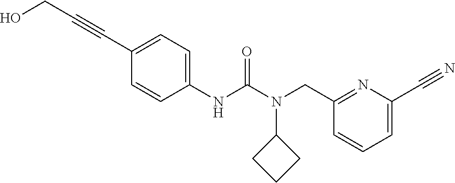 [SMILES: N#Cc1cccc(CN(C(=O)Nc2ccc(C#CCO)cc2)C2CCC2)n1] | 361.16 | | 1H NMR (500 MHz, CDCl3) δ: 7.86 (t, J = 8.2 Hz, 1H), 7.68-7.58 (m, 2H), 7.41-7.36 (m, 4H), 4.66 (s, 2H), 4.48 (d, J = 5.9 Hz, 2H), 4.47-4.41 (m, 1H), 2.32-2.22 (m, 2H), 2.19-2.08 (m, 2H), 1.83-1.68 (m, 2H), 1.65 (t, J = 6.3 Hz, 1H) ppm. | 6-formyl- pyridine-2- carbonitrile | General method A using cyclobutanamine and NaBH₄, method F using 4-iodoaniline, prop-2-yn-1-ol, Pd(PPh₃)₂Cl₂, Cul and TEA, method M using tert-butyl-chloro- dimethyl silane, method R using isopropenyl chloroformate method D6, method N using TBAFxH₂O | 1-[(6-cyano-2- pyridyl)methyl- 1-cyclobutyl-3- [4-(3- hydroxyprop-1- ynyl)phenyl]urea |
| 320 | 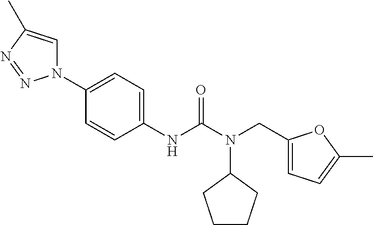 [SMILES: Cc1cn(-c2ccc(NC(=O)N(Cc3ccc(C)o3)C3CCCC3)cc2)nn1] | 380.22 | | 1H NMR (500 MHz, DMSO-d6) δ: 8.53 (s, 1H), 8.41 (s, 1H), 7.72- 7.67 (m, 2H), 7.66-7.62 (m, 2H), 6.11 (s, J = 3.1 Hz, 1H), 5.98-5.96 (m, 1H), 4.48- 4.41 (m, 3H), 2.31 (s, 3H), 2.21 (s, 3H), 1.82-1.74 (m, 2H), 1.71-1.62 (m, 2H), 1.61- 1.46 (m, 4H) ppm. | 5- methylfuran- 2- carbaldehyde | General method A using cyclopentanamine and NaBH₄, method L.2 using 4-(fluoromethyl)- 1-(4- nitrophenyl)triazole, method U using Deoxo- Fluor, method L.2, method R using isopropenyl chloroformate, method D6 | 1-cyclopentyl-1- [(5-methyl-2- furyl)methyl]-3- [4-(4- methyltriazol-1- yl)phenyl]urea |
| 321 | 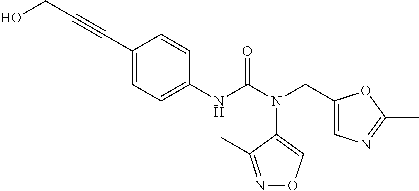 [SMILES: Cc1ncc(CN(C(=O)Nc2ccc(C#CCO)cc2)c2conc2C)o1] | 367.15 | | 1H NMR (500 MHz, DMSO-d6) δ: 8.96 (s, 1H), 8.52 (s, 1H), 7.46 (d, J = 8.4 Hz, 2H), 7.30 (d, J = 8.4 Hz, 2H), 6.85 (s, 1H), 5.27 (t, J = 4.8 Hz, 1H), 4.75 (s, 2H), 4.27 (d, J = 4.8 Hz, 2H), 2.36 (s, 3H), 1.99 (s, 3H) ppm. | 2-methyl- oxazole-5- carbaldehyde | General method A using 3- methylisooxazol-4- amine hydrochloride, NaOAc and NaCNBH₃, method F using 3- methoxyprop-1-yne, Pd(PPh₃)₂Cl₂, CuI and TEA, method M using tert-butyl-chloro- dimethyl silane, method D2.2 using 4-[3-[tert- butyl(dimethyl)silyl] | 3-[4-(3- hydroxyprop-1- ynyl)phenyl]-1- (3- methylisoxazol- 4-yl)-1-[(2- methyloxazol-5- yl)methyl]urea |
| | | | | | | oxyprop-1-ynyl]aniline, | |
| | | | | | | method N using | |
| | | | | | | TBAFxH₂O | |
| 322 | 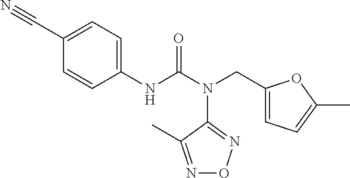 [SMILES: Cc1ccc(CN(C(=O)Nc2ccc(C#N)cc2)c2nonc2C)o1] | | 336.1 | 1H NMR (600 MHz, DMSO-d6) δ: 9.65 (br. s, 1H), 7.78-7.74 (m, 2H), 7.67-7.64 (m, 2H), 6.20 (d, J = 3.0 Hz, 1H), 5.99-5.97 (m, 1H), 4.95 (s, 2H), 2.17 (d, J = 0.6 Hz, 3H), 2.13 (s, 3H) ppm. | 5- methylfuran- 2- carbaldehyde | General method A using 4-methyl-1,2-5- oxadiazol-3-amine, toluene, MgSO₄ and NaBH₄, method D1 using 4- cyanophenylisocyanate | 3-(4- cyanophenyl)-1- [(5-methyl-2- furyl)methyl]-1- (4-methyl-1,2,5- oxadiazol-3- yl)urea |
| 323 | 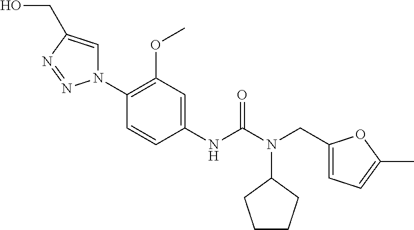 [SMILES: COc1cc(NC(=O)N(Cc2ccc(C)o2)C2CCCC2)ccc1-n1cc(CO)nn1] | 426.16 | | 1H NMR (600 MHz, DMSO-d6) δ: 8.58 (s, 1H), 8.17 (s, 1H), 7.53 (d, J = 2.1 Hz, 1H), 7.41 (d, J = 8.4 Hz, 1H), 7.24 (dd, J = 8.6 Hz, 2.1 Hz, 1H), 6.11 (d, J = 2.9 Hz, 1H), 5.99- 5.96 (m, 1H), 5.26 (br. s, 1H), 4.58 (s, 2H), 4.50- 4.44 (m, 1H), 4.47 (s, 2H), 3.79 (s, 3H), 2.22 (d, J = 0.6 Hz, 3H), 1.82-1.75 (m, 2H), 1.70-1.62 (m, 2H), 1.61-1.42 (m, 4H) ppm. | 5- methylfuran- 2- carbaldehyde | General method A using cyclopentanamine and NaBH₄, method V.1 using sodium nitrite, aq. HCl and sodium azide, method W using CuSO₄x5H₂O, sodium ascorbate and prop-2-yn-1-ol, method L using palladium/C, method M using tert- butyl-chloro-dimethyl silane, method R using isopropenyl chloroformate, method | 1-cyclopentyl- [4-[4- (hydroxymethyl) triazol-1-yl]-3- methoxy- phenyl]-1-[(5- methyl-2- furyl)methyl]urea |
| | | | | | | D6, method N using | |
| | | | | | | TBAFxH₂O | |
| 324 | 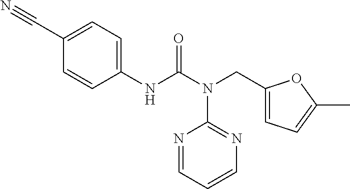 [SMILES: Cc1ccc(CN(C(=O)Nc2ccc(C#N)cc2)c2ncccn2)o1] | 334.15 | | 1H NMR (300 MHz, DMSO-d6) δ: 12.73 (s, 1H), 8.80 (d, J = 4.9 Hz, 2H), 7.87-7.76 (m, 4H), 7.26 (t, J = 4.0 Hz, 1H), 6.07 (d, J = 3.0 Hz, 1H), 5.94-5.89 (m, 1H), 5.32 (s, 2H), 2.17 (s, 3H) ppm. | 2-fluoro- pyrimidine | General method B.1 using (5-methyl-2- furyl)methanamine and DIPEA, method D1 using 4- cyanophenylisocyanate, TEA in toluene | 3-(4- cyanophenyl)-1- [(5-methyl-2- furyl)methyl]-1- pyrimidin-2-yl- urea |
| 325 | 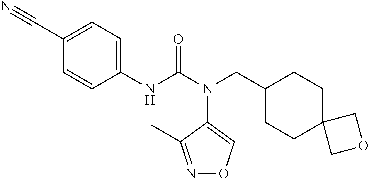 [SMILES: Cc1nocc1N(CC1CCC2(CC1)COC2)C(=O)Nc1ccc(C#N)cc1] | 381.24 | | 1H NMR (500 MHz, DMSO-d6) δ: 9.12 (s, 1H), 8.65 (s, 1H), 7.70- 7.63 (m, 4H), 4.24 (s, 2H), 4.18 (s, 2H), 3.37 (d, J = 6.8 Hz, 2H), 2.11 (s, 3H), 2.00 (d, J = 13.4 Hz, 2H), 1.57 (dd, J = 13.4, 3.0 Hz, 2H), 1.42-1.30 (m, 3H), 0.96-0.86 (m, 2H) ppm. | 2- oxaspiro[3.5] nonane-7- carbaldehyde | General method A using 3- methylisooxazol-4- amine hydrochloride, NaOAc and NaCNBH₃, method D1 using 4- cyanophenylisocyanate | 3-(4- cyanophenyl)- (3- methylisoxazol- 4-yl)-1-(2- oxaspiro[3.5] nonan-7- ylmethyl)urea |
| 326 | 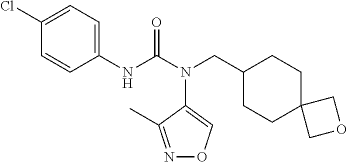 [SMILES: Cc1nocc1N(CC1CCC2(CC1)COC2)C(=O)Nc1ccc(Cl)cc1] | 390.2 | | 1H NMR (500 MHz, DMSO-d6) δ: 9.09 (s, 1H), 8.32 (s, 1H), 7.46- 7.42 (m, 2H), 7.29-7.25 (m, 2H), 4.27 (s, 2H), 4.18 (s, 2H), 2.37- 2.33 (m, 2H, overlapped with H2O), 2.11 (s, 3H), 2.00 (d, J = 13.2 Hz, 2H), 1.57 (dd, J = 13.2, 3.2 Hz, 2H), 1.41-1.31 (m, 3H), 0.96-0.86 (m, 2H) ppm. | 2- oxaspiro[3.5] nonane-7- carbaldehyde | General method A using 3- methylisooxazol-4- amine hydrochloride, NaOAc and NaCNBH₃, method D1 using 4- chlorophenylisocyanate | 3-(4- chlorophenyl)-1- (3- methylisoxazol- 4-yl)-1-(2- oxaspiro[3.5] nonan-7- ylmethyl)urea |
| 327 | 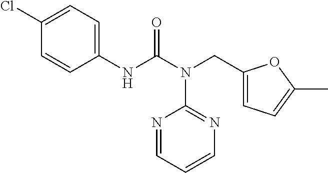 [SMILES: Cc1ccc(CN(C(=O)Nc2ccc(Cl)cc2)c2ncccn2)o1] | 343.02 | | 1H NMR (600 MHz, DMSO-d6) δ: 12.49 (s, 1H), 8.81 (d, J = 4.9 Hz, 2H), 7.70-7.66 (m, 2H), 7.42- 7.38 (m, 2H), 7.25 (t, J = 4.1 Hz, 1H), 6.07 (d, J = 3.0 Hz, 1H), 5.93 (dd, J = 3.0, 1.1 Hz, 1H), 5.34 (s, 2H), 2.19 (s, 3H) ppm. | 2-fluoro- pyrimidine | General method B.1 using (5-methyl-2- furyl)methanamine and DIPEA, method D1 using 4- chlorophenylisocyanate, TEA in toluene | 3-(4- chlorophenyl)-1- [(5-methyl-2- furyl)methyl]-1- pyrimidin-2-yl- urea |
| 328 | 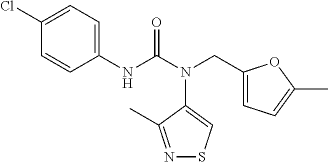 [SMILES: Cc1ccc(CN(C(=O)Nc2ccc(Cl)cc2)c2csnc2C)o1] | 362.13 | | 1H NMR (500 MHz, DMSO-d6) δ: 8.80 (s, 1H), 8.27 (s, 1H), 7.49- 7.42 (m, 2H), 7.30-7.25 (m, 2H), 6.03 (d, J = 3.0 Hz, 1H), 5.96-5.92 (m, 1H), 4.71 (s, 2H), 2.19 (s, 3H), 2.08 (s, 3H) ppm. | 5- methylfuran- 2- carbaldehyde | General method A using 3- methylisothiazol-4- amine, molecular sieves and NaBH₄, method D1 using 4- cyanophenylisocyanate | 3-(4- chlorophenyl)-1- [(5-methyl-2- furyl)methyl]-1- (3- methylisothiazol- 4-yl)urea |

### Biological Assays

### Example 1—Effect of Compounds on Tryptophan Catabolism in SK-OV-3 Cell Line

SK-OV-3 cells are seeded in 96-well plates in McCoy's 5A medium supplemented with 10% FBS and incubated overnight at 37° C., 5% CO2, 95% humidity. The next 5 day growth medium is replaced with DMEM F-12 medium supplemented with 6 mg/mL L-tryptophan. Compounds are added to the wells and incubated for 24 h at 37° C., 5% CO2, 95% humidity. Compounds are tested at 8 consecutive 3-fold dilutions starting from 30 μM in duplicate.

At the end of incubation, concentration of kynurenine in cell supernatants is 10 determined by LC-MS/MS. In parallel, cell viability is assessed by measurement of ATP using CellTiter Glo assay (Promega), according to manufacturer's protocol.

Inhibition of Kyn production is calculated using the following formula:

$$( 1 - \frac{Kyn conc . cmpd . - Kyn conc . medium}{Kyn conc . medium with cells - Kyn conc . medium} ) \cdot 100$$

IC50 values are determined using GraphPad Prism software 5.04 for Windows by plotting percent of inhibition against log 10 concentrations of compounds using a four-parametric sigmoidal curve with a variable slope.

Cell viability results are analysed in Microsoft Excel and expressed as fold change over untreated control.

IC50 ranges for compounds of the invention are presented in Table 2, whereby the IC50 ranges are as follows:
A: IC₅₀<100 nM
B: IC₅₀=100-500
C: IC₅₀=500-1000 nM
D: IC₅₀=1000-5000 nM
E: IC₅₀>5000 nM

TABLE 2
Effect of compounds on tryptophan catabolism in SK-OV-3 cell
line (ic50 values for inhibition of Kynurenine production)

| Cpd# | IC₅₀ Ranges |
|---|---|
| 1 | D |
| 2 | D |
| 3 | B |
| 4 | C |
| 5 | D |
| 6 | D |
| 7 | D |
| 8 | E |
| 9 | D |
| 10 | D |
| 11 | D |
| 12 | D |
| 13 | D |
| 14 | E |
| 15 | D |
| 16 | B |
| 17 | B |
| 18 | D |
| 19 | C |
| 20 | E |
| 21 | D |
| 22 | E |
| 23 | E |
| 24 | E |
| 25 | B |
| 26 | A |
| 27 | B |
| 28 | C |
| 29 | E |
| 30 | B |
| 31 | B |
| 32 | E |
| 33 | E |
| 34 | B |
| 35 | D |
| 36 | D |
| 37 | D |
| 38 | C |
| 39 | E |
| 40 | B |
| 41 | D |
| 42 | B |
| 43 | D |
| 44 | C |
| 45 | B |
| 46 | D |
| 47 | B |
| 48 | B |
| 49 | D |
| 50 | B |
| 51 | E |
| 52 | A |
| 53 | B |
| 54 | D |
| 55 | C |
| 56 | A |
| 57 | E |
| 58 | D |
| 59 | C |
| 60 | C |
| 61 | E |
| 62 | E |
| 63 | D |
| 64 | D |
| 65 | D |
| 66 | B |
| 67 | D |
| 68 | D |
| 69 | B |
| 70 | E |
| 71 | C |
| 72 | E |
| 73 | D |
| 74 | E |
| 75 | E |
| 76 | C |
| 77 | B |
| 78 | B |
| 79 | B |
| 80 | A |
| 81 | C |
| 82 | B |
| 83 | A |
| 84 | A |
| 85 | D |
| 86 | D |
| 87 | E |
| 88 | C |
| 89 | A |
| 90 | A |
| 91 | A |
| 92 | A |
| 93 | D |
| 94 | D |
| 95 | D |
| 96 | E |
| 97 | D |
| 98 | B |
| 99 | C |
| 100 | E |
| 101 | E |
| 102 | A |
| 103 | D |
| 104 | E |
| 105 | B |
| 106 | C |
| 107 | A |
| 108 | B |
| 109 | D |
| 110 | B |
| 111 | A |
| 112 | C |
| 113 | D |
| 114 | B |
| 115 | B |
| 116 | B |
| 117 | D |
| 118 | D |
| 119 | C |
| 120 | B |
| 121 | C |
| 122 | D |
| 123 | E |
| 124 | B |
| 125 | D |
| 126 | C |
| 127 | E |
| 128 | C |
| 129 | D |
| 130 | A |
| 131 | A |
| 132 | B |
| 133 | B |
| 134 | C |
| 135 | D |
| 136 | E |
| 137 | A |
| 138 | D |
| 139 | A |
| 140 | A |
| 141 | A |
| 142 | D |
| 143 | D |
| 144 | B |
| 145 | D |
| 146 | A |
| 147 | A |
| 148 | A |
| 149 | C |
| 150 | B |
| 151 | C |
| 152 | B |
| 153 | A |
| 154 | A |
| 155 | C |
| 156 | C |
| 157 | B |
| 158 | C |
| 159 | D |
| 160 | D |
| 161 | A |
| 162 | A |
| 163 | A |
| 164 | A |
| 165 | D |
| 166 | C |
| 167 | D |
| 168 | C |
| 169 | D |
| 170 | D |
| 171 | D |
| 172 | B |
| 173 | A |
| 174 | B |
| 175 | C |
| 176 | B |
| 177 | A |
| 178 | D |
| 179 | E |
| 180 | E |
| 181 | D |
| 182 | B |
| 183 | D |
| 184 | B |
| 185 | A |
| 186 | B |
| 187 | B |
| 188 | B |
| 189 | B |
| 190 | B |
| 191 | C |
| 192 | A |
| 193 | C |
| 194 | B |
| 195 | B |
| 196 | D |
| 197 | D |
| 98 | D |
| 199 | C |
| 200 | D |
| 201 | D |
| 202 | D |
| 203 | D |
| 204 | E |
| 205 | B |
| 206 | A |
| 207 | B |
| 208 | C |
| 209 | C |
| 210 | D |
| 211 | E |
| 212 | D |
| 213 | A |
| 214 | A |
| 215 | E |
| 216 | A |
| 217 | B |
| 218 | C |
| 219 | B |
| 220 | E |
| 221 | C |
| 222 | B |
| 223 | B |
| 224 | B |
| 225 | B |
| 226 | B |
| 227 | A |
| 228 | B |
| 229 | B |
| 230 | B |
| 231 | D |
| 232 | A |
| 233 | D |
| 234 | A |
| 235 | A |
| 236 | C |
| 237 | B |
| 238 | B |
| 239 | B |
| 240 | A |
| 241 | C |
| 242 | B |
| 243 | B |
| 244 | B |
| 245 | B |
| 246 | C |
| 247 | B |
| 248 | B |
| 249 | E |
| 250 | D |
| 251 | C |
| 252 | E |
| 253 | B |
| 254 | C |
| 255 | B |
| 256 | A |
| 257 | A |
| 258 | E |
| 259 | B |
| 260 | A |
| 261 | A |
| 262 | A |
| 263 | B |
| 264 | E |
| 265 | D |
| 266 | D |
| 267 | B |
| 268 | C |
| 269 | B |
| 270 | C |
| 271 | B |
| 272 | C |
| 273 | B |
| 274 | D |
| 275 | D |
| 276 | D |
| 277 | A |
| 278 | A |
| 279 | A |
| 280 | D |
| 281 | D |
| 282 | C |
| 283 | D |
| 284 | B |
| 285 | B |
| 286 | D |
| 287 | E |
| 288 | D |
| 289 | A |
| 290 | B |
| 291 | C |
| 292 | B |
| 293 | B |
| 294 | A |
| 295 | A |
| 296 | B |
| 297 | A |
| 298 | A |
| 299 | B |
| 300 | D |
| 301 | A |
| 302 | D |
| 303 | E |
| 304 | B |
| 305 | A |
| 306 | D |
| 307 | E |
| 308 | C |
| 309 | B |
| 310 | D |
| 311 | B |
| 312 | D |
| 313 | B |
| 314 | B |
| 315 | A |
| 316 | E |
| 317 | D |
| 318 | A |
| 319 | A |
| 320 | B |
| 321 | D |
| 322 | A |
| 323 | B |
| 324 | E |
| 325 | E |
| 326 | E |
| 327 | D |
| 328 | A |

### Example 2—IDO1 Enzyme Inhibition Assay

The effect of compounds on IDO1 enzymatic activity was assessed by use of a hIDO assay kit (Netherlands Translational Research Center B.V., Cat #NTRC-hIDO-1K). IDO1 enzyme and tryptophan substrate are diluted in assay buffer. Compounds are tested at 8 consecutive 3-fold dilutions starting from 30 μM. 267 nL of compound is added per well of a 384-well plate followed by addition of 10 μL of assay buffer. IDO1 enzyme is added at a final concentration of 25 nM and pre-incubated for 30 min at room temperature in the dark. Tryptophan substrate solution is then added at final concentration of 100 μM to all wells and the plate incubated for 60 min at RT in the dark. Finally NFK-green reagent is added at ⅕ of total reaction volume and incubated for 4 h at RT in the dark. After incubation fluorescence is measured at Ex405 nm-Em535 nm using EnVision plate reader.

IC50 values are determined using GraphPad Prism software 5.04 for Windows by plotting percent of inhibition against log 10 concentrations of compounds using a four-parametric sigmoidal curve with a variable slope.

TABLE 3
Inhibition of IDO1 activity by a selection
of compounds of invention 1

| | IDO1 Biochemical | | IDO1 Biochemical |
|---|---|---|---|
| Compound | assay IC50 (μM) | Compound | assay IC50 (μM) |
| 3 | >30 | 213 | >30 |
| 137 | >30 | 215 | >30 |
| 144 | >30 | 220 | >30 |
| 80 | >30 | 256 | >30 |
| 83 | >30 | 257 | >30 |
| 109 | >30 | 269 | >30 |
| 111 | >30 | 270 | >30 |
| 154 | >30 | 276 | >30 |
| 158 | >30 | 279 | >30 |
| 162 | >30 | 277 | >30 |

### Example 3—TDO2 Enzyme Inhibition Assay

The effect of compounds on TDO2 enzymatic activity was assess by use of an hTDO2 assay kit (Netherlands Translational Research Center B.V., Cat #NTRC-hTDO-1K). TDO2 enzyme and tryptophan substrate are diluted in assay buffer. Compounds are tested at 8 consecutive 3-fold dilutions starting from 30 μM. 267 nL of compound is added per well of a 384-well plate followed by addition of 10 μL of assay buffer. TDO2 enzyme is added at a final concentration of 50 nM and pre-incubated for 60 min at room temperature in the dark. Tryptophan substrate solution is then added at final concentration of 200 μM to all wells and the plate incubated for 15 min at RT in the dark. Finally NFK-green reagent is added at ⅕ of total reaction volume and incubated for 4 h at RT in the dark. After incubation fluorescence is measured at Ex405 nm-Em535 nm using EnVision plate reader.

IC50 values are determined using GraphPad Prism software 5.04 for Windows by plotting percent of inhibition against log 10 concentrations of compounds using a four-parametric sigmoidal curve with a variable slope.

TABLE 4
Inhibition of TDO2 activity by a selection
of compounds of invention 1

| | TDO2 Biochemical | | TDO2 Biochemical |
|---|---|---|---|
| Compound | assay IC50 | Compound | assay IC50 |
| 3 | >30 | 130 | >30 |
| 21 | >30 | 154 | >30 |
| 137 | >30 | 185 | >30 |
| 144 | >30 | 74 | >30 |
| 80 | >30 | 104 | >30 |
| 83 | >30 | 123 | >30 |
| 147 | >30 | 105 | >30 |
| 90 | >30 | 109 | 6.0 |
| 92 | >30 | 110 | >30 |
| 102 | >30 | 158 | 6.4 |
| 111 | 4.8/2.2 | 162 | >30 |

### Example 4—Effect of Compounds on IDO1 Expression in SK-OV-3 Cell Line

SK-OV-3 cells are seeded in 24-well plates in McCoy's 5A medium supplemented with 10% FBS at density of 250000 cells per well and incubated overnight at 37° C., 5% CO2, 95% humidity. The following day growth medium is replaced with DMEM F-12 medium containing 6 mg/mL L-tryptophan. Compounds are added to the wells and the plate is incubated for 24 h at 37° C., 5% CO2, 95% humidity. At the end of incubation medium is removed, cells are washed and then lysed in RIPA buffer. Protein concentrations in the samples are determined using BCA method (Thermo Scientific) and adjusted to 0.5 mg/mL. Western analysis is performed on the Wes system (Protein Simple) using IDO1 (Cell Signalling; Cat #12006S) and GAPDH (Abcam, Cat #ab9485) antibodies. Expression of IDO1 is normalised to GAPDH.

### Example 5—Effect of Compounds on Tryptophan Catabolism in Monocyte-Derived Dendritic Cells

Peripheral blood mononuclear cells are isolated from buffy coats from healthy volunteers by Lymphoprep density gradient centrifugation, followed by lysis of residual erythrocytes with isotonic buffer solution of ammonium chloride. CD14+ cells are isolated by positive selection using MACS® technology and CD14 MicroBeads (Miltenyi Biotec) according to manufacturer's instructions. The isolated monocytes are differentiated into dendritic cells by incubation for 5 days in RPMI-1640 medium supplemented with 10% fetal bovine serum (FBS), 50 ng/mL GM-CSF and 35 ng/mL IL-4. After 5 days the DCs are seeded in 96-well plates in DMEM supplemented with 10% charcoal stripped FBS, GM-CSF and IL-4. The following day cells are triggered with LPS and IFNγ. After 24 h of incubation medium is replaced with DMEM F-12 medium supplemented with 6 mg/mL L-tryptophan and 10% charcoal stripped FBS. Compounds are added to the cells and incubated for 24 h at 37° C., 5% CO2, 95% humidity. Compounds are tested at 7 consecutive 4-fold dilutions starting from 30 μM in duplicate.

At the end of incubation concentration of kynurenine in cell supernatants is determined by LC-MS/MS. In parallel, cell viability is assessed by measurement of ATP by use of CellTiter Glo assay (Promega) according to manufacturer's protocol.

Inhibition of Kyn production is calculated using the following formula:

(1−(Kyn conc. cmpd.−Kyn conc. medium)/(Kyn conc. medium with cells−Kyn conc. medium))·100

IC50 values are determined using GraphPad Prism software 5.04 for Windows, by plotting percent of inhibition against log 10 concentrations of compounds using a four-parametric sigmoidal curve with a variable slope.

Cell viability results are analysed in Microsoft Excel and expressed as fold change over untreated control.

TABLE 5
inhibition of kynurenine production and cytotoxicity
of compounds of the invention in dendritic cells

| Compound | Kynurenine IC50 | Cytotoxicity at 30 uM |
|---|---|---|
| 26 | 0.10 | 19 |
| 56 | 0.25 | 94 |
| 137 | 0.040 | 88 |
| 139 | 0.27 | 77 |
| 140 | 0.32 | 92 |
| 141 | 0.47 | 90 |
| 144 | 0.38 | 109 |
| 146 | 0.12 | 103 |
| 80 | 0.026 | 112 |
| 82 | 0.30 | 85 |
| 83 | 0.13 | 89 |
| 84 | 0.18 | 108 |
| 147 | 0.070 | 122 |
| 148 | 0.15 | 103 |
| 89 | 0.065 | 81 |
| 90 | 0.066 | 67 |
| 91 | 0.036 | 71 |

### Example 6—Tumour Cell Killing Assay

Human PBMCs from healthy donors are prepared from buffy coat and cryopreserved. For the killing assay, on day −1, NucLight™ Red transfected SK-OV-3 ovarian cancer cells are seeded in to a Flat-bottomed 96-well plate at 2×103 cells/well (100 μL per well) and incubated in the Incucyte Zoom® overnight. The following day (day 0) media is aspirated from wells containing SK-OV-3 cells. Caspase 3/7 reagent (1:2000) is prepared and added to wells at 50 μL per well, together with 50 μL test substances. Anti-CD3, (final concentration 0.1 μg/mL), anti-CD28 (final concentration 0.5 μg/mL) and rhIL-2 (final concentration 10 ng/mL) are prepared in complete media and added in a final volume of 50 μL per well. Untouched PBMCs are prepared from cryopreserved stock and added to wells at 1×104 cells per well in a volume of 50 μL per well, such that the final well volume is 200 μL. Cells are incubated/monitored in the Incucyte Zoom® for a period of seven days.

Wells are imaged at 3-hour intervals in phase, green and red channels. Automated image analysis enables selective quantitation of SK-OV-3 nuclei (Red) per well, apoptotic SK-OV-3 nuclei (Green/Red colocalised) to enable the effect of test substance on apoptosis to be determined and quantified graphically over time.

1-54. (canceled)

55. A compound of Formula (I): 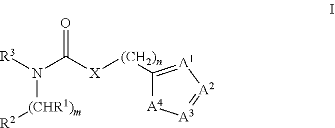
or a pharmaceutically acceptable salt, or a solvate, or a solvate of the salt thereof, wherein:
m is 0 or 1;
n is 0, 1 or 2;
X is —NR⁸;
R¹ is H, C₁₋₆alkyl or a 6-10 membered aryl;
R² is a 5-6-membered heteroaryl, a fused 9-10 membered bicyclic heteroaryl, a 6-10 membered aryl, a 5-6 membered monocyclic heterocycloalkyl or a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy or phenyl;
R³ is H or C₁₋₆alkyl; or a 3-10 membered cycloalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆alkyl, halogen, —CN or —C(═O)OC₁₋₆alkyl;
A¹ is —N— or —CR⁶—;
A² is —N— or —CR⁵—;
A³ is —N— or —CR⁷—;
A⁴ is —N—, —O—, —S—, —CH═N— or —CH═CR⁴—;
R⁴, R⁵, R⁶ and R⁷, which may be the same or different, are each selected from —H, —OH, —C₁₋₆alkyl, halogen, haloC₁₋₆alkyl, —CN, —C₁₋₆alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, —C(═O)C₁₋₆alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;
or R⁵ and R⁷ together form a ring —CH═CH—CH═CH—, —OCH₂O— or —CH₂CH₂CH₂—;
or the moiety 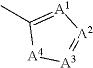
may be fused with oxopyrrolidine; and
R⁸, R⁹, R¹⁰, R¹¹, R¹², and R¹³, which may be the same or different, are each selected from H or C₁₋₆alkyl;
provided that the compound of formula I is not 1-(4-chlorobenzyl)-1-cyclopentyl-3-phenylurea;
N-(3,5-dimethylphenyl)-3-ethyl-2-methyl-7-phenyl-5,7-dihydro-4H-thieno[2,3-c]pyridine-6-carboxamide; [194]
1-cyclopentyl-3-phenyl-1-(2-thienylmethyl)urea; [195]
1-(4-chlorophenyl)-3-phenyl-1-(2-thienylmethyl)urea; [196]
1-[1-(4-fluorophenyl)ethyl]-3-phenyl-urea; [197]
1-(4-chlorophenyl)-3-[1-(5-chloro-2-thienyl)ethyl]urea; [199]
3-(3,4-dichlorophenyl)-1-methyl-1-(2-thienylmethyl)urea; [200]
1-[(5-methyl-2-phenyl-oxazol-4-yl)methyl]-3-phenyl-urea; [203] and
1-(3-chlorophenyl)-3-[(3-chloro-2-thienyl)methyl]urea; [204].

56-57. (canceled)

58. The compound according to claim 55, wherein m is 1.

59. The compound according to claim 55, wherein n is 0.

60. The compound according to claim 55, wherein n is 2.

61. The compound according to claim 55, wherein R¹ is H.

62. The compound according to claim 55, wherein X is —NH—.

63. The compound according to claim 55, wherein R² is a 5-6-membered heteroaryl or a fused 9-10 membered bicyclic heteroaryl.

64-66. (canceled)

67. The compound according to claim 55, wherein R³ is a 5-6-membered heteroaryl.

68. (canceled)

69. The compound according to claim 55, wherein R³ is a 4-6 membered monocyclic heterocycloalkyl.

70. (canceled)

71. The compound according to claim 55, wherein the moiety 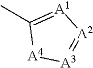
is selected from the group consisting of benzothiazole, indane, oxadiazole, phenyl, pyridine, pyrimidine, thiazole and thiophene; each of which may independently be optionally substituted by one or more groups independently selected from OH, —C₁₋₆alkyl, C₃₋₆cycloalkyl halogen, haloC₁₋₆alkyl, —CN, —C₁₋₆alkyl-CN, —C₁₋₆alkyl-OH, —OC₁₋₆alkyl, —C₂₋₆alkynyl, —C₁₋₆alkyl-OC₁₋₆alkyl, haloC₁₋₆alkyl-O—, —C₁₋₆alkyl-O—NH₂, C₂₋₆alkynyl-OC₁₋₆alkyl; a 3-10 membered cycloalkyl, a 6-10 membered aryl, a 5-6 membered heteroaryl, a 4-6 membered monocyclic heterocycloalkyl, a fused 8-10 membered partially unsaturated bicyclic heterocyclyl or a fused 9-10 membered bicyclic heteroaryl, each of which may independently be optionally substituted by one or more groups independently selected from —C₁₋₆alkyl, C₁₋₆alkyl-NR⁹R¹⁰, —C(═O)C₁₋₆alkyl, —C(═O)OC₁₋₆alkyl, —C₁₋₆alkyl-OH, C₂₋₆alkynyl-C₁₋₆alkyl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, C₂₋₆alkynyl-aryl, C₂₋₆alkynyl-C₁₋₆alkyl-aryl, —C(═O)NH₂ or —C(═O)OC₁₋₆alkyl.

72. The compound according to claim 55, wherein the moiety 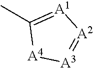
is phenyl, which may independently be optionally substituted by one or more groups independently selected from OH, —C₁₋₆alkyl, C₃₋₆cycloalkyl halogen, haloC₁₋₆alkyl, —CN, —C₁₋₆alkyl-CN, —C₁₋₆alkyl-OH, —OC₁₋₆alkyl, —C₂₋₆alkynyl, —C₁₋₆alkyl-OC₁₋₆alkyl, haloC₁₋₆alkyl-O—, —C₁₋₆alkyl-O—NH₂, C₂₋₆alkynyl-OC₁₋₆alkyl; a 3-10 membered cycloalkyl, a 6-10 membered aryl, a 5-6 membered heteroaryl, a 4-6 membered monocyclic heterocycloalkyl, a fused 8-10 membered partially unsaturated bicyclic heterocyclyl or a fused 9-10 membered bicyclic heteroaryl, each of which may independently be optionally substituted by one or more groups independently selected from —C₁₋₆alkyl, C₁₋₆alkyl-NR⁹R¹⁰, —C(═O)C₁₋₆alkyl, —C(═O)OC₁₋₆alkyl, —C₁₋₆alkyl-OH, C₂₋₆alkynyl-C₁₋₆alkyl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, C₂₋₆alkynyl-aryl, C₂₋₆alkynyl-C₁₋₆alkyl-aryl, —C(═O)NH₂ and —C(═O)OC₁₋₆alkyl.

73. The compound according to claim 55, wherein A³ is —CR⁷—, wherein R⁷ is selected from the group consisting of the following ring structures:

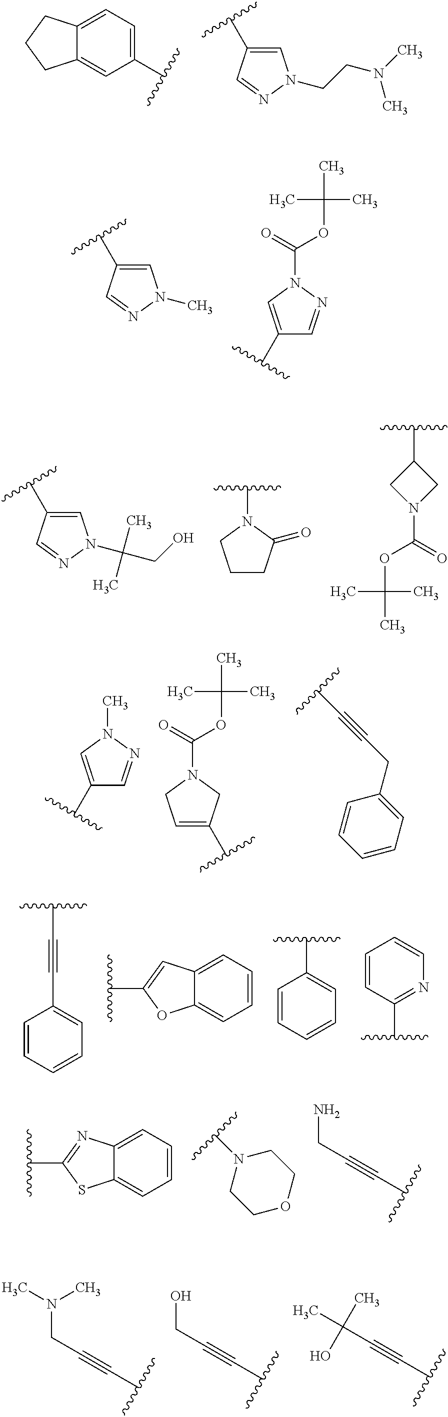

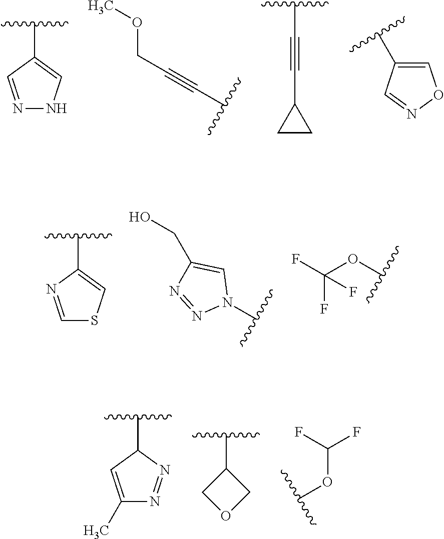

74. The compound according to claim 55, wherein R⁵ is H or halogen.

75. The compound according to claim 55, wherein R⁶ is H or —C₁₋₆alkyl.

76. The compound according to claim 55 wherein the compound is selected from the group consisting of:
1-cyclopentyl-3-(2-phenylethyl)-1-(2-thienylmethyl)urea; [1]
3-(2-chlorophenyl)-1-cyclopentyl-1-(2-thienylmethyl)urea; [2]
1-cyclopentyl-3-(4-ethylphenyl)-1-(2-thienylmethyl)urea; [3]
1-cyclopentyl-3-(3,4-difluorophenyl)-1-(2-thienylmethyl)urea; [4]
1-cyclopentyl-3-(2,4-dimethylphenyl)-1-(2-thienylmethyl)urea; [5]
3-[4-(cyanomethyl)phenyl]-1-cyclopentyl-1-(2-thienylmethyl)urea; [6]
3-(1,3-benzodioxol-5-yl)-1-cyclopentyl-1-(2-thienylmethyl)urea; [7]
1-cyclopentyl-3-[(4-fluorophenyl)methyl]-1-(2-thienylmethyl)urea; [8]
1-cyclopentyl-3-indan-5-yl-1-(2-thienylmethyl)urea; [9]
“1-cyclopentyl-3-(2,6-dichloro-4-pyridyl)-1-(2-thienylmethyl)urea; [10]”
1-cyclopentyl-3-(4-pyridyl)-1-(2-thienylmethyl)urea; [11]
1-cyclopentyl-1-(2-thienylmethyl)-3-[4-(trifluoromethyl)phenyl]urea; [13]
1-cyclopentyl-3-(4-methoxyphenyl)-1-(2-thienylmethyl)urea; [14]
3-allyl-1-cyclopentyl-1-(2-thienylmethyl)urea; [15]
“1-cyclopentyl-3-(5-ethynyl-2-pyridyl)-1-[(5-methyl-2-furyl)methyl]urea; [16]”
“1-cyclopentyl-3-(5-ethynylpyrimidin-2-yl)-1-[(5-methyl-2-furyl)methyl]urea; [17]”
1-cyclopentyl-3-(2,4-dimethoxyphenyl)-1-(2-thienylmethyl)urea; [18]
1-cyclopentyl-3-phenyl-1-(2-thienylmethyl)urea; [19]
1-cyclohexyl-3-(2-phenylethyl)-1-(2-pyridylmethyl)urea; [20]
1-cyclohexyl-3-(4-ethylphenyl)-1-(2-pyridylmethyl)urea; [21]
3-(4-acetylphenyl)-1-cyclopentyl-1-(2-thienylmethyl)urea; [22]
1-cyclopentyl-3-methyl-3-phenyl-1-(2-thienylmethyl)urea; [23]
1-cyclopentyl-1-[(5-methyl-2-thienyl)methyl]-3-phenyl-urea; [24]
1-cyclopentyl-3-(4-ethylphenyl)-1-[(5-methyl-2-thienyl)methyl]urea; [25]
1-cyclopentyl-1-[(5-methyl-2-thienyl)methyl]-3-(2-phenylethyl)urea; [26]
“1-cyclopentyl-3-(3,4-difluorophenyl)-1-[(5-methyl-2-thienyl)methyl]urea; [27]”
1-cyclopentyl-1-(2-furylmethyl)-3-phenyl-urea; [28]
1-cyclopentyl-1-(2-furylmethyl)-3-(2-phenylethyl)urea; [29]
1-cyclopentyl-3-(3,4-difluorophenyl)-1-(2-furylmethyl)urea; [30]
1-cyclopentyl-3-(3,4-difluorophenyl)-1-[(5-methyl-2-furyl)methyl]urea; [31]
1-cyclohexyl-3-phenyl-1-(2-pyridylmethyl)urea; [32]
1-cyclohexyl-3-(3,4-difluorophenyl)-1-(2-pyridylmethyl)urea; [33]
1-cyclopentyl-3-(4-fluorophenyl)-1-(2-thienylmethyl)urea; [34]
1-cyclopentyl-3-phenyl-1-(thiazol-2-ylmethyl)urea; [35]
1-cyclopentyl-3-(4-ethylphenyl)-1-(thiazol-2-ylmethyl)urea; [36]
1-cyclopentyl-3-(3,4-difluorophenyl)-1-(thiazol-2-ylmethyl)urea; [37]
1-[(2-chlorophenyl)methyl]-1-cyclopentyl-3-phenyl-urea; [38]
1-[(2-chlorophenyl)methyl]-1-cyclopentyl-3-(4-ethylphenyl)urea; [39]
1-[(2-chlorophenyl)methyl]-1-cyclopentyl-3-(3,4-difluorophenyl)urea; [40]
1-[(5-chloro-1-methyl-pyrazol-4-yl)methyl]-1-cyclopentyl-3-phenyl-urea; [41]
1-[(5-chloro-1-methyl-pyrazol-4-yl)methyl]-1-cyclopentyl-3-(4-ethylphenyl)urea; [42]
1-[(5-chloro-1-methyl-pyrazol-4-yl)methyl]-1-cyclopentyl-3-(3,4-difluorophenyl) urea; [43]
1-[(5-chloro-1-methyl-pyrazol-4-yl)methyl]-1-cyclopentyl-3-(4-fluorophenyl) urea; [44]
1-cyclopentyl-1-[(4-methoxy-3-methyl-phenyl)methyl]-3-phenyl-urea; [45]
1-cyclopentyl-1-[(4-methoxy-3-methyl-phenyl)methyl]-3-(2-phenylethyl)urea; [46]
1-cyclopentyl-3-(3,4-difluorophenyl)-1-[(4-methoxy-3-methyl-phenyl)methyl]urea; [47]
1-cyclopentyl-3-(4-fluorophenyl)-1-[(4-methoxy-3-methyl-phenyl)methyl]urea; [48]
1-cyclopentyl-1-[(2-methoxythiazol-5-yl)methyl]-3-phenyl-urea; [49]
1-[(3-cyano-4-fluoro-phenyl)methyl]-1-cyclopentyl-3-phenyl-urea; [50]
1-[(3-cyano-4-fluoro-phenyl)methyl]-1-cyclopentyl-3-(2-phenylethyl)urea; [51]
1-[(3-cyano-4-fluoro-phenyl)methyl]-1-cyclopentyl-3-(3,4-difluorophenyl)urea; [52]
1-[(3-cyano-4-fluoro-phenyl)methyl]-1-cyclopentyl-3-(4-fluorophenyl)urea; [53]
1-cyclopentyl-3-(2-fluorophenyl)-1-(2-thienylmethyl)urea; [54]
1-cyclopentyl-3-(3-fluorophenyl)-1-(2-thienylmethyl)urea; [55]
3-(4-chlorophenyl)-1-cyclopentyl-1-(2-thienylmethyl)urea; [56]
1-cyclopentyl-3-(3-pyridyl)-1-(2-thienylmethyl)urea; [57]
1-cyclopentyl-1-phenyl-3-(2-thienyl)urea; [58]
1-cyclopentyl-3-(2,4-dichlorophenyl)-1-(2-thienylmethyl)urea; [59]
1-[(5-cyano-2-furyl)methyl]-1-cyclopentyl-3-phenyl-urea; [60]
1-[(5-cyano-2-furyl)methyl]-1-cyclopentyl-3-(4-fluorophenyl)urea; [61]
1-cyclopentyl-3-(4-fluorophenyl)-1-(isoxazol-4-ylmethyl)urea; [62]
3-(4-chlorophenyl)-1-cyclopentyl-1-(isoxazol-4-ylmethyl)urea; [63]
1-cyclopentyl-1-(3-furylmethyl)-3-phenyl-urea; [64]
1-cyclopentyl-3-phenyl-1-(3-pyridylmethyl)urea; [65]
3-(4-chlorophenyl)-1-cyclopentyl-1-(3-pyridylmethyl)urea; [66]
1-cyclopentyl-3-phenyl-1-(2-pyridylmethyl)urea; [67]
1-cyclopentyl-3-(4-fluorophenyl)-1-(2-pyridylmethyl)urea; [68]
3-(4-chlorophenyl)-1-cyclopentyl-1-(2-pyridylmethyl)urea; [69]
“1-cyclopentyl-3-(4-fluorophenyl)-1-(pyrazin-2-ylmethyl)urea; [70]”
3-(4-chlorophenyl)-1-cyclopentyl-1-(pyrazin-2-ylmethyl)urea; [71]
1-cyclopentyl-3-(4-fluorophenyl)-1-(pyrimidin-2-ylmethyl)urea; [72]
3-(4-chlorophenyl)-1-cyclopentyl-1-(pyrimidin-2-ylmethyl)urea; [73]
1-cyclopentyl-3-phenyl-1-(4-pyridylmethyl)urea; [74]
“1-cyclopentyl-3-(4-fluorophenyl)-1-(4-pyridylmethyl)urea; [75]”
3-(4-chlorophenyl)-1-cyclopentyl-1-(4-pyridylmethyl)urea; [76]
tert-butyl 4-[[cyclopentyl(phenylcarbamoyl)amino]methyl]-4-methyl-piperidine-1-carboxylate; [77]
“tert-butyl 4-[[cyclopentyl-[(4-fluorophenyl)carbamoyl]amino]methyl]-4-methyl-piperidine-1-carboxylate; [78]”
tert-butyl 4-[[(4-chlorophenyl)carbamoyl-cyclopentyl-amino]methyl]-4-methyl-piperidine-1-carboxylate; [79]
3-(4-cyanophenyl)-1-cyclopentyl-1-[(5-methyl-2-furyl)methyl]urea; [80]
1-cyclopentyl-1-[(5-methyl-2-furyl)methyl]-3-(4-pyridyl)urea; [81]
1-cyclobutyl-3-(4-fluorophenyl)-1-[(5-methyl-2-furyl)methyl]urea; [82]
3-(4-chlorophenyl)-1-cyclobutyl-1-[(5-methyl-2-furyl)methyl]urea; [83]
3-(4-cyanophenyl)-1-cyclobutyl-1-[(5-methyl-2-furyl)methyl]urea; [84]
1-cyclopentyl-1-[(4-methyl-4-piperidyl)methyl]-3-phenyl-urea; [85]
1-cyclopentyl-3-(4-fluorophenyl)-1-[(4-methyl-4-piperidyl)methyl]urea; [86]
3-(4-chlorophenyl)-1-cyclopentyl-1-[(4-methyl-4-piperidyl)methyl]urea; [87]
“1-cyclopentyl-3-(4-pyridyl)-1-[[3-(trifluoromethyl)phenyl]methyl]urea; [88]”
“3-(4-cyanophenyl)-1-cyclopentyl-1-[[3-(trifluoromethyl)phenyl]methyl]urea; [89]”
3-(4-chlorophenyl)-1-cyclobutyl-1-[[3-(trifluoromethyl)phenyl]methyl]urea; [90]
3-(4-cyanophenyl)-1-cyclobutyl-1-[[3-(trifluoromethyl)phenyl]methyl]urea; [91]
3-(6-chloro-3-pyridyl)-1-cyclopentyl-1-[(5-methyl-2-furyl)methyl]urea; [92]
3-(3-cyanophenyl)-1-cyclopentyl-1-[(5-methyl-2-furyl)methyl]urea; [93]
3-(4-acetylphenyl)-1-cyclopentyl-1-[(5-methyl-2-furyl)methyl]urea; [94]
3-(2-cyanophenyl)-1-cyclopentyl-1-[(5-methyl-2-furyl)methyl]urea; [95]
“3-[(4-cyanophenyl)methyl]-1-cyclopentyl-1-[(5-methyl-2-furyl)methyl]urea; [96]”
1-[(1-acetyl-4-methyl-4-piperidyl)methyl]-1-cyclopentyl-3-phenyl-urea; [97]
1-cyclopentyl-1-[(5-methyl-2-furyl)methyl]-3-[4-(1-methylpyrazol-4-yl)phenyl] urea; [98]
“tert-butyl 4-[4-[[cyclopentyl-[(5-methyl-2-furyl)methyl]carbamoyl]amino]phenyl] pyrazole-1-carboxylate; [99]”
1-cyclopentyl-3-[4-[1-[2-(dimethylamino)ethyl]pyrazol-4-yl]phenyl]-1-[(5-methyl-2-furyl)methyl]urea; [100]
1-cyclopentyl-3-[4-[1-(2-hydroxy-1,1-dimethyl-ethyl)pyrazol-4-yl]phenyl]-1-[(5-methyl-2-furyl)methyl]urea; [101]
“1-cyclopentyl-3-(4-ethynylphenyl)-1-[(5-methyl-2-furyl)methyl]urea; [102]”
“1-cyclopentyl-1-[(5-methyl-2-furyl)methyl]-3-[4-(2-oxopyrrolidin-1-yl)phenyl] urea; [103]”
“1-cyclopentyl-3-(4-fluoro-3-hydroxy-phenyl)-1-[(5-methyl-2-furyl)methyl] urea; [104]”
“1-cyclopentyl-3-(4-isoxazol-4-ylphenyl)-1-[(5-methyl-2-furyl)methyl]urea; [105]”
1-cyclopentyl-1-[(5-methyl-2-furyl)methyl]-3-(4-thiazol-4-ylphenyl)urea; [106]
1-cyclopentyl-3-[4-(2-cyclopropylethynyl)phenyl]-1-[(5-methyl-2-furyl)methyl] urea; [107]
1-cyclopentyl-1-[(5-methyl-2-furyl)methyl]-3-[4-(1H-pyrazol-4-yl)phenyl]urea; [108]
1-cyclopentyl-3-[4-(3-hydroxy-3-methyl-but-1-ynyl)phenyl]-1-[(5-methyl-2-furyl) methyl]urea; [109]
3-[4-(3-aminoprop-1-ynyl)phenyl]-1-cyclopentyl-1-[(5-methyl-2-furyl)methyl] urea; [110]
1-cyclopentyl-3-[4-(3-hydroxyprop-1-ynyl)phenyl]-1-[(5-methyl-2-furyl)methyl] urea; [111]
1-cyclopentyl-3-[4-[3-(dimethylamino)prop-1-ynyl]phenyl]-1-[(5-methyl-2-furyl) methyl]urea; [112]
“1-cyclopentyl-3-(4-fluorophenyl)-1-[(4-methyl-1-methylsulfonyl-4-piperidyl)methyl]urea; [113]”
3-(4-chlorophenyl)-1-[(2-cyano-4-pyridyl)methyl]-1-cyclopentyl-urea; [114]
3-(4-chlorophenyl)-1-[(5-cyano-3-pyridyl)methyl]-1-cyclopentyl-urea; [115]
3-(4-chlorophenyl)-1-[(4-cyano-2-pyridyl)methyl]-1-cyclopentyl-urea; [116]
4-[[cyclopentyl-[(5-methyl-2-furyl)methyl]carbamoyl]amino]benzamide; [117]
tert-butyl 4-[[cyclopentyl(phenylcarbamoyl)amino]methyl]piperidine-1-carboxylate; [118]
tert-butyl 4-[[cyclopentyl-[(4-fluorophenyl)carbamoyl]amino]methyl]piperidine-1-carboxylate; [119]
tert-butyl 4-[[(4-chlorophenyl)carbamoyl-cyclopentyl-amino]methyl]piperidine-1-carboxylate; [120]
3-(5-cyano-2-pyridyl)-1-cyclopentyl-1-[(5-methyl-2-furyl)methyl]urea; [121]
5-[[cyclopentyl-[(4-fluorophenyl)carbamoyl]amino]methyl]-2-fluoro-benzamide; [122]
1-cyclopentyl-1-[(5-methyl-2-furyl)methyl]-3-(4-morpholinophenyl)urea; [123]
3-(6-cyano-3-pyridyl)-1-cyclopentyl-1-[(5-methyl-2-furyl)methyl]urea; [124]
1-[(6-cyano-2-pyridyl)methyl]-1-cyclopentyl-3-phenyl-urea; [125]
1-[(6-cyano-2-pyridyl)methyl]-1-cyclopentyl-3-(4-fluorophenyl)urea; [126]
1-cyclopentyl-3-(4-fluorophenyl)-1-(isoxazol-5-ylmethyl)urea; [127]
3-(4-chlorophenyl)-1-cyclopentyl-1-(isoxazol-5-ylmethyl)urea; [128]
1-cyclopentyl-1-[(5-methyl-2-furyl)methyl]-3-(4-phenylthiazol-2-yl)urea; [129]
1-(benzofuran-2-ylmethyl)-3-(4-chlorophenyl)-1-cyclopentyl-urea; [130]
1-(benzofuran-2-ylmethyl)-1-cyclopentyl-3-(4-ethynylphenyl)urea; [131]
“1-(benzofuran-2-ylmethyl)-3-(4-cyanophenyl)-1-cyclopentyl-urea; [132]”
“1-cyclopentyl-1-[(5-methyl-2-furyl)methyl]-3-[4-(2-pyridyl)thiazol-2-yl]urea; [133]”
3-(1,3-benzothiazol-2-yl)-1-cyclopentyl-1-[(5-methyl-2-furyl)methyl]urea; [134]
“3-(4-cyanothiazol-2-yl)-1-cyclopentyl-1-[(5-methyl-2-furyl)methyl]urea; [135]”
“tert-butyl 4-[(5-methyl-2-furyl)methyl-(phenylcarbamoyl)amino]piperidine-1-carboxylate; [136]”
3-(4-chlorophenyl)-1-cyclopentyl-1-[(5-methyl-2-furyl)methyl]urea; [137]
1-isopropyl-1-[(5-methyl-2-furyl)methyl]-3-phenyl-urea; [138]
1-cyclopentyl-3-(3,4-difluorophenyl)-1-[[4-fluoro-3-(trifluoromethyl)phenyl]methyl] urea; [139]
1-cyclopentyl-3-(3,4-difluorophenyl)-1-[[3 (trifluoromethyl)phenyl]methyl]urea; [140]
1-cyclopentyl-3-(3,4-difluorophenyl)-1-[[4-fluoro-3 (trifluoromethoxy)phenyl]methyl]urea; [141]
1-cyclopentyl-3-(3,4-difluorophenyl)-1-[[3 (trifluoromethoxy)phenyl]methyl] urea; [142]
1-cyclopentyl-1-[(2-methyloxazol-5-yl)methyl]-3-phenyl-urea; [143]
3-(4-chlorophenyl)-1-cyclopentyl-1-[(2-methyloxazol-5-yl)methyl]urea; [144]
1-cyclopentyl-3-(4-fluorophenyl)-1-[(2-methyloxazol-5-yl)methyl]urea; [145]
3-(4-chlorophenyl)-1-[(3-cyano-4-fluoro-phenyl)methyl]-1-cyclopentyl-urea; [146]
3-(4-chlorophenyl)-1-[(3-cyanophenyl)methyl]-1-cyclopentyl-urea; [147]
3-(4-chlorophenyl)-1-cyclopentyl-1-[[2-(trifluoromethyl)-4-pyridyl]methyl]urea; [148]
3-(4-chlorophenyl)-1-cyclopentyl-1-(isoxazol-3-ylmethyl)urea; [149]
1-cyclopentyl-1-[(5-methyl-2-furyl)methyl]-3-(4-phenylphenyl)urea; [150]
3-[5-(benzofuran-2-yl)-1,3,4-oxadiazol-2-yl]-1-cyclopentyl-1-[(5-methyl-2-furyl) methyl]urea; [151]
3-(4-cyanophenyl)-1-cyclobutyl-1-[[2-(trifluoromethyl)-4-pyridyl]methyl]urea; [152]
1-cyclobutyl-3-(4-ethynylphenyl)-1-[[2-(trifluoromethyl)-4-pyridyl]methyl]urea; [153]
1-cyclobutyl-3-(4-prop-1-ynylphenyl)-1-[[2-(trifluoromethyl)-4-pyridyl]methyl] urea; [154]
1-cyclopentyl-1-[(5-methyl-2-furyl)methyl]-3-[4-(2-phenylethynyl)phenyl]urea; [155]
1-cyclopentyl-1-[(5-methyl-2-furyl)methyl]-3-[4-(3-phenylprop-1-ynyl)phenyl] urea; [156]
3-(4-chlorophenyl)-1-[(6-cyano-2-pyridyl)methyl]-1-cyclopentyl-urea; [157]
tert-butyl 3-[4-[[cyclopentyl-[(5-methyl-2-furyl)methyl]carbamoyl]amino]phenyl]-2,5-dihydropyrrole-1-carboxylate; [158]
tert-butyl 3-[4-[[cyclopentyl-[(5-methyl-2-furyl)methyl]carbamoyl]amino]phenyl] azetidine-1-carboxylate; [159]
1-cyclopentyl-1-[(5-methyl-2-furyl)methyl]-3-[6-(1-methylpyrazol-4-yl)-3-pyridyl] urea; [160]
3-(5-bromothiazol-2-yl)-1-cyclopentyl-1-[(5-methyl-2-furyl)methyl]urea; [161]
1-cyclopentyl-3-[4-(3-methoxyprop-1-ynyl)phenyl]-1-[(5-methyl-2-furyl)methyl] urea; [162]
1-cyclopentyl-3-(4-ethynylphenyl)-1-(isoxazol-5-ylmethyl)urea; [163]
1-cyclopentyl-3-[4-[4-(hydroxymethyl)triazol-1-yl]phenyl]-1-[(5-methyl-2-furyl) methyl]urea; [164]
“1-ethyl-1-[(5-methyl-2-furyl)methyl]-3-phenyl-urea; [165]”
“1-[(5-methyl-2-furyl)methyl]-3-phenyl-1-propyl-urea; [166]”
“1-cyclopropyl-1-[(5-methyl-2-furyl)methyl]-3-phenyl-urea; [167]”
1-cyclopentyl-1-[(5-methyl-2-furyl)methyl]-3-[5-(2-thienyl)-1,3,4-oxadiazol-2-yl] urea; [168]
3-(5-cyclohexyl-1,3,4-oxadiazol-2-yl)-1-cyclopentyl-1-[(5-methyl-2-furyl)methyl] urea; [169]
“1-cyclopentyl-1-[(2,4-dimethylthiazol-5-yl)methyl]-3-phenyl-urea; [170]”
“1-cyclopentyl-1-[(2,4-dimethylthiazol-5-yl)methyl]-3-(3,4-difluorophenyl)-urea; [171]”
3-(4-chloro-2-fluoro-phenyl)-1-cyclopentyl-1-[(5-methyl-2-furyl)methyl]urea; [172]
3-(4-chlorophenyl)-1-[1-(3-cyanophenyl)ethyl]-1-cyclopentyl-urea; [173]
3-(4-chlorophenyl)-1-cyclopentyl-1-[1-(2-pyridyl)ethyl]urea; [174]
1-cyclopentyl-1-[(5-methyl-2-furyl)methyl]-3-(5-phenyl-1,3,4-oxadiazol-2-yl)urea; [175]
“1-cyclopentyl-1-[(5-methyl-2-furyl)methyl]-3-phenyl-urea; [176]”
“1-cyclopentyl-3-(4-ethylphenyl)-1-[(5-methyl-2-furyl)methyl]urea; [177]”
1-cyclobutyl-1-[(5-methyl-2-furyl)methyl]-3-phenyl-urea; [178]
1-[(5-methyl-2-furyl)methyl]-1-oxazol-2-yl-3-phenyl-urea; [179]
3-(4-fluorophenyl)-1-[(5-methyl-2-furyl)methyl]-1-oxazol-2-yl-urea; [180]
“1-cyclopentyl-1-[(5-methyl-2-furyl)methyl]-3-(2-phenylethyl)urea; [181]”
3-(4-chlorophenyl)-1-[(5-methyl-2-furyl)methyl]-1-(1-methylimidazol-2-yl) urea; [182]
“3-(4-chlorophenyl)-1-(1H-imidazol-5-yl)-1-[(5-methyl-2-furyl)methyl]urea; [183]”
3-(4-cyanophenyl)-1-[1-(3-cyanophenyl)ethyl]-1-cyclobutyl-urea; [184]
3-(4-chlorophenyl)-1-cyclopentyl-1-[1-(5-methyl-2-furyl)ethyl]urea; [185]
1-[1-(3-cyanophenyl)ethyl]-1-cyclopentyl-3-phenyl-urea; [186]
1-[1-(3-cyanophenyl)ethyl]-1-cyclopentyl-3-(4-fluorophenyl)urea; [187]
3-(4-cyanophenyl)-1-[1-(3-cyanophenyl)ethyl]-1-cyclopentyl-urea; [188]
1-[1-(3-cyanophenyl)ethyl]-1-cyclobutyl-3-(4-fluorophenyl)urea; [189]
3-(4-cyanophenyl)-1-cyclopentyl-1-[1-(5-methyl-2-furyl)ethyl]urea; [190]
1-cyclopentyl-3-(4-fluorophenyl)-1-[1-(5-methyl-2-furyl)ethyl]urea; [191]
3-(4-chlorophenyl)-1-[1-(3-cyanopheny)ethy]-1-cyclobutyl-urea; [192]
3-(4-chlorophenyl)-1-[(5-methyl-2-furyl)methyl]-1-(3-methylimidazol-4-yl)urea; [193]
3-(4-chlorophenyl)-1-[(2-cyano-4-pyridyl)methyl]-1-(3-methylisoxazol-4-yl)urea[198]
3-(4-cyano-3-methoxy-phenyl)-1-cyclopentyl-1-[(5-methyl-2-furyl)methyl]urea[205];
3-(4-chlorophenyl)-1-(2,2-difluorocyclopentyl)-1-[(5-methyl-2-furyl)methyl] urea; [206]
3-(4-cyanophenyl)-1-cyclobutyl-1-(pyrazolo[1,5-a]pyridin-2-ylmethyl)urea; [207]
3-(4-cyanophenyl)-1-(3-fluorocyclobutyl)-1-[(5-methyl-2-furyl)methyl]urea; [208]
1-(1,3-benzoxazol-6-ylmethyl)-3-(4-cyanophenyl)-1-cyclobutyl-urea; [209]
3-(4-cyanophenyl)-1-cyclobutyl-1-(imidazo[1,2-a]pyridin-2-ylmethyl)urea; [210]
3-(4-cyanophenyl)-1-cyclobutyl-1-(imidazo[1,2-a]pyrazin-2-ylmethyl)urea; [211]
3-(4-chlorophenyl)-1-cyclobutyl-1-(imidazo[1,2-a]pyrazin-2-ylmethyl)urea; [212]
1-(1,3-benzothiazol-6-ylmethyl)-3-(4-cyanophenyl)-1-cyclobutyl-urea; [213]
3-(4-cyanophenyl)-1-(2,2-difluorocyclopentyl)-1-[(5-methyl-2-furyl)methyl] urea; [214]
3-(4-cyanophenyl)-1-[(5-methyl-2-furyl)methyl]-1-(2-methylpyrazol-3-yl)urea; [215]
3-(4-cyanophenyl)-1-(3-methoxycyclobutyl)-1-[(5-methyl-2-furyl)methyl]urea; [216]
3-(4-cyanophenyl)-1-cyclobutyl-1-[(1-methylindazol-6-yl)methyl]urea; [217]
3-(4-cyanophenyl)-1-[(5-methyl-2-furyl)methyl]-1-(1-methylpyrazol-4-yl)urea; [218]
1-(2-cyanocyclopentyl)-3-(4-cyanophenyl)-1-[(5-methyl-2-furyl)methyl]urea; [219]
3-(4-chlorophenyl)-1-[(5-methyl-2-furyl)methyl]-1-(2-methylpyrazol-3-yl)urea; [220]
3-(4-cyanophenyl)-1-[(5-methyl-2-furyl)methyl]-1-(1-methylpyrazol-3-yl)urea; [221]
3-(4-chlorophenyl)-1-(3-fluorocyclobutyl)-1-[(5-methyl-2-furyl)methyl]urea; [222]
1-(1,3-benzoxazol-5-ylmethyl)-3-(4-cyanophenyl)-1-cyclobutyl-urea; [223]
1-(3-cyanocyclopentyl)-3-(4-cyanophenyl)-1-[(5-methyl-2-furyl)methyl]urea; [224]
3-(4-chlorophenyl)-1-[(5-methyl-2-furyl)methyl]-1-(1-methylpyrazol-4-yl)urea; [225]
3-(4-chlorophenyl)-1-[(5-methyl-2-furyl)methyl]-1-(1-methylpyrazol-3-yl)urea; [226]
3-(4-chlorophenyl)-1-(2-cyanocyclopentyl)-1-[(5-methyl-2-furyl)methyl]urea; [227]
1-cyclopentyl-1-[(5-methyl-2-furyl)methyl]-3-[6-(trifluoromethyl)-3-pyridyl] urea; [228]
1-(1,3-benzothiazol-2-ylmethyl)-3-(4-cyanophenyl)-1-cyclobutyl-urea; [229]
3-(4-cyanophenyl)-1-(3,5-dimethylisoxazol-4-yl)-1-[(5-methyl-2-furyl)methyl] urea; [230]
3-(4-cyanophenyl)-1-[(5-methyl-2-furyl)methyl]-1-(3-methylimidazol-4-yl)urea; [231]
1-(1,3-benzoxazol-5-ylmethyl)-3-(4-chlorophenyl)-1-cyclobutyl-urea; [232]
3-(4-cyanophenyl)-1-[(5-methyl-2-furyl)methyl]-1-(1-methylimidazol-2-yl)urea; [233]
1-(benzofuran-2-ylmethyl)-3-(4-cyanophenyl)-1-cyclobutyl-urea; [234]
3-(4-chlorophenyl)-1-cyclobutyl-1-[(1-methylindazol-6-yl)methyl]urea; [235]
3-(4-ethynylphenyl)-1-[(5-methyl-2-furyl)methyl]-1-(oxetan-3-yl)urea; [236]
1-(1,3-benzoxazol-6-ylmethyl)-3-(4-chlorophenyl)-1-cyclobutyl-urea; [237]
3-(4-chlorophenyl)-1-(3,5-dimethylisoxazol-4-yl)-1-[(5-methyl-2-furyl)methyl] urea; [238]
3-(4-chlorophenyl)-1-cyclobutyl-1-(pyrazolo[1,5-a]pyridin-2-ylmethyl)urea; [239]
1-(2,1,3-benzothiadiazol-5-ylmethyl)-3-(4-chlorophenyl)-1-cyclobutyl-urea; [240]
3-(4-cyanophenyl)-1-(3,3-difluorocyclobutyl)-1-[(5-methyl-2-furyl)methyl]urea; [241]
3-(4-chlorophenyl)-1-[(5-methyl-2-furyl)methyl]-1-(3-methylimidazol-4-yl) urea; [242]
1-(1,3-benzothiazol-6-ylmethyl)-3-(4-chlorophenyl)-1-cyclobutyl-urea; [243]
3-(4-cyanophenyl)-1-(3,3-difluorocyclopentyl)-1-[(5-methyl-2-furyl)methyl] urea; [244]
3-(4-chlorophenyl)-1-(3,3-difluorocyclopentyl)-1-[(5-methyl-2-furyl)methyl] urea; [245]
1-cyclopentyl-1-[(5-methyl-2-furyl)methyl]-3-(6-methyl-3-pyridyl)urea; [246]
3-(4-chlorophenyl)-1-(3,3-difluorocyclobutyl)-1-[(5-methyl-2-furyl)methyl] urea; [247]
1-(1,3-benzothiazol-2-ylmethyl)-3-(4-chlorophenyl)-1-cyclobutyl-urea; [248]
3-(4-cyanophenyl)-1-cyclobutyl-1-(furo[3,2-b]pyridin-2-ylmethyl)urea; [249]
3-(4-chlorophenyl)-1-cyclobutyl-1-(imidazo[1,2-a]pyridin-2-ylmethyl)urea; [250]
3-(4-chlorophenyl)-1-(3-methoxycyclobutyl)-1-[(5-methyl-2-furyl)methyl]urea; [251]
3-(4-chlorophenyl)-1-cyclobutyl-1-(furo[3,2-b]pyridin-2-ylmethyl)urea; [252]
1-cyclopentyl-3-(6-methoxy-3-pyridyl)-1-[(5-methyl-2-furyl)methyl]urea; [253]
3-(4-chlorophenyl)-1-(3-cyanocyclopentyl)-1-[(5-methyl-2-furyl)methyl]urea; [254]
1-(2,1,3-benzothiadiazol-5-ylmethyl)-3-(4-cyanophenyl)-1-cyclobutyl-urea; [255]
3-(4-chlorophenyl)-1-[(5-methyl-2-furyl)methyl]-1-(5-methylisoxazol-4-yl)urea; [256]
3-(4-cyanophenyl)-1-[(5-methyl-2-furyl)methyl]-1-(5-methylisoxazol-4-yl)urea; [257]
3-(4-ethynylphenyl)-1-[(5-methyl-2-furyl)methyl]-1-(1-methyl-4-piperidyl)urea; [258]
3-(4-ethynylphenyl)-1-[(5-methyl-2-furyl)methyl]-1-tetrahydropyran-4-yl-urea; [259]
1-(1,3-benzothiazol-5-ylmethyl)-3-(4-chlorophenyl)-1-cyclobutyl-urea; [260]
1-(1,3-benzothiazol-5-ylmethyl)-3-(4-cyanophenyl)-1-cyclobutyl-urea; [261]
1-(1,3-benzothiazol-7-ylmethyl)-3-(4-chlorophenyl)-1-cyclobutyl-urea; [262]
1-(1,3-benzothiazol-7-ylmethyl)-3-(4-cyanophenyl)-1-cyclobutyl-urea; [263]
1-(1,3-benzothiazol-6-ylmethyl)-3-(4-cyanophenyl)-1-(1-methyl-4-piperidyl) urea; [264]
1-(1,3-benzothiazol-6-ylmethyl)-3-(4-cyanophenyl)-1-tetrahydropyran-4-yl-urea; [265]
3-(4-cyanophenyl)-1-[(5-methyl-2-furyl)methyl]-1-(2-methyl-1,2,4-triazol-3-yl) urea[266];
3-(4-chlorophenyl)-1-[(5-methyl-2-furyl)methyl]-1-(3-methyltriazol-4-yl)urea[267];
3-(4-cyanophenyl)-1-[(5-methyl-2-furyl)methyl]-1-(3-methyltriazol-4-yl)urea[268];
3-(4-chlorophenyl)-1-cyclobutyl-1-(imidazo[2,1-b]thiazol-6-ylmethyl)urea[269];
3-(4-cyanophenyl)-1-cyclobutyl-1-(imidazo[2,1-b]thiazol-6-ylmethyl)urea[270];
3-(4-chlorophenyl)-1-(3-fluorocyclobutyl)-1-[(5-methyl-2-furyl)methyl]urea[271];
3-(4-cyanophenyl)-1-(3-fluorocyclobutyl)-1-[(5-methyl-2-furyl)methyl]urea[272];
3-(4-chlorophenyl)-1-(3-fluorocyclobutyl)-1-[(5-methyl-2-furyl)methyl]urea[273];
3-(4-cyanophenyl)-1-cyclobutyl-1-(imidazo[1,2-a]pyrimidin-7-ylmethyl)urea[274];
3-(4-chlorophenyl)-1-cyclobutyl-1-(imidazo[1,2-a]pyrimidin-7-ylmethyl)urea[275];
1-(1,3-benzothiazol-6-ylmethyl)-3-(4-cyanophenyl)-1-(2-methyl-1H-pyrazol-3-yl) urea[276];
3-(4-chlorophenyl)-1-[(5-methyl-2-furyl)methyl]-1-(3-methylisoxazol-4-yl)urea[277];
3-(4-cyanophenyl)-1-[(5-methyl-2-furyl)methyl]-1-(3-methylisoxazol-4-yl)urea[278] and
3-[4-(3-methoxyprop-1-ynyl)phenyl]-1-[(5-methyl-2-furyl)methyl]-1-(5-methyl isoxazol-4-yl)urea[279];
1-(1,3-benzothiazol-6-ylmethyl)-3-[4-(3-hydroxyprop-1-ynyl)phenyl]-1-(2-methylpyrazol-3-yl)urea[280];
1-(1,3-benzothiazol-5-ylmethyl)-3-(4-cyanophenyl)-1-(5-methylisoxazol-4-yl)urea[281];
3-(4-chlorophenyl)-1-[(5-methyl-2-furyl)methyl]-1-(2-pyridyl)urea[282];
3-(4-cyanophenyl)-1-[(5-methyl-2-furyl)methyl]-1-(2-pyridyl)urea[283];
1-(1-bicyclo[1.1.1]pentanyl)-3-(4-chlorophenyl)-1-[(5-methyl-2-furyl)methyl]urea[284];
1-(1-bicyclo[1.1.1]pentanyl)-3-(4-cyanophenyl)-1-[(5-methyl-2-furyl)methyl]urea[285];
3-(4-chlorophenyl)-1-[(5-methyl-2-furyl)methyl]-1-(2-oxaspiro[3.3]heptan-6-yl)urea[286];
3-(4-cyanophenyl)-1-[(5-methyl-2-furyl)methyl]-1-(2-oxaspiro[3.3]heptan-6-yl)urea[287];
3-(4-cyanophenyl)-1-(5-methylisoxazol-4-yl)-1-[[2-(trifluoromethyl)-4-pyridyl]methyl]urea[288];
1-cyclopentyl-1-[(5-methyl-2-furyl)methyl]-3-[4-(trifluoromethoxy)phenyl]urea[289];
1-cyclopentyl-3-(4-methoxyphenyl)-1-[(5-methyl-2-furyl)methyl]urea[290];
3-(4-cyanophenyl)-1-cyclopentyl-1-[(2-methyloxazol-5-yl)methyl]urea[291];
1-[(5-methyl-2-furyl)methyl]-1-(5-methylisoxazol-4-yl)-3-[4-(trifluoromethoxy)phenyl]urea[292];
3-(4-cyanophenyl)-1-(5-methylisoxazol-4-yl)-1-[[3-(trifluoromethyl)phenyl]methyl]urea[293];
1-cyclopentyl-3-[4-(3-hydroxyprop-1-ynyl)phenyl]-1-[(2-methyloxazol-5-yl)methyl]urea[294];
3-(4-cyanophenyl)-1-cyclopentyl-1-[(5-ethyl-2-furyl)methyl]urea[295];
1-cyclobutyl-3-[4-(3-hydroxyprop-1-ynyl)phenyl]-1-[[2-(trifluoromethyl)-4-pyridyl]methyl]urea[296];
1-cyclobutyl-3-[4-(3-hydroxyprop-1-ynyl)phenyl]-1-[(5-methyl-2-furyl)methyl]urea[297];
3-(4-chlorophenyl)-1-[(5-methyl-2-furyl)methyl]-1-(4-methyl-1,2,5-oxadiazol-3-yl)urea[298];
3-(4-cyanophenyl)-1-[(5-ethyl-2-furyl)methyl]-1-(5-methylisoxazol-4-yl)urea[299];
1-(1,3-benzothiazol-2-yl)-3-(4-chlorophenyl)-1-[(5-methyl-2-furyl)methyl]urea[300];
3-[4-(difluoromethoxy)phenyl]-1-[(5-methyl-2-furyl)methyl]-1-(5-methylisoxazol-4-yl)urea[301];
3-(4-chlorophenyl)-1-(2-methylpyrazol-3-yl)-1-[[2-(trifluoromethyl)-4-pyridyl]methyl]urea[302];
1-cyclopentyl-3-(4-hydroxyphenyl)-1-[(5-methyl-2-furyl)methyl]urea[303];
3-(4-cyanophenyl)-1-isoxazol-4-yl-1-[(5-methyl-2-furyl)methyl]urea[304];
3-(4-chlorophenyl)-1-isoxazol-4-yl-1-[(5-methyl-2-furyl)methyl]urea[305];
1-cyclobutyl-3-[4-(3-hydroxyprop-1-ynyl)phenyl]-3-methyl-1-[(5-methyl-2-furyl)methyl]urea[306];
1-cyclopentyl-1-[(5-methyl-2-furyl)methyl]-3-(1-oxoisoindolin-5-yl)urea[307];
3-[4-[4-(hydroxymethyl)triazol-1-yl]phenyl]-1-(3-methylisoxazol-4-yl)-1-[[3-(trifluoromethyl)phenyl]methyl]urea[308];
3-[4-(3-hydroxyprop-1-ynyl)phenyl]-1-[(5-methyl-2-furyl)methyl]-1-(1-methylimidazol-2-yl)urea[309];
3-(4-cyanophenyl)-1-cyclopentyl-1-(6,7-dihydro-4H-pyrano[4,3-d]thiazol-2-ylmethyl)urea[310];
3-(4-chlorophenyl)-1-cyclopentyl-1-(6,7-dihydro-4H-pyrano[4,3-d]thiazol-2-ylmethyl)urea[311]1-cyclopentyl-1-[(5-methyl-2-furyl)methyl]-3-(2-methyl-1-oxo-isoindolin-5-yl)urea[312];
3-(4-chlorophenyl)-1-[(6-cyano-2-pyridyl)methyl]-1-(3-methylisoxazol-4-yl)urea[313];
3-(4-cyanophenyl)-1-[(5-methyl-2-furyl)methyl]-1-(3-methyl-2-pyridyl)urea[314];
3-[4-(3-hydroxyprop-1-ynyl)phenyl]-1-(3-methylisoxazol-4-yl)-1-[[3-(trifluoromethyl)phenyl]methyl]urea[315];
3-(4-cyanophenyl)-1-(3-methylisoxazol-4-yl)-1-[(2-methyloxazol-5-yl)methyl]urea[316];
1-cyclopentyl-1-[(5-methyl-2-furyl)methyl]-3-[4-(oxetan-3-yl)phenyl]urea[317];
3-(4-chlorophenyl)-1-[(5-methyl-2-furyl)methyl]-1-(3-methyl-2-pyridyl)urea[318];
1-[(6-cyano-2-pyridyl)methyl]-1-cyclobutyl-3-[4-(3-hydroxyprop-1-ynyl)phenyl]urea[319];
1-cyclopentyl-1-[(5-methyl-2-furyl)methyl]-3-[4-(4-methyltriazol-1-yl)phenyl]urea[320];
3-[4-(3-hydroxyprop-1-ynyl)phenyl]-1-(3-methylisoxazol-4-yl)-1-[(2-methyloxazol-5-yl)methyl]urea[321];
3-(4-cyanophenyl)-1-[(5-methyl-2-furyl)methyl]-1-(4-methyl-1,2,5-oxadiazol-3-yl)urea[322];
1-cyclopentyl-3-[4-[4-(hydroxymethyl)triazol-1-yl]-3-methoxy-phenyl]-1-[(5-methyl-2-furyl)methyl]urea[323];
3-(4-cyanophenyl)-1-[(5-methyl-2-furyl)methyl]-1-pyrimidin-2-yl-urea[324];
3-(4-cyanophenyl)-1-(3-methylisoxazol-4-yl)-1-(2-oxaspiro[3.5]nonan-7-ylmethyl)urea[325];
3-(4-chlorophenyl)-1-(3-methylisoxazol-4-yl)-1-(2-oxaspiro[3.5]nonan-7-ylmethyl)urea[326];
3-(4-chlorophenyl)-1-[(5-methyl-2-furyl)methyl]-1-pyrimidin-2-yl-urea[327]; and
3-(4-chlorophenyl)-1-[(5-methyl-2-furyl)methyl]-1-(3-methylisothiazol-4-yl)urea[328];
or a pharmaceutically acceptable salt, or a solvate, or a solvate of the salt thereof.

77. The compound according to claim 55 comprising compounds of formula I: 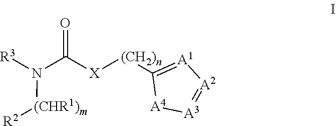
or a pharmaceutically acceptable salt, or a solvate, or a solvate of the salt thereof, wherein:
X, n and R¹ are each as herein defined;
m is 1;
R² is a 5-6-membered heteroaryl, a fused 9-10 membered bicyclic heteroaryl, phenyl; each of which may independently be optionally substituted by one or more groups independently selected from C₁₋₆alkyl, halogen, haloC₁₋₆alkyl, —CN and haloC₁₋₆alkyloxy;
R³ is a 4 or 5 membered cycloalkyl, a 5-6-membered heteroaryl or an isoxazole; each of which may independently be optionally substituted by one or more groups independently selected from —C₁₋₆alkyl, —OC₁₋₆alkyl, halogen and —CN;
the moiety 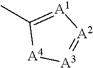
is phenyl or thiazole, each of which may independently be optionally substituted by one or more groups independently selected from —C₁₋₆alkyl, halogen, haloC₁₋₆alkyl, haloC₁₋₆alkyl-O—, —CN, —C₁₋₆alkyl-OH, —C₂₋₆alkynyl, C₂₋₆alkynyl-C₁₋₆alkyl, —C₂₋₆alkynyl-C₃₋₆cycloalkyl, —C₂₋₆alkynyl-C₁₋₆alkyl-OR¹³ and a 5-6 membered heteroaryl; and
R¹³ is H or C₁₋₆alkyl.

78. The compound according to claim 55 comprising compounds of formula I: 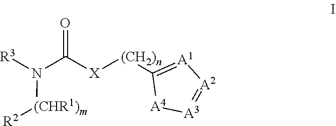
or a pharmaceutically acceptable salt, or a solvate, or a solvate of the salt thereof, wherein:
X and R¹ is as herein defined;
m is 1;
n is 0;
R² is a 5-6-membered heteroaryl, a fused 9-10 membered bicyclic heteroaryl, a 6 membered aryl, a 5-6 membered monocyclic heterocycloalkyl or a fused 8-10 membered partially unsaturated bicyclic heterocyclyl; each of which may independently be optionally substituted by one or more groups independently selected from C₁₋₆alkyl, haloC₁₋₆alkyl, halogen, —OC₁₋₆alkyl, —CN and —C(═O)OC₁₋₆alkyl;
R³ is a 4 or 5 membered cycloalkyl, a 5-6 membered heteroaryl or a 4-6 membered monocyclic heterocycloalkyl each of which may independently be optionally substituted by one or more groups independently selected from C₁₋₆alkyl, —OC₁₋₆alkyl, halogen, and —CN;
the moiety 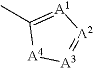
is phenyl, pyridine, pyrimidine or thiazole, each of which may independently be optionally substituted by one or more groups independently selected from —C₁₋₆alkyl, halogen, haloC₁₋₆alkyl, haloC₁₋₆alkyl-O—, —CN, —OC₁₋₆alkyl, —C₂₋₆alkynyl, —C₂₋₆alkynyl-C₁₋₆alkyl-NR¹¹R¹², —C₂₋₆alkynyl-C₁₋₆alkyl-OR¹³, a 6-10 membered aryl, a 5-6 membered heteroaryl, each of which may independently be optionally substituted by one or more groups independently selected from —C₁₋₆alkyl or —C₁₋₆alkyl-OH; and
R¹¹, R¹² and R¹³, which may be the same or different, are each selected from H and C₁₋₆alkyl.

79. The compound according to claim 55 comprising compounds of formula I: 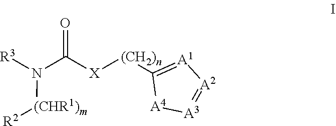
or a pharmaceutically acceptable salt, or a solvate, or a solvate of the salt thereof, wherein:
X is as herein defined;
m is 1;
n is 0;
R¹ is H or C₁₋₆alkyl;
R² is a thiophene, furan, pyrazine, pyridine, isoxazole, benzoxazole, imidazothiazole or phenyl;
each of which may independently be optionally substituted by one or more groups independently selected from C₁₋₆alkyl, halogen, haloC₁₋₆alkyl, —CN;
R³ is H, 4 or 5 membered cycloalkyl, imidazole, or oxetane; each of which may independently be optionally substituted by one or more groups independently selected from —C₁₋₆alkyl, —OC₁₋₆alkyl, halogen and —CN;
the moiety 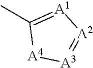
is phenyl, pyridine, benzothiazole, benzofuran, each of which may independently be optionally substituted by one or more groups independently selected from —C₁₋₆alkyl, halogen, —CN, —C₂₋₆alkynyl, —C₂₋₆alkynyl-aryl, —C₂₋₆alkynyl-C₁₋₆alkyl-aryl, —C₂₋₆alkynyl-C₁₋₆alkyl-NR¹¹R¹²; or a 5-6 membered heteroaryl, which may be optionally substituted by one or more groups independently selected from —C(═O)OC₁₋₆alkyl, thiophene, phenyl and —C₁₋₆alkyl-OH; and
R¹¹ and R¹², which may be the same or different, are each selected from H and C₁₋₆alkyl.

80. The compound according to claim 55 comprising compounds of formula I: 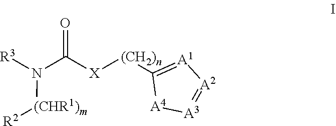
or a pharmaceutically acceptable salt, or a solvate, or a solvate of the salt thereof, wherein:
X is as herein defined;
m is 1;
n is 0 or 2;
R¹ is H or C₁₋₆alkyl;
R² is a 5 or 6 membered heteroaryl, a fused 9 or 10 membered bicyclic heteroaryl, a 6 membered aryl or a 5 or 6 membered monocyclic heterocycloalkyl or a fused 8-10 membered partially unsaturated bicyclic heterocyclyl; each of which may independently be optionally substituted by one or more groups independently selected from C₁₋₆alkyl, halogen, —OC₁₋₆alkyl, —CN, —C(═O)C₁₋₆alkyl —C(═O)OC₁₋₆alkyl, —SO₂—C₁₋₆alkyl, —C(═O)NH₂, haloC₁₋₆alkyloxy and phenyl;
R³ is H or C₁₋₆alkyl; or a 3-6 membered cycloalkyl, a 6 membered aryl, a 5-6 membered heteroaryl, a fused 9-10 membered bicyclic heteroaryl, a 4-6 membered monocyclic heterocycloalkyl or a 5-11 membered spiroheteroalkyl a 5-11 membered spiroheteroalkyl; each of which may independently be optionally substituted by one or more —C₁₋₆alkyl;
the moiety 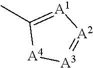
is phenyl, benzodioxole, indane, pyridine, thiophene or thiazole, each of which may independently be optionally substituted by one or more groups independently selected from —C₁₋₆alkyl, halogen, haloC₁₋₆alkyl, —CN, —OC₁₋₆alkyl, —C₁₋₆alkyl-CN, —C₂₋₆alkynyl-C₁₋₆alkyl-OR¹³, —C(═O)C₁₋₆alkyl, —C(═O)NH₂, —C(═O)OC₁₋₆alkyl and oxopyrrolidine a 5 or 6 membered cycloalkyl, a 4-6 membered monocyclic heterocycloalkyl, a 6 membered aryl, a 5 or 6 membered heteroaryl, a 5 or 6 membered heteroC₃₋₆cycloalkyl, each of which may independently be optionally substituted by one or more groups independently selected from —C₁₋₆alkyl, —C(═O)OC₁₋₆alkyl; and
R¹³ is each selected from H and C₁₋₆alkyl.

81. The compound according to claim 55 comprising compounds of formula I: 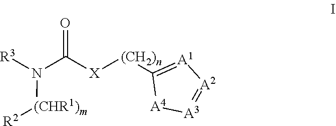
or a pharmaceutically acceptable salt, or a solvate, or a solvate of the salt thereof, wherein:
X and n are each as herein defined;
m is 1;
R¹ is H or C₁₋₆alkyl;
R² is a 5-6-membered heteroaryl, a fused 9-10 membered bicyclic heteroaryl, a 6 membered aryl or a 5-6 membered monocyclic heterocycloalkyl or a 5-11 membered spiroheteroalkyl; each of which may independently be optionally substituted by one or more groups independently selected from C₁₋₆alkyl, halogen, —CN;
R³ is H or a 5 or 6 membered cycloalkyl, a 5 membered heteroaryl, a 6 membered monocyclic heterocycloalkyl, a 5-11 membered spiroheteroalkyl or a —C₁₋₆alkyl-heteroaryl; each of which may independently be optionally substituted by one or more groups independently selected from —C₁₋₆alkyl, halogen and —C(═O)OC₁₋₆alkyl;
the moiety 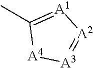
is phenyl, pyridine or phenyl fused with oxopyrrolidine, each of which may independently be optionally substituted by one or more groups independently selected from —OH, —C₁₋₆alkyl, halogen, —CN, —OC₁₋₆alkyl, —C₂₋₆alkynyl, —C(═O)C₁₋₆alkyl, a 5-6 membered heteroaryl, a 5-6 membered heteroC₃₋₆cycloalkyl, each of which may independently be optionally substituted by one or more groups independently selected from —C₁₋₆alkyl-NR⁹R¹⁰ and —C₁₋₆alkyl-OH;
R⁹ and R¹⁰, which may be the same or different, are each selected from H and C₁₋₆alkyl.

82-116. (canceled)

117. A method of treatment of a disease or condition associated with, abnormal or elevated catabolism of tryptophan, reduced levels of tryptophan, or elevated levels of kynurenine, which comprises the administration of a therapeutically effective amount of a compound of Formula (I) to a patient suffering from such a disease or condition: 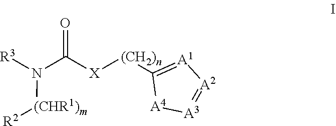
or a pharmaceutically acceptable salt, or a solvate, or a solvate of the salt thereof, wherein:
m is 0 or 1;
n is 0, 1 or 2;
X is —NR⁸;
R¹ is H, C₁₋₆alkyl or a 6-10 membered aryl;
R² is a 5-6-membered heteroaryl, a fused 9-10 membered bicyclic heteroaryl, a 6-10 membered aryl or a 5-6 membered monocyclic heterocycloalkyl, a 5-11 membered spiroheteroalkyl or a fused 8-10 membered partially unsaturated bicyclic heterocyclyl; each of which may independently be optionally substituted by one or more groups independently selected from C₁₋₆alkyl, halogen, haloC₁₋₆alkyl, —OC₁₋₆alkyl, —CN, —C(═O)C₁₋₆alkyl, —C(═O)OC₁₋₆alkyl, —SO₂—C₁₋₆alkyl, —C(═O)NH₂, haloC₁₋₆alkyloxy or phenyl;
R³ is H or C₁₋₆alkyl; or a 3-10 membered cycloalkyl, a 5-11 membered spiroalkyl, a 6-10 membered aryl, a 5-6 membered heteroaryl, a fused 9-10 membered bicyclic heteroaryl, a 4-6 membered monocyclic heterocycloalkyl, a —C₁₋₆alkyl-heteroaryl or a 5-11 membered spiroheteroalkyl; each of which may independently be optionally substituted by one or more groups independently selected from —C₁₋₆alkyl, —OC₁₋₆alkyl, halogen, —CN or —C(═O)OC₁₋₆alkyl;
A¹ is —N— or —CR⁶—;
A² is —N— or —CR—;
A³ is —N— or —CR⁷—;
A⁴ is —N—, —O—, —S—, —CH═N— or —CH═CR⁴—;
R⁴, R⁵, R⁶ and R⁷, which may be the same or different, are each selected from —H, —OH, —C₁₋₆alkyl, halogen, haloC₁₋₆alkyl, —CN, —C₁₋₆alkyl-CN, —OC₁₋₆alkyl, —C₂₋₆alkynyl, —C₂₋₆alkynyl-C₁₋₆alkyl, —C₂₋₆alkynyl-aryl, —C₂₋₆alkynyl-C₁₋₆alkyl-aryl, —C₂₋₆alkynyl-C₃₋₆cycloalkyl, —C₂₋₆alkynyl-C₁₋₆alkyl-NR¹¹R¹², —C₂₋₆alkynyl-C₁₋₆alkyl-OR¹³, —C(═O)C₁₋₆alkyl, —C(═O)NH₂, a 3-10 membered cycloalkyl, a 5-11 membered spiroalkyl, a 4-6 membered monocyclic heterocycloalkyl, a 6-10 membered aryl, a 5-6 membered heteroaryl, a 5-6 membered heteroC₃₋₆cycloalkyl, a fused 9-10 membered bicyclic heteroaryl, each of which may independently be optionally substituted by one or more groups independently selected from —C₁₋₆alkyl, C₁₋₆alkyl-NR⁹R¹⁰, —C₁₋₆alkyl-OH, —C(═O)OC₁₋₆alkyl or oxopyrrolidine;
or R⁵ and R⁷ together form a ring —CH═CH—CH═CH—, —OCH₂O— or —CH₂CH₂CH₂—;
or the moiety 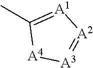
may be fused with oxopyrrolidine; and
R⁸, R⁹, R¹⁰, R¹¹, R¹², and R¹³, which may be the same or different, are each selected from H or C₁₋₆alkyl.

118-135. (canceled)

136. The method according to claim 117, for the treatment of diseases and/or conditions associated with the abnormal or elevated catabolism of tryptophan.

137. The method according to claim 136, wherein the disease or condition associated with the abnormal or elevated catabolism of tryptophan is one or more of cancer, immunosuppression, viral infection, depression, a neurodegenerative disorder, trauma, age-related cataracts, organ transplant rejection, or an autoimmune disorder in a patient.

138-171. (canceled)
